(19) United States
(12) Patent Application Publication
(10) Pub. No.: US 2006/0247320 A1
(43) Pub. Date: Nov. 2, 2006

(54) Compounds for inhibiting KSP kinesin activity

(75) Inventors: Jayaram R. Tagat, Westfield, NJ (US); Timothy J. Guzi, Chatham, NJ (US); Marc Labroli, Mount Laurel, NJ (US); Cory Poker, Springfield, NJ (US); Yushi Xiao, Edison, NJ (US); Angela D. Kerekes, Scotch Plains, NJ (US); Tao Yu, Edision, NJ (US); Sunil Paliwal, Monroe Township, NJ (US); Hon-Chung Tsui, East Brunswick, NJ (US); Neng-Yang Shih, Warren, NJ (US); Stuart W. McCombie, Caldwell, NJ (US); Vincent S. Madison, Mountain Lakes, NJ (US); Charles A. Lesburg, Short Hills, NJ (US); Jose S. Duca, Cranford, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/369,643

(22) Filed: Mar. 7, 2006

Related U.S. Application Data
(60) Provisional application No. 60/659,888, filed on Mar. 9, 2005.
(60) Provisional application No. 60/712,274, filed on Aug. 29, 2005.

(51) Int. Cl. A61K 31/12

(52) U.S. Cl. 514/683

(57) ABSTRACT

The present invention provides compounds of Formula I (wherein R¹, R³, X, W, Z and ring Y are as defined herein). The present invention also provides compositions comprising these compounds that are useful for treating cellular proliferative diseases or disorders associated with KSP kinesin activity and for inhibiting KSP kinesin activity.

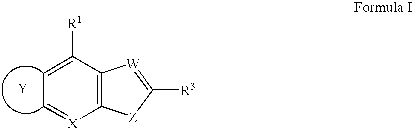

This Application claims the benefit of U. S. Provisional Application Ser. No. 60/659,888 filed Mar. 9, 2005, and U.S. Provisional Application Ser. No. 60/712,274 filed Aug. 29, 2005, both of which are incorporated herein by reference in their entirety.

### FIELD OF THE INVENTION

The present invention relates to compounds and compositions that are useful for treating cellular proliferative diseases or disorders associated with Kinesin Spindle Protein (“KSP”) kinesin activity and for inhibiting KSP kinesin activity.

### BACKGROUND OF THE INVENTION

Cancer is a leading cause of death in the United States and throughout the world. Cancer cells are often characterized by constitutive proliferative signals, defects in cell cycle checkpoints, as well as defects in apoptotic pathways. There is a great need for the development of new chemotherapeutic drugs that can block cell proliferation and enhance apoptosis of tumor cells.

Conventional therapeutic agents used to treat cancer include taxanes and vinca alkaloids, which target microtubules. Microtubules are an integral structural element of the mitotic spindle, which is responsible for the distribution of the duplicated sister chromatids to each of the daughter cells that result from cell division. Disruption of microtubules or interference with microtubule dynamics can inhibit cell division and induce apoptosis.

However, microtubules are also important structural elements in non-proliferative cells. For example, they are required for organelle and vesicle transport within the cell or along axons. Since microtubule-targeted drugs do not discriminate between these different structures, they can have undesirable side effects that limit usefulness and dosage. There is a need for chemotherapeutic agents with improved specificity to avoid side effects and improve efficacy.

Microtubules rely on two classes of motor proteins, the kinesins and dyneins, for their function. Kinesins are motor proteins that generate motion along microtubules. They are characterized by a conserved motor domain, which is approximately 320 amino acids in length. The motor domain binds and hydrolyses ATP as an energy source to drive directional movement of cellular cargo along microtubules and also contains the microtubule binding interface (Mandelkow and Mandelkow, Trends Cell Biol. 2002, 12:585-591).

Kinesins exhibit a high degree of functional diversity, and several kinesins are specifically required during mitosis and cell division. Different mitotic kinesins are involved in all aspects of mitosis, including the formation of a bipolar spindle, spindle dynamics, and chromosome movement. Thus, interference with the function of mitotic kinesins can disrupt normal mitosis and block cell division. Specifically, the mitotic kinesin KSP (also termed EG5), which is required for centrosome separation, was shown to have an essential function during mitosis. Cells in which KSP function is inhibited arrest in mitosis with unseparated centrosomes (Blangy et al., Cell 1995, 83:1159-1169). This leads to the formation of a monoastral array of microtubules, at the end of which the duplicated chromatids are attached in a rosette-like configuration. Further, this mitotic arrest leads to growth inhibition of tumor cells (Kaiser et al., J. Biol. Chem. 1999, 274:18925-18931). Inhibitors of KSP would be desirable for the I treatment of proliferative diseases, such as cancer.

Kinesin inhibitors are known, and several molecules have recently been described in the literature. For example, adociasulfate-2 inhibits the microtubule-stimulated ATPase activity of several kinesins, including CENP-E (Sakowicz et al., Science 1998, 280:292-295). Rose Bengal lactone, another non-selective inhibitor, interferes with kinesin function by blocking the microtubule binding site (Hopkins et al., Biochemistry 2000, 39:2805-2814). Monastrol, a compound that has been isolated using a phenotypic screen, is a selective inhibitor of the KSP motor domain (Mayer et al., Science 1999, 286:971-974). Treatment of cells with monastrol arrests cells in mitosis with monopolar spindles.

KSP, as well as other mitotic kinesins, are attractive targets for the discovery of novel chemotherapeutics with anti-proliferative activity. There is a need for compounds useful in the inhibition of KSP, and in the treatment of proliferative diseases, such as cancer.

### SUMMARY OF THE INVENTION

In one embodiment, the present invention provides a compound represented by the structural Formula l: 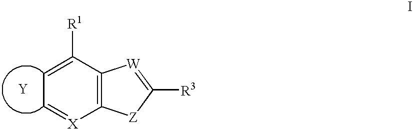
or a pharmaceutically acceptable salt, solvate or ester thereof, wherein:

ring Y is a 5- to 7-membered ring selected from the group consisting of cycloalkyl, cycloalkenyl, heterocyclyl or heterocyclenyl fused as shown in Formula I, wherein in each of said 5- to 7-membered ring, each substitutable ring carbon is independently substituted with 1-2 R² moieties and each substitutable ring heteroatom is independently substituted with R⁶;

W is N or C(R¹²);

X is N or N-oxide;

Z is S, S(═O) or S(═O)₂;

R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;

each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;

or two R²s on the same carbon atom are optionally taken together with the carbon atom to which they are attached to form a C═O, a C═S or an ethylenedioxy group;

R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)N R⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(═NR⁷)NR⁴R⁵, —C(O)N(R⁷)(CR⁴⁰R⁴¹)₁₋₅—NR⁴R⁵, —C(O)N(R⁷)(CR⁴⁰R⁴¹)₁₋₅—C(O)—NR⁴R⁵, —C(O)N(R⁷)(CR⁴⁰R⁴¹)₁₋₅—OR⁷, —C(S)NR⁷(CH₂)₁₋₅NR⁴R⁵, and —C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;

each of R⁴ and R⁵is independently selected from the group consisting of H, alkyl, cycloalkyl, cycloalkenyl, heterocyclyl, heterocyclenyl, aryl, heteroaryl, —OR⁷, —C(O)R⁷, and —C(O)OR⁷, wherein each of said alkyl, cycloalkyl, cycloalkenyl, heterocyclyl, heterocyclenyl, aryl, and heteroaryl, is optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S;

each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;

each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R¹ except H is optionally substituted with 1-4 R⁸ moieties;

each R⁸ is independently selected from the group consisting of halo, alkyl, cycloalkyl, cycloalkenyl, heterocyclyl, heterocyclenyl, aryl, heteroaryl, —NO₂, —OR¹⁰, —(C₁-C₆ alkyl)—OR¹⁰, —CN, —NR¹⁰R¹¹, —C(O)R¹⁰, —C(O)OR¹⁰, —C(O)NR¹⁰OR¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring;

or two R⁸ groups, when attached to the same carbon, are optionally taken together with the carbon atom to which they are attached to form a C═O or a C═S group;

each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;

each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;

each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; wherein each of said R¹¹ alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;

each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)N R⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;

R⁴⁰ and R⁴¹ can be the same or different, each being independently selected from the group consisting of H, alkyl, aryl, heteroaryl, heterocyclyl, heterocyclenyl, cycloalkyl and cycloalkenyl;

each R⁴² is independently selected from the group consisting of halo, alkyl, cycloalkyl, heterocyclyl, aryl, heteroaryl, —NO₂, —OR¹⁰, —(C₁-C₆ alkyl)—OR¹⁰, —CN, —NR¹⁰R¹¹, —C(O)R¹⁰, —C(O)OR¹⁰, —C(O)NR¹⁰R¹¹, —CF₃, —OCF₃, —N(R¹⁰)C(O)R¹¹, and —NR¹⁰C(O)OR¹¹;

with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹, —C(O)OR¹¹, —C(O)N(R¹⁰)(R¹¹), or —N(R¹⁰)C(O)R¹¹;

with the further proviso that the compound of Formula (I) is other than any of the following: 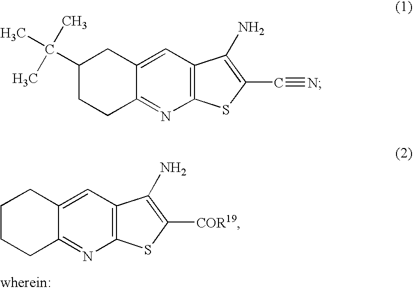
R¹⁹ is —NHOH, —OMe, —OEt, —O-n-propyl, or —O-i-propyl; 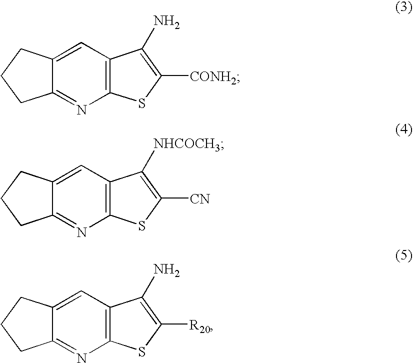
wherein:

R²⁰ is —CN, —C(O)C₆H₅, —CO₂C₂H₅, —CO₂H, or —C(O)NH₂;

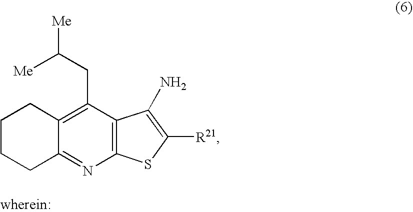

R²¹ is 4-ClC₆H₄C(O)— or 4-PhC₆H₄C(O)—;

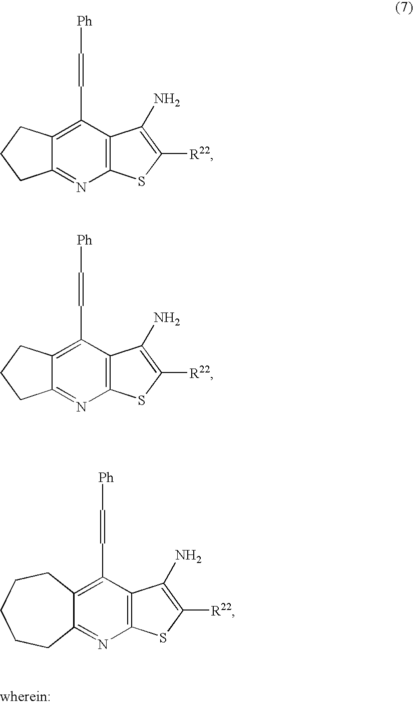

R²² is —CN, —C(O)CH₃ or —CO₂C₂H₅;

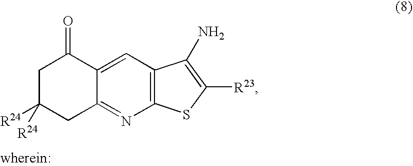

R²³ is —C(O)NH₂, —C(O)NHPh, or benzoyl and R²⁴ is H or methyl;

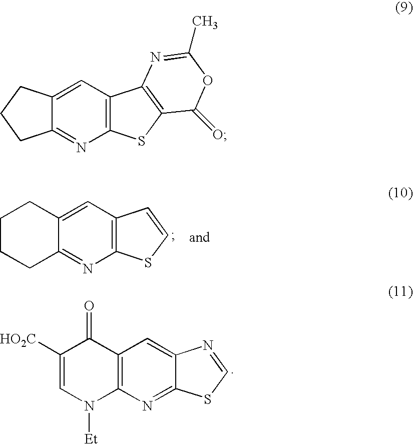

In another embodiment, the present invention provides a compound represented by the structural Formula I, or a pharmaceutically acceptable salt, solvate, or ester thereof, wherein in Formula I:

ring Y is a 5- to 7-membered ring selected from the group consisting of cycloalkyl, cycloalkenyl, heterocyclyl or heterocyclenyl fused as shown in Formula I, wherein in each of said 5- to 7-membered ring, each substitutable ring carbon is independently substituted with 1-2 R² moieties and each substitutable ring heteroatom is independently substituted with R⁶;

W is N or C(R¹²);

X is N or N-oxide;

Z is S, S(═O) or S(═O)₂;

R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;

each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(=NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;

or two R²s on the same carbon atom are optionally taken together with the carbon atom to which they are attached to form a C═O, a C═S or an ethylenedioxy group;

R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(═NR⁷)NR⁴R⁵, —C(O)N(R⁷)(CR⁴⁰R⁴¹)₁₋₅NR⁴R⁵, —C(O)N(R⁷)(CR⁴⁰R⁴¹)₁₋₅C(O)—NR⁴R , —C(O)N(R⁷)(CR⁴⁰R⁴¹)₁₋₅—OR⁷, —C(S)NR⁷(CH₂)₁₋₅NR⁴R⁵, and —C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;

each of R⁴ and R⁵ is independently selected from the group consisting of H, alkyl, cycloalkyl, cycloalkenyl, heterocyclyl, heterocyclenyl, aryl, heteroaryl, —OR⁷, —C(O)R⁷, and —C(O)OR⁷₁ wherein each of said alkyl, cycloalkyl, cycloalkenyl, heterocyclyl, heterocyclenyl, aryl, and heteroaryl, is optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S;

each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;

each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;

each R⁸ is independently selected from the group consisting of halo, alkyl, cycloalkyl, cycloalkenyl, heterocyclyl, heterocyclenyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring;

or two R⁸ groups, when attached to the same carbon, are optionally taken together with the carbon atom to which they are attached to form a C═O or a C═S group;

each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;

each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;

each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; and

each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;

R⁴⁰ and R⁴¹ can be the same or different, each being independently selected from the group consisting of H, alkyl, aryl, heteroaryl, heterocyclyl, heterocyclenyl, cycloalkyl and cycloalkenyl;

each R⁴² is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —N(R¹⁰)C(O)R¹¹, and —NR¹⁰C(O)OR¹¹, wherein

with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹, —C(O)OR¹¹, —C(O)N(R¹⁰)(R¹¹), or —N(R¹⁰)C(O)R¹¹;

with the further proviso that the compound of Formula (I) is other than any of the following: 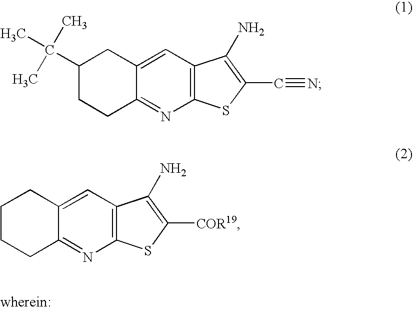
R¹⁹ is —NHOH, —OMe, —OEt, —O-n-propyl, or —O-i-propyl; 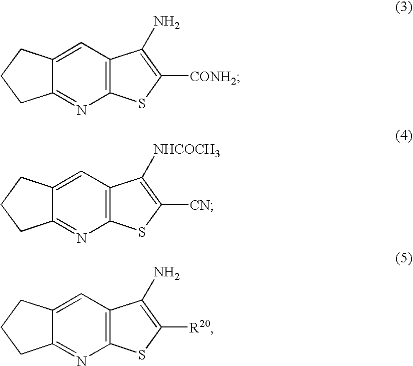
wherein:

R²⁰ is —CN, —C(O)C₆H₅, —CO₂C₂H₅, —CO₂H, or —C(O)NH₂;

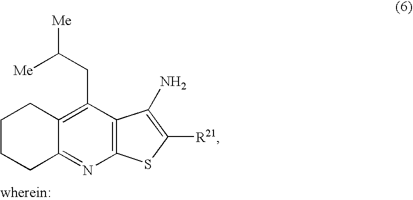

R²¹ is 4-ClC₆H₄C(O)— or 4-PhC₆H₄C(O)—;

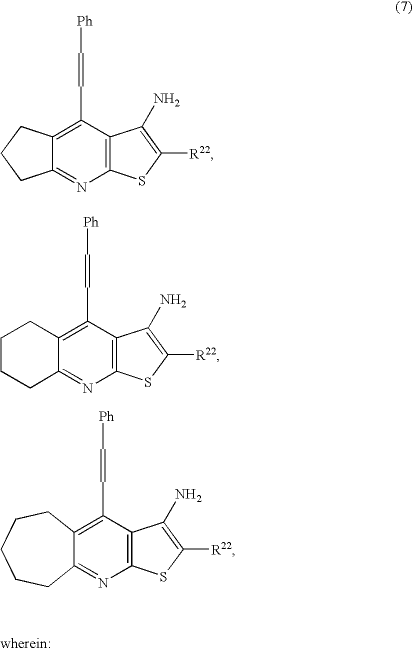

R²² is —CN, —C(O)CH₃ or —CO₂C₂H₅;

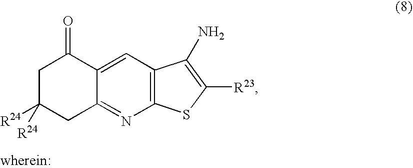

R²³ is —C(O)NH₂, —C(O)NHPh, or benzoyl and R²⁴ is H or methyl;

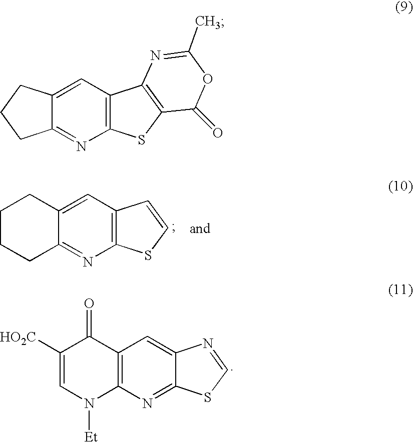

Pharmaceutical formulations or compositions for the treatment of cellular proliferative diseases, disorders associated with KSP kinesin activity and/or for inhibiting KSP kinesin activity in a subject comprising administering a therapeutically effective amount of at least one of the inventive compounds and a pharmaceutically acceptable carrier to the subject also are provided.

Methods of treating cellular proliferative diseases, disorders associated with KSP kinesin activity and/or for inhibiting KSP kinesin activity in a subject comprising administering to a subject in need of such treatment an effective amount of at least one of the inventive compounds also are provided.

Other than in the operating examples, or where otherwise indicated, all numbers expressing quantities of ingredients, reaction conditions, and so forth used in the specification and claims are to be understood as being modified in all instances by the term “about.”

### DETAILED DESCRIPTION

In one embodiment, the present invention discloses compounds represented by structural Formula I or a pharmaceutically acceptable salt or ester thereof, wherein the various moieties are as described above.

In one embodiment, the present invention discloses compounds represented by Formula II: 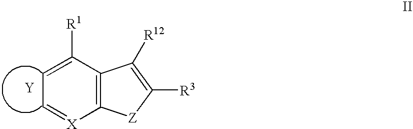
wherein ring Y, X, Z, R¹, R³ and R¹² are as set forth in formula I above.

In one embodiment, the present invention discloses compounds represented by Formula III: 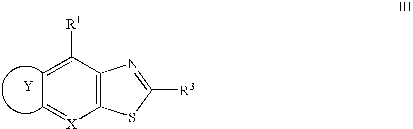
wherein ring Y, X, R¹, and R³ are as set forth in formula I above.

In another embodiment, in formula I, II, or III, X is N.

In another embodiment, in formula I, II, or III, X is N-oxide.

In another embodiment, in formula I or II, Z is S.

In another embodiment, in formula I or II, Z is S(═O).

In another embodiment, in formula I or II, Z is S(═O)₂.

In another embodiment, ring Y in formula I, II, or III is a 5- to 7-membered cycloalkyl, wherein each substitutable ring carbon is independently substituted with 1-2 R² moieties.

In another embodiment, ring Y in formula I, II, or III is a 5- to 7-membered cycloalkenyl, wherein each substitutable ring carbon is independently substituted with 1-2 R² moieties.

In another embodiment, ring Y in formula I, II, or III is a 6-membered cycloalkyl ring, wherein each substitutable ring carbon is independently substituted with 1-2 R² moieties.

In another embodiment, ring Y in formula I, II or III is a 6-membered cycloalkenyl, wherein each substitutable ring carbon is independently substituted with 1-2 R² moieties.

In another embodiment, in formula I, II, or III, ring Y is a 5- to 7-membered heterocyclyl, wherein in said ring Y, each substitutable ring carbon is independently substituted with 1-2 R² moieties and each substitutable ring heteroatom, when nitrogen, is independently substituted with R⁶.

In another embodiment, in formula I, II, or III, ring Y is a 5- to 7-membered heterocyclenyl, wherein in said ring Y, each substitutable ring carbon is independently substituted with 1-2 R² moieties and each substitutable ring heteroatom, when nitrogen, is independently substituted with R⁶.

In another embodiment, in formula I, II, or III, ring Y is a 5- to 7-membered heterocyclenyl, wherein in said ring Y, at least one heteroatom is S, and each substitutable ring carbon is independently substituted with 1-2 R² moieties.

In another embodiment, in formula I, II, or III, R² is H, alkyl, aryl, aralkyl, cycloalkyl, cycloalkylalkyl, —CF₃, alkylsilyl, alkoxy or —NR⁴R⁵; or two R²s attached to the same ring carbon are taken together with the carbon to form a C═O, a C═S or an ethylenedioxy group.

In another embodiment, in formula I, II or III, R⁶ is selected from the group consisting of H, alkyl, cycloalkyl, cycloalkylalkyl, aryl, aralkyl, heteroaryl, —(CH₂)₁₋₆CF₃, and —C(O)OR⁷ wherein R⁷ is alkyl.

In another embodiment, in formula I, II or III, R⁶ is selected from the group consisting of H, alkyl, cycloalkylalkyl, aralkyl, —(CH₂)₁₋₆CF₃, and —C(O)OR⁷ wherein R⁷ is alkyl.

In another embodiment, in formula I or II, R¹² is H, halo, —NR⁴R⁵ or —OR⁷.

In another embodiment, in formula I, II, or III, R³ is H, alkyl, cycloalkyl, cycloalkenyl, heterocyclyl, heterocyclenyl, heteroaryl, —C(O)OR⁷, —C(O)NR⁴R⁵, —C(S)NR⁴R⁵, —C(O)NR⁴OR⁷, —NR⁴R⁵, —NR⁴C(O)R⁵, —NR⁴C(O)NR⁴R⁵, —(CR¹⁰R¹¹)₀₋₆SR⁷, S(O₂)R⁷, —S(O₂)NR⁴R⁵, —CN, or —C(═NR⁷)NR⁴R⁵ wherein said alkyl, heterocyclyl or heteroaryl is optionally substituted with 1-3 R⁹ moieties.

In another embodiment, in formula I, II or III, R¹ is H, halo, —S-alkyl, alkoxy or hydroxy.

In another embodiment, in formula I, II or III, R¹ is H, Cl, OH or —SCH₃.

In another embodiment, in formula II:

Y is a 5- to 7-membered cycloalkyl ring, wherein each substitutable ring carbon atom is independently substituted with 1-2 R² moieties;

X is N; and

Z is S.

In another embodiment, in formula II:

Y is a 5- to 7-membered cycloalkyl ring, wherein each substitutable ring carbon atom is independently substituted with 1-2 R² moieties;

X is N; and

Z is S;

R¹ is selected from the group consisting H, hydroxy, halo, and

—S(O)ₘ-alkyl, wherein m is 0;

each R² independently is selected from the group consisting of H, alkyl, alkenyl, aryl, alkylsilyl, cycloalkyl, and —CF₃; wherein said alkyl or alkenyl is either unsubstituted or optionally substituted with aryl or cycloalkyl;

or two R²s on the same carbon atom are optionally taken together with the carbon atom to which they are attached to form a C═O, a C═S or an ethylenedioxy group;

R³ is selected from the group consisting of H, alkyl, cycloalkyl, cycloalkenyl, heterocyclyl, heterocyclenyl, heteroaryl, —C(O)OR⁷, —C(O)NR⁴R⁵, —C(S)NR⁴R⁵, —C(O)NR⁴OR⁷, —NR⁴R⁵, —NR⁴C(O)R⁵, —NR⁴C(O)NR⁴R⁵, —(CR¹⁰R¹¹)₀₋₆SR⁷, S(O₂)R⁷, —S(O₂)NR⁴R⁵, —CN, or —C(═NR⁷)NR⁴R⁵ wherein said alkyl, cycloalkyl, cycloalkenyl, heterocyclyl, heterocyclenyl, or heteroaryl is optionally substituted with 1-3 R9 moieties; and

R¹² is H, halo, —NR⁴R⁵, or —OR⁷.

In another embodiment, the present invention discloses compounds represented by Formula II-a:

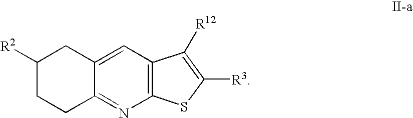

In another embodiment, in formula II-a, R³is —CN.

In another embodiment, in formula 11-a:

R³ is —C(O)NR⁴R⁵ wherein:

each of R⁴ and R⁵ is independently selcted from the group consisting of H, alkyl, cycloalkyl, aryl, heterocyclyl, and heteroaryl; wherein each of said alkyl, cycloalkyl, aryl, heterocyclyl and heteroaryl is unsubstituted or optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S.

In another embodiment, in formula II-a:

R³ is —C(O)NR⁴R⁵ wherein:

each of R⁴ and R⁵ is independently selcted from the group consisting of H, alkyl, cycloalkyl, aryl, heterocyclyl, and heteroaryl; wherein each of said alkyl, cycloalkyl, aryl, heterocyclyl and heteroaryl is unsubstituted or optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S;

each of said R⁴ and R⁵ alkyl is unsubstituted or optionally substituted with 1-3 R⁸ moieties independently selected from the group consisting of —OR¹⁰, —C(O)NR¹⁰R¹¹, —C(O)OR¹⁰, —NR¹⁰R¹¹, —CN, —C(═NR¹⁰)NR¹⁰R¹¹, heterocyclyl, and aryl; wherein each of said R⁸ heterocyclyl and aryl moieties is unsubstituted or optionally substituted with 1-3 R⁴² moieties selected from the group consisting of halo, alkyl, aryl, heteroaryl, —NO₂, —CN, —NR¹⁰R¹¹, —OR¹⁰, —N(R¹⁰)C(O)R¹¹, —N(R¹⁰)C(O)OR¹¹, —C(O)NR¹⁰R¹¹, and —C(O)OR¹⁰; wherein when each of said R⁴² aryl and heteroaryl contains two radicals on-adjacent carbon atoms anywhere within said aryl or heteroaryl, such radicals may optionally and independently in each occurrence, be taken together with the carbon atoms to which they are attached, to form a five to six membered carbocyclic or heterocyclic ring;

each of said R⁴ and R⁵ cycloalkyl is unsubstituted or is optionally substituted with 1-3 R⁸ moieties independently selected from the group consisting of halo, hydroxy, and alkyl;

each of said R⁴ and R⁵ heterocyclyl is unsubstituted or is optionally substituted with 1-3 R⁸ moieties independently selected from the group consisting of halo, hydroxy, —C(O)OH, and —C(O)O-alkyl;

each of said R⁴ and R⁵ aryl is unsubstituted or optionally substituted with 1-3 R⁸ moieties independently selected from the group consisting of —OR¹⁰, —NR¹⁰R¹¹, halo, and alkyl;

each of said R⁴ and R⁵ heteroaryl is unsubstituted or is optionally substituted with 1-3 R⁸ moieties independently selected from the group consisting of —OR¹⁰, —NR¹⁰R¹¹, halo, and alkyl;

said 3-6 membered heterocyclic ring formed by R⁴, R⁵, and the nitrogen atom to which R⁴ and R⁵ are attached, is unsubstituted or is optionally substituted with 1-3 substitutents selected from the group consisting of hydroxy,halo, alkyl —C(O)OH, and —C(O)O-alkyl.

In another embodiment, in formula II-a:

R³ is —C(O)NR⁴R⁵ wherein:

each of R⁴ and R⁵ is independently selcted from the group consisting of H and alkyl; wherein said alkyl is optionally substituted with 1-4 R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities;

each R¹⁰ is independently H or alkyl;

each R¹¹ is independently H, alkyl, heterocyclyl, aryl, or heteroaryl; wherein each of said R¹¹ alkyl, aryl, heterocyclyl and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is independently selected from the group consisting of halo, alkyl, heterocyclyl, aryl, heteroaryl, —NO₂, —NR¹⁰R¹¹, —OR¹⁰, —CN, —C(O)NR¹⁰R¹¹, —CF₃, —OCF₃, —N(R¹⁰)C(O)R¹¹, and —NR¹⁰C(O)OR¹¹.

In another embodiment, in formula II-a, R³ is —C(O)NR⁴R⁵ wherein:

each of R⁴ and R⁵ is independently selcted from the group consisting of H and alkyl; wherein said alkyl is optionally substituted with 1-4 R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, heterocyclyl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and each R⁴² is independently selected from the group consisting of halo, alkyl, heterocyclyl, aryl, heteroaryl, —NO₂, —NR¹⁰R¹¹, —OR¹⁰, —CN, —C(O)NR¹⁰R¹¹, —CF₃, —OCF₃, —N(R¹⁰)C(O)R¹¹, and —NR¹⁰C(O)OR¹¹.

In another embodiment, in formula II-a, R³ is —C(O)NR⁴R⁵ wherein:

each of R⁴ and R⁵ is independently selcted from the group consisting of H and alkyl; wherein said alkyl is optionally substituted with 1-4 R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, heterocyclyl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is —N(R¹⁰)C(O)R¹¹, wherein R¹⁰ of said is is —N(R¹⁰)C(O)R¹¹ is H, and R¹¹ of said —N(R¹⁰)C(O)R¹¹ is selected from the group consisting of heterocyclyl and heteroaryl, each of which is optionally substituted.

In another embodiment, in formula II-a, R³ is —C(O)NR⁴R⁵ wherein:

each of R⁴ and R⁵ is independently selcted from the group consisting of H and alkyl; wherein said alkyl is optionally substituted with 1-4 R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, heterocyclyl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is —N(R¹⁰)C(O)R¹¹, wherein R¹⁰ of said is is —N(R¹⁰)C(O)R¹¹ is H, and R¹¹ of said —N(R¹⁰)C(O)R¹¹ is selected from the group consisting of heterocyclyl and heteroaryl, each of which is optionally substituted; wherein said R¹¹ heterocyclyl of said —N(R¹⁰)C(O)R¹¹ is selected from the group consisting of pyrrolidinyl, piperidinyl, piperizinyl, and morpholinyl, each of which is optionally substituted.

In another embodiment, in formula II-a, R³ is —C(O)NR⁴R⁵ wherein:

each of R⁴ and R⁵ is independently selcted from the group consisting of H and alkyl; wherein said alkyl is optionally substituted with 1-4 R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, heterocyclyl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is —N(R¹⁰)C(O)R¹¹, wherein R¹⁰ of said is is —N(R¹⁰)C(O)R¹¹ is H, and R¹¹ of said —N(R¹⁰)C(O)R¹¹ is selected from the group consisting of heterocyclyl and heteroaryl, each of which is optionally substituted; wherein said R¹¹ heteroaryl of said —N(R¹⁰)C(O)R¹¹ is selected from the group consisting of benzopyrazinyl, pyrazinyl, oxazolyl, isoxazolyl, thiazolyl, isothizolyl, pyrazolyl, imidazolyl, pyrrolyl, triazolyl, 1,2,3-triazolyl, thiadiazolyl, tetrazolyl, furanyl, thiophenyl, pyrrolyl, and pyrimidyl, each of which is optionally substituted.

In another embodiment, in formula II-a, R³ is alkyl, wherein said alkyl is unsubstituted or optionally substituted with 1-3 R⁹ moieties independently selected from the group consisting of —OH, —CN, halo, alkoxy, —OC(O)NR⁴R⁵, —C(O)NR⁴R⁵, —(CR¹⁰R¹¹)₀₋₄NR⁴R⁵, —NR⁴C(O)R⁵ and —NR⁴C(O)NR⁴R⁵.

In another embodiment, the compound of formula III is represented by formula III-a:

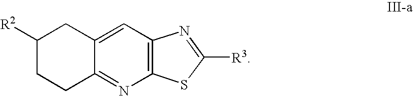

In another embodiment, in the compound of formula III-a:

R² is alkyl; and

R³ is selected from the group consisting of —(CR¹⁰R¹¹)₀₋₆SR⁷, —CN, —C(O)NR⁴R⁵, —NR⁴C(O)NR⁴R⁵, —NR⁴R⁵, and —NR⁴C(O)R⁵.

another embodiment, in the compound of formula III-a:

R³is —C(O)NR⁴R⁵ wherein:

each of R⁴ and R⁵ is independently selcted from the group consisting of H, alkyl, cycloalkyl, aryl, heterocyclyl, and heteroaryl; wherein each of said alkyl, cycloalkyl, aryl, heterocyclyl and heteroaryl is unsubstituted or optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S.

In another embodiment, in the compound of formula IIIa, R³ is —C(O)NR⁴R⁵ wherein:

each of R⁴ and R⁵ is independently selcted from the group consisting of H, alkyl, cycloalkyl, aryl, heterocyclyl, and heteroaryl; wherein each of said alkyl, cycloalkyl, aryl, heterocyclyl and heteroaryl is unsubstituted or optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S;

each of said R⁴ and R⁵ alkyl is unsubstituted or optionally substituted with 1-3 R⁸ moieties independently selected from the group consisting of —OR¹⁰, —C(O)NR¹⁰R¹¹, —C(O)OR¹⁰, —NR¹⁰R¹¹, —CN, —C(═NR¹⁰)NR¹⁰R¹¹, heterocyclyl, aryl, and heteroaryl; wherein each of said R⁸ heterocyclyl, aryl, and heteroaryl moieties is unsubstituted or optionally substituted with 1-3 R⁴² moieties selected from the group consisting of halo, alkyl, aryl, heteroaryl, —NO₂, —CN, —NR¹⁰R¹¹, —OR¹⁰, —N(R¹⁰)C(O)R¹¹, —N(R¹⁰)C(O)OR¹¹, —C(O)NR¹⁰R¹¹, and —C(O)OR¹⁰; wherein when each of said R⁴² aryl and heteroaryl contains two radicals on adjacent carbon atoms anywhere within said aryl or heteroaryl, such radicals may optionally and independently in each occurrence, be taken together with the carbon atoms to which they are attached, to form a five to six membered carbocyclic or heterocyclic ring;

each of said R⁴ and R⁵ cycloalkyl is unsubstituted or is optionally substituted with 1-3 R⁸ moieties independently selected from the group consisting of halo, hydroxy, and alkyl;

each of said R⁴ and R⁵ heterocyclyl is unsubstituted or is optionally substituted with 1-3 R⁸ moieties independently selected from the group consisting of halo, hydroxy, —C(O)OH, and —C(O)O-alkyl;

each of said R⁴ and R⁵ aryl is unsubstituted or optionally substituted with 1-3 R⁸ moieties independently selected from the group consisting of —OR¹⁰, —NR¹⁰R¹¹, halo, and alkyl;

each of said R⁴ and R⁵ heteroaryl is unsubstituted or is optionally substituted with 1-3 R⁸ moieties independently selected from the group consisting of —OR¹⁰, —NR¹⁰R¹¹, halo, and alkyl;

said 3-6 membered heterocyclic ring formed by R⁴, R⁵, and the nitrogen atom to which R⁴ and R⁵ are attached, is unsubstituted or is optionally substituted with 1-3 substitutents selected from the group consisting of hydroxy, halo, alkyl —C(O)OH, and —C(O)O-alkyl.

In another embodiment, in the compound of formula IIIa, R³ is —C(O)NR⁴R⁵ wherein:

each of R⁴ and R⁵ is independently selcted from the group consisting of H and alkyl; wherein said alkyl is optionally substituted with 1-4R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4R⁴² moities;

each R¹⁰ is independently H or alkyl;

each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is independently selected from the group consisting of halo, alkyl, heterocyclyl, aryl, heteroaryl, —NO₂, —NR¹⁰R¹¹, —OR¹⁰, —CN, —C(O)NR¹⁰R¹¹, —CF₃, —OCF₃, —N(R¹⁰)C(O)R¹¹, and —NR¹⁰C(O)OR¹¹.

In another embodiment, in the compound of formula IIIa, R³ is —C(O)NR⁴R⁵ wherein:

each of R⁴ and R⁵ is independently selcted from the group consisting of H and alkyl; wherein said alkyl is optionally substituted with 1-4R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is independently selected from the group consisting of halo, alkyl, heterocyclyl, aryl, heteroaryl, —NO₂, —NR¹⁰R¹¹, —OR¹⁰, —CN, —C(O)NR¹⁰R¹¹, —CF₃, —OCF₃, —N(R¹⁰)C(O)R¹¹, and —NR¹⁰C(O)O^{R}¹¹.

In another embodiment, in the compound of formula IIIa, R³ is —C(O)NR⁴R⁵ wherein:

each of R⁴ and R⁵ is independently selcted from the group consisting of H and alkyl; wherein said alkyl is optionally substituted with 1-4R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

R⁴² is —N(R¹⁰)C(O)R¹¹, wherein R¹⁰ in said —N(R¹⁰)C(O)R¹¹ is H and R¹¹ in said —N(R¹⁰)C(O)R¹¹ is selected from the group consisting of heterocyclyl and heteroaryl, each of which is optionally substituted.

In another embodiment, in the compound of formula IIIa, R³ is —C(O)NR⁴R⁵ wherein:

each of R⁴ and R⁵ is independently selcted from the group consisting of H and alkyl; wherein said alkyl is optionally substituted with 1-4R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

R⁴² is —N(R¹⁰)C(O)R¹¹, wherein R¹⁰ in said —N(R¹⁰)C(O)R¹¹ is H and R¹¹ in said —N(R¹⁰)C(O)R¹¹ is selected from the group consisting of heterocyclyl and heteroaryl, each of which is optionally substituted; wherein said R¹¹ heterocyclyl in said —N(R¹⁰)C(O)R¹¹ is selected from the group consisting of pyrrolidinyl, piperidinyl, piperizinyl, and morpholinyl, each of which is optionally substituted. In another embodiment, in the compound of formula IIIa, R³ is —C(O)NR⁴R⁵ wherein:

each of R⁴ and R⁵ is independently selcted from the group consisting of H and alkyl; wherein said alkyl is optionally substituted with 1-4 R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

R⁴² is —N(R¹⁰)C(O)R¹¹, wherein R¹⁰ in said —N(R¹⁰)C(O)R¹¹ is H and R¹¹ in said —N(R¹⁰)C(O)R¹¹ is selected from the group consisting of heterocyclyl and heteroaryl, each of which is optionally substituted; wherein said R¹¹ heteroaryl in said —N(R¹⁰)C(O)R¹¹ is selected from the group consisting of benzopyrazinyl, pyrazinyl, oxazolyl, isoxazolyl, thiazolyl, isothizolyl, pyrazolyl, imidazolyl, pyrrolyl, triazolyl, 1,2,3-triazolyl, thiadiazolyl, tetrazolyl, furanyl, thiophenyl, pyrrolyl, and pyrimidyl, each of which is optionally substituted.

In another embodiment, in the compound of formula II or III:

ring Y is a 5- to 7-membered heterocyclyl, wherein in said ring Y, each substitutable ring carbon is independently substituted with 1-2 R² moieties and each substitutable ring heteroatom, when nitrogen, is independently substituted with R⁶; and wherein said ring Y is representd by formula IV:

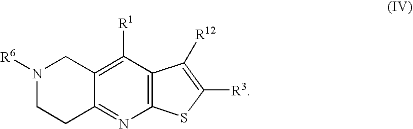

In another embodiment, in the compound of formula IV:

R¹ is H;

R³ is —CN;

R⁶ is selected from the group consisting of H, alkyl, cycloalkylalkyl, aralkyl, —(CH₂)₁₋₆CF₃, and —C(O)OR⁷ wherein R⁷ is alkyl; and

R¹² is —NR⁴R⁵, wherein both R⁴ and R⁵ are H.

In another embodiment, in formula (IV), R³ is —C(O)NR⁴R⁵ wherein:

each of R⁴ and R⁵ is independently selcted from the group consisting of H, alkyl, cycloalkyl, aryl, heterocyclyl, and heteroaryl; wherein each of said alkyl, cycloalkyl, aryl, heterocyclyl and heteroaryl is unsubstituted or optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S.

In another embodiment, in formula (IV), R³ is —C(O)NR⁴R⁵ wherein:

each of R⁴ and R⁵ is independently selcted from the group consisting of H, alkyl, cycloalkyl, aryl, heterocyclyl, and heteroaryl; wherein each of said alkyl, cycloalkyl, aryl, heterocyclyl and heteroaryl is unsubstituted or optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S;

each of said R⁴ and R⁵ alkyl is unsubstituted or optionally substituted with 1-3 R⁸ moieties independently selected from the group consisting of —OR¹⁰, —C(O)NR¹⁰R¹¹, —C(O)OR¹⁰, —NR¹⁰R¹¹, —CN, —C(═NR¹⁰)NR¹⁰R¹¹, heterocyclyl, aryl, and heteroaryl; wherein each of said R⁸ heterocyclyl, aryl, and heteroaryl moieties is unsubstituted or optionally substituted with 1-3 R⁴² moieties selected from the group consisting of halo, alkyl, aryl, heteroaryl, —NO₂, —CN, —NR¹⁰R¹¹, —OR¹⁰, —N(R¹⁰)C(O)R¹¹, —N(R¹⁰)C(O)OR¹¹, —C(O)NR¹⁰R¹¹, and —C(O)OR¹⁰; wherein when each of said R⁴² aryl and heteroaryl contains two radicals on adjacent carbon atoms anywhere within said aryl or heteroaryl, such radicals may optionally and independently in each occurrence, be taken together with the carbon atoms to which they are attached, to form a five to six membered carbocyclic or heterocyclic ring;

each of said R⁴ and R⁵ cycloalkyl is unsubstituted or is optionally substituted with 1-3 R⁸ moieties independently selected from the group consisting of halo, hydroxy, and alkyl;

each of said R⁴ and R⁵ heterocyclyl is unsubstituted or is optionally substituted with 1-3 R⁸ moieties independently selected from the group consisting of halo, hydroxy, —C(O)OH, and —C(O)O-alkyl;

each of said R⁴ and R⁵ aryl is unsubstituted or optionally substituted with 1-3 R⁸ moieties independently selected from the group consisting of —OR¹⁰, —NR¹⁰R¹¹, halo, and alkyl;

each of said R⁴ and R⁵ heteroaryl is unsubstituted or is optionally substituted with 1-3 R⁸ moieties independently selected from the group consisting of —OR¹⁰, —NR¹⁰R¹¹, halo, and alkyl;

said 3-6 membered heterocyclic ring formed by R⁴, R⁵, and the nitrogen atom to which R⁴ and R⁵ are attached, is unsubstituted or is optionally substituted with 1-3 substitutents selected from the group consisting of hydroxy,halo, alkyl —C(O)OH, and —C(O)O-alkyl.

In another embodiment, in formula (IV), R³ is —C(O)NR⁴R⁵ wherein:

each of R⁴ and R⁵ is independently selcted from the group consisting of H and alkyl; wherein said alkyl is optionally substituted with 1-4R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities;

each R¹⁰ is independently H or alkyl;

each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is independently selected from the group consisting of halo, alkyl, heterocyclyl, aryl, heteroaryl, —NO₂, —NR¹⁰R¹¹, —OR¹⁰, —CN, —C(O)NR¹⁰R¹¹, —CF₃, —OCF₃, —N(R¹⁰)C(O)R¹¹, and —NR¹⁰C(O)OR¹¹.

In another embodiment, in formula (IV), R³ is —C(O)NR⁴R⁵ wherein:

each of R⁴ and R⁵ is independently selcted from the group consisting of H and alkyl; wherein said alkyl is optionally substituted with 1-4 R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is independently selected from the group consisting of halo, alkyl, heterocyclyl, aryl, heteroaryl, —NO₂, —NR¹⁰R¹¹, —OR¹⁰, —CN, —C(O)NR¹⁰R¹¹, —CF₃, —OCF₃, —N(R¹⁰)C(O)R¹¹, and —NR¹¹C(O)OR¹¹.

In another embodiment, in formula (IV), R³ is —C(O)NR⁴R⁵ wherein:

each of R⁴ and R⁵ is independently selcted from the group consisting of H and alkyl; wherein said alkyl is optionally substituted with 1-4 R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is —N(R¹⁰)C(O)R¹¹, wherein R¹⁰ in said —N(R¹⁰)C(O)R¹¹ is H, and R¹¹ in said —N(R¹⁰)C(O)R¹¹ is selected from the group consisting of heterocyclyl and heteroaryl, each of which is optionally substituted.

In another embodiment, in formula (IV), R³is —C(O)NR⁴R⁵ wherein:

each of R⁴ and R⁵ is independently selcted from the group consisting of H and alkyl; wherein said alkyl is optionally substituted with 1-4R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is —N(R¹⁰)C(O)R¹¹, wherein R¹⁰ in said —N(R¹⁰)C(O)R¹¹ is H, and R¹¹ in said —N(R¹⁰)C(O)R¹¹ is selected from the group consisting of heterocyclyl and heteroaryl, each of which is optionally substituted; wherein said R¹¹ heterocyclyl in said —N(R¹⁰)C(O)R¹¹ is selected from the group consisting of pyrrolidinyl, piperidinyl, piperizinyl, and morpholinyl, each of which is optionally substituted.

In another embodiment, in formula (IV), R³ is —C(O)NR⁴R⁵ wherein:

each of R⁴ and R⁵ is independently selcted from the group consisting of H and alkyl; wherein said alkyl is optionally substituted with 1-4 R⁸ moieties;

R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities; wherein said R⁸ aryl is phenyl, and said R⁸ heteroaryl is selected from the group consisting of pyridyl and thiophenyl;

each R¹⁰ is independently H or alkyl;

each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and

each R⁴² is —N(R¹⁰)C(O)R¹¹, wherein R¹⁰ in said -N(R¹⁰)C(O)R¹¹ is H, and R¹¹ in said —N(R¹⁰)C(O)R¹¹ is selected from the group consisting of heterocyclyl and heteroaryl, each of which is optionally substituted; wherein said R¹¹ heteroaryl in said —N(R¹⁰)C(O)R¹¹ is selected from the group consisting of benzopyrazinyl, pyrazinyl, oxazolyl, isoxazolyl, thiazolyl, isothizolyl, pyrazolyl, imidazolyl, pyrrolyl, triazolyl, 1,2,3-triazolyl, thiadiazolyl, tetrazolyl, furanyl, thiophenyl, pyrrolyl, and pyrimidyl, each of which is optionally substituted.

Representative compounds of the present invention include those selected from the group consisting of: 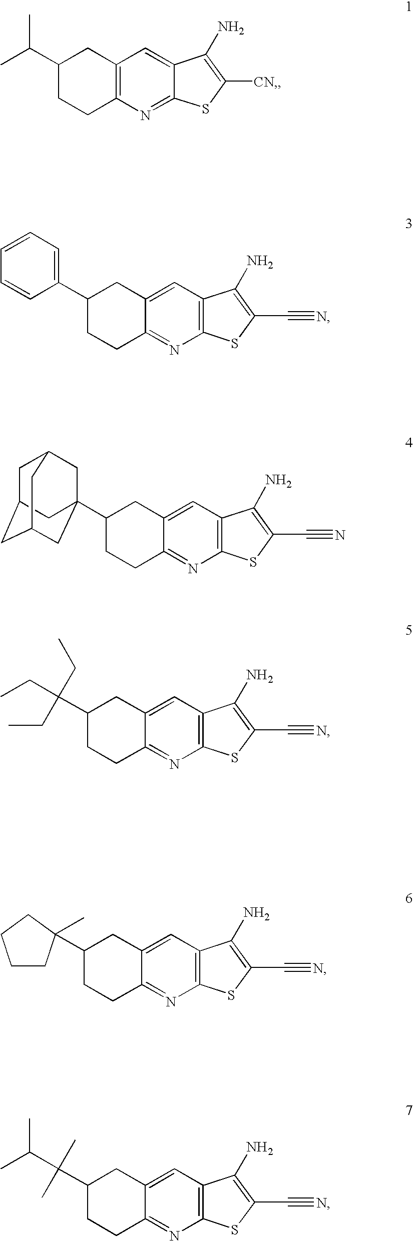 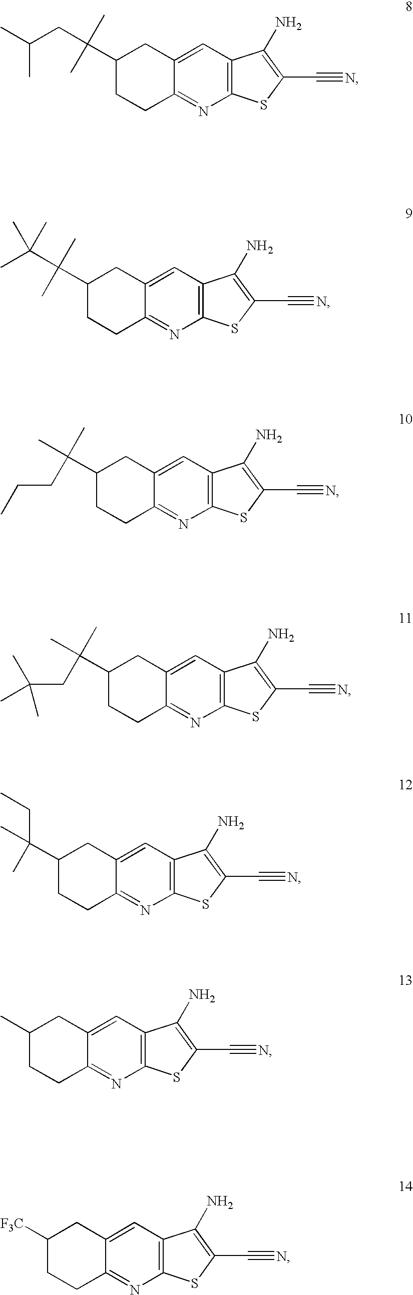 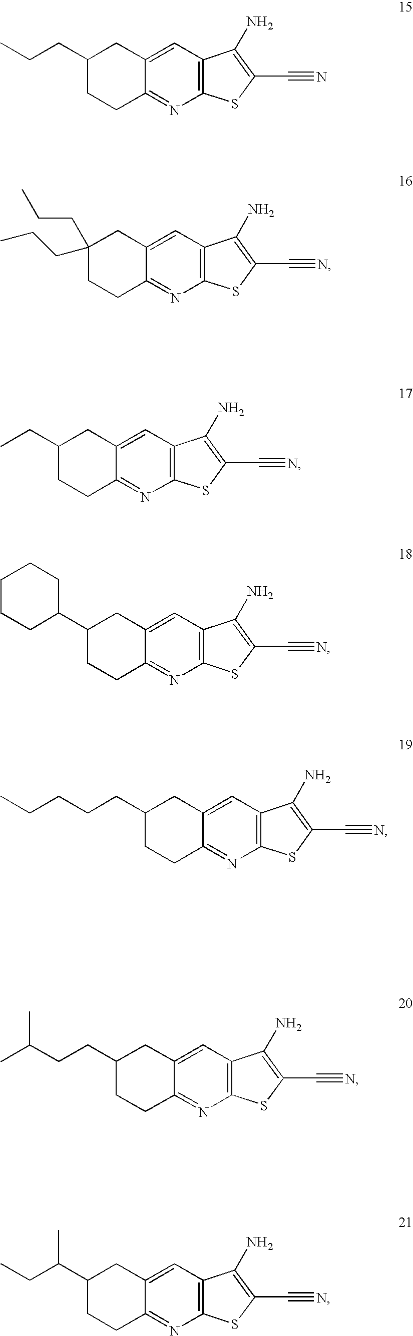 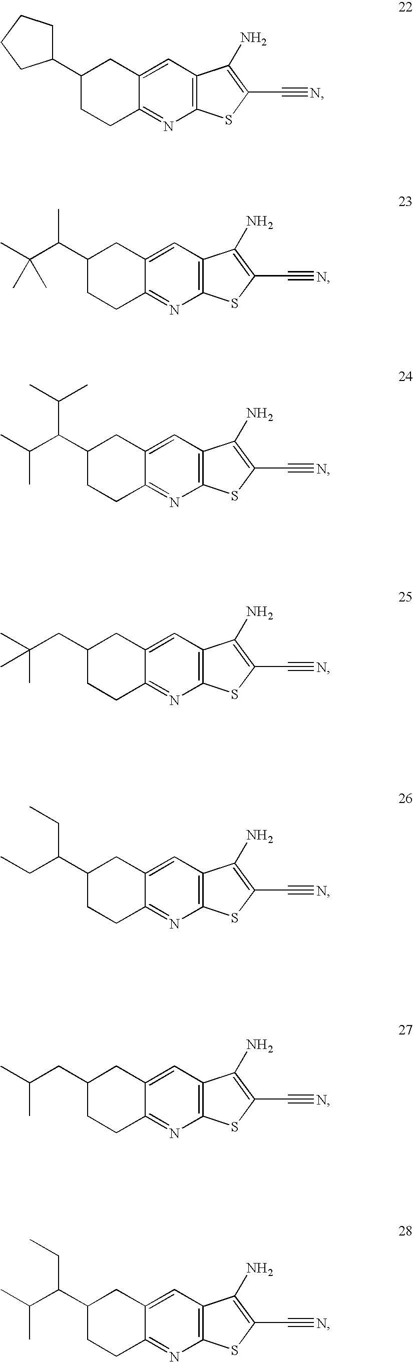 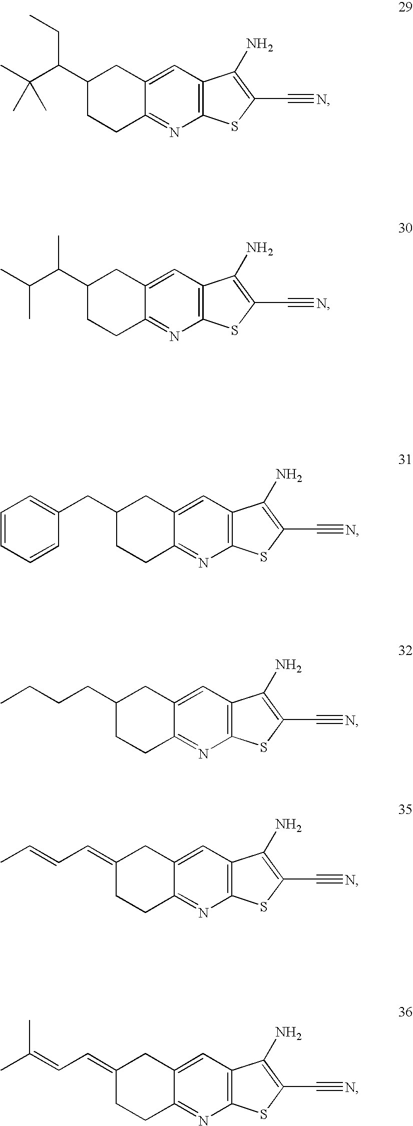 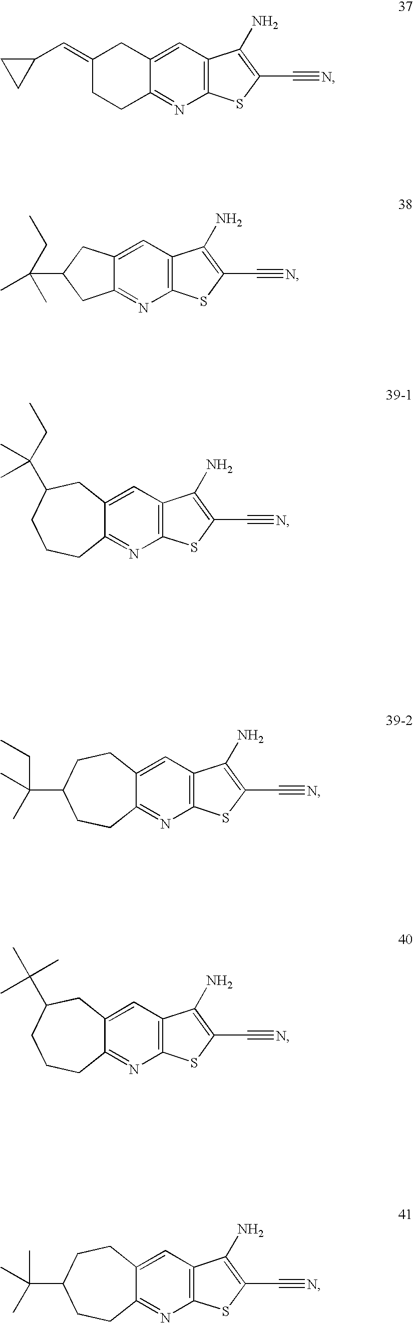 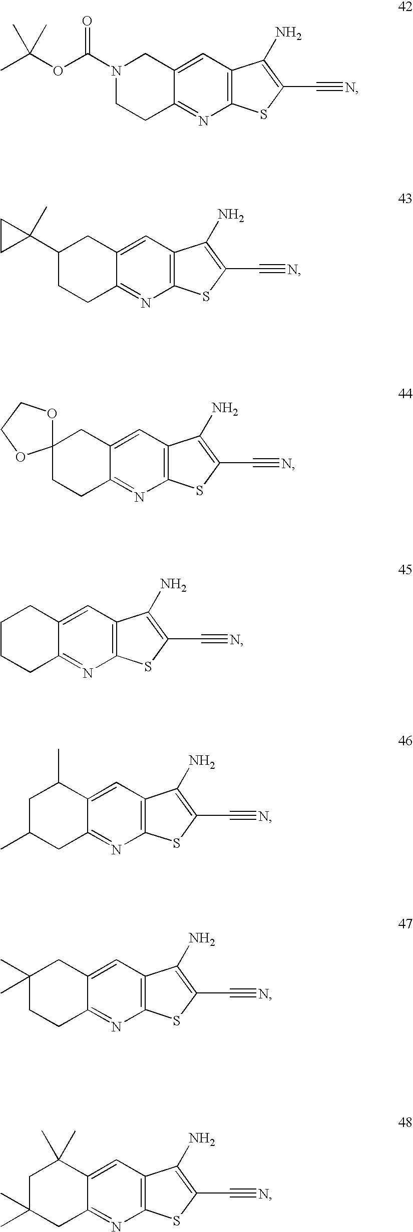 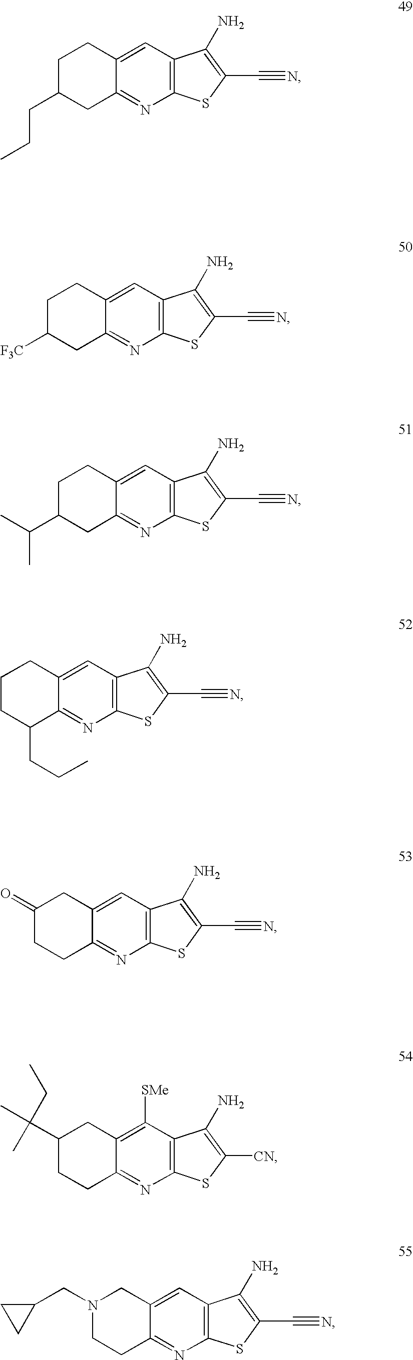 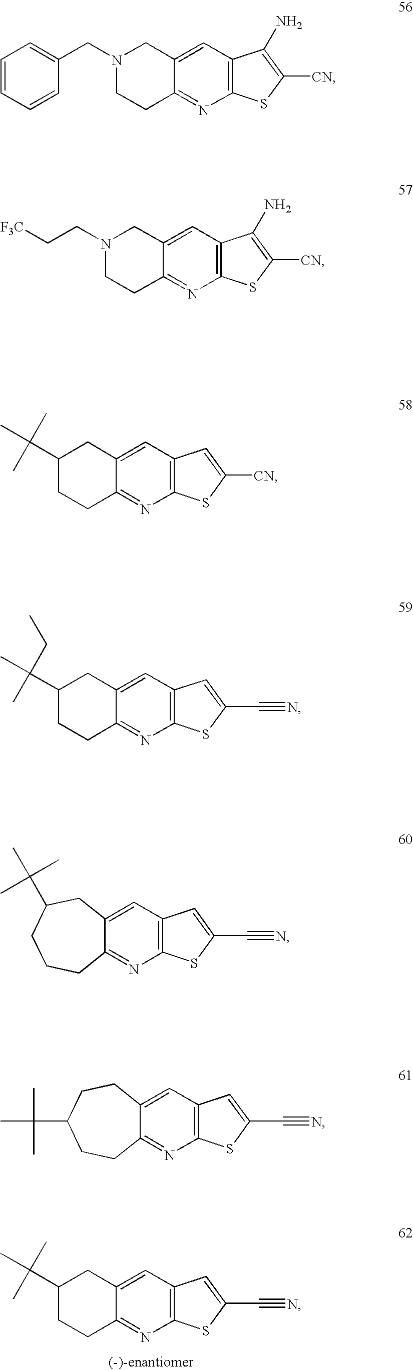 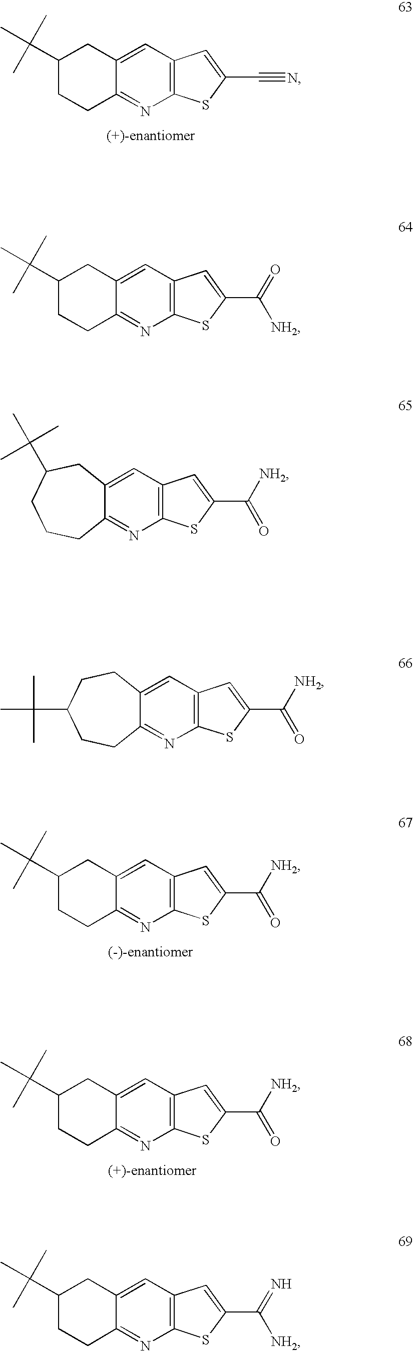 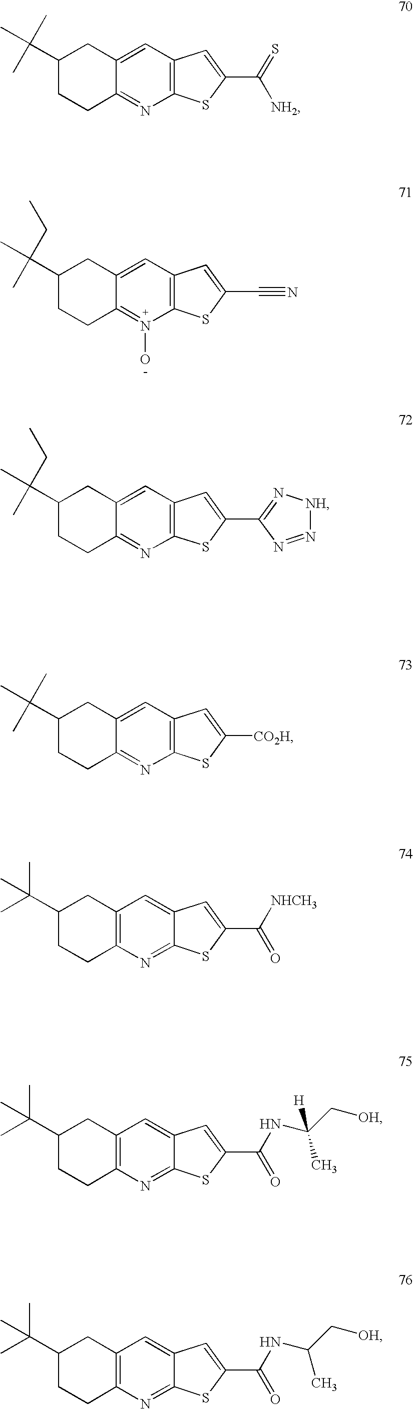 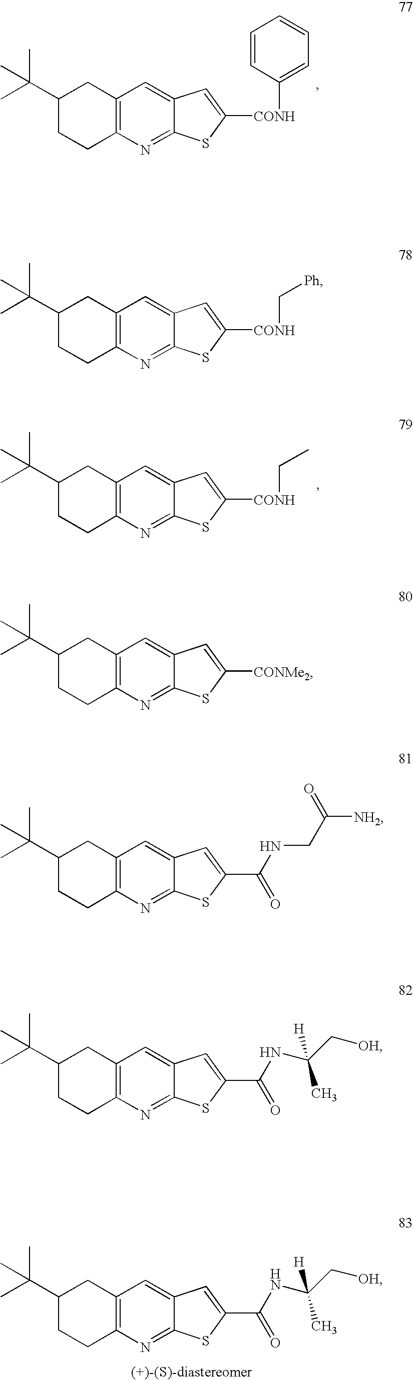 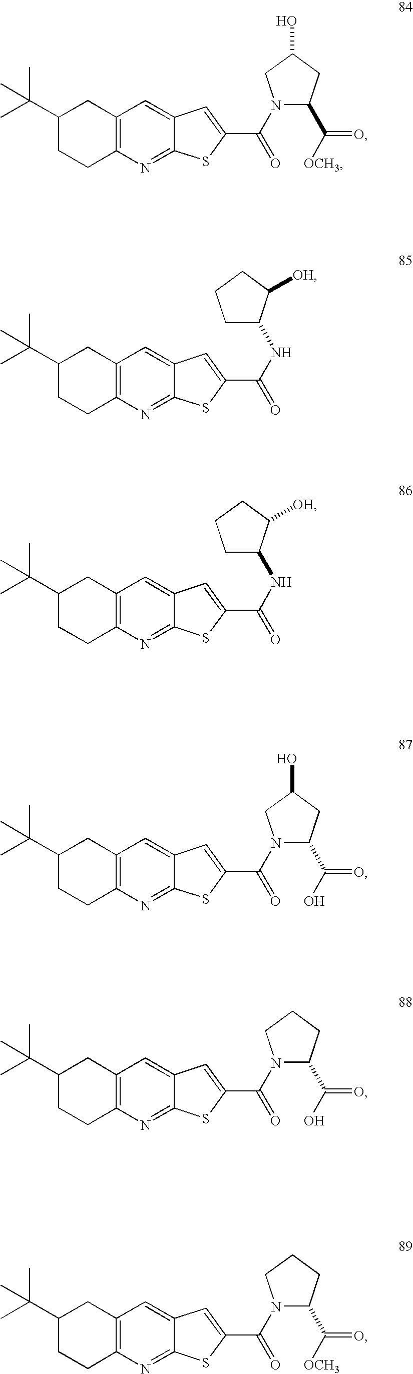 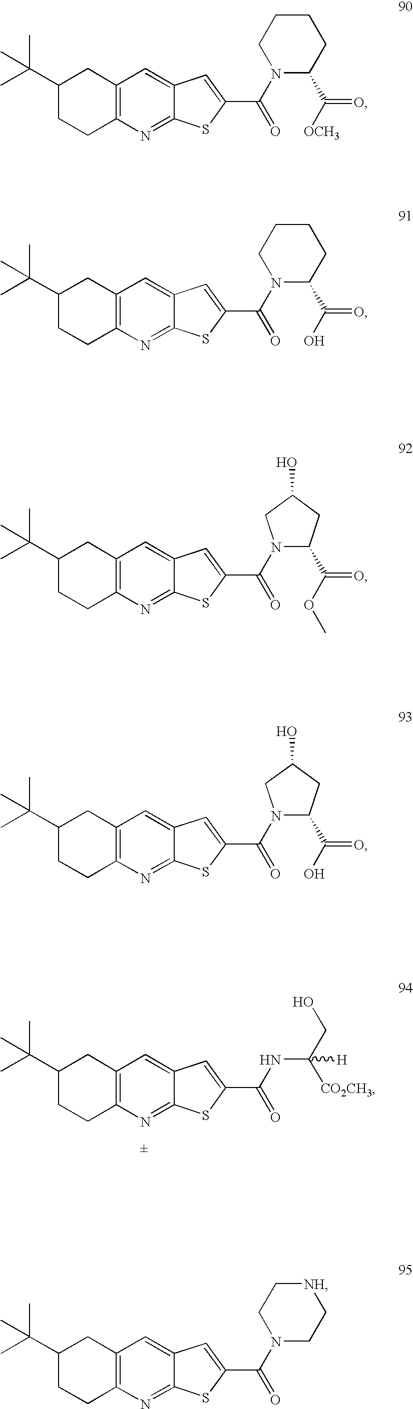 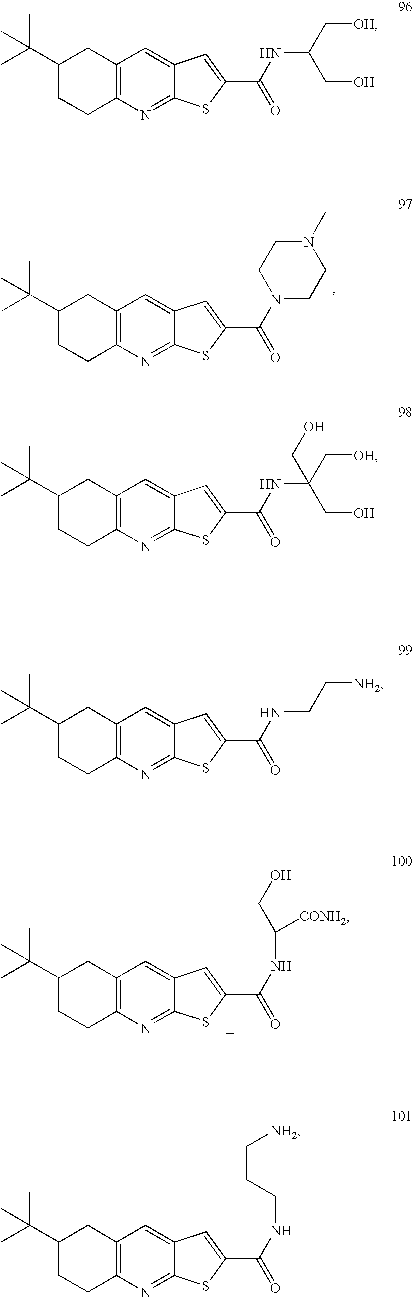 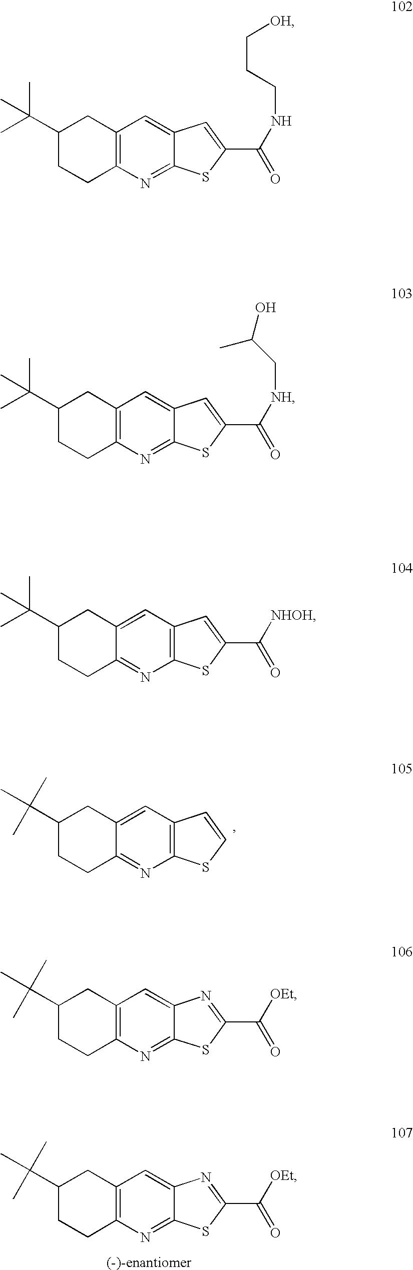 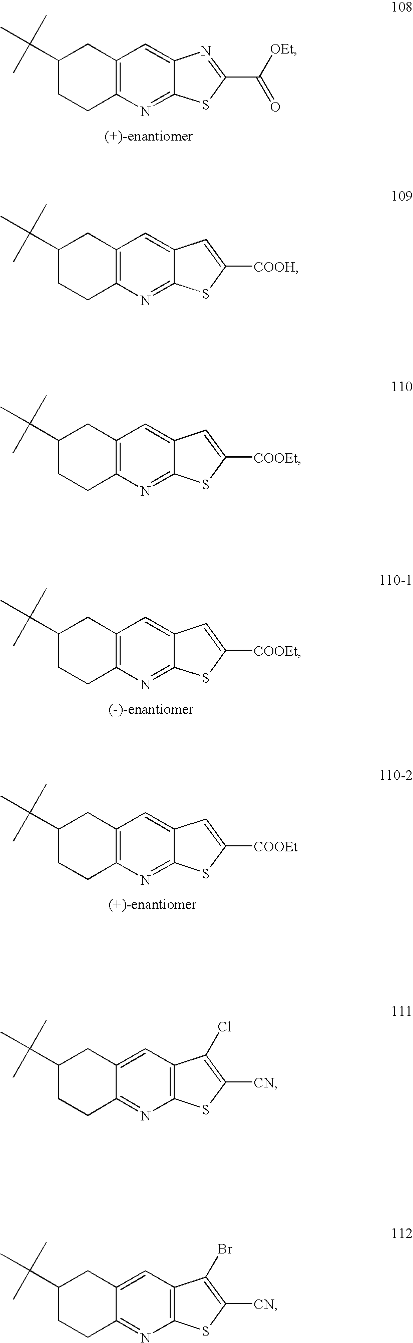 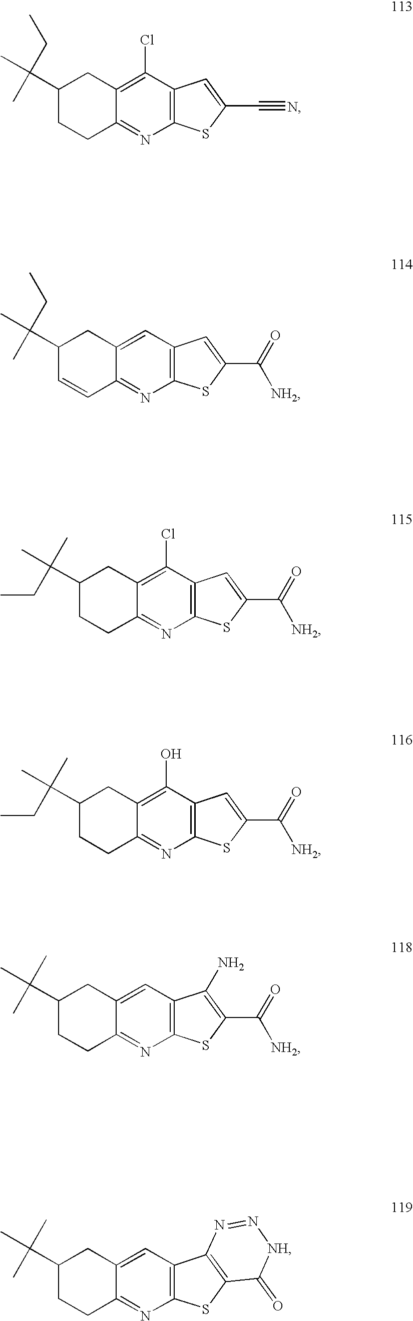 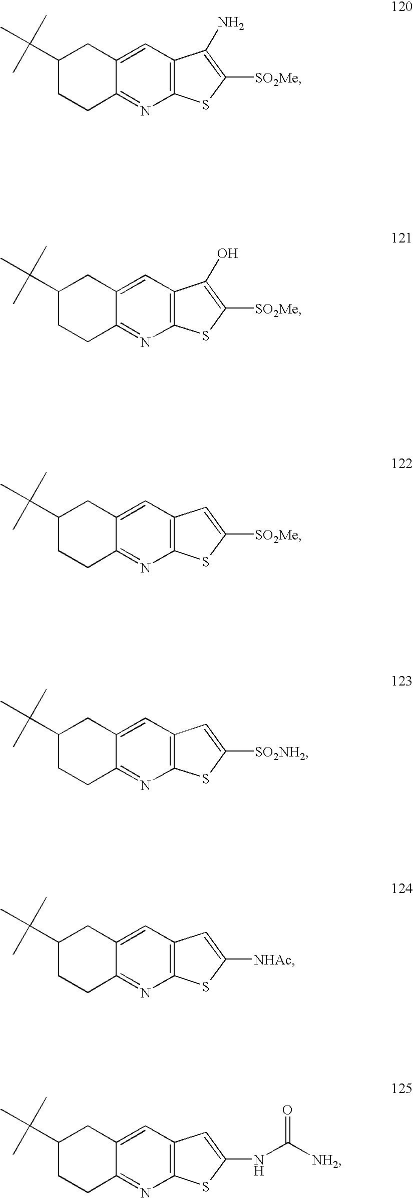 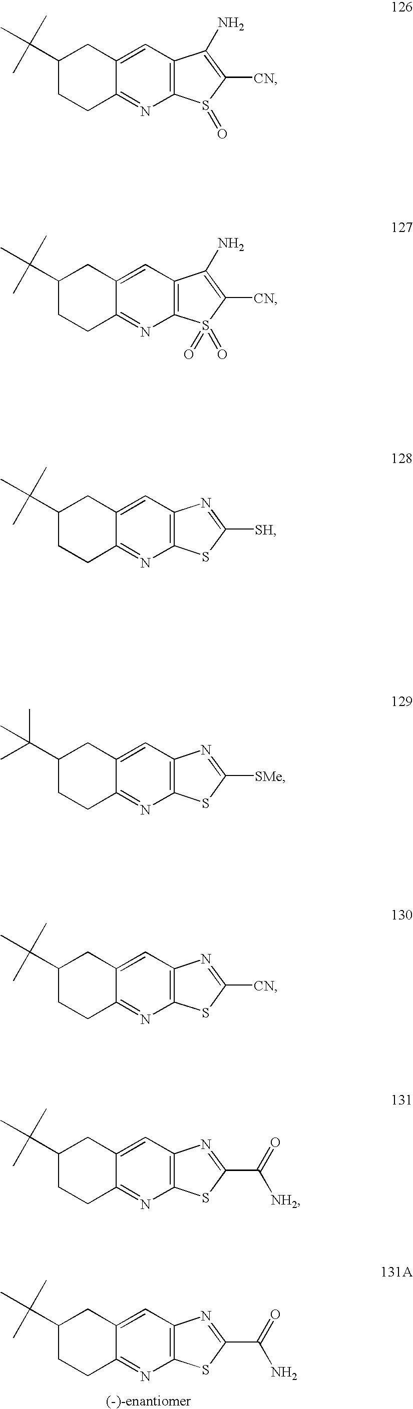 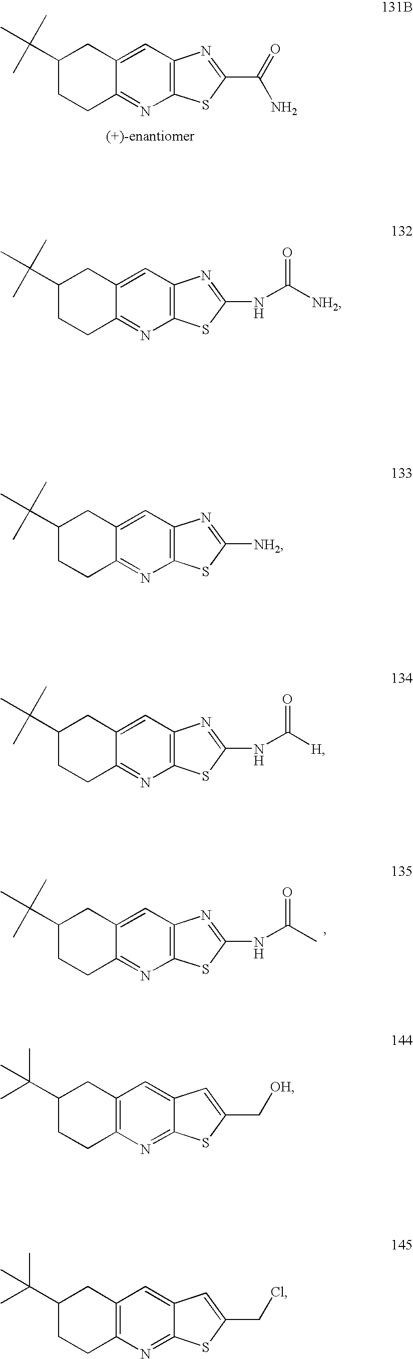 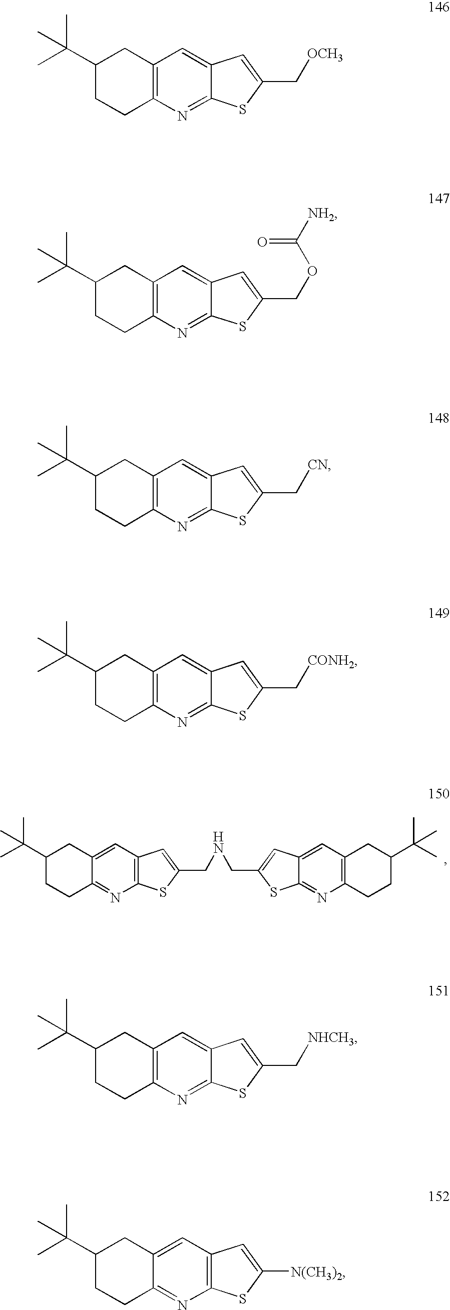 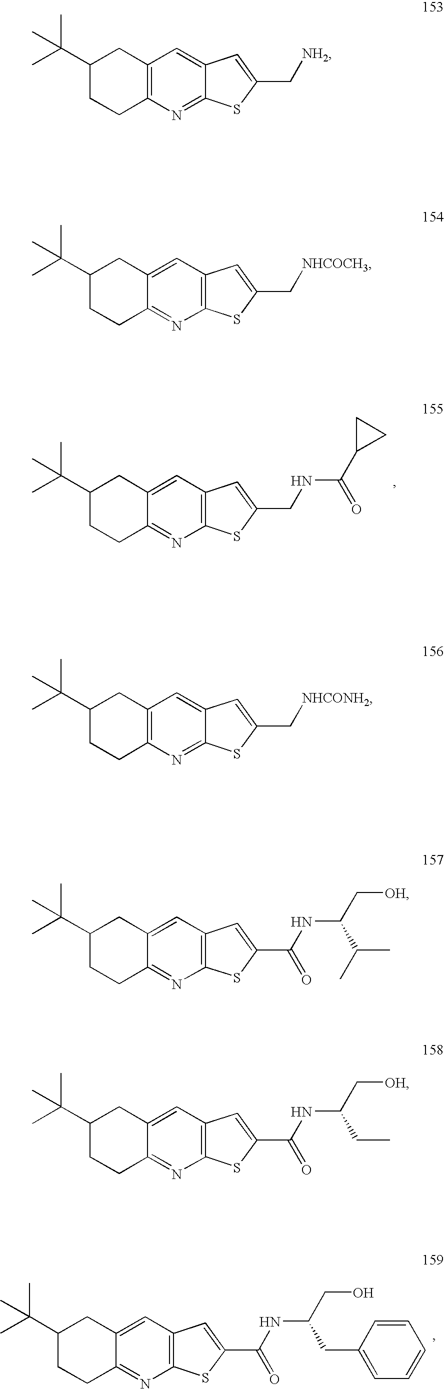 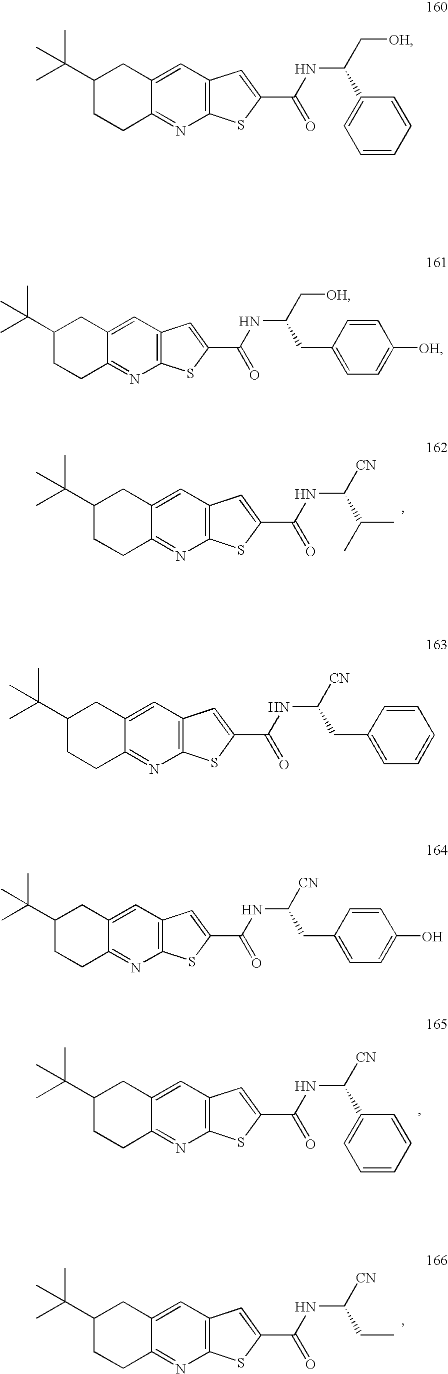 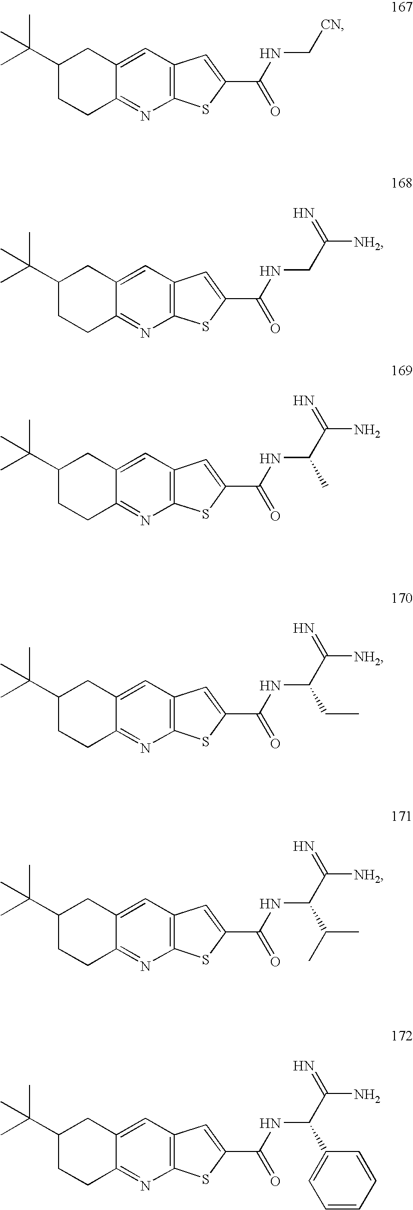 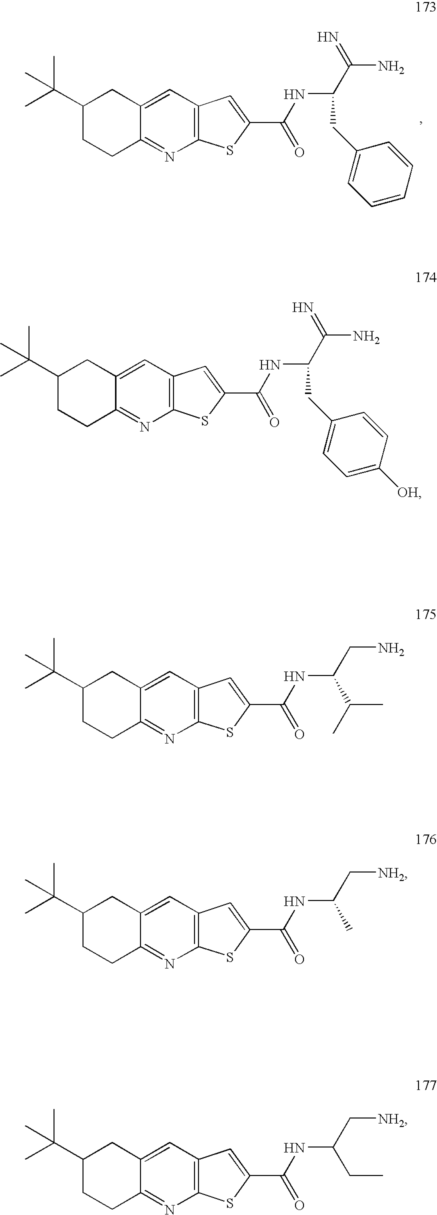 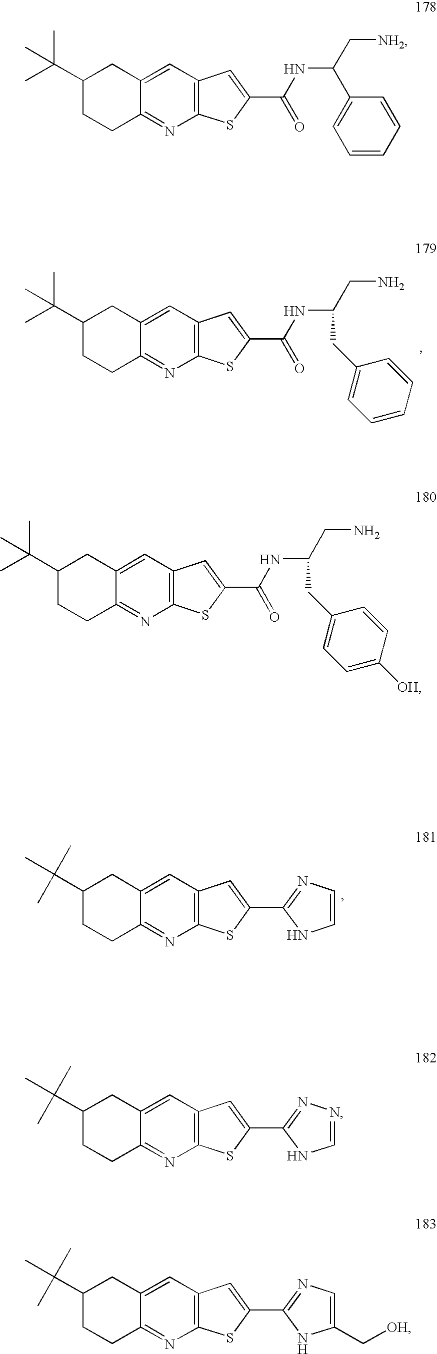 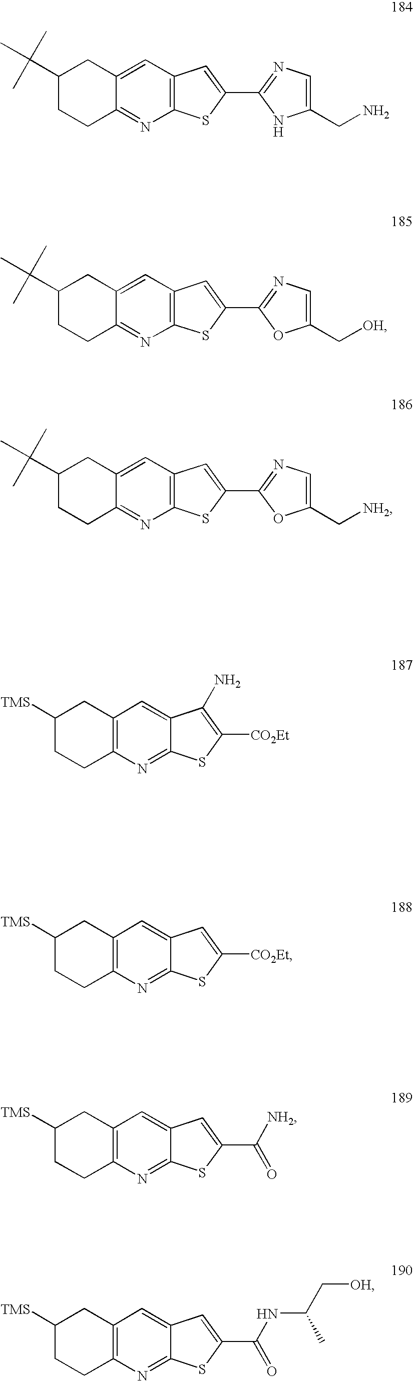 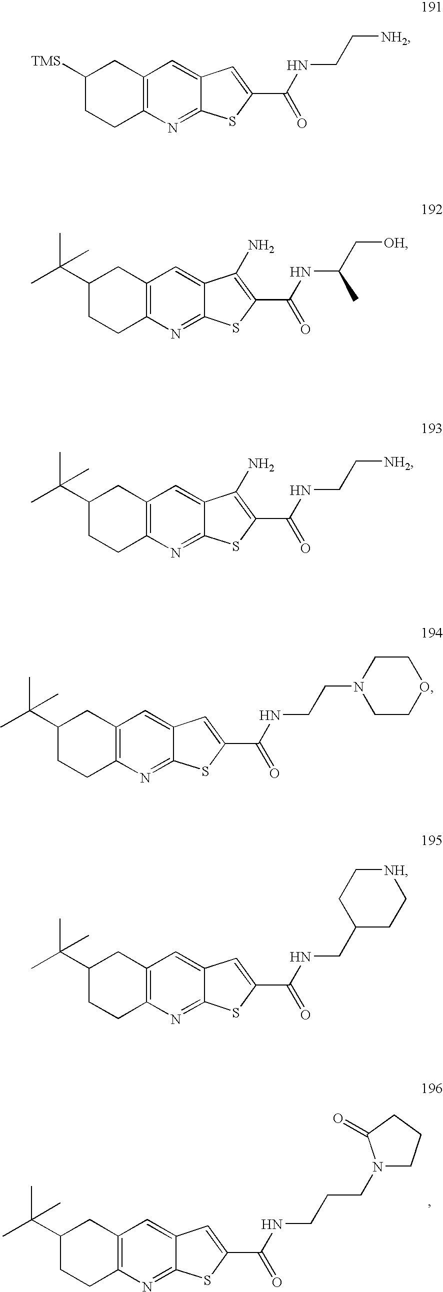 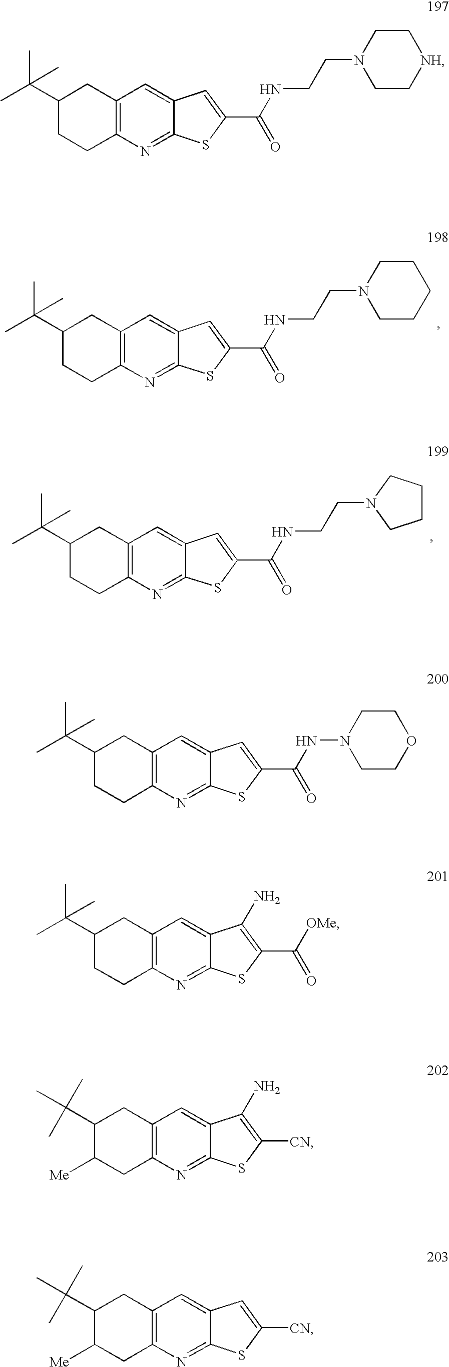 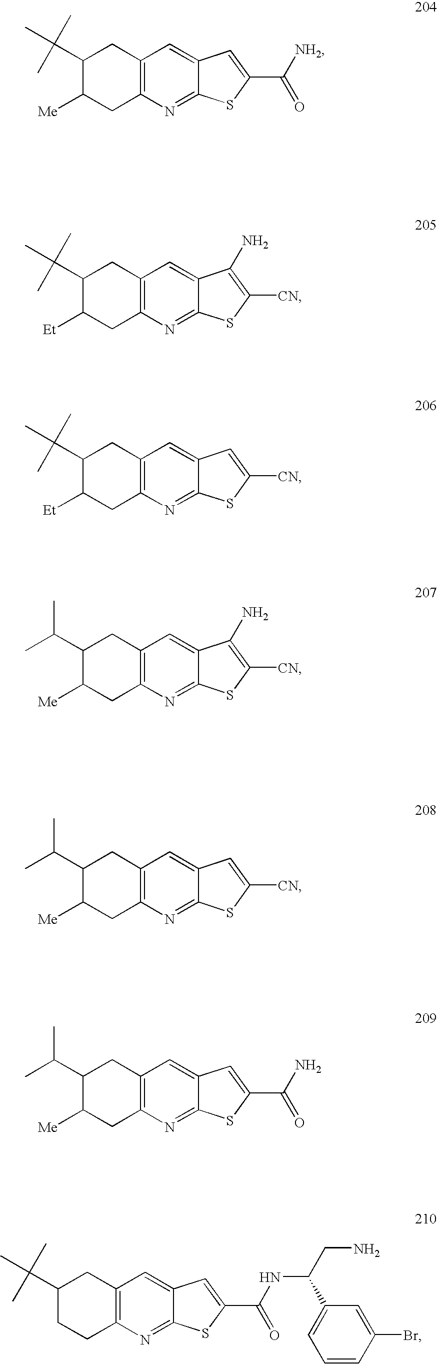 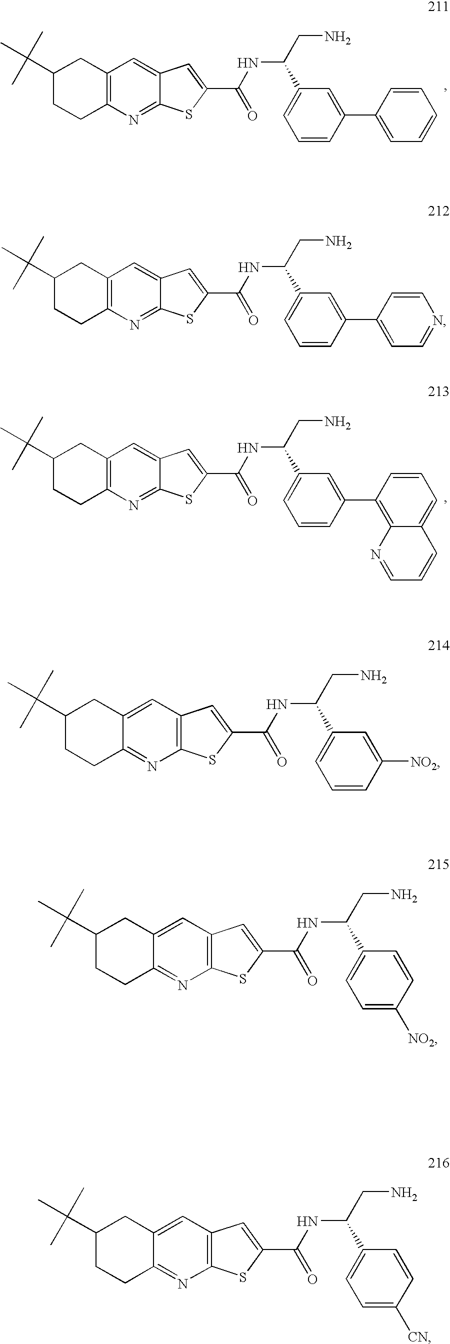 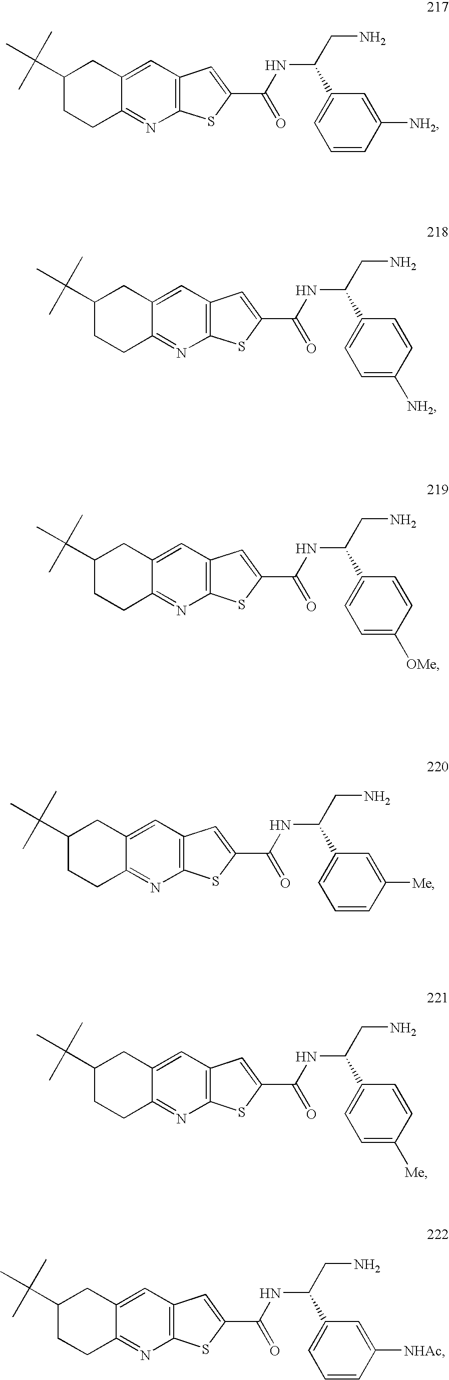 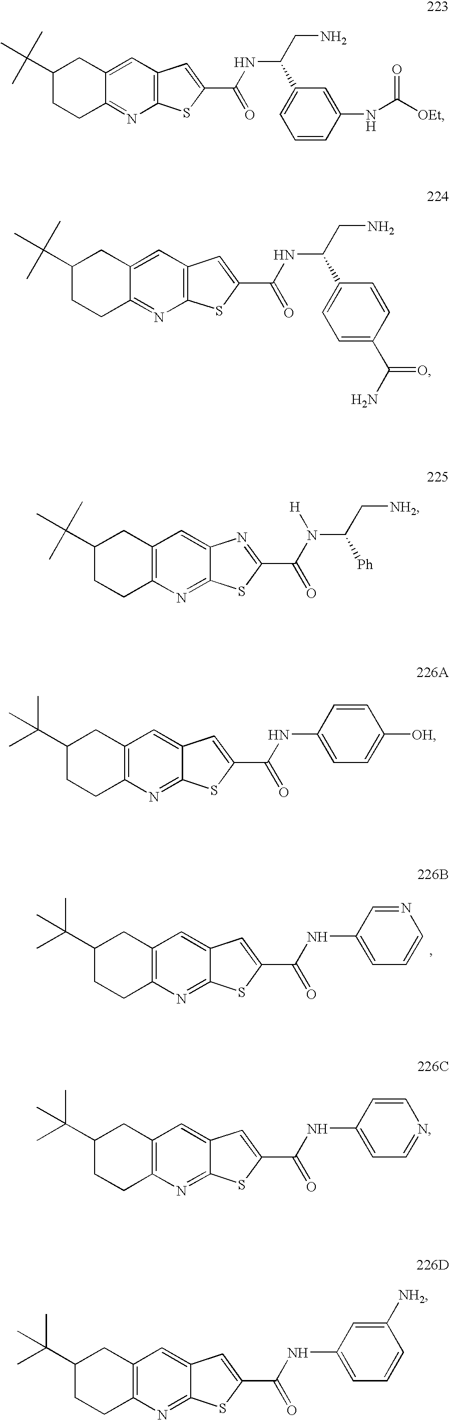 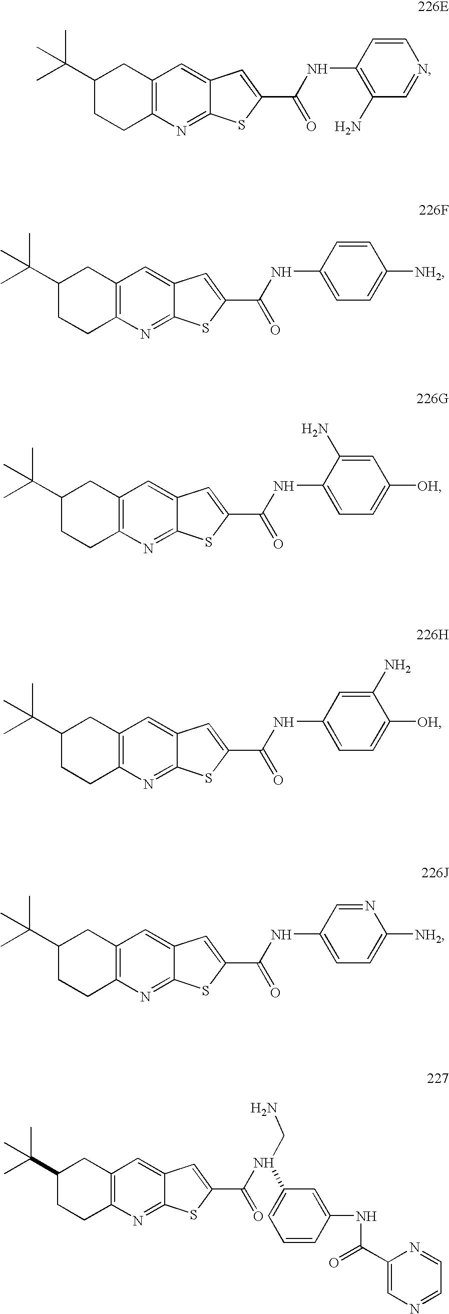 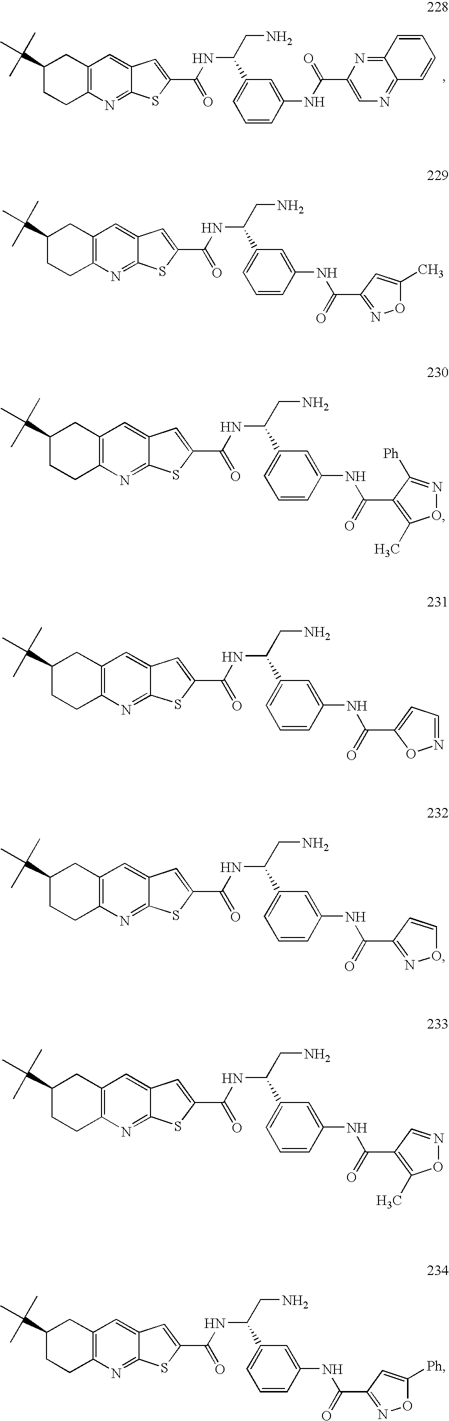 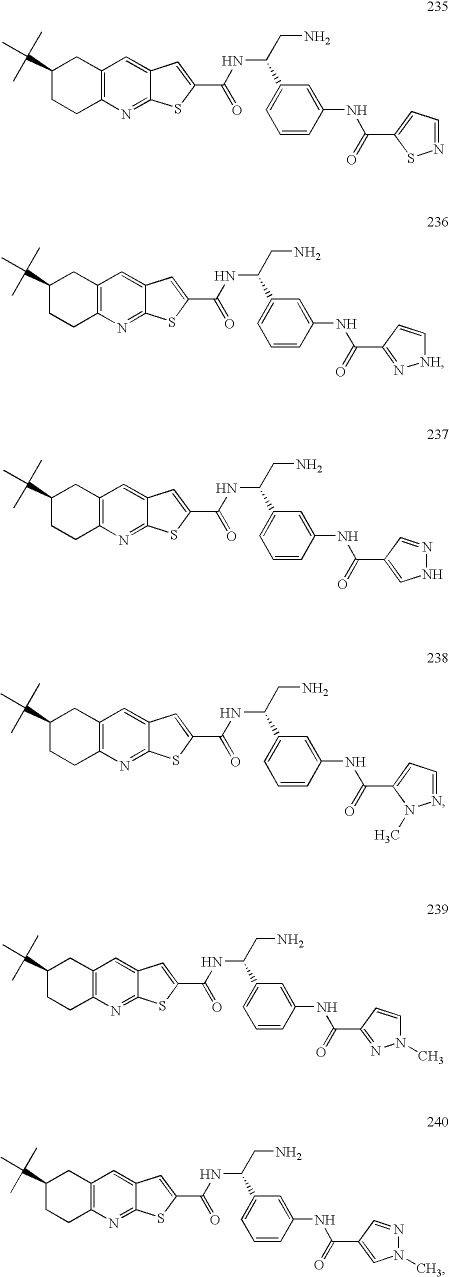 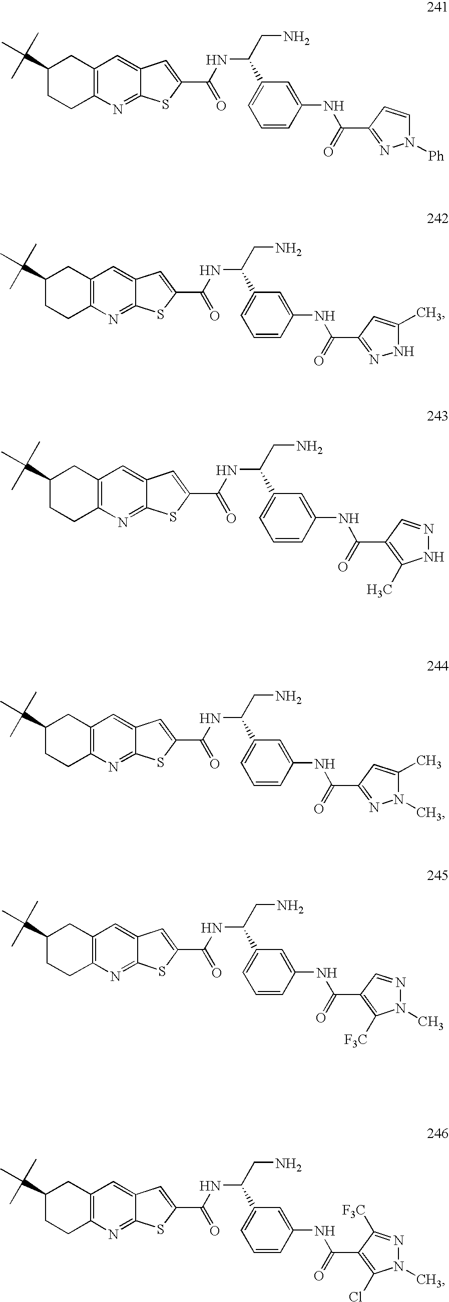 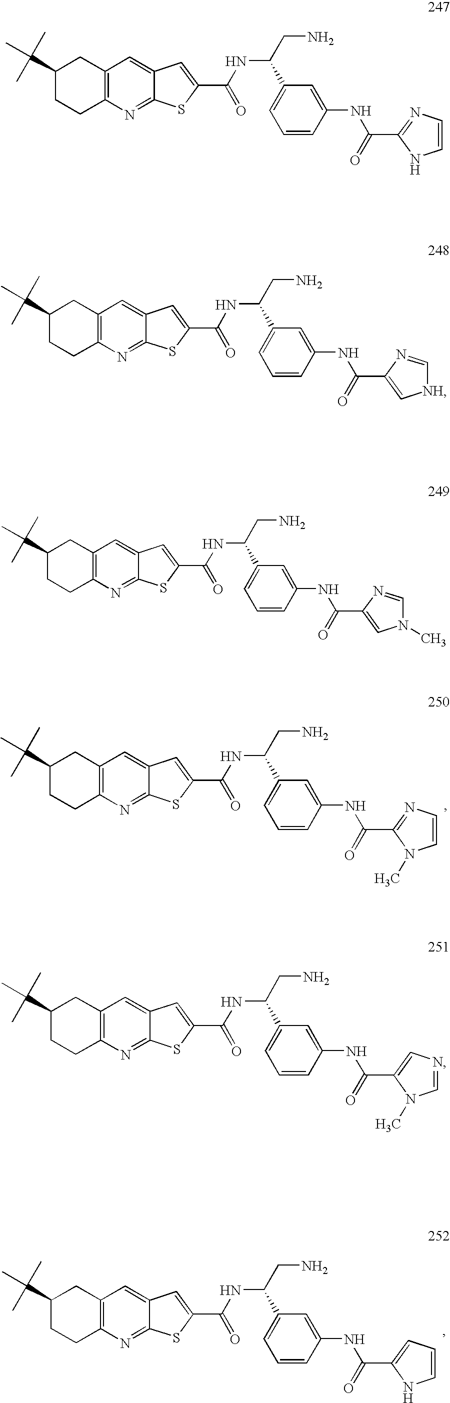 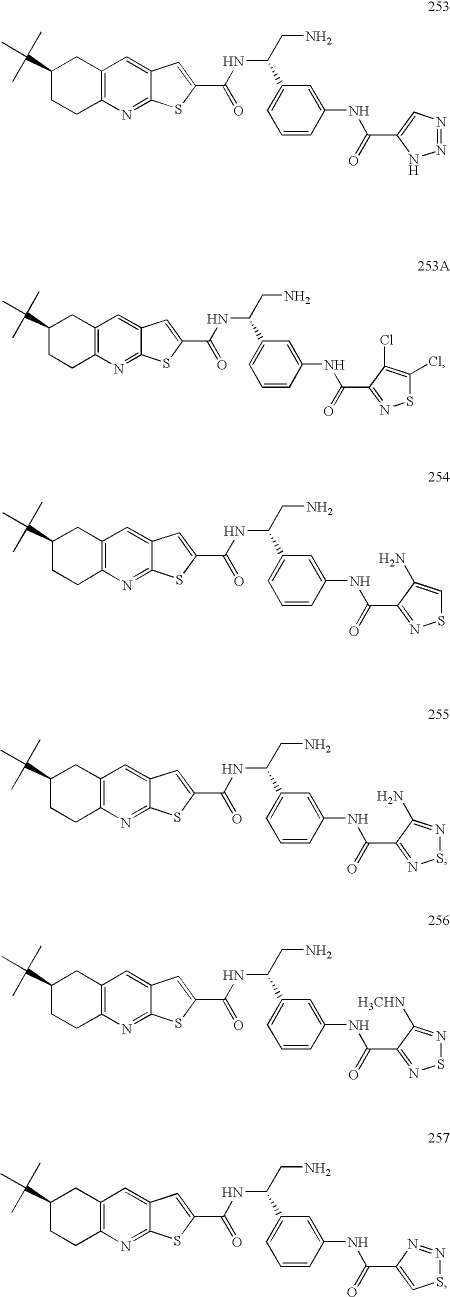 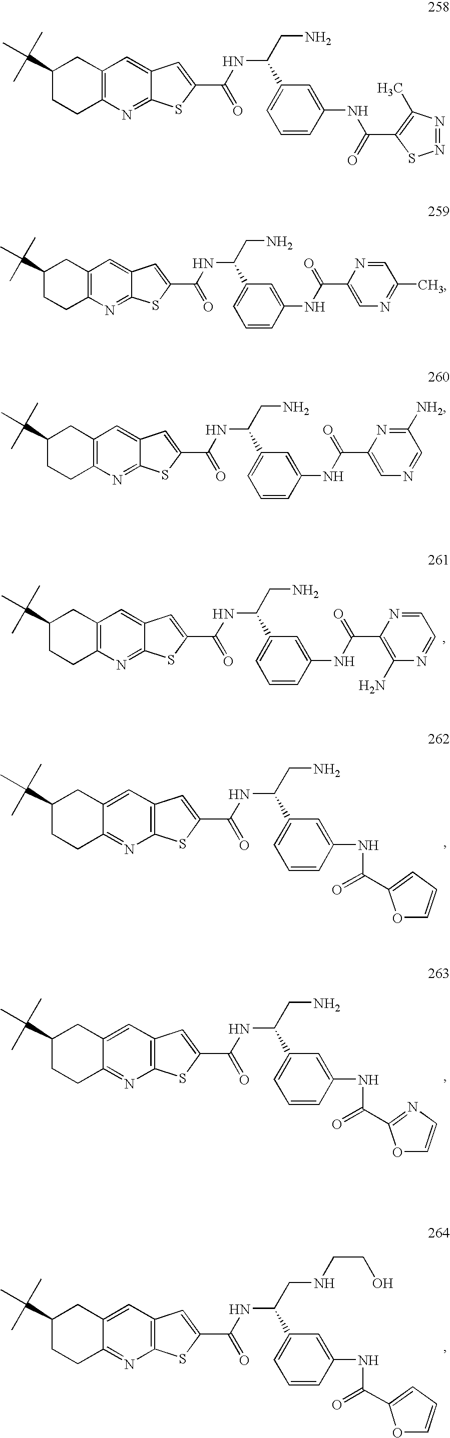 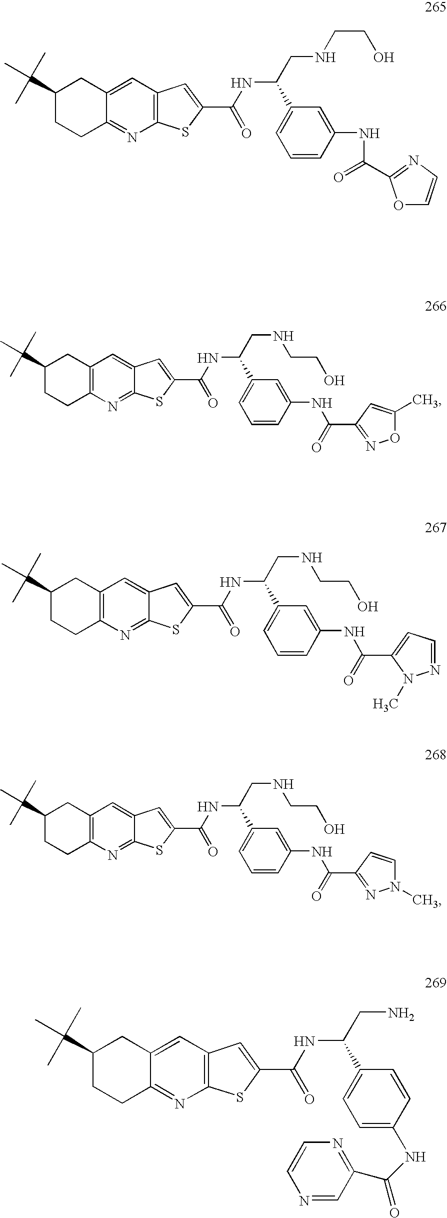 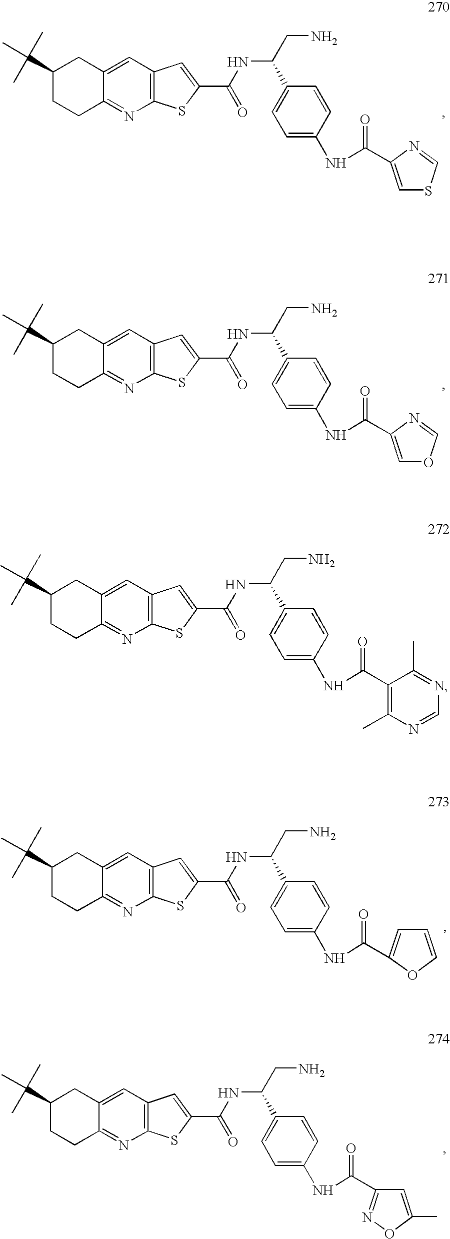 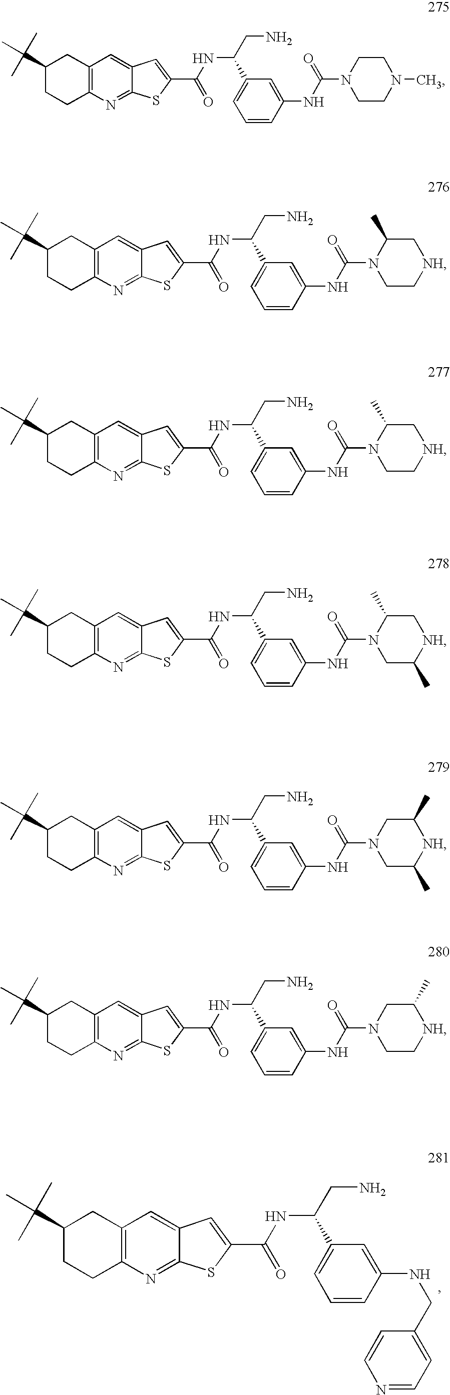 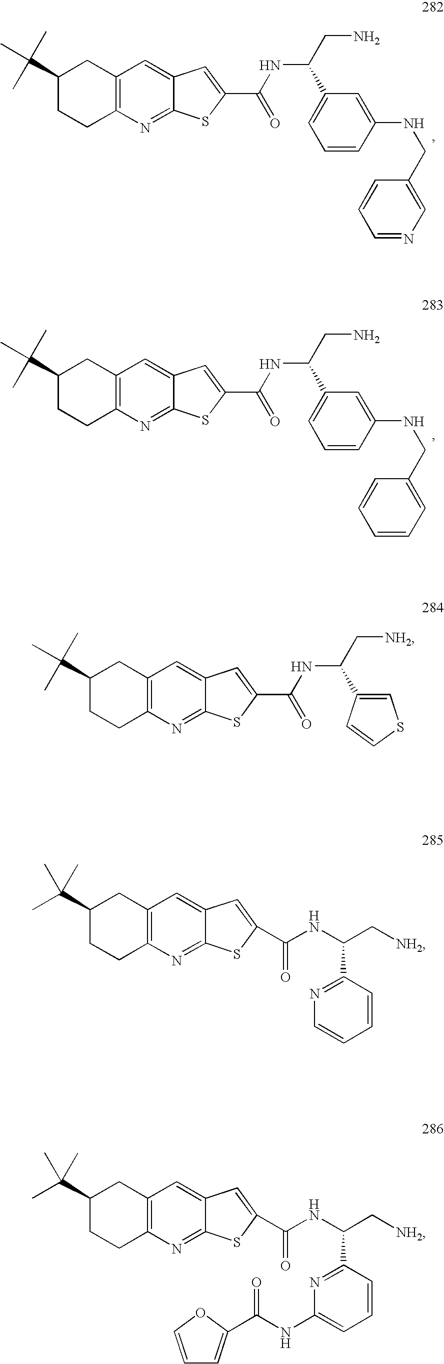 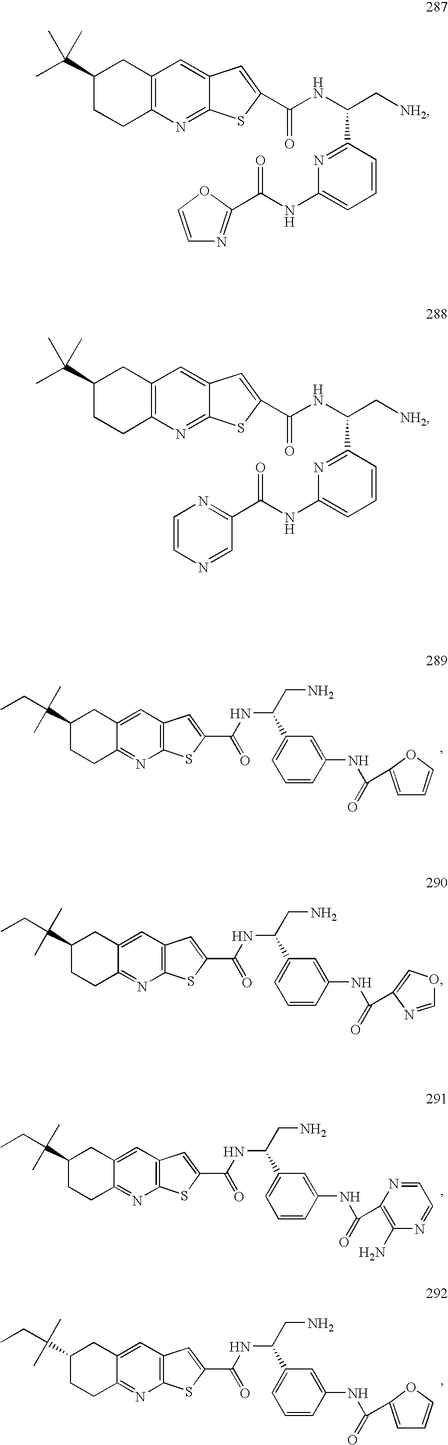 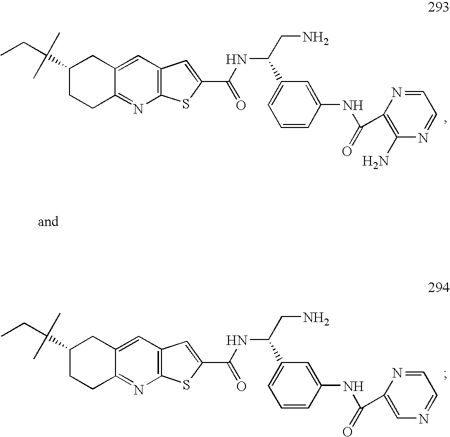
or a pharamaceutically acceptable salt or solvate thereof.

In another embodiment, the compounds of the present invention are selected from the group consisting of compound #s 6, 10, 12,25, 26, 28, 30, 40, 43, 58, 59, 62, 63, 64, 65, 67, 68, 74, 75, 79, 83, 85, 86, 99, 104, 123, 131, 131A, 131B, 144, 157, 158, 160, 167, 168, 169, 170, 177, 178, 179, 180, 181, 183, 184, 189, 191, 210, 211, 212, 217, 218, 222, 223, 224, 225, 226A, 226B, 226C, 226D, 226E, 226F, 226J, 227, and 228-284; or a pharmaceutically acceptable salt or solvate thereof.

In another embodiment, the compounds of the present invention are selected from the group consisting of compound #s 40, 59, 63, 64, 65, 67, 68, 99, 144, 168, 177, 178, 189, 191, 210, 211, 212, 217, 218, 222, 223, 224, 225, 226A, 226B, 226C, 226D, 226E, 226F, 226J, 227, and 228-284; or a pharmaceutically acceptable salt or solvate thereof.

In other embodiments, the present invention provides processes for producing such compounds, pharmaceutical formulations or compositions comprising one or more of such compounds, and methods of treating or preventing one or more conditions or diseases associated with KSP kinesin activity such as those discussed in detail below.

As used above, and throughout the specification, the following terms, unless otherwise indicated, shall be understood to have the following meanings:

“Subject” includes both mammals and non-mammalian animals.

“Mammal” includes humans and other mammalian animals.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties. It should be noted that any atom with unsatisfied valences in the text, schemes, examples and tables herein is assumed to have the hydrogen atom(s) to satisfy the valences.

The following definitions apply regardless of whether a term is used by itself or in combination with other terms, unless otherwise indicated. Therefore, the definition of “alkyl” applies to “alkyl” as well as the “alkyl” portions of “hydroxyalkyl”, “haloalkyl”, “alkoxy”, etc.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl. “Alkyl” includes “Alkylene” which refers to a difunctional group obtained by removal of a hydrogen atom from an alkyl group that is defined above. Non-limiting examples of alkylene include methylene (—CH₂—), ethylene (—CH₂CH₂—) and propylene (—C₃H₆—; which may be linear or branched).

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

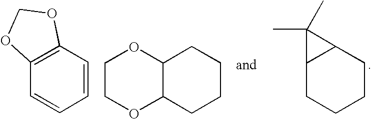

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a single moiety (e.g., carbonyl) which simultaneously replaces two available hydrogens on the same carbon atom on a ring system. Example of such moiety is pyrrolidone:

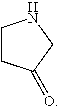

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, dihydroimidazole, dihydrooxazole, dihydrooxadiazole, dihydrothiazole, 3,4-dihydro-2H-pyran, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

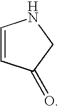

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring: 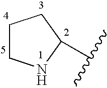
there is no —OH attached directly to carbons marked 2 and 5.

It should also be noted that tautomeric forms such as, for example, the moieties: 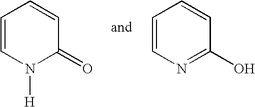
are considered equivalent in certain embodiments of this invention.

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1-or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkylsilyl” means an alkyl-Si— group in which alkyl is as previously defined and the point of attachment to the parent moiety is on Si. Preferred alkylsilyls contain lower alkyl. An example of an alkylsilyl group is trimethylsilyl (—Si(CH₃)₃).

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in any one of Formula I-IV, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C1-C2)alkyl and piperidino-, pyrrolidino-or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formulae I-IV can form salts which are also within the scope of this invention. Reference to a compound of Formulae I-IV herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of any one of Formulae I-IV contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formulae I-IV may be formed, for example, by reacting a compound of Formulae I-IV with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di-or triphosphate esters. The phosphate esters may be further esterified by, for example, a C₁₋₂₀ alcohol or reactive derivative thereof, or by a 2,3-di (C₆₋₂₄)acyl glycerol.

Compounds of Formulae I-IV, and salts, solvates, esters and prodrugs thereof, may exist in their tautomeric form (for example, as an amide or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formulae I-IV, and of the salts, solvates, esters and prodrugs of the compounds of Formulae I-IV, are intended to be included in the present invention.

Generally, the compounds of Formula I-IV can be prepared by a variety of methods well known to those skilled in the art, for example, by the methods as outlined in Scheme 1 below and in the examples disclosed herein:

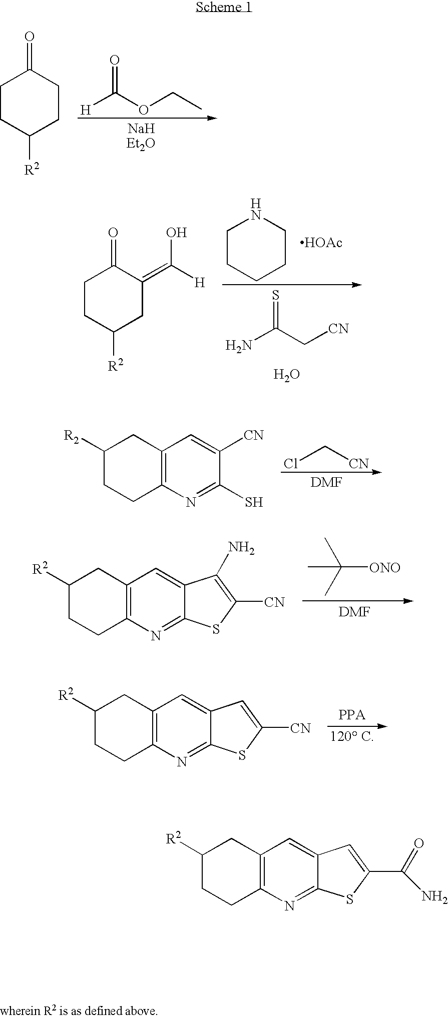

The compounds of the invention can be useful in a variety of applications involving alteration of mitosis. As will be appreciated by those skilled in the art, mitosis may be altered in a variety of ways; that is, one can affect mitosis either by increasing or decreasing the activity of a component in the mitotic pathway. Mitosis may be affected (e.g., disrupted) by disturbing equilibrium, either by inhibiting or activating certain components. Similar approaches may be used to alter meiosis.

In a particular embodiment, the compounds of the invention can be used to inhibit mitotic spindle formation, thus causing prolonged cell cycle arrest in mitosis. By “inhibit” in this context is meant decreasing or interfering with mitotic spindle formation or causing mitotic spindle dysfunction. By “mitotic spindle formation” herein is meant organization of microtubules into bipolar structures by mitotic kinesins. By “mitotic spindle dysfunction” herein is meant mitotic arrest and monopolar spindle formation.

The compounds of the invention can be useful for binding to, and/or inhibiting the activity of, a mitotic kinesin, KSP. In one embodiment, the KSP is human KSP, although the compounds may be used to bind to or inhibit the activity of KSP kinesins from other organisms. In this context, “inhibit” means either increasing or decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP (see U.S. Pat. No. 6,437,115). In addition, the present compounds are also useful for binding to or modulating other mitotic kinesins.

The compounds of the invention can be used to treat cellular proliferation diseases. Such disease states which can be treated by the compounds, compositions and methods provided herein include, but are not limited to, cancer (further discussed below), hyperplasia, cardiac hypertrophy, autoimmune diseases, fungal disorders, arthritis, graft rejection, inflammatory bowel disease, immune disorders, inflammation, cellular proliferation induced after medical procedures, including, but not limited to, surgery, angioplasty, and the like. Treatment includes inhibiting cellular proliferation. It is appreciated that in some cases the cells may not be in a hyper- or hypoproliferation state (abnormal state) and still require treatment. For example, during wound healing, the cells may be proliferating “normally”, but proliferation enhancement may be desired. Thus, in one embodiment, the invention herein includes application to cells or subjects afflicted or subject to impending affliction with any one of these disorders or states.

The compounds, compositions and methods provided herein are particularly useful for the treatment of cancer including solid tumors such as skin, breast, brain, colon, gall bladder, thyroid, cervical carcinomas, testicular carcinomas, etc. More particularly, cancers that may be treated by the compounds, compositions and methods of the invention include, but are not limited to:

Cardiac: sarcoma (angiosarcoma, fibrosarcoma, rhabdomyosarcoma, liposarcoma), myxoma, rhabdomyoma, fibroma, lipoma and teratoma;

Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver: hepatoma (hepatocellular carcinoma), cholangiocarcinoma, hepatoblastoma, angiosarcoma, hepatocellular adenoma, hemangioma;

Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;

Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);

Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);

Hematologic: blood (myeloid leukemia (acute and chronic), acute lymphoblastic leukemia, acute and chronic lymphocytic leukemia, myeloproliferative diseases, multiple myeloma, myelodysplastic syndrome), Hodgkin's disease, non-Hodgkin's lymphoma (malignant lymphoma), B-cell lymphoma, T-cell lymphoma, hairy cell lymphoma, Burkett's lymphoma, promyelocytic leukemia;

Skin: malignant melanoma, basal cell carcinoma, squamous cell carcinoma, Karposi's sarcoma, moles dysplastic nevi, lipoma, angioma, dermatofibroma, keloids, psoriasis;

Adrenal glands: neuroblastoma; and

Other tumors: including xenoderoma pigmentosum, keratoctanthoma and thyroid follicular cancer.

As used herein, treatment of cancer includes treatment of cancerous cells, including cells afflicted by any one of the above-identified conditions.

The compounds of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The compounds of the present invention may also be useful in inhibiting tumor angiogenesis and metastasis.

The compounds of the present invention may also be useful as antifungal agents, by modulating the activity of the fungal members of the bimC kinesin subgroup, as is described in U.S. Pat. No. 6,284,480.

The present compounds are also useful in combination with one or more other known therapeutic agents and anti-cancer agents. Combinations of the present compounds with other anti-cancer or chemotherapeutic agents are within the scope of the invention. Examples of such agents can be found in Cancer Principles and Practice of Oncology by V. T. Devita and S. Heliman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The present compounds are also useful when co-administered with radiation therapy.

The phrase “estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-I-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-ydrazone, aid SH646.

The phrase “androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

The phrase “retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a difluoromethylomithine, ILX23-7553, trans-N-(4′-hydroxyphenyl) retinamide, and N-4-carboxyphenyl retinamide.

The phrase “cytotoxic/cytostatic agents” refer to compounds which cause cell death or inhibit cell proliferation primarily by interfering directly with the cell's functioning or inhibit or interfere with cell mycosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, monoclonal antibody therapeutics, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)]tetrachloride, diarizidinylspermine, arsenic trioxide, 1-(11-dodecylamino-10-hydroxyundecyl)-3,7-dimethylxanthine, zonubicin, idarubicin, daunorubicin, bisantrene, mitoxantrone, pirarubicin, pinafide, valrubicin, amrubicin, antineoplaston, 3′-deansino-3′-morpholino-13-deoxo-10-hydroxycarminomycin, annamycin, galarubicin, elinafide, MEN10755, 4-demethoxy-3-deamino-3-aziridinyl-4-methylsulphonyl-daunombicin (see WO 00/50032), methoxtrexate, gemcitabine, and mixture thereof.

An example of a hypoxia activatable compound is tirapazamine.

Examples of proteasome inhibitors include, but are not limited to, lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H, 12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, dimesna, and camptostar.

Other useful anti-cancer agents that can be used in combination with the present compounds include thymidilate synthase inhibitors, such as 5-fluorouracil.

In one embodiment, inhibitors of mitotic kinesins include, but are not limited to, inhibitors of KSP, inhibitors of MKLP1, inhibitors of CENP-E, inhibitors of MCAK, inhibitors of Kif14, inhibitors of Mphosph1 and inhibitors of Rab6-KIFL.

The phrase “inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

The phrase “antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, neizarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

Examples of monoclonal antibody therapeutics useful for treating cancer include Erbitux (Cetuximab).

The phrase “HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin(ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open acid and lactone forms is included in the scope of this invention.

The phrase “prenyl-protein transferase inhibitor” refers to a compound which inhibits any one or any combination of the prenyl-protein transferase enzymes, including famesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO, 97/30053, WO 97/44350, WO 98/02436, and U.S. Pat. No. 5,532,359. For an example of the role of a prenyl-protein transferase inhibitor on angiogenesis see European of Cancer, Vol. 35, No. 9, pp. 1394-1401(1999).

Examples of famesyl protein transferase inhibitors include SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoehtyl]-1-piperidinecarboxamide from Schering-Plough Corporation, Kenilworth, N.J.), tipifarnib (Zarnestra® or R115777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.).

The phrase “angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α (for example Intron and Peg-Intron), interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin. Orthop. Vol. 313, p. 76 (1995); J. Mol. Endocnnol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). Examples of TAFIa inhibitors have been described in PCT Publication WO 03/013,526.

The phrase “agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

The phrase “inhibitors of cell proliferation and survival signaling pathway” refers to agents that inhibit cell surface receptors and signal transduction cascades downstream of those surface receptors. Such agents include inhibitors of EGFR (for example gefitinib and erlotinib), antibodies to EGFR (for example C225), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEEK (for example CI-1040 and PD-098059), inhibitors of mTOR (for example Wyeth CCI-779), and inhibitors of C-abl kinase (for example GLEEVEC™, Novartis Pharmaceuticals). Such agents include small molecule inhibitor compounds and antibody antagonists.

The phrase “apoptosis inducing agents” includes activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5 pyridinyl)pyridine; or a pharmaceutically acceptable salt thereof.

Compounds that have been described as specific inhibitors of COX-2 and are therefore useful in the present invention include, but are not limited to, parecoxib, CELIEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some examples of tyrosine kinase inhibitors include N-(trifluoromethylphenyl)-5-methylisoxazol-4-carboxamide, 3-[(2,4-dimethylpyrrol-5-yl)methylidenyl)indolin-2-one,17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the present compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999;274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid, and 2(R)-7-(3-(2-chloro-4-(4-fluorophenoxy)phenoxy)propoxy)-2-ethylchromane-2-carboxylic acid.

In one embodiment, useful anti-cancer (also known as anti-neoplastic) agents that can be used in combination with the present compounds include, but are not limited, to Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaeuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin (adriamycin), cyclophosphamide (cytoxan), gemcitabine, interferons, pegylated interferons, Erbitux and mixtures thereof.

Another embodiment of the present invention is the use of the present compounds in combination with gene therapy for the treatment of cancer. For an overview of genetic strategies to treating cancer, see Hall et al (Am J Hum Genet 61:785-789,1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998;5(8):1105-13), and interferon gamma (J Immunol 2000; 164:217-222).

The present compounds can also be administered in combination with one or more inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

The present compounds can also be employed in conjunction with one or more anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with one or more other anti-emetic agents, especially neurokinin-1 receptor antagonists, 5HT3 receptor, antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or those as described in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In one embodiment, an anti-emesis agent selected from a neurokinin-1 receptor antagonist, a 5HT3 receptor antagonist and a corticosteroid is administered as an adjuvant for the treatment or prevention of emesis that may result upon administration of the present compounds.

Examples of neurokinin-1 receptor antagonists that can be used in conjunction with the present compounds are described in U.S. Pat. Nos. 5,162,339, 5,232,929, 5,242,930, 5,373,003, 5,387,595, 5,459,270, 5,494,926, 5,496,833, 5,637,699, and 5,719,147, content of which are incorporated herein by reference. In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the present invention may also be administered with one or more immunologic-enhancing drug, such as for example, levamisole, isoprinosine and Zadaxin.

Thus, the present invention encompasses the use of the present compounds (for example, for treating or preventing cellular proliferative diseases) in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug, resistance, an anti-emetic agent, an immunologic-enhancing drug, an inhibitor of cell proliferation and survival signaling, an agent that interfers with a cell cycle checkpoint, and an apoptosis inducing agent.

In one embodiment, the present invention empassesses the composition and use of the present compounds in combination with a second compound selected from: a cytostatic agent, a cytotoxic agent, taxanes, a topoisomerase II inhibitor, a topoisomerase I inhibitor, a tubulin interacting agent, hormonal agent, a thymidilate synthase inhibitors, anti-metabolites, an alkylating agent, a farnesyl protein transferase inhibitor, a signal transduction inhibitor, an EGFR kinase inhibitor, an antibody to EGFR, a C-abl kinase inhibitor, hormonal therapy combinations, and aromatase combinations.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In one embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MW (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-(O-chloroacetylcarbonyl)-fumagillol, thalidomide, angiostatin, troponin-1, or an antibody to VEGF. In an embodiment, the estrogen receptor modulator is tamoxifen or raloxifene.

Also included in the present invention is a method of treating cancer comprising administering a therapeutically effective amount of at least one compound of Formulae I-IV in combination with radiation therapy and at least one compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drag, an inhibitor of cell proliferation and survival signaling, an agent that interfers with a cell cycle checkpoint, and an apoptosis inducing agent.

Yet another embodiment of the invention is a method of treating cancer comprising administering a therapeutically effective amount of at least one compound of Formulae I-IV in combination with paclitaxel or trastuzumab.

The present invention also includes a pharmaceutical composition useful for treating or preventing cellular proliferation diseases (such as cancer, hyperplasia, cardiac hypertrophy, autoimmune diseases, fungal disorders, arthritis, graft rejection, inflammatory bowel disease, immune disorders, inflammation, and cellular proliferation induced after medical procedures) that comprises a therapeutically effective amount of at least one compound of Formulae I-IV and at least one compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of cell proliferation and survival signaling, an agent that interfers with a cell cycle checkpoint, and an apoptosis inducing agent.

Another aspect of this invention relates to a method of selectively inhibiting KSP kinesin activity in a subject (such as a cell, animal or human) in need thereof, comprising contacting said subject with at least one compound of Formulae I-IV or a pharmaceutically acceptable salt or ester thereof.

Preferred KSP kinesin inhibitors are those which can specifically inhibit KSP kinesin activity at low concentrations, for example, those that cause a level of inhibition of 50% or greater at a concentration of 50 μM or less, more preferably 100 nM or less, most preferably 50 nM or less.

Another aspect of this invention relates to a method of treating or preventing a disease or condition associated with KSP in a subject (e.g., human) in need thereof comprising administering a therapeutically effective amount of at least one compound of Formulae I-IV or a pharmaceutically acceptable salt or ester thereof to said subject.

A preferred dosage is about 0.001 to 500 mg/kg of body weight/day of a compound of Formulae I-IV or a pharmaceutically acceptable salt or ester thereof. An especially preferred dosage is about 0.01 to 25 mg/kg of body weight/day of a compound of Formulae I-IV or a pharmaceutically acceptable salt or ester thereof.

The phrases “effective amount” and “therapeutically effective amount” mean that amount of a compound of Formulae I-IV, and other pharmacological or therapeutic agents described herein, that will elicit a biological or medical response of a tissue, a system, or a subject (e.g., animal or human) that is being sought by the administrator (such as a researcher, doctor or veterinarian) which includes alleviation of the symptoms of the condition or disease being treated and the prevention, slowing or halting of progression of one or more cellular proliferation diseases. The formulations or compositions, combinations and treatments of the present invention can be administered by any suitable means which produce contact of these compounds with the site of action in the body of, for example, a mammal or human.

For administration of pharmaceutically acceptable salts of the above compounds, the weights indicated above refer to the weight of the acid equivalent or the base equivalent of the therapeutic compound derived from the salt.

As described above, this invention includes combinations comprising an amount of at least one compound of Formulae I-IV or a pharmaceutically acceptable salt or ester thereof, and an amount of one or more additional therapeutic agents listed above (administered together or sequentially) wherein the amounts of the compounds/treatments result in desired therapeutic effect.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for illustration purposes, a compound of Formulae I-IV and an additional therapeutic agent may be present in fixed amounts (dosage amounts) in a single dosage unit (e.g., a capsule, a tablet and the like). A commercial example of such single dosage unit containing fixed amounts of two different active compounds is VYTORIN® (available from Merck Schering-Plough Pharmaceuticals, Kenilworth, N.J.).

If formulated as a fixed dose, such combination products employ the compounds of this invention within the dosage range described herein and the other pharmaceutically active agent or treatment within its dosage range. Compounds of Formulae I-IV may also be administered sequentially with known therapeutic agents when a combination formulation is inappropriate. The invention is not limited in the sequence of administration; compounds of Formulae I-IV may be administered either prior to or after administration of the known therapeutic agent. Such techniques are within the skills of persons skilled in the art as well as attending physicians.

The pharmacological properties of the compounds of this invention may be confirmed by a number of pharmacological assays. The inhibitory activity of the present compounds towards KSP may be assayed by methods known in the art, for example, by using the methods as described in the examples.

While it is possible for the active ingredient to be administered alone, it is preferable to present it as a pharmaceutical composition. The compositions of the present invention comprise at least one active ingredient, as defined above, together with one or more acceptable carriers, adjuvants or vehicles thereof and optionally other therapeutic agents. Each carrier, adjuvant or vehicle must be acceptable in the sense of being compatible with the other ingredients of the composition and not injurious to the mammal in need of treatment.

Accordingly, this invention also relates to pharmaceutical compositions comprising at least one compound of Formulae I-IV, or a pharmaceutically acceptable salt or ester thereof and at least one pharmaceutically acceptable carrier, adjuvant or vehicle.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

The term pharmaceutical composition is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a subject by administering a pharmaceutical composition of the present invention is also intended to encompass the administration of the afore-said bulk composition and individual dosage units.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts or esters thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formulae I-IV or a pharmaceutically acceptable salt or ester thereof and at least one pharmaceutically acceptable carrier, adjuvant or vehicle.

Yet another aspect of this invention is a kit comprising an amount of at least one compound of Formulae I-IV or a pharmaceutically acceptable salt or ester thereof and an amount of at least one additional therapeutic agent listed above, wherein the amounts of the two or more ingredients result in desired therapeutic effect.

The invention disclosed herein is exemplified by the following preparations and examples which should not be construed to limit the scope of the disclosure. Alternative mechanistic pathways and analogous structures will be apparent to those skilled in the art.

The following solvents and reagents may be referred to by their abbreviations in parenthesis:
Thin layer chromatography: TLC
dichloromethane: CH₂Cl₂
ethyl acetate: AcOEt or EtOAc
methanol: MeOH
trifluoroacetate: TFA
triethylamine: Et₃N or TEA
butoxycarbonyl: n-Boc or Boc
nuclear magnetic resonance spectroscopy: NMR
liquid chromatography mass spectrometry: LCMS
high resolution mass spectrometry: HRMS
milliliters: mL
millimoles: mmol
microliters: μl
grams: g
milligrams: mg
room temperature or rt (ambient): about 25° C.
dimethoxyethane: DME

### EXAMPLES

Illustrating the invention are the following examples which, however, are not to be considered as limiting the invention to their details. Unless otherwise indicated, all parts and percentages in the following examples, as well as throughout the specification, are by weight.

### Preparative Example 1

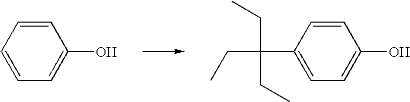 Step A:

A solution of phenol (1.0 g, 10.62 mmol)) in TFA (6.6 mL) at 25° C. was treated with 3-ethyl-3-pentanol (1.4 mL, 1.1 equiv.) followed by conc. H₂SO₄ (0.14 mL). Stirring was continued at 25° C. for 18 h. The solution was concentrated and the residue was diluted with CH₂Cl₂ (25 mL). The organic layer was washed with H₂O (50 mL), saturated NaHCO₃ (50 mL) and saturated NaCl (50 mL). The combined organic layer was dried (Na₂SO₄), filtered and concentrated under reduced pressure to yield 1.92 g (94%) 4-(1,1-diethyl-propyl)phenol.

### Preparative Examples 2-6

By essentially the same procedure set forth in Preparative Example 1, only substituting the alcohol shown in Column 2 of Table 1, the compounds in Column 3 were prepared:

TABLE 1

| Prep. | | |
|---|---|---|
| Example | Column 2 | Column 3 |
| 2 | 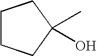 | 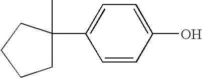 |
| 3 | 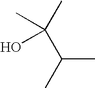 | 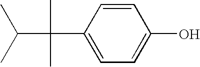 |
| 4 | 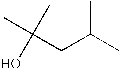 | 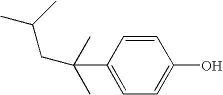 |
| 5 | 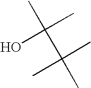 | 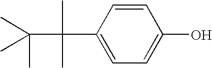 |
| 6 | 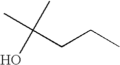 | 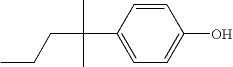 |

### Preparative Example 7

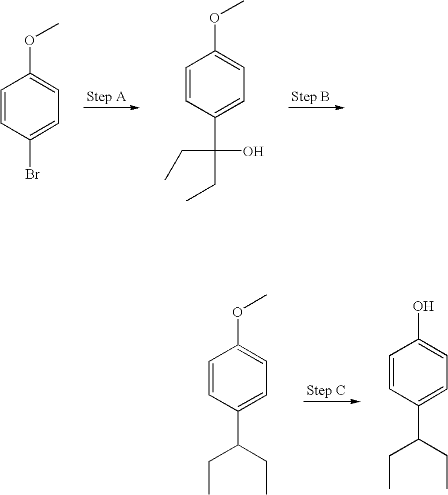 Step A:

4-Bromoanisole (3.01 g, 16.11 mmol) was dissolved in anhydrous THF (15 mL) and cooled to −78° C. n-Butyllithium (7.1 mL, 2.5 M in hexanes, 1.10 equiv.) was added dropwise and the reaction was stirred for 45 min. 3-Pentanone (1.45 g, 1.04 equiv.) was dissolved in anhydrous THF (3 mL) and added dropwise to the reaction. After 2.15 hours at −78° C., the reaction was quenched with H₂O (30 mL) and warmed to room temperature. The mixture was extracted once with ether (30 mL) and the organic layer was washed with H₂O and brine, dried (Na₂SO₄), filtered and concentrated under reduced pressure. Yield 2.68 g 4-(1-ethyl-1-hydroxypropyl)anisole (86%).

Step B:

The alcohol (2.66 g, 13.73 mmol) was dissolved in anhydrous dichloromethane (25 mL) and cooled to 0° C. Triethylsilane (4.3 mL, 1.96 equiv.) and boron trifluoride-etherate complex (3.4 mL, 1.95 equiv.) were added consecutively. The reaction was stirred for 15 h, warming to room temperature. Saturated sodium bicarbonate (25 mL) was added, and the mixture was extracted with ether (1×50 mL, 1×25 mL). The combined organic layers were washed with brine, dried (Na₂SO₄), filtered and concentrated under reduced pressure. Yield 2.45 g 4-(1-ethylpropyl)anisole (100%).

Step C:

The anisole (2.44 g, 13.7 mmol) was dissolved in anhydrous dichloromethane (60 mL) and cooled to −78° C. Boron tribromide (2.8 mL, 2.16 equiv.) was added slowly, and the reaction was stirred 15 h, warming to room temperature. After cooling to 0° C., the reaction was slowly quenched with saturated sodium bicarbonate (20 mL) and H₂O (10 mL). After 5 min., the organic layer was separated, and the aqueous layer was extracted with dichloromethane (1×40 mL). The combined organic layers were washed with saturated sodium bicarbonate, H₂O and brine, and dried (Na₂SO₄), filtered and concentrated under reduced pressure. Yield 2.013 g 4-(1-ethylpropyl)phenol (90%).

### Preparative Examples 8-13

By essentially the same procedure set forth in Preparative Example 7, only substituting the ketone or aldehyde shown in Column 2 of Table 2 in Preparative Example 7, Step A, the compounds in Column 3 were prepared:

TABLE 2

| Prep. | | |
|---|---|---|
| Example | Column 2 | Column 3 |
| 8 | 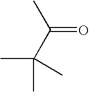 | 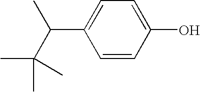 |
| 9 | 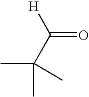 | 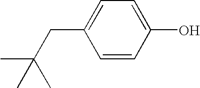 |
| 10 | 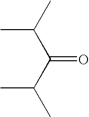 | 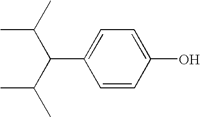 |
| 11 | 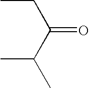 | 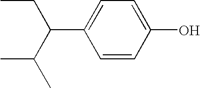 |
| 12 | 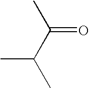 | 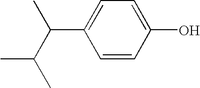 |
| 13 | 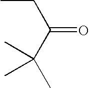 | 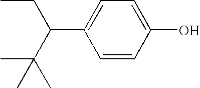 |

### Preparative Example 14

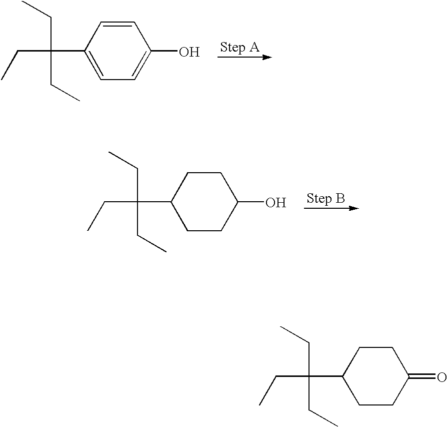 Step A:

The product from Preparative Example 1, Step A (1.0 g, 5.21 mmol) in hexanes (10 mL) and pH 7.4 phosphate buffer (10 mL) at 25° C. was treated with rhodium chloride hydrate (38% Rh w/w, 0.068 g, 0.323 mmol) and tetra-n-butylammonium sulfate (0.19 g, 0.55 mmol). The solution was hydrogenated for 20 h at 60 psi. The solution was filtered through a pad of Celite. The two layers were separated. The aqueous layer was extracted with EtOAc (3×25 mL) and the combined organic layers were washed with saturated NaCl (2×25 mL), dried (Na₂SO₄), filtered and concentrated under reduced pressure to yield a mixture of cis and trans isomeric products.

Step B:

A solution of Dess-Martin periodinane (2.16 g, 1.10 equiv.) in CH₂Cl₂ (13 mL) at 25° C. was treated with the product from Preparative Example 14, Step A (0.92 g, 4.64 mmol) in CH₂Cl₂ (5 mL). Trifluoroacetic acid (0.36 mL, 1.0 equiv.) was added and the solution was stirred 25° C. for 2 h. The solution was diluted with CH₂Cl₂ (18 mL) and Et₂O (60 mL). 1N aqueous NaOH (27 mL) was added dropwise and the mixture was stirred for 1 hour and the organic layer was separated. The organic layer was washed with 1N NaOH (30 mL) and H₂O (30 mL). The organic layer was dried (Na₂SO₄), filtered and concentrated under reduced pressure to give the ketone as an oil.

### Preparative Examples 15-32

By essentially the same procedure set forth in Preparative Example 14, only substituting the phenol shown in Column 2 of Table 3 in Step A, the compounds in Column 3 of Table 3 were prepared:

TABLE 3

| Prep. | | |
|---|---|---|
| Example | Column 2 | Column 3 |
| 15 | 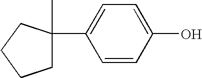 | 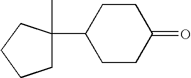 |
| 16 | 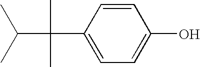 | 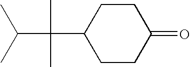 |
| 17 | 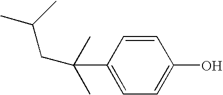 | 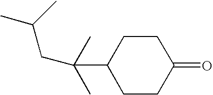 |
| 18 | 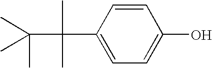 | 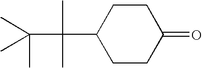 |
| 19 | 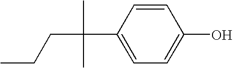 | 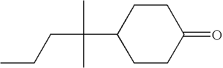 |
| 20 | 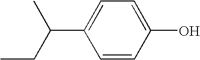 | 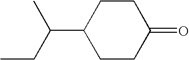 |
| 21 | 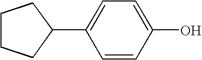 | 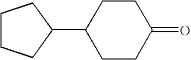 |
| 22 | 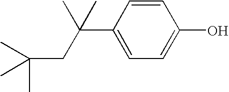 | 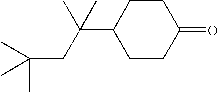 |
| 23 | 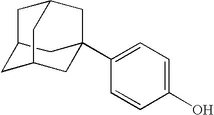 | 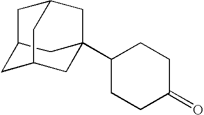 |
| 24 | 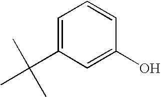 | 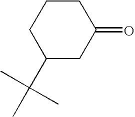 |
| 25 | 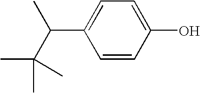 | 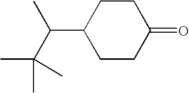 |
| 26 | 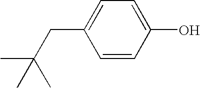 | 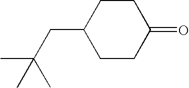 |
| 27 | 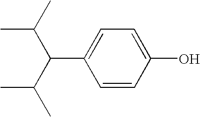 | 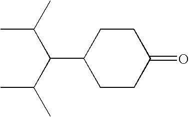 |
| 28 | 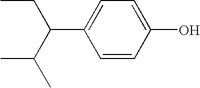 | 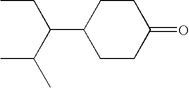 |
| 29 | 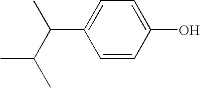 | 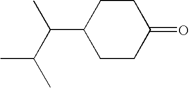 |
| 30 | 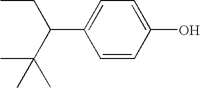 | 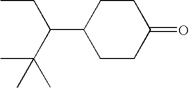 |
| 31 | 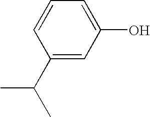 | 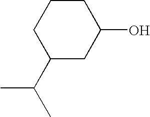 |
| 32 | 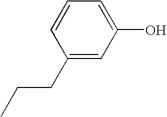 | 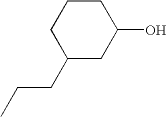 |

### Preparative Example 33

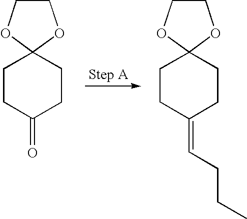 Step A:

Butyltriphenylphosphonium bromide (5.11 g, 1.98 equiv.) was suspended in anhydrous 1,2-dimethoxyethane (25 mL). n-Butyllithium (4.9 mL, 2.5M in hexanes, 1.9 equiv.) was added dropwise and the reaction was stirred for 60 min. Cyclohexadione-mono-ethylene ketal (1.01 g, 6.45 mmol) was dissolved in anhydrous DME (3 mL) and added to the reaction mixture, and the reaction was stirred 15 h at room temperature. The reaction was then heated to 70° C. and stirred for 2 days. After cooling, the reaction was evaporated to dryness under reduced pressure. The residue was suspended in dichloromethane, and purified by flash chromatography to yield (4-(2-butylidene)cyclohexanone ethylene ketal (56% yield).

### Preparative Examples 34-39

By essentially the same procedure set forth in Preparative Example 33, only substituting the triphenylphosphonium halide shown in Column 2 of Table 4 in Step A, the compounds in Column 3 of Table 4 were prepared:

TABLE 4

| Prep. | | |
|---|---|---|
| Example | Column 2 | Column 3 |
| 34 | 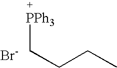 | 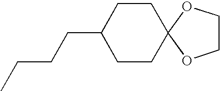 |
| 35 | 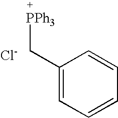 | 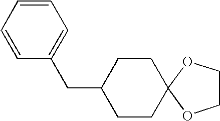 |
| 36 | 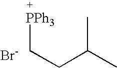 | 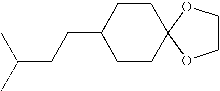 |
| 37 | 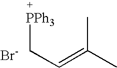 | 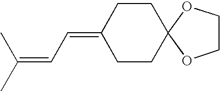 |
| 38 | 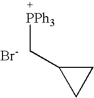 | 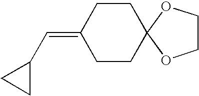 |
| 39 | 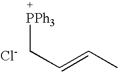 | 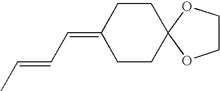 |

### Preparative Example 40

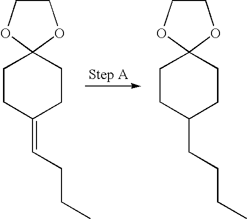 Step A:

The product from Preparative Example 33 (0.70 g, 3.55 mmol) dissolved in EtOAc (40 mL) was treated with 10% palladium on carbon (0.429 g). The mixture was hydrogenated at 1 atmosphere for 14 h. The mixture was filtered through Celite, and EtOAc was removed under reduced pressure to yield 4-butylcyclohexanone ethylene ketal (0.673 g) in 95% yield.

### Preparative Examples 41-42

By essentially the same procedure set forth in Preparative Example 40, only substituting the ketal shown in Column 2 of Table 5 in Step A, the compounds in Column 3 were prepared:

TABLE 5

| Prep. | | |
|---|---|---|
| Example | Column 2 | Column 3 |
| 41 | 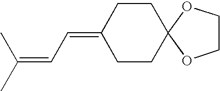 | 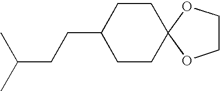 |
| 42 | 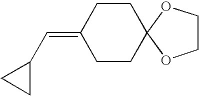 | 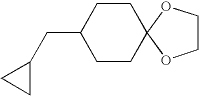 |

### Preparative Example 43

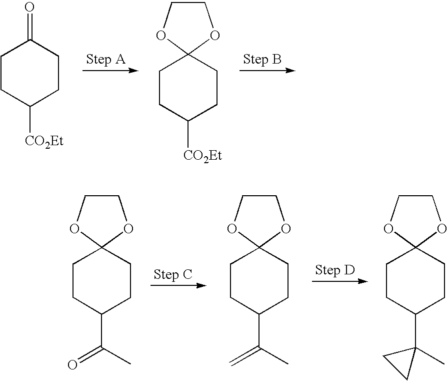 Step A:

Ethyl 4-oxocyclohexanecarboxylate (15.01 g, 88.16 mmol) was combined with ethylene glycol (21 mL, 4.27 equiv.) and p-toluenesulfonic acid monohydrate (0.200 g, 0.012 equiv.) in anhydrous toluene (50 mL), and the mixture was stirred 14 h at room temperature. The reaction was diluted with ether (200 mL) and was washed with H₂O (2×200 mL), saturated sodium bicarbonate (100 mL) and brine (80 mL). The organic layer was dried (Na₂SO₄), filtered and concentrated under reduced pressure to yield 18.15 g ethyl 4-oxocyclohexanecarboxylate ethylene ketal (96% yield).

Step B:

Ethyl 4-oxocyclohexanecarboxylate ethylene ketal (5.01 g, 23.42 mmol) was dissolved in anhydrous THF (50 mL). N,O-Dimethylhydroxylamine hydrochloride (2.971 g, 1.30 equiv.) was added and the suspension was cooled to −20° C. Methylmagnesium chloride (25 mL, 3M in THF, 3.2 equiv.) was added dropwise, and the reaction was stirred 1 hour at −20° C. to −10° C. Methylmagnesium chloride (40 mL, 3M in THF, 5.1 equiv.) was added, and the reaction was stirred 1.5 h at −10° C. to 0° C. The reaction was quenched with saturated ammonium chloride (50 mL) and H₂O (50 mL), and 4N aqueous HCl (30 mL) was then added to break up magnesium salt complexes. The mixture was extracted with ether (2×200 mL), and the combined ether extracts were washed with brine, dried (Na₂SO₄), filtered and concentrated under reduced pressure to yield 4.26 g, 4-acetylcyclohexanone ethylene ketal (99% yield).

Step C:

Methyltriphenylphosphonium bromide (10.34 g, 1.25 equiv.) was dissolved in anhydrous dimethylsulfoxide (35 mL) and n-butyllithium (12 mL, 2.5M in hexanes, 1.3 equiv.) was added dropwise at room temperature. After stirring 45 min, 4-acetylcyclohexanone ethylene ketal (4.273 g, 23.2 mmol) in dimethylsulfoxide (10 mL) was added dropwise. The reaction was stirred 14 h at 50° C. The reaction was cooled to 5° C., quenched slowly with H₂O (100 mL) and extracted with ether (2×150 mL). The combined organic extracts were washed with brine, dried (Na₂SO₄), filtered and concentrated under reduced pressure. The crude product was purified by flash chromatography to yield 3.61 g 4-isopropenylcyclohexanone ethylene ketal (85% yield).

Step D:

4-Isopropenylcyclohexanone ethylene ketal (1.18 g, 6.5 mmol), diiodomethane (2.7 mL, 5.15 equiv.), zinc-copper couple (3.88 g), and iodine (2 flakes) were combined in anhydrous 1,3-dimethoxyethane (70 mL), and stirred for 4 days at 70° C. After cooling to room temperature, the mixture was filtered through Celite. Saturated ammonium chloride (60 mL) and H₂O (60 mL) were added, and the organic layer was separated. The aqueous layer was extracted with EtOAc (100 mL), and the combined organic layers were dried (Na₂SO₄), filtered and concentrated under reduced pressure. The crude product was purified by flash chromatography to yield 0.98 g of an inseparable mixture of starting material (34% recovery) and 4-(1-methyl cyclopropyl)cyclohexanone ethylene ketal (45% yield) in a 1:1.33 ratio.

The ketal mixture (containing 2.19 mmol alkene and 2.92 mmol cyclopropane) was dissolved in acetone (40 mL) and H₂O (10 mL), and 4-methylmorpholine-N-oxide (1.01 g, 8.6 mmol, 3.9 equiv. based on alkene) and 4 wt % osmium tetroxide in H₂O (1.0 mL, 0.157 mmol, 0.07 equiv. based on alkene) were added. The reaction was stirred for 4 h at room temperature. Sodium bisulfite (1.03 g) was added and the reaction was stirred an additional 45 min. The reaction was diluted with brine (40 mL) and extracted with EtOAc (40 mL). EtOAc was washed with H₂O, washed with brine, dried (Na₂SO₄), filtered and concentrated under reduced pressure. The crude material was purified by flash chromatography to yield 0.525 g pure 4-(1-methyl cyclopropyl)cyclohexanone ethylene ketal (92% yield).

### Preparative Example 44

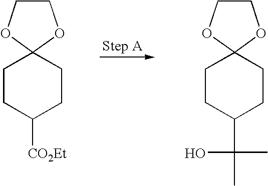 Step A:

Ethyl 4-oxocyclohexanecarboxylate ethylene ketal (1.203 g, 5.62 mmol) was dissolved in anhydrous ether (25 mL), and methylmagnesium bromide (5.6 mL, 3M in ether, 3.0 equiv.) was added dropwise at room temperature. The reaction was refluxed 3.5 h, and then quenched with saturated ammonium chloride (10 mL) and H₂O (10 mL). The mixture was extracted with EtOAc (3×20 mL), and the combined extracts were washed with brine, dried (Na₂SO₄), filtered and concentrated under reduced pressure to yield 1.12 g of 4-(1-hydroxy-1-methylethyl)cyclohexanone ethylene ketal (99% yield).

### Preparative Example 45

By essentially the same procedure set forth in Preparative Example 44, only substituting the Grignard shown in Column 2 of Table 6 in Step B, the compound in Column 3 was prepared:

TABLE 6

| Prep. | | |
|---|---|---|
| Example | Column 2 | Column 3 |
| 45 | 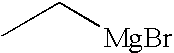 | 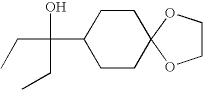 |

### Preparative Example 46

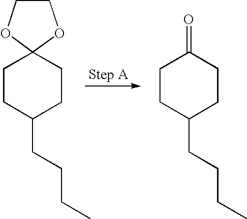 Step A:

The product from Preparative Example 40 (0.67 g, 3.37 mmol) was stirred for 14 h in THF (4 mL) and 4N aqueous HCl (4 mL). The reaction was quenched with saturated sodium bicarbonate (12 mL) and extracted with EtOAc (3×25 mL). The combined organic layers were washed with brine, dried (Na₂SO₄), filtered and concentrated under reduced pressure to yield 4-butylcyclohexanone (0.49 g, 94% yield).

### Preparative Examples 47-55

By essentially the same procedure set forth in Preparative Example 46, only substituting the ketal shown in Column 2 of Table 7 in Step A, the compounds in Column 3 were prepared:

TABLE 7

| Prep. Example | Column 2 | Column 3 |
|---|---|---|
| 47 | 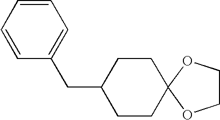 | 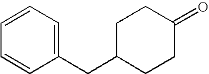 |
| 48 | 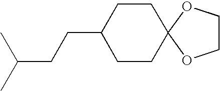 | 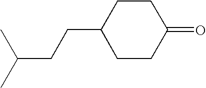 |
| 49 | 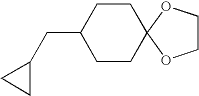 | 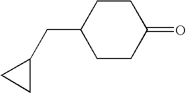 |
| 50 | 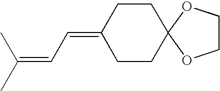 | 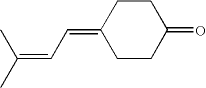 |
| 51 | 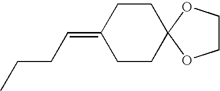 | 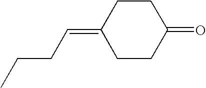 |
| 52 | 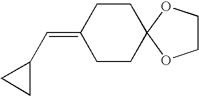 | 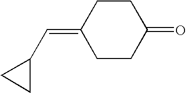 |
| 53 | 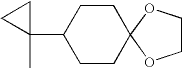 | 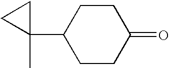 |
| 54 | 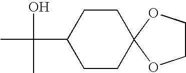 | 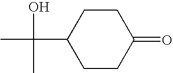 |
| 55 | 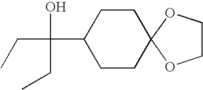 | 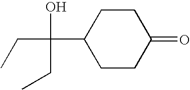 |

### Preparative Example 56

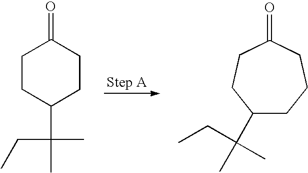 Step A:

A solution of 4-tert-amyl-cyclohexanone (5.94 mmol) in CH₂Cl₂ (60 mL) at −13° C. was treated with boron trifluoride diethyl etherate (1.5 equiv.). Trimethylsilyl diazomethane (2M solution in hexanes, 1.5 equiv.) was added dropwise over the period of 20 min. The solution was stirred −13° C. to −10° C. for 2 h and gradually warmed to 25° C. The solution was poured into ice-H₂O and extracted with CH₂Cl₂ (3×10 mL). The organic extracts were combined, washed with aqueous saturated NaCl (20 mL), dried (Na₂SO₄) and concentrated under reduced pressure. The oily residue was used without further purification.

### Preparative Example 57

By essentially the same procedure set forth in Preparative Example 56, only substituting the ketone shown in Column 2 of Table 8 in Step A, the compound in Column 3 was prepared:

TABLE 8

| Prep. | | |
|---|---|---|
| Example | Column 2 | Column 3 |
| 57 | 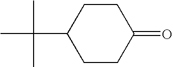 | 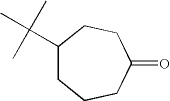 |

### Preparative Example 58

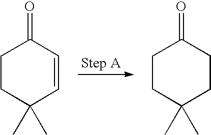 Step A:

4,4-Dimethylcyclohexenone (2.01 g, 16.2 mmol) was dissolved in pentane (50 mL) and hydrogenated 14 h at 1 atmosphere with 10% palladium on carbon catalyst (0.05 g). The reaction mixture was filtered through Celite and concentrated under reduced pressure to yield 1.54 g of 4,4-dimethylcyclohexanone (75% yield).

### Example 1

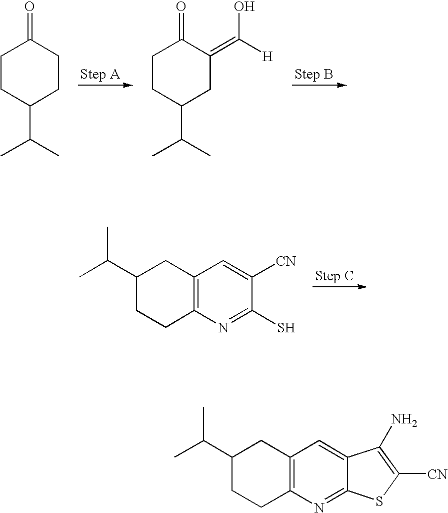 Step A:

Sodium hydride 60% dispersion in mineral oil (0.225 g, 1.54 equiv.) was suspended in anhydrous ether (12 mL) and cooled to 0° C. 4-isopropylcyclohexanone (0.511 g, 3.64 mmol) and ethyl formate (0.45 mL, 1.53 equiv.) were dissolved in anhydrous ether (5 mL) and added to the NaH suspension. Ethanol (0.15 mL, 0.7 equiv.) was added and the reaction was stirred at 0° C. for 5 h and gradually warmed to 25° C. The suspension was extracted with H₂O (1×15 mL, 2×10 mL), and the combined aqueous extracts were acidified to pH 3 with 4N aqueous HCl (1.15 mL). The resulting suspension was extracted with ether (1×25 mL, 1×15 mL, 1×10 mL), and the combined ether extracts were washed with brine, dried (Na₂SO₄), filtered and concentrated under reduced pressure to yield 0.537 g 2-formyl-4-isopropyl cyclohexanone (88% yield).

Step B:

2-Formyl-4-isopropylcyclohexanone (0.526 g, 3.13 mmol) was suspended in H₂O (6.5 mL), and a solution of piperidine acetate [prepared from piperidine (0.94 mL, 3 equiv.), acetic acid (0.54 mL, 3 equiv.) and H₂O (1.8 mL)] was added, followed by 2-cyanothioacetamide (0.323 g, 1.03 equiv.). The mixture was heated to 100° C. over 15 min., and then stirred for 40 min. at 100° C. Acetic acid (2 mL) was added, and the reaction mixture was slowly cooled to room temperature. The reaction was filtered and the resulting solid was dried under vacuum. The crude 2-mercapto-6-isopropyl-5,6,7,8-tetrahydroquinoline-3-carbonitrile product (0.275 g) was used without further purification.

Step C:

The crude mercapto-nitrile (0.265 g) was dissolved in dimethylformamide (3 mL) and 2-chloroacetonitrile (0.075 mL, 1.19 mmol) was added. The solution was cooled to 0° C., and 20% aqueous potassium hydroxide (0.52 mL, 1.85 mmol) was added. The reaction was stirred for 3 h at 0° C. to 4° C., then diluted with ice-water (16 mL). After the ice had melted, the resulting suspension was filtered, and the filter residue was taken up in acetone and concentrated under reduced pressure. The residue was purified by flash chromatography to yield 0.159 g of 3-amino-6-isopropyl-5,6,7,8-tetrahydrothieno[2,3-b]quinoline-2-carbonitrile in 51% yield (from formylcyclohexanone).

### Examples 3-52

By essentially the same procedure set forth in Example 1, only substituting the ketone shown in Column 2 of Table 9 in Step A, the compounds in Column 3 were prepared:

TABLE 9

| Example | Column 2 | Column 3 | CMPD |
|---|---|---|---|
| 3 | 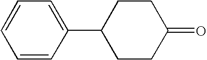 | 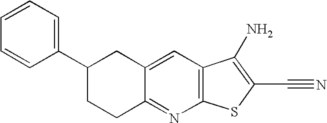 | MS: MH⁺ =306; mp (° C.) =230-232 |
| 4 | 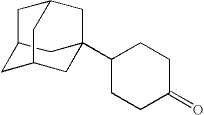 | 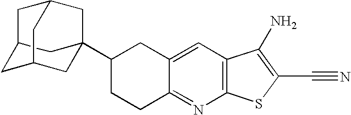 | MS: MH⁺ =364; mp (° C.) =>275(dec.) |
| 5 | 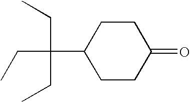 | 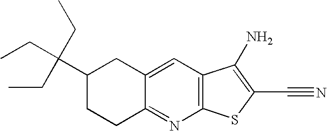 | MS: MH⁺ =328; mp (° C.) =208-211 |
| 6 | 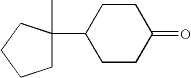 | 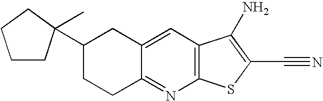 | MS: MH⁺ =312; mp (° C.) =234-236 |
| 7 | 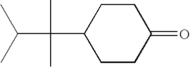 | 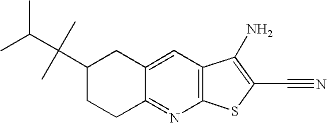 | MS: MH⁺ =314; mp (° C.) =203-205 |
| 8 | 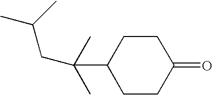 | 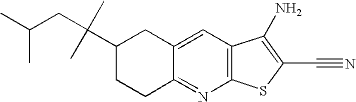 | MS: MH⁺ =328; mp (° C.) =180-182 |
| 9 | 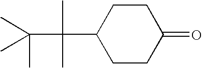 | 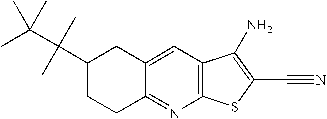 | MS: MH⁺ =328; mp (° C.) = 190 (dec.) |
| 10 | 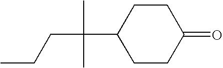 | 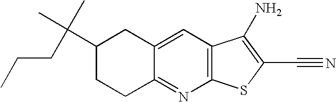 | MS: MH⁺ =314; mp (° C.) =175-179 |
| 11 | 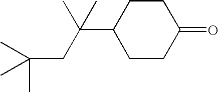 | 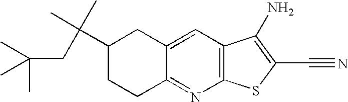 | ¹H NMR (DMSO-d₆): δ8.176(s, 1H), 7.134(br s, 2H), 2.75-3.1 (m, 3H), 2.6-2.7 (m, 1H), 1.95-2.10(m, 1H), 1.55-1.70 (m, 1H), 1.3-1.55(m, 1H), 1.334(s, 2H), 0.974(s, 15H); MS: MH+ = 341 |
| 12 | 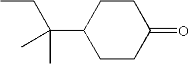 | 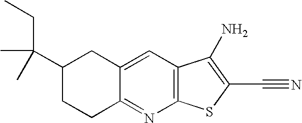 | MS: MH⁺ =300; mp (° C.) =211-213 |
| 13 | 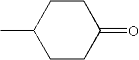 | 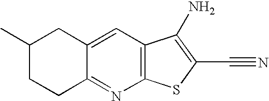 | MS: MH⁺ =244; mp (° C.) =228-230 |
| 14 | 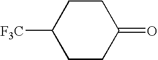 | 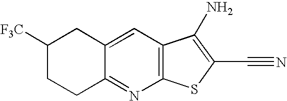 | ¹H NMR (DMSO-d₆): δ8.224(s, 1H), 7.201(br s, 2H), 2.8-3.1 (m, 5H), 2.1-2.2 (m, 1H), 1.7-1.9(m, 1H); MS: MH⁺ =298 |
| 15 | 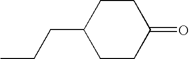 | 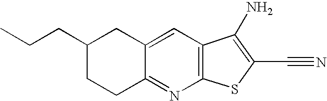 | MS: MH⁺ =272; mp (° C.) =171-173 |
| 16 | 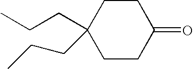 | 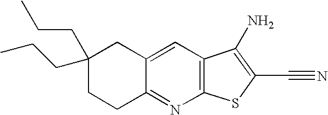 | MS: MH⁺ =314; mp (° C.) =176-178 |
| 17 | 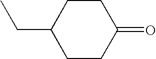 | 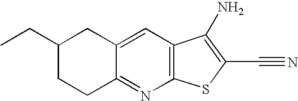 | MS: MH⁺ =258; mp (° C.) =189-193 |
| 18 | 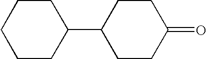 | 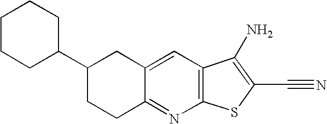 | MS: MH⁺ =311; mp (° C.) =166-170 |
| 19 | 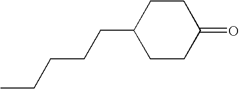 | 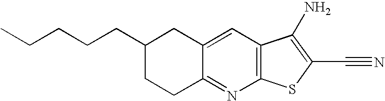 | MS: MH⁺ =300; mp (° C.) =156-159 |
| 20 | 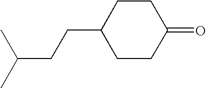 | 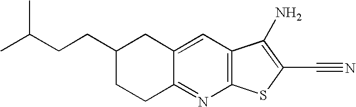 | ¹H NMR (DMSO-d₆): δ8.131(s, 1H), 7.156(br s, 2H), 2.8-3.0 (m, 3H), 2.4-2.6 (m, 1H), 1.9-2.0(m, 1H), 1.6-1.8 (m, 1H), 1.2-1.6 (m, 6H), 0.858(d, J=6.8 Hz, 6H); MS: MH⁺ =300 |
| 21 | 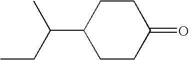 | 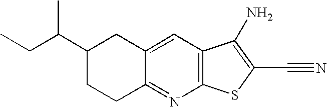 | ¹H NMR (DMSO-d₆): δ8.138(s, 1H), 7.142(br s, 2H), 2.5-3.0(m, 4H), 1.85-1.95 (m, 1H), 1.6-1.7(m, 1H), 1.3-1.6 (m, 3H), 1.1-1.3 (m, 1H), 0.868(d, J=6.8 Hz, 3H), 0.860(t, J=7.6 Hz, 3H); MS: MH⁺ =286 |
| 22 | 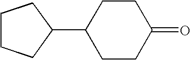 | 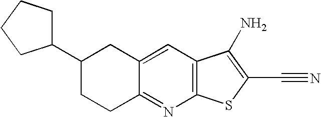 | ¹H NMR (DMSO-d₆): δ8.144(s, 1H), 7.147(br s, 2H), 2.8-3.0 (m, 3H), 2.5-2.6 (m, 1H), 1.9-2.1(m, 1H), 1.7-1.9 (m, 1H), 1.25-1.65 (m, 8H), 1.05-1.15(m, 2H); MS: MH⁺ =298 |
| 23 | 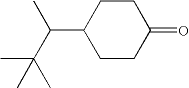 | 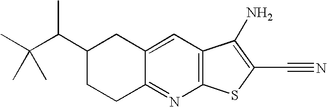 | MS: MH⁺ =314; mp (° C.) = 195 (dec.) |
| 24 | 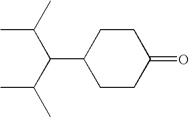 | 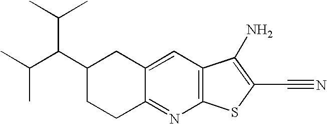 | ¹H NMR (DMSO-d₆): δ8.152(s, 1H), 7.140(br s, 2H), 2.85-3.0 (m, 2H), 2.7-2.8 (m, 2H), 1.8-2.0(m, 5H), 1.5-1.7 (m, 1H), 0.8-1.0 (m, 12H), MS: MH⁺ =328 |
| 25 | 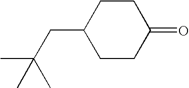 | 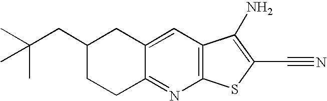 | MS: MH⁺ =300; mp (° C.) =196-199 |
| 26 | 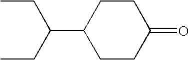 | 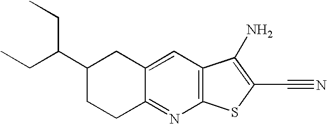 | MS: MH⁺ =300; mp (° C.) =198-200 |
| 27 | 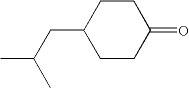 | 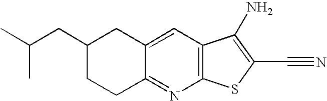 | MS: MH⁺ =286; mp =184-187° C. |
| 28 | 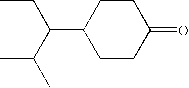 | 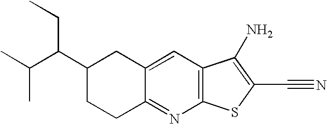 | MS: MH⁺ =314; mp (° C.) = 185 (dec.) |
| 29 | 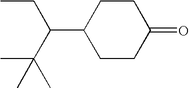 | 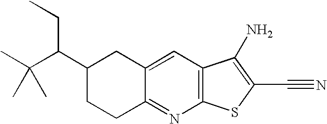 | MS: MH⁺ =328; mp (° C.) = 185 (dec.) |
| 30 | 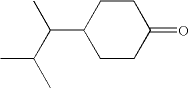 | 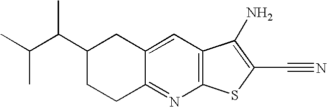 | MS: MH⁺ =300; mp (° C.) = 120 (dec.) |
| 31 | 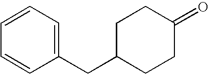 | 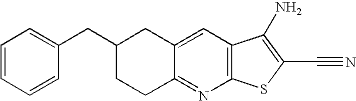 | ¹H NMR (DMSO-d₆): δ8.106(s, 1H), 7.15-7.4(m, 5H), 7.124(br s, 2H), 2.5-3.1 (m, 6H), 1.9-2.1 (m, 2H), 1.4-1.6(m, 1H); MS: MH⁺ =320 |
| 32 | 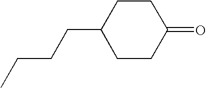 | 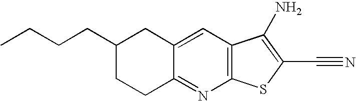 | ¹H NMR (DMSO-d₆): δ8.131(s, 1H), 7.155(br s, 2H), 2.8-3.0(m, 3H), 2.4-2.55 (m, 1H), 1.9-2.0(m, 1H), 1.6-1.8 (m, 1H), 1.2-1.5 (m, 7H), 0.870(t, J=7.0 Hz, 3H); MS: MH⁺ =286 |
| 33 | 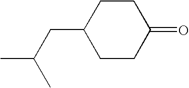 | 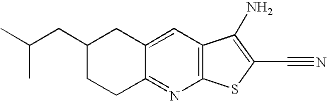 | MS: MH⁺ =286; mp (° C.) =184-187 |
| 34 | 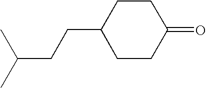 | 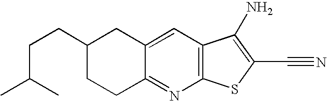 | ¹H NMR (DMSO-d₆): δ8.131(s, 1H), 7.156(br s, 2H), 2.8-3.0(m, 3H), 2.4-2.6 (m, 1H), 1.9-2.0(m, 1H), 1.6-1.8 (m, 1H), 1.2-1.6 (m, 6H), 0.858(d, J=6.8 Hz, 6H); MS: MH⁺ =300 |
| 35 | 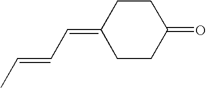 | 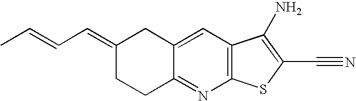 | MS: MH⁺ =282; mp (° C.) =145-153 |
| 36 | 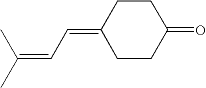 | 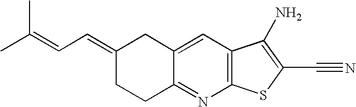 | MS: MH⁺ =296; mp =140° C. (dec.) |
| 37 | 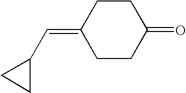 | 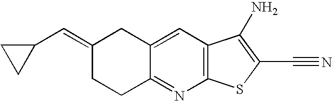 | MS: MH⁺ =282; mp (° C.) = 130 (dec.) |
| 38 | 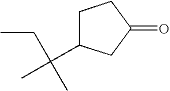 | 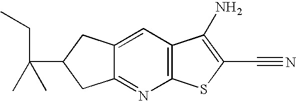 | MS: MH⁺ =286; mp (° C.) =225-228 |
| 39 | 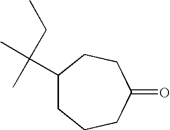 | 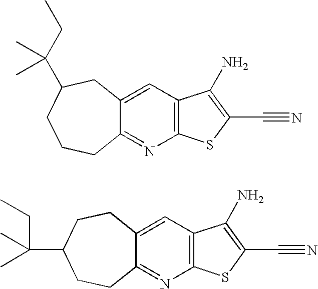 | ¹H NMR (CDCl₃, inseparable mixture of regioisomers): δ 7.61-7.52 (m, 1H), 4.76-4.75 (m, 2H), 3.26-2.65(m, 4H), 2.18-2.08 (m, 2H), 1.52-1.03(m, 7H), 0.84-0.78 (m, 9H); MS: MH⁺ =314 |
| 40 | 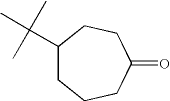 | 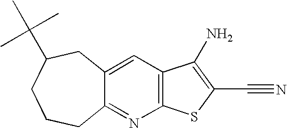 | MS: MH⁺ =300; mp (° C.) =213-215 |
| 41 | 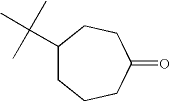 | 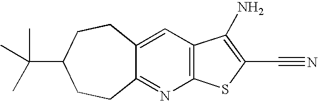 | MS: MH⁺ =300; mp (° C.) =193-195 |
| 42 | 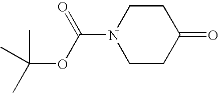 | 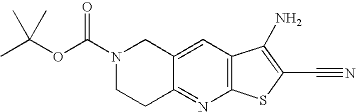 | MS: MH⁺ =300; mp (° C.) =218-220 |
| 43 | 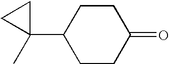 | 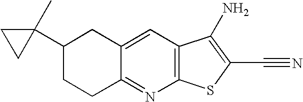 | MS: MH⁺ =284; mp (° C.) =241-242 |
| 44 | 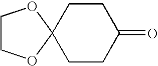 | 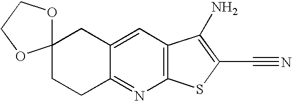 | MS: MH⁺ =288; mp (° C.) =248-250 |
| 45 | 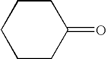 | 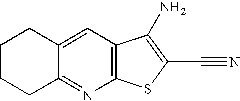 | MS: MH⁺ =230; mp (° C.) =263-265 |
| 46 | 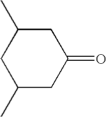 | 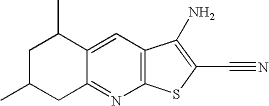 | ¹H NMR (DMSO-d₆): δ8.426(s, 1H), 7.219(br s, 1H), 2.8-3.0(m, 2H), 2.5-2.6 (m, 1H), 1.8-2.0(m, 2H), 1.31(d, J=6.8 Hz, 3H), 1.05-1.2(m, 1H), 1.02(d, J=6.0 Hz, 3H); MS: MH⁺ = 258 |
| 47 | 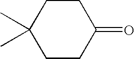 | 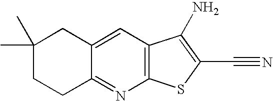 | MS: MH⁺ =258; mp (° C.) =218-220 |
| 48 | 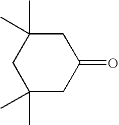 | 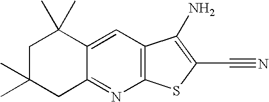 | ¹H NMR (DMSO-d₆): δ8.661(s, 1H), 7.219(br s, 2H), 2.714(s, 2H), 1.595(s, 2H), 1.311(s, 6H), 0.940(s, 6H); MS: MH⁺ =286 |
| 49 | 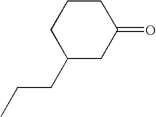 | 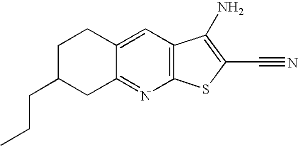 | ¹H NMR (DMSO-d₆): δ8.150(s, 1H), 7.153(br s, 2H), 2.9-3.0(m, 1H), 2.8-2.9 (m, 1H), 2.5-2.6(m, 1H), 1.7-1.9 (m, 2H), 1.25-1.45 (m, 5H), 0.877(t, J=7 Hz, 3H); MS: MH⁺ = 272 |
| 50 | 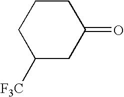 | 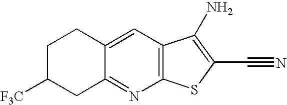 | ¹H NMR (DMSO-d₆): δ8.222(s, 1H), 7.207(br s, 2H), 3.05-3.15 (m, 1H), 2.85-3.0(m, 4H), 2.05-2.20 (m, 4H), 1.6-1.8(m, 1H); MS: MH⁺ =298 |
| 51 | 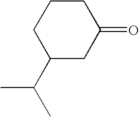 | 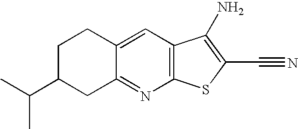 | ¹H NMR (DMSO-d₆): δ8.133(s, 1H), 7.140(br s, 2H), 2.7-3.0(m, 3H), 2.5-2.7 (m, 1H), 1.8-2.0(m, 1H), 1.5-1.6 (m, 2H), 1.3-1.4 (m, 1H), 0.908(d, J=6.0 Hz, 6H); MS: MH⁺ = 272 |
| 52 | 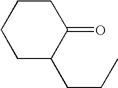 | 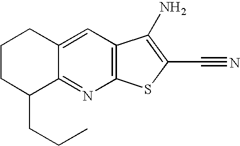 | MS: MH⁺ =272; mp (° C.) =130-133 |

### Example 53

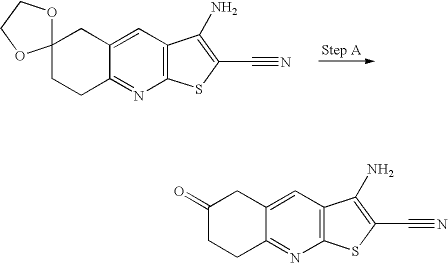 Step A:

Example 53 was prepared according to the conditions listed in Preparative Example 46.

### Example 54

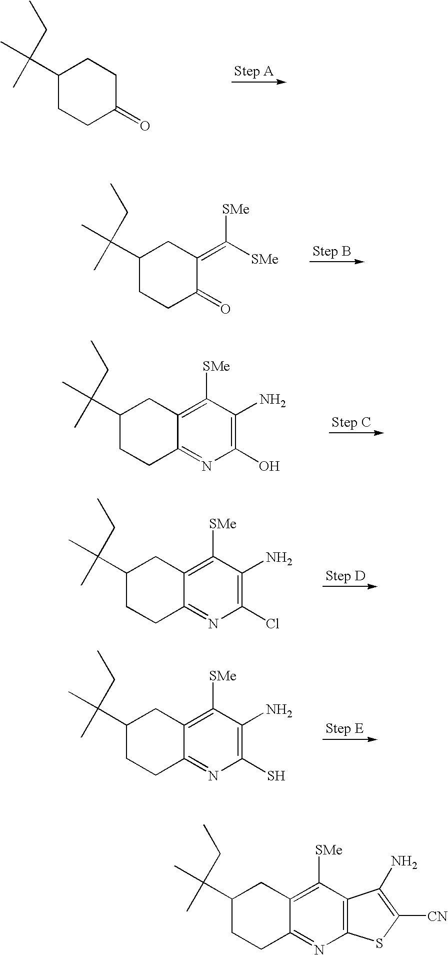 Step A:

A solution of 4-tert-amyl cyclohexanone (1.0 g, 5.94 mmol) in THF 24 mL at −78° C. was treated with NaHMDS (11.9 mL, 2 equiv.). The solution was stirred at −78° C. for 1 h. CS₂ (0.36 mL, 1 equiv.) was added dropwise over several min. and stirring was continued at −78° C. for 0.5 h. Mel (0.81 mL, 2.2 equiv.) was added dropwise and stirring was continued at −78° C. for 2 h. The solution was gradually warmed to 25° C. and stirring was continued for 10 h. The solution was quenched by the addition of H₂O (50 mL). The aqueous layer was treated with aqueous saturated NH₄Cl. The aqueous layer was extracted with CH₂Cl₂ (3×20 mL). The combined organic layers were extracted with saturated aqueous NaCl (10 mL), dried (Na₂SO₄) and concentrated under reduced pressure. The residue was purified by flash chromatography eluting with 10% EtOAc-hexanes to give 0.339 g (21%).

Step B:

A solution of Na^{o} (0.018 g, 1.0 equiv.) dissolved in EtOH (3 mL) was treated with 2-cyanoacetamide (0.067 g, 1.0 equiv.). The solution was stirred at 25° C. for 0.25 h. The product prepared in Step A of Example 54 (0.21 g, 0.793 mmol) in EtOH (1 mL) was added dropwise. The solution was heated at reflux for 18 h. The solution was concentrated in vacuo and the residue was diluted with H₂O (6 mL). The aqueous layer was adjusted to pH=4 with AcOH (0.5 mL). The yellow precipitate was filtered and dried under vacuum. The residue was purified by flash chromatography eluting with 50% EtOAc-hexanes to give 0.055 g (24%).

Step C:

A solution of product prepared from Step B in Example 54 (0.055 g, 0.189 mmol) in phenylphosphonic dichloride (0.5 mL) was heated at 180° C. for 1 h. The solution was gradually cooled to 25° C. and diluted with ice (5 g). The pH was adjusted to ˜9-10 with concentrated NH₄OH (˜1 mL). The precipitate was filtered and dried under vacuum to provide 0.0511 g of a crude product that was used directly in the next step.

Step D:

A solution of product prepared from Step C in Example 54 (0.051 g, 0.165 mmol) in H₂O/EtOH (1:2, 1.65 mL) at 25° C. was treated with thiourea (0.19 g, 15 equiv.). The solution was heated at reflux for 17 h and cooled to 25° C. The solution was diluted with H₂O (6 ml). The aqueous layer was extracted with EtOAc (3×5 mL). The combined organic layers were washed with saturated aqueous NaCl (10 mL), dried (Na₂SO₄) and concentrated under reduced pressure to provide 0.0493 g of a crude product that was used directly in the next step.

Step E:

The product was prepared by essentially the same procedure in Step C in Example 1. The residue was purified by flash chromatography eluting with CH₂Cl₂. MS: MH⁺=346; mp (° C.)=169 (dec.).

### Example 55

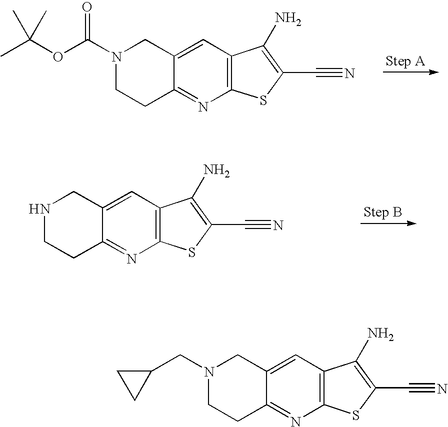 Step A:

A solution of Example 42 (0.202 g, 0.611 mmol) in CH₂Cl₂ (2.4 mL) at 25° C. was treated with trifluoroactic acid (1 mL). The solution was stirred at 25° C. for 1 h and concentrated in vacuo. The crude residue was diluted with Et₂O (6 mL) and the precipitate was filtered and dried under vacuum. The crude precipitate was used directly in the next step (79%).

Step B:

A solution of product prepared in Step A of Example 55 (0.050 g, 0.22 mmol) in CH₃CN (2.2 mL) at 25° C. was treated with K₂CO₃ (0.09 g, 3.0 equiv.) and (bromomethyl)cyclopropane (0.023 mL, 1.1 equiv.). The solution was heated at 70° C. for 60 h. The solution was cooled to 25° C. and diluted with H₂O (10 mL). The aqueous layer was extracted with CH₂Cl₂ (3×3 mL). The combined organic layers were extracted with saturated aqueous NaCl (10 mL), dried (Na₂SO₄) and concentrated under reduced pressure. MS: MH⁺=285; mp (° C.)=175 (dec.).

### Examples 56-57

By essentially the same procedure set forth in Example 55, only substituting the alkyl halide shown in Column 2 of Table 10 in Step A, the compounds in Column 3 were prepared:

TABLE 10

| Example | Column 2 | Column 3 | CMPD |
|---|---|---|---|
| 56 | 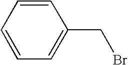 | 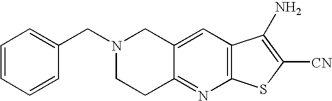 | MS: MH⁺ =321; mp (° C.) = 194 (dec.) |
| 57 | 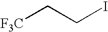 | 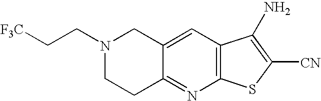 | MS: MH⁺ =327; mp (° C.) =158-160 |

### Example 58

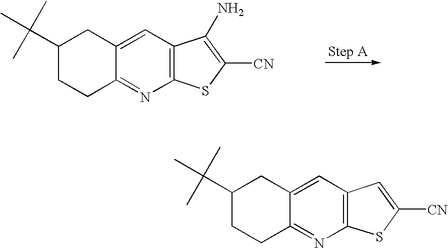 Step A:

6-tert-Butyl-5,6,7,8-tetrahydrothieno[2,3-b]quinoline-2-carbonitrile: To a solution of 90% t-butylnitrite (526 mg, 4.60 mmol) in 6 mL of DMF stirred at 65° C., was added a solution of 3-amino-6-tert-butyl-5,6,7,8-tetrahydrothieno[2,3-b]quinoline-2-carbonitrile (820 mg, 2.87 mmol) in 6 mL of DMF dropwise. The reaction was stirred at 65° C. for 30 min. Upon cooling to room temperature, it was added into 100 mL of H₂O. This was extracted by 100 mL of EtOAc. The organic phase was dried over anhydrous Na₂SO₄ and then concentrated. The residue was purified by flash chromatography eluting with 15% EtOAc/hexanes to give 500 mg (64%) of 6-tert-butyl-5,6,7,8-tetrahydrothieno[2,3-b]quinoline-2-carbonitrile. LCMS: MH⁺=271; mp (° C.)=133-135.

### Examples 59-63

By essentially the same procedure set forth in Example 58, only substituting the compound shown in Column 2 of Table 11 in Step A, the compounds in Column 3 were prepared. For compounds 62 and 63, the initial racemic mixture of enantiomers (compound 58) resulting after the performance of essentially the same procedure of Step A (Example 58) was passed through a chiral column to give compound 62, the (−)-enantiomer and compound 63, the (+)-enantiomer set forth in Table 11 below. The chiral separation conditions were as follows: Column: Chiralpak AD-H (3 cm i.d×25 cm L); Eluent: CO₂/MeOH (85/15); Temperature: 30° C.; Detection: UV 220 nm.

TABLE 11

| Example | Column 2 | Column 3 | CMPD |
|---|---|---|---|
| 59 | 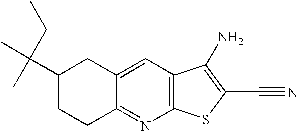 | 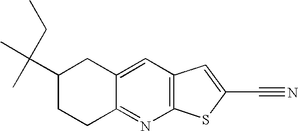 | MS: MH⁺ =285; mp (° C.) =90-93 |
| 60 | 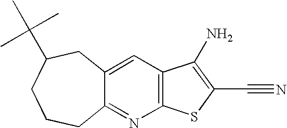 | 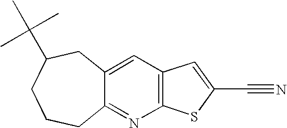 | MS: MH⁺ =285; mp (° C.) =145-147 |
| 61 | 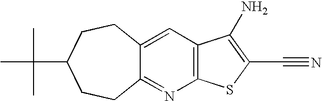 | 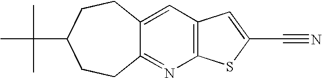 | MS: MH⁺ =285; mp (° C.) =141-143 |
| 62 | 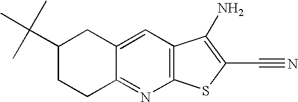 | 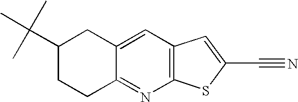 | ¹H NMR (DMSO- d₆): δ 8.29 (s, 1H), 8.14(s, 1H), 3.13-3.07 (m, 1H), 3.01-2.91 (s, 2H), 2.69-2.62 (m, 1H), 2.07-2.03 (m, 1H), 1.55-1.38 (m, 2H), 0.95 (s, 9H); MS: MH⁺ =271. |
| 63 | 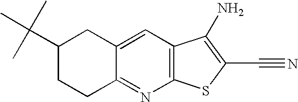 | 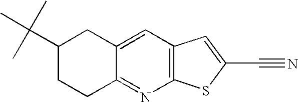 | ¹H NMR (DMSO- d₆): δ 8.29 (s, 1H), 8.14(s, 1H), 3.13-3.07 (m, 1H), 3.01-2.91 (s, 2H), 2.69-2.62 (m, 1H), 2.07-2.03 (m, 1H), 1.55-1.38 (m, 2H), 0.95 (s, 9H); MS: MH⁺ =271. |

### Example 64

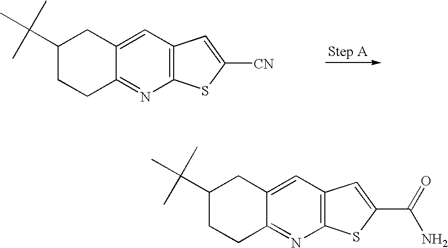 Step A:

6-tert-Butyl-5,6,7,8-tetrahydrothieno[2,3-b]quinoline-2-carboxylic acid amide: A mixture of 6-tert-butyl-5,6,7,8-tetrahydrothieno[2,3-b]quinoline-2-carbonitrile (25 mg, 0.092 mmol) in 0.8 mL of polyphosphoric acid was stirred at 120° C. for 4 h. After it was cooled to room temperature, 20 mL of ice H₂O was added. The solid was collected by filtration and washed with H₂O to give 20 mg (75%) of 6-tert-butyl-5,6,7,8-tetrahydrothieno[2,3-b]quinoline-2-carboxylic acid amide. LCMS: MH⁺=289; mp (° C.)=243-245.

### Examples 65-68

By essentially the same procedure set forth in Example 64, only substituting the compound shown in Column 2 of Table 12 in Step A, the compounds in Column 3 were prepared.

Compounds 67 and 68 can also be prepared as follows: The less polar ethyl 6-(1,1-dimethylethyl)-5,6,7,8-tetrahydro-thieno[2,3-b]quinoline-2-carboxylate (375 mg, 1.18 mmol; compound 110-1; see Examples 109-110) was dissolved in methanol and was cooled at 0° C. Ammonia was bubbled through the solution for 20 min. The mixture was then stirred in a sealed-tube for 2 days at r.t. Removal of solvents in vacuum gave a white solid. The solid was washed extensively with ether and dried under high vacuum gave (−)-6-(1,1-dimethylethyl)-5,6,7,8-tetrahydrothieno[2,3-b]quinoline-2-carboxamide (300 mg, 88%) (compound 67) as white solid. [α]_{D}²⁰−106° (MeOH, c=0.82), electrospray MS [M+1]⁺=289.

Similarly, the more polar ethyl 6-(1,1-dimethylethyl)-5,6,7,8-tetrahydro-thieno[2,3-b]quinoline-2-carboxylate (350 mg, 1.10 mmol; compound 110-2; see Examples 109-110) was converted to (+)-6-(1,1-dimethylethyl)-5,6,7,8-tetrahydrothieno[2,3-b]quinoline-2-carboxamide (273 mg, 85%) as white solid. [α]_{D}²⁰+105° (MeOH, c=0.70), electrospray MS [M+1]⁺=289.

TABLE 12

| Example | Column 2 | Column 3 | CMPD |
|---|---|---|---|
| 65 | 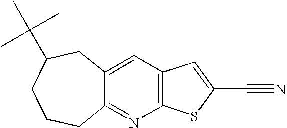 | 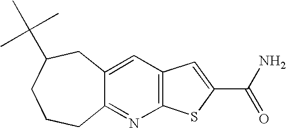 | MS: MH⁺ =303; mp (° C.) =240(dec.) |
| 66 | 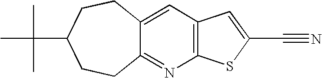 | 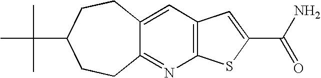 | MS: MH⁺ =303; mp (° C.) =230(dec.) |
| 67 | 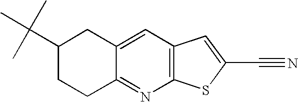 | 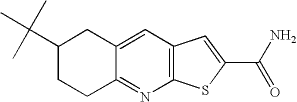 | ¹H NMR (DMSO- d₆): δ 8.22 (br s, 1H), 8.03(s, 1H), 7.93 (s, 1H), 7.64(br s, 1H), 3.08-3.03 (m, 1H), 2.98-2.87 (m, 2H), 2.67-2.60 (m, 1H), 2.07-2.03 (m, 1H), 1.55-1.38 (m, 2H), 0.96(s, 9H); MS: MH⁺ =289. |
| 68 | 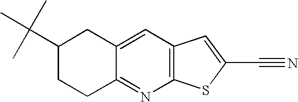 | 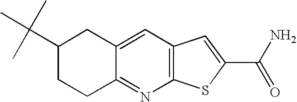 | ¹H NMR (DMSO- d₆): δ 8.22 (br s, 1H), 8.03(s, 1H), 7.93 (s, 1H), 7.64(br s, 1H), 3.08-3.03 (m, 1H), 2.98-2.87 (m, 2H), 2.67-2.60 (m, 1H), 2.07-2.03 (m, 1H), 1.55-1.38 (m, 2H), 0.96(s, 9H); MS: MH⁺ =289. |

### Example 69

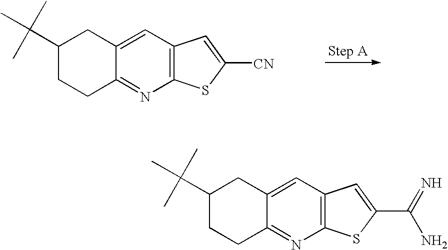 Step A:

6-tert-Butyl-5,6,7,8-tetrahydrothieno[2,3-b]quinoline-2-carboxamidine: A mixture of 6-tert-butyl-5,6,7,8-tetrahydrothieno[2,3 -b]quinoline-2-carbonitrile (160 mg, 0.593 mmol) and NH₄Cl (120 mg, 2.24 mmol) in 5 mL of 7 N NH₃ in MeOH was heated at 90° C. in a sealed tube for 16 h. Upon cooling to room temperature, it was diluted with 30 mL of CH₂Cl₂. The solution was washed with 15 mL of saturated aqueous NaHCO₃ and dried over anhydrous Na₂SO₄. The solvent was removed under vacuum to give 150 mg (88%) of 6-tert-butyl-5,6,7,8-tetrahydrothieno[2,3-b]quinoline-2-carboxamidine. LCMS: MH⁺=288; mp (° C.)=86-210 (dec.).

### Example 70

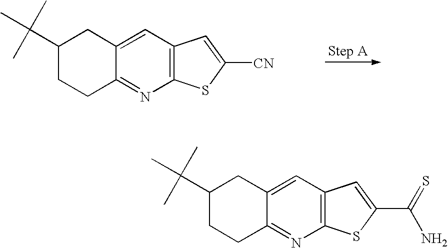 Step A:

6-tert-Butyl-5,6,7,8-tetrahydrothieno[2,3-b]quinoline-2-carbothioic acid amide: A mixture of 6-tert-butyl-5,6,7,8-tetrahydrothieno[2,3-b]quinoline-2-carbonitrile (60 mg, 0.22 mmol), NH₄Cl (20 mg, 0.37 mmol) and NaHS (60 mg, 1.1 mmol) in 2.5 mL of EtOH/H₂O (2:1) was refluxed under an atmosphere of N₂ for 0.5 h. Upon cooling to room temperature, 8 mL of H₂O was added. The resulting mixture was filtered. The yellow solid was washed with H₂O (5 mL), MeOH (3 mL) and hexanes (10 mL), then dried under vacuum to give 45 mg (60%) of 6-tert-butyl-5,6,7,8-tetrahydrothieno[2,3-b]quinoline-2-carbothioic acid amide. LCMS: MH⁺=305; mp (° C.)=252-258 (dec.).

### Example 71

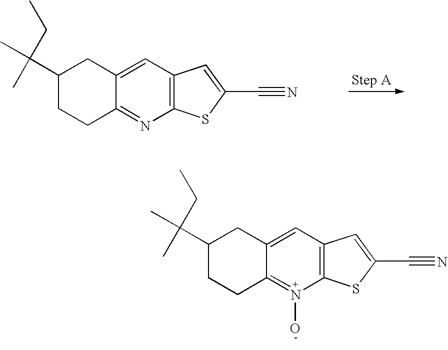 Step A:

A solution of the product from Example 59 (0.04 g, 0.14 mmol) in CH₂Cl₂ (1.4 mL) at 0° C. was treated with 3-chloroperoxybenzoic acid (0.05 g, 1.5 equiv.). The solution was stirred at 0° C. for 2 h and warmed to 25° C. The solution was diluted with CH₂Cl₂ (5.0 mL) and washed with aqueous saturated NaCl (3×5 mL). The combined organic layers were dried (Na₂SO₄), filtered and concentrated under reduced pressure to yield 0.039 g of the product (92%). The crude product was used in the next step without further purification. MS: MH⁺=301; mp=217-219° C.

### Example 72

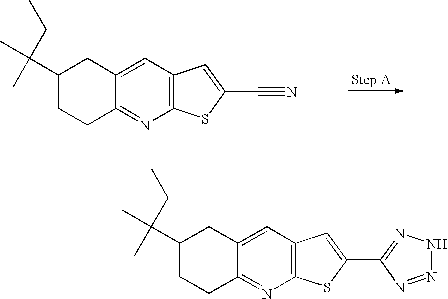 Step A:

A solution of the product from Example 59 (0.135 g, 0.475 mmol) in DMF (0.5 mL) at 25° C. was treated with NaN₃ (0.034 g, 1.1 equiv.) and NH₄Cl (0.028 g, 1.1 equiv.). The solution was heated at 100° C. for 68 h. The solution was cooled to 25° C. and treated with 1M HCl (2 mL). The solution was filtered and dried. MS: MH⁺=328; mp=207° C. (dec.).

### Example 73

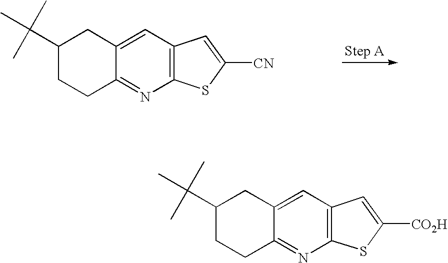 Step A:

6-tert-Butyl-5,6,7,8-tetrahydrothieno[2,3-b]quinoline-2-carboxylic acid: A mixture of 6-tert-butyl-5,6,7,8-tetrahydrothieno[2,3-b]quinoline-2-carbonitrile (195 mg, 0.72 mmol) in 3 mL of 85% phosphoric acid was stirred at 160° C. for 4 h. After it was cooled to room temperature, 20 mL of ice H₂O was added. The solid was collected by filtration, washed with H₂O and then dried under vacuum. The mother liquor was extracted with CH₂Cl₂. The organic phase was dried over anhydrous Na₂SO₄ and then concentrated under vacuum. The solid residue was combined with the solid from the previous filtration to give a total yield of 205 mg (98%) of 6-tert-butyl-5,6,7,8-tetrahydrothieno[2,3-b]quinoline-2-carboxylic acid. LCMS: MH⁺=290; mp (° C.)=269-272.

### Examples 74-76

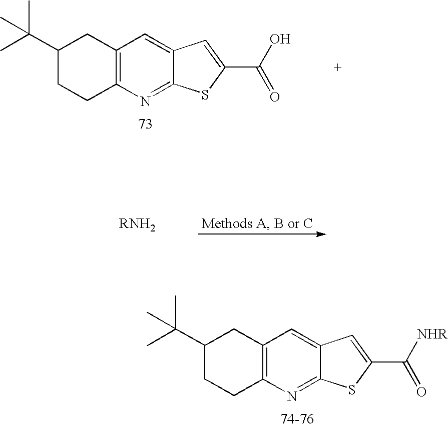

Method-A: EDCl/HOBt/NMM/CH₂Cl₂; Method-B: HATU/NMM/DMF; Methods: a) SOCl₂ b) RNH₂

Method-A: A solution of the carboxylic acid 73 (32.5 mg, 0.11 mmol), 3-ethyl-1(3-dimethyl aminopropyl)-carbodiimide hydrochloride (EDCl, 64.8 mg, 0.34 mmol), 1-hydroxy benzotriazole hydrate (HOBt, 45.5 mg, 0.34 mmol) and N-methylmorpholine (68.2 mg; 0.67 mmol) in CH₂Cl₂ was treated with methyl amine (2M solution in THF, 0.22 ml, 0.45 mmol). The resulting solution was stirred at room temperature (RT) for 16-20 hours. The reaction mixture was diluted with, CH₂Cl₂, washed with water, saturated NaHCO₃ solution and brine. The organic extract was dried over anhydrous MgSO₄ and concentrated in vacuo to obtain yellow oil. Flash Silica Gel Chromatography using 25-30% EtOAc in hexanes gave the N-methyl amide 74 (R═CH₃) as a white solid (18 mg, 53%). mp: 186-189° C. HRMS (MH⁺): Calc for C₁₇H₂₃N₂OS 303.0786. Found 303.0784.

Method-B: A solution of the carboxylic acid 73 (60.6 mg, 0.21 mmol) and (S)-(+)-2-amino-1-propanol (47.4 mg, 0.63 mmol) in 2 mL of DMF was treated with HATU (240 mg, 0.63 mmol) and N-methyl morpholine (0.14 mL, 1.25 mmol) and stirred at RT for 18 hours. Most of the DMF was removed on the rotary evaporator and the residue was dissolved in CH₂Cl₂ and washed with water, 1M aqueous HCl solution, saturated NAHCO₃ solution and brine. Concentration to crude product followed by FSGC (2% methanol in CH₂Cl₂) provided 37 mg (50%) of white solid 75 which is a mixture of diastereomers, i.e., mixture of (R)—(S) and (S)—(S) isomers {R=1-[1(S)-Methyl]-2-hydroxyethyl}. mp: 204° C. (dec). HRMS (M+1) Calc for C₁₉H₂₇N₂O₂S 347.1794. Found 347.1791.

Method-C: The tricyclic carboxylic acid 73 (48 mg, 0.17 mmol) was dissolved in 1.7 mL of thionyl chloride and heated at reflux (80° C.) for 4 hours. Thionyl chloride was removed by evaporation and last traces were removed via azeotrope formation with toluene. The residue was dissolved in CH₂Cl₂ and treated with racemic(dl) 2-amino-1-propanol (50 mg; 0.67 mmol) and stirred at RT for 30 minutes. The reaction mixture was diluted with CH₂Cl₂ and washed with 1N aqueous HCl solution, water, saturated NaHCO₃ solution and brine. Concentration in vacuo gave crude yellow oil. FSGC (2% methanol in CH₂Cl₂) served to isolate the desired amide 76 [R═CH (CH₃) CH₂OH] as yellow solid. mp: 190° C. (dec). HRMS (M+1) Calc for C₁₉H₂₇N₂O₂S 347.1794. Found 347.1791

### Examples 77-103

By essentially the same procedures set forth in Example 74-76, the compounds in Column 4 of Table 13 were prepared:

TABLE 13

| | Method of | | | |
|---|---|---|---|---|
| Example | Preparation | Structure | mp (° C.) | LCMS (M + 1) |
| 77 | A | 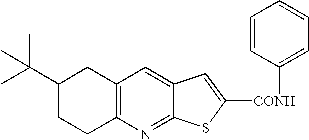 | 269-272 | 365 |
| 78 | A | 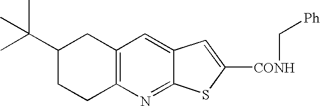 | 192 | 379 |
| 79 | A | 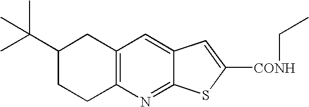 | 198(dec) | 317 |
| 80 | A | 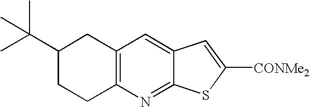 | 135-137 | 317. |
| 81 | A | 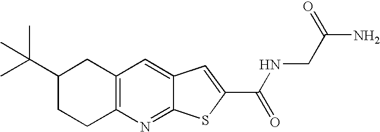 | 280(dec) | 346 |
| 82 | C | 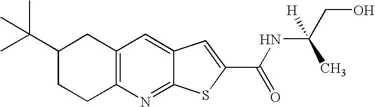 | 202(dec) | 347 |
| 83 | C | 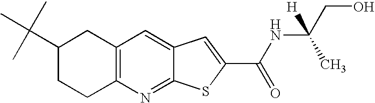 | 183 | 347 |
| | | (+)-(S)-diastereomer | | |
| 84 | B | 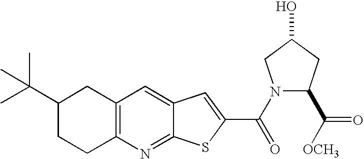 | 105-108 | 417 |
| 85 | B | 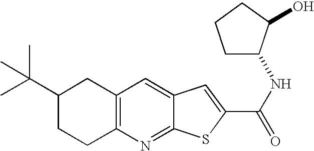 | 123-126 | 373 |
| 86 | B | 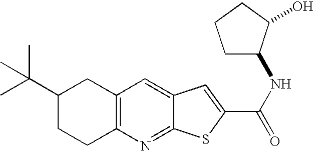 | 123-126 | 373 |
| 87 | B | 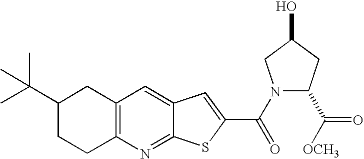 | 168(dec) | 403 |
| 88 | B | 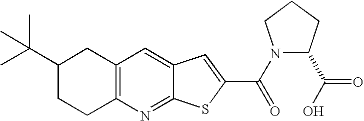 | 234(dec) | 387 |
| 89 | B | 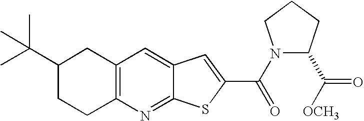 | 153-155 | 401 |
| 90 | B | 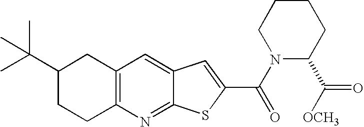 | 63-65 | 415 |
| 91 | B | 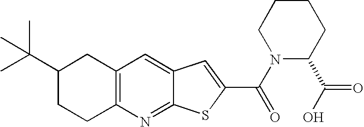 | 215(dec) | 401 |
| 92 | B | 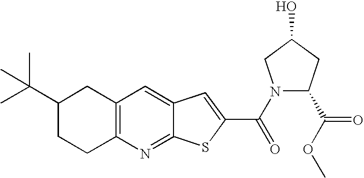 | 130-133 | 417 |
| 93 | B | 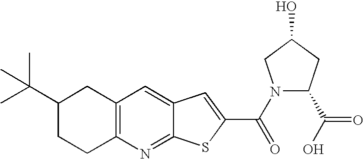 | 230 (dec) | 403 |
| 94 | B | 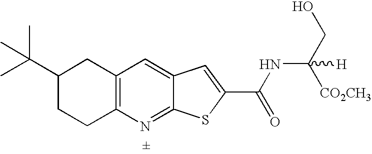 | 148-151 | 391 |
| 95 | B | 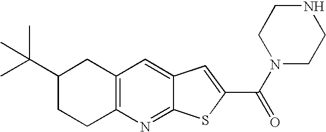 | 277(dec) | 358 |
| 96 | B | 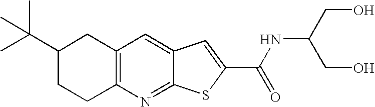 | 188-191 | 363 |
| 97 | B | 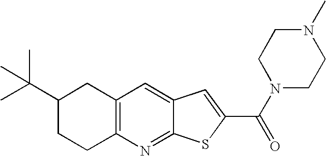 | 171-173 | 372 |
| 98 | B | 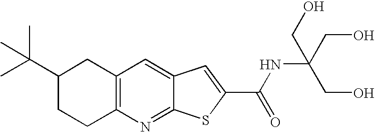 | 199-201 | 393 |
| 99 | B | 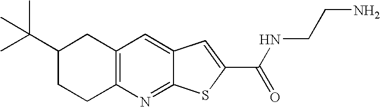 | 182(dec) | 332 |
| 100 | B | 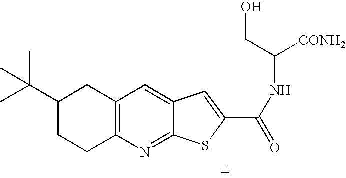 | 213(dec) | 376 |
| 101 | B | 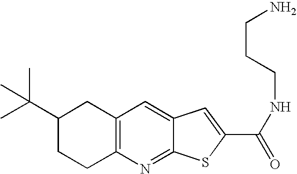 | 155(dec) | 346 |
| 102 | B | 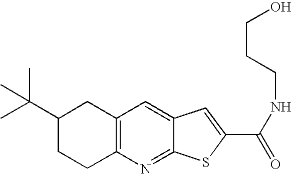 | 221(dec) | 347 |
| 103 | B | 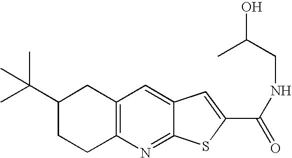 | 188-192 | 347 |

### Example 104

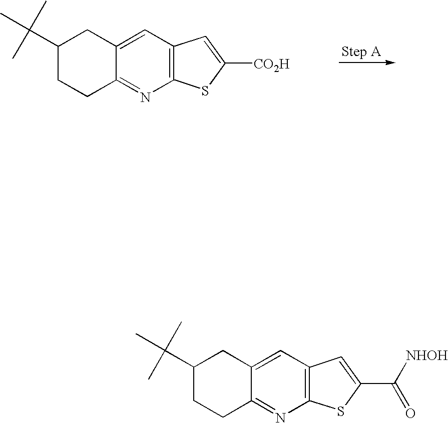 Step A:

6-tert-Butyl-5,6,7,8-tetrahydrothieno[2,3-b]quinoline-2-carboxylic acid hydroxyamide: To a mixture of tert-butyl-5,6,7,8-tetrahydrothieno[2,3-b]quinoline-2-carboxylic acid (100 mg, 0.35 mmol) in 1 mL of methyl chloroformate, was added triethylamine (100 mg, 0.99 mmol). The reaction was stirred at room temperature for 2 h. It was diluted with 3 mL of CH₂Cl₂ and then filtered. The filtrate was concentrated under vacuum and diluted with 2 mL of THF. The resulting solution was added into a solution of hydroxylamine hydrochloride (120 mg, 1.73 mmol), KOH (97 mg, 1.73 mmol) in 4 mL of MeOH. The reaction was stirred at room temperature for 1 h. H₂O was then added slowly until the titled compound precipitated out from the reaction solution. This solid material was collected by filtration, and washed with H₂O and MeOH to give 23 mg (22%) of 6-tert-butyl-5,6,7,8-tetrahydrothieno[2,3-b]quinoline-2-carboxylic acid hydroxyamide. LCMS: MH⁺=305; mp (° C.)=210-236 (dec.).

### Example 105

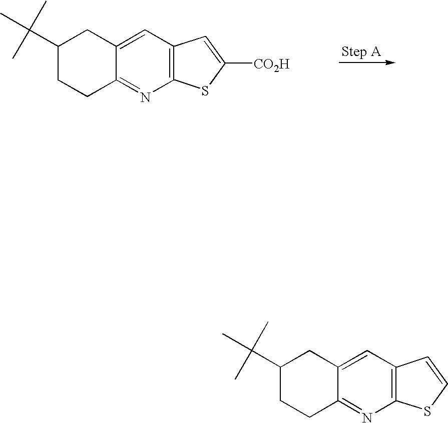 Step A:

6-tert-Butyl-5,6,7,8-tetrahydrothieno[2,3-b]quinoline: A mixture of tert-butyl-5,6,7,8-tetrahydrothieno[2,3-b]quinoline-2-carboxylic acid (84 mg, 0.29 mmol), cupper powder (28 mg, 0.44 mmol) in 2.5 mL of quinoline was stirred at 185° C. for 1.5 h. It was cooled to room temperature. It was diluted with 40 mL of CH₂Cl₂ and washed by 2 N aqueous HCl. The organic phase was dried over anhydrous Na₂SO₄ and then concentrated under vacuum. The residue was purified by flash chromatography eluting with 5% EtOAc/CH₂Cl₂ to give 60 mg (84%) of 6-tert-butyl-5,6,7,8-tetrahydrothieno[2,3-b]quinoline. LCMS: MH⁺=246; mp (° C.)=70-73.

### Example 106

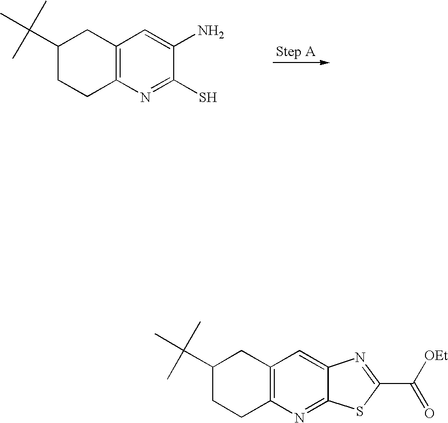

Step A:

(±)-7-tert-Butyl-5,6,7,8-tetrahydro-thiazolo[5,4-b]quinoline-2-carboxylic acid ethyl ester (compound 106): To a solution of 3-amino-6-tert-butyl-5,6,7,8-tetrahydroquinoline-2-thiol (see Example 128, step D) (0.30 g, 1.27 mmol) in dichloromethane (6 mL) at room temperature was added ethyl oxalyl chloride (1.0 mL, 8.9 mmol). The reaction was stirred at room temperature for 1 h. The reaction was concentrated in vacuo and purified via silica gel chromatography (5%-10% EtOAc/hexanes) provided (±)-7-tert-butyl-5,6,7,8-tetrahydro-thiazolo[5,4-b]quinoline-2-carboxylic acid ethyl ester as a white solid (0.2 g, 51% yield). LCMS [M+1]⁺=319; mp (° C.)=84-86.

### Examples 107-108

The enantiomers of compound 106 were separated by chiral HPLC using Chiralpak OD column (10% isopropanol/hexanes). The less polar enantiomer, (−)-7-tert-butyl-5,6,7,8-tetrahydro-thiazolo[5,4-b]quinoline-2-carboxylic acid ethyl ester (compound 107), was obtained as a white solid; [α]_{D}=−70.2 (MeOH, c=1.35), LCMS [M+1]⁺=319, mp (° C.)=84-88. The more polar enantiomer, (+)-7-tert-Butyl-5,6,7,8-tetrahydro-thiazolo[5,4-b]quinoline-2-carboxylic acid ethyl ester (compound 108) was obtained as a white solid; [α]_{D}=+64.2 (MeOH, c=1.04), LCMS [M+1]⁺=319, mp (° C.)=85-88.

### Example 109

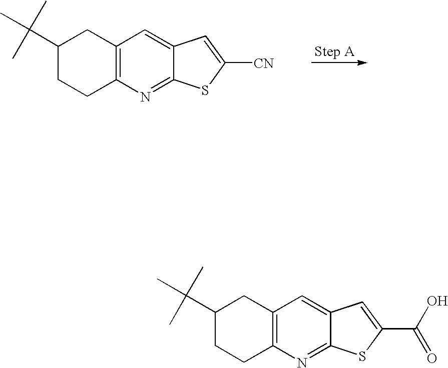 Step A:

A solution of the tricyclic cyanide 58 (4.93 g; 18.3 mmol; see example 58) in 120 mL of phosphoric acid was refluxed at 100° C. for 4 hr. The reaction mixture was cooled to room temperature (RT) and poured over ice and water (800 mL). Most of the phosphoric acid was neutralized by the addition of 100 mL of 1M NaOH solution. The precipitated tricyclic acid was collected via filtration, washed with more water and dried to obtain 5.2 g (˜100%) of yellow solid, which was 6-tert-butyl-5,6,7,8,8-tetrahydrothieno[2,3-b]quinoline-2-carboxylic acid (compound 109)

### Example 110

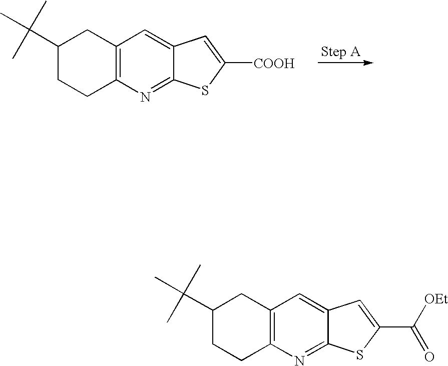 Step A:

The compound 6-tert-butyl-5,6,7,8,8-tetrahydrothieno[2,3-b]quinoline-2-carboxylic acid (compound 109; Example 109) was dissolved in DMF (70 mL). Potassium carbonate (3.79 g; 27.4 mmol), cesium fluoride (4.16 g; 27.4 mmol) and ethyl iodide (2.2 mL; 27.4 mmol) were added in sequence and stirred at RT overnight. The reaction mixture was diluted with water and ethyl acetate. The separated aqueous layer was back extracted with EtOAc. The combined EtOAc extracts were diluted hexanes and washed with water, brine and dried. Concentration gave a brown solid which was purified by flash chromatography (5% EtOAc in hexanes) to obtain the ethyl ester (6-tert-Butyl-5,6,7,8-tetrahydrothieno[2,3-b]-quinoline-2-ethyl carboxylate; compound 110) as a yellow solid (4.72 g; 82%).

Chiral HPLC separation of racemic compound 110 using Chiralpak OD (9:1 v/v=hexanes-isopropanol) gave first the less polar ethyl 6-(1,1-dimethylethyl)-5,6,7,8-tetrahydro-thieno[2,3-b]quinoline-2-carboxylate (the (−)enantiomer; compound 110-1) as white solid. Electrospray MS [M+1]⁺=318. The more polar ethyl 6-(1,1-dimethylethyl)-5,6,7,8-tetrahydro-thieno[2,3-b]quinoline-2-carboxylate (the (+)-enantiomer; compound 110-2) was also obtained as white solid. Electrospray MS [M+1]⁺=318.

### Example 111

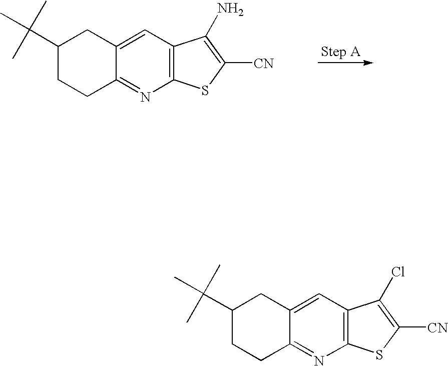 Step A:

6-tert-Butyl-3-chloro-5,6,7,8-tetrahydrothieno[2,3-b]quinoline-2-carbonitrile: To a solution of 90% t-butyinitrite (30 mg, 0.26 mmol) in 1 mL of acetonitrile, was added CuCl₂ (28 mg, 0.21 mmol). The resulting mixture was heated at 65° C. when 3-amino-6-tert-butyl-5,6,7,8-tetrahydrothieno[2,3-b]quinoline-2-carbonitrile (50 mg, 0.18 mmol) was added. The reaction was stirred at 65° C. for 20 min. It was diluted with EtOAc and washed with 1 N aqueous NaOH. The organic phase was concentrated and the residue was purified by flash chromatography eluting with CH₂Cl₂ to give 21 mg (39%) of 6-tert-butyl-3-chloro-5,6,7,8-tetrahydrothieno[2,3-b]quinoline-2-carbonitrile. LCMS: MH⁺=305; mp (° C.)=108-110.

### Example 112

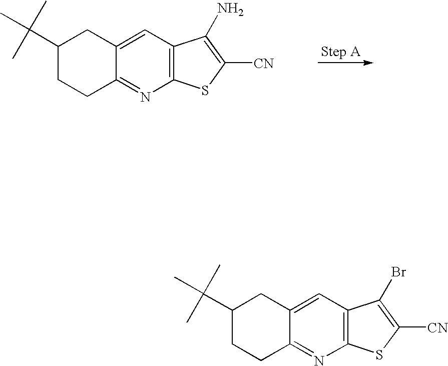 Step A:

6-tert-Butyl-3-bromo-5,6,7,8-tetrahydrothieno[2,3-b]quinoline-2-carbonitrile: To a solution of 90% t-butylnitrite (30 mg, 0.26 mmol) in 1 mL of acetonitrile, was added CuBr₂ (47 mg, 0.21 mmol). The resulting mixture was heated at 65° C. when 3-amino-6-tert-butyl-5,6,7,8-tetrahydrothieno[2,3-b]quinoline-2-carbonitrile (50 mg, 0.18 mmol) was added. The reaction was stirred at 65° C. for 20 min. It was diluted with EtOAc and washed with 1 N aqueous NaOH. The organic phase was concentrated and the residue was dissolved in minimum amount of CH₂Cl₂. To the solution was added hexanes so that the starting material was precipitated out. After filtration, the mother liquor was concentrated and the residue was purified by flash chromatography eluting with CH₂Cl₂ to give 20 mg (33%) of 6-tert-butyl-3-bromo-5,6,7,8-tetrahydrothieno[2,3-b]quinoline-2-carbonitrile. LCMS: MH⁺=349; mp (° C.)=146-149.

### Example 113

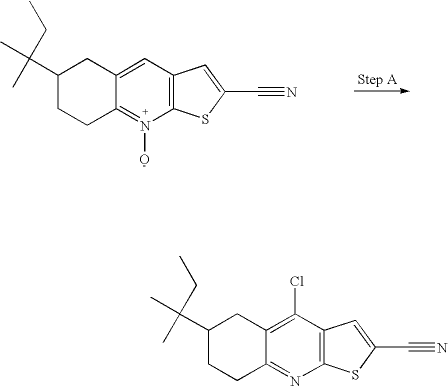 Step A:

A solution of the product from Example 71 (1.0 g, 3.334 mmol) in POCl₃ (6.6 mL) at 0° C. was heated at reflux for 2.5 h. The solution was cooled to 25° C. and diluted with CH₂Cl₂ (50 mL). The organic layer was washed with aqueous saturated NaHCO₃ (30 mL). The aqueous layer was extracted with CH₂Cl₂ (2×15 mL). The combined organic layers were dried (Na₂SO₄), filtered and concentrated under reduced pressure. The crude product was purified by flash chromatography using a 25% EtOAc-hexanes solution as eluent (0.032 g, 97%). ¹H NMR (CDCl₃, 400 MHz) δ 7.88 (s, 1H), 3.24-3.17 (m, 1H), 3.09-2.96 (m, 2H), 2.56-2.49 (m, 1H), 2.13-2.08 (m, 1H), 1.68-1.60 (m, 1H), 1.51-1.38 (m, 3H), 0.95 (s, 9H), 0.88 (t, J=7.3 Hz, 3H); MS: MH⁺=319.

### Example 114

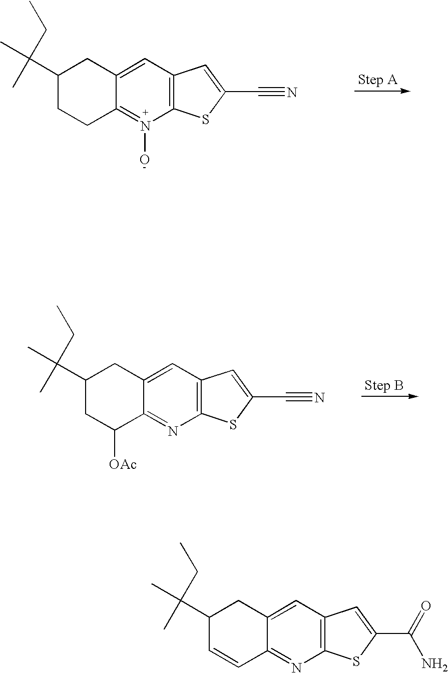 Step A:

A solution of product from Example 71 (0.20 g, 0.66 mmol) in acetic anhydride (0.13 mL) was heated at 100° C. for 2.5 h and cooled to 25° C. The solution was concentrated in vacuo. The residue was diluted with CH₂Cl₂ (15 mL). Aqueous saturated NaHCO₃ (20 mL) was added and the solution was stirred at 25° C. for 0.2 h. The aqueous layer was extracted with CH₂Cl₂ (3×10 mL). The combined organic layers were dried (Na₂SO₄), filtered and concentrated under reduced pressure. The crude product was purified by flash chromatography using a 25% EtOAc-hexanes solution as eluent (0.18 g, 79%).

Step B:

A solution of the product from Step A, Example 114 (0.075 g, 0.22 mmol) in poly phosphoric acid (1 mL) was heated at 120° C. for 4 h. The solution was cooled to 25° C. and diluted with H₂O (10 mL). The precipitate was filtered and dried under vacuum. The crude product was purified by flash chromatography using a 10% MeOH—CH₂Cl₂ solution as eluent (0.034 g, 52%). ¹H NMR (CDCl₃, 400 MHz) δ 7.78 (s, 1H), 7.69 (s, 1H), 6.76 (dd, J=9.6 Hz, J=2.2 Hz, 1H), 6.45 (dd, J=10.2 Hz, J=3.7 Hz, 1H), 5.85 (br s, 2H), 2.97-2.92 (m, 2H), 2.50-2.44 (m, 1H), 1.42-1.36 (m, 2H), 0.91-0.90 (m, 6H), 0.86 (t, J=7.3 Hz, 3H); MS: MH⁺=301.

### Example 115

By essentially the same procedure set forth in Example 114, only substituting the compound shown in Column 2 of Table 14 in Step A, the compound in Column 3 was prepared:

TABLE 14

| Example | Column 2 | Column 3 | CMPD |
|---|---|---|---|
| 115 | 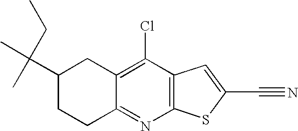 | 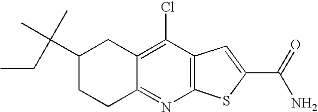 | ¹H NMR (DMSO-d₆): δ 8.39(br s, 1H), 8.12 (s, 1H), 7.67(br s, 1H), 3.11-3.05 (m, 1H), 3.01-2.92 (m, 2H), 2.02-1.98 (m, 1H), 1.66-1.60 (m, 1H), 1.45-1.33 (m, 3H), 0.91 (s, 9H), 0.84(t, J=7.3 Hz, 3H); MS: MH⁺ =337 |

### Example 116

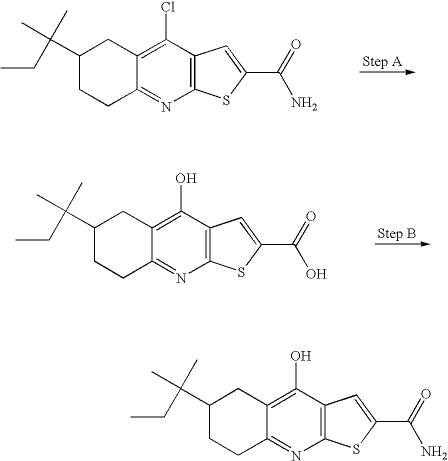 Step A:

A solution of the compound prepared in Example 115 (0.10 g, 0.30 mmol) in H₂O/MeOH (1:3, 2 mL) at 25° C. was treated with LiOH (0.036 g, 5 equiv.). The solution was heated at 100° C. for 60 h. The solution was concentrated in vacuo and the residue was diluted with 48% HBr (4 ml) and heated at 100° C. for 0.5 h. AcOH (1 mL) was added and heating at 100° C. was continued for 2 h. The solution was concentrated in vacuo and dried under vacuum. The crude product was used directly in the next step.

Step B:

The product from Step A in Example 116 was diluted with thionyl chloride (5 mL) and stirred at 25° C. for 1 h. The residue was concentrated in vacuo. The residue was treated with 7N NH₃/MeOH (10 mL) and stirred for 60 h. The solution was concentrated in vacuo. The crude product was purified by flash chromatography using a 10% MeOH—CH₂Cl₂ solution as eluent (0.007 g, 7%). ¹H NMR (DMSO-d₆, 400 MHz) δ 12.29 (br s, 1H), 8.10 (br s, 1H), 8.02 (s, 1H), 7.45 (br s, 1H), 2.75-2.50 (m, 4H), 1.96-1.89 (m, 1H), 1.36-1.19 (m, 4H), 0.86-0.81 (m, 9H); MS: MH⁺=319.

### Example 118

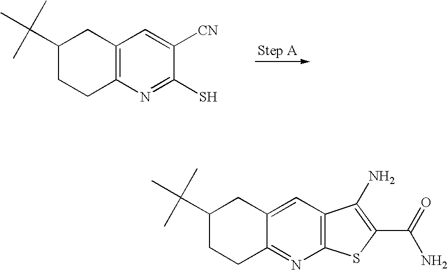 Step A:

3-Amino-6-tert-butyl-5,6,7,8-tetrahydrothieno[2,3-b]quinoline-2-carboxylic acid amide: To a mixture of 6-tert-butyl-2-mercapto-5,6,7,8-tetrahydroquinoline-3-carbonitrile (60 mg, 0.24 mmol) in 2 mL of DMF, was added 2-bromoacetamide (40 mg, 0.29 mmol) followed by 0.25 mL of 20% aqueous KOH. The reaction was stirred at room temperature for 0.5 h. The reaction content was diluted by 20 mL of H₂O. The solid thus formed was collected by filtration and washed with H₂O to give 57 mg (77%) of 3-amino-6-tert-butyl-5,6,7,8-tetrahydrothieno[2,3-b]quinoline-2-carboxylic acid amide. LCMS: MH⁺=304; mp (° C.)=278-280 (dec.).

### Example 119

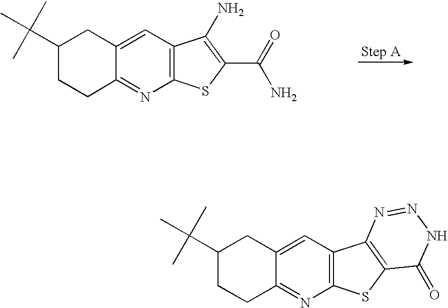 Step A:

7-tert-Butyl-6,7,8,9-tetrahydro2H-11-thia-2,3,4,10-tetraazabenzo[b]fluoren-1-one: To a solution of 3-amino-6-tert-butyl-5,6,7,8-tetrahydrothieno[2,3-b]quinoline-2-carboxylic acid amide (30 mg, 0.10 mmol) in 2 mL of 12 N aqueous HCl, was added sodium nitrite (14 mg, 0.20 mmol). The reaction was stirred at room temperature for 10 min. To this solution was added 10 mL of H₂O. The resulting mixture was filtered. The solid was washed with dilute aqueous NaHCO₃ and H₂O, then dried under vacuum to give 18 mg (58%) of 7-tert-butyl-6,7,8,9-tetrahydro2H-11-thia-2,3,4,10-tetraazabenzo[b]fluoren-1-one. LCMS: MH⁺=315; mp (° C.)=120-259 (dec.).

### Example 120

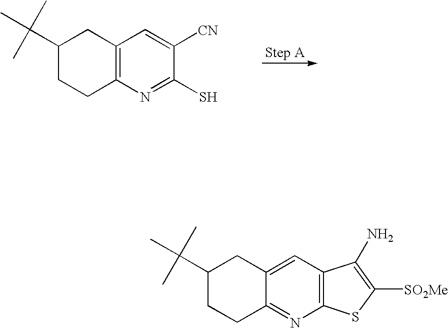 Step A:

6-tert-Butyl-2-methanesulfonyl-5,6,7,8-tetrahydrothieno[2,3-b]quinolin-3-ylamine: To a mixture of 6-tert-butyl-2-mercapto-5,6,7,8-tetrahydroquinoline-3-carbonitrile (100 mg, 0.41 mmol) in 1.5 mL of DMF, was added 0.2 mL of 20% aqueous KOH followed by chloromethylsulfonylmethane (100 mg, 0.78 mmol). The reaction mixture was deoxygenated by passing through a stream of N₂. It was then stirred at 110° C. under N₂ for 3 h. Upon cooling to room temperature, the mixture was poured into 30 mL of H₂O and neutralized by 2 N aqueous HCl. The solid was collected by filtration and washed with H₂O. It was further purified by flash chromatography eluting with 6% EtOAc/CH₂Cl₂ to give 97 mg (71%) of 6-tert-butyl-2-methanesulfonyl-5,6,7,8-tetrahydrothieno[2,3-b]quinolin-3-ylamine. LCMS: MH⁺=339; mp (° C.)=212-213.

### Example 121

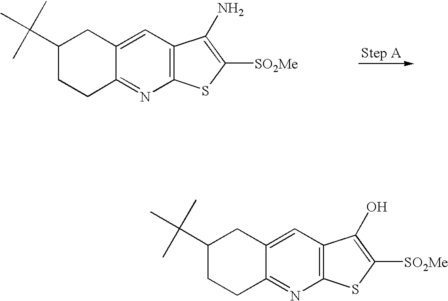 Step A:

6-tert-Butyl-2-methanesulfonyl-5,6,7,8-tetrahydrothieno[2,3-b]quinolin-3-ol: A mixture of 6-tert-butyl-2-methanesulfonyl-5,6,7,8-tetrahydrothieno[2,3-b]quinolin-3-ylamine (115 mg, 0.34 mmol) in 4.3 g of 85% phosphoric acid was stirred at 80° C. for 2.5 h. Upon cooling to room temperature, it was poured into 75 mL of ice H₂O. The solid was collected by filtration, washed with H₂O. It was further purified by flash chromatography eluting with 10% MeOH/CH₂Cl₂ to give 115 mg (100%) of 6-tert-butyl-2-methanesulfonyl-5,6,7,8-tetrahydrothieno[2,3-b]quinolin-3-ol. LCMS: MH⁺=340; mp (° C.)=76-120 (dec.).

### Example 122

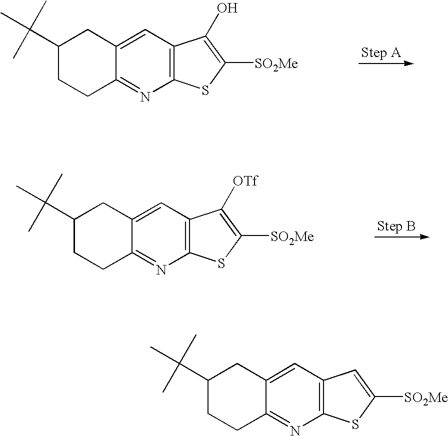 Step A:

Trifluoromethanesulfonic acid 6-tert-butyl-2-methanesulfonyl-5,6,7,8-tetrahydrothieno[2,3-b]quinolin-3-yl ester: To a solution of 6-tert-butyl-2-methanesulfonyl-5,6,7,8-tetrahydrothieno[2,3-b]quinolin-3-ol (97 mg, 0.29 mmol) in 2 mL of CH₂Cl₂ stirred at −78° C., was added diisopropylethylamine (74 mg, 0.57 mmol) followed by Tf₂O (145 mg, 0.51 mmol). The reaction was stirred at −78° C. for 10 min. It was quenched by adding 3 mL of H₂O and diluted with 50 mL of CH₂Cl₂. The mixture was washed with 1 N aqueous NaOH (20 mL), 1 N aqueous HCl (20 mL), and dried over anhydrous Na₂SO₄. The solvent was removed under vacuum, and the residue was purified by flash chromatography eluting with 5% EtOAc/CH₂Cl₂ to give 104 mg (78%) of trifluoromethanesulfonic acid 6-tert-butyl-2-methanesulfonyl-5,6,7,8-tetrahydrothieno[2,3-b]quinolin-3-yl ester.

Step B:

6-tert-Butyl-2-methanesulfonyl-5,6,7,8-tetrahydrothieno[2,3-b]quinoline: To a mixture of trifluoromethanesulfonic acid 6-tert-butyl-2-methanesulfonyl-5,6,7,8-tetrahydrothieno[2,3-b]quinolin-3-yl ester (104 mg, 0.22 mmol), Pd(PPh₃)₄ (25 mg, 0.022 mmol), and LiCl (46 mg, 1.1 mmol) in 3 mL of THF stirred at 65° C., was added a solution of Bu₃SnH (97 mg, 0.33 mmol) in 2 mL of THF slowly over 3 min. The reaction was stirred at 65° C. for 15 min. The solvent was removed under vacuum. The residue was diluted with 30 mL of CH₂Cl₂ and washed with H₂O. The organic phase was dried over anhydrous Na₂SO₄, and then concentrated under vacuum. The residue was purified by flash chromatography eluting with 35% EtOAc/hexanes to give 50 mg (70%) of 6-tert-butyl-2-methanesulfonyl-5,6,7,8-tetrahydrothieno[2,3-b]quinoline. LCMS: MH⁺=324; mp (° C.)=153-154.

### Example 123

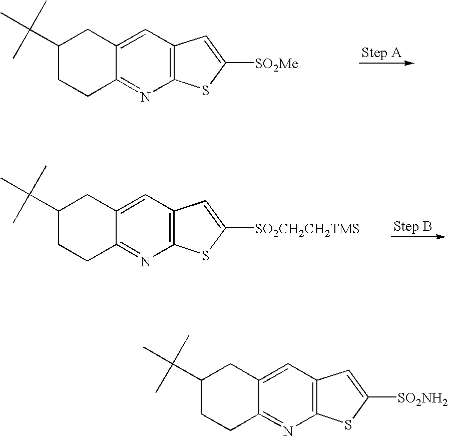 Step A:

6-tert-Butyl-2-(2-trimethylsilanylethanesulfonyl)-5,6,7,8-tetrahydrothieno[2,3-b]quinoline: To a solution of 6-tert-butyl-2-methanesulfonyl-5,6,7,8-tetrahydrothieno[2,3-b]quinoline (48 mg, 0.15 mmol) in 1.5 mL of THF stirred at −78° C., was added a solution of 2 M lithium diisopropylamide in THF (0.16 mL, 0.33 mmol). The reaction was stirred at −78° C. for 0.5 h when (iodomethyl)trimethylsilane (70 mg, 0.33 mmol) was added. The reaction was stirred at −78° C. for 1 h and then warmed up to room temperature over a period of 1 h. It was quenched by adding 2 mL of 1 N aqueous HCl and the resulting mixture was extracted by 50 mL of CH₂Cl₂. The organic phase was dried over anhydrous Na₂SO₄ and then concentrated. The residue was purified by flash chromatography eluting with 25% EtOAc/hexanes to give 13 mg (21%) of 6-tert-butyl-2-(2-trimethylsilanylethanesulfonyl)-5,6,7,8-tetrahydrothieno[2,3-b]quinoline.

Step B:

6-tert-Butyl-5,6,7,8-tetrahydrothieno[2,3-b]quinoline-2-sulfonic acid amide: To a solution of 6-tert-butyl-2-(2-trimethylsilanylethanesulfonyl)-5,6,7,8-tetrahydrothieno[2,3-b]quinoline (21 mg, 0.05 mmol) in 0.5 mL of THF, was added a solution of 1 M tetrabutylammonium fluoride in THF (0.20 mL, 0.20 mmol). The reaction was refluxed for 1 h. It was cooled to room temperature. To the mixture was added sodium acetate (160 mg, 1.95 mmol), 1 mL of H₂O, and hydroxylamine-O-sulfonic acid (180 mg, 1.59 mmol) sequentially. The reaction mixture was stirred at room temperature for 24 h. It was extracted by EtOAc (20 mL), and dried over anhydrous Na₂SO₄. The solvent was removed under vacuum and the residue was purified by flash chromatography eluting with 4% MeOH/CH₂Cl₂ to give a crude material, which was recrystallized from EtOAc/hexanes to give 5 mg (30%) of 6-tert-butyl-5,6,7,8-tetrahydrothieno[2,3-b]quinoline-2-sulfonic acid amide. LCMS: MH⁺=325; mp (° C.)=140-225 (dec.).

### Example 124

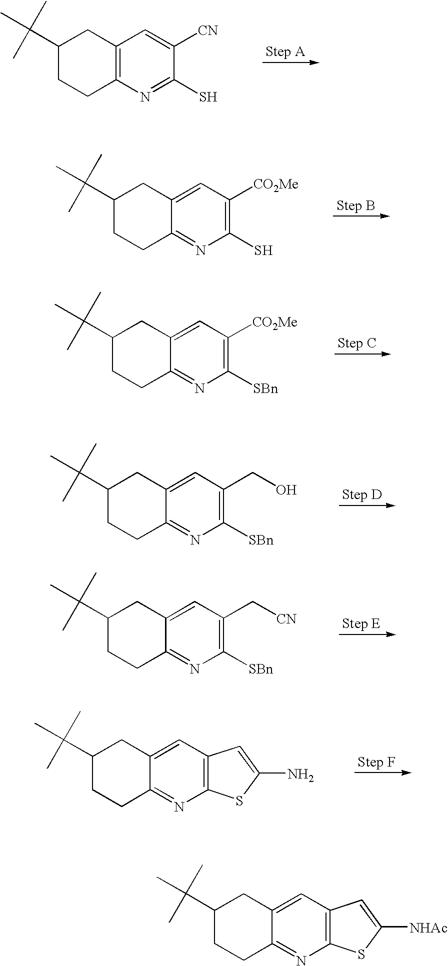 Step A:

6-tert-Butyl-2-mercapto-5,6,7,8-tetrahydroquinoline-3-carboxylic acid methyl ester: A mixture of 6-tert-butyl-2-mercapto-5,6,7,8-tetrahydroquinoline-3-carbonitrile (1.00 g, 4.07 mmol) in 6 mL of AcOH and 6 mL of 95% H₂SO₄ was heated at 130° C. for 24 h. Upon cooling to room temperature, it was poured into 500 mL of ice H₂O. The solid was collected by filtration, washed with H₂O and then dried under vacuum. To this solid material was added 10 mL of DMF followed by K₂CO₃ (462.3 mg, 3.35 mmol) and iodomethane (952 mg, 6.70 mmol). The reaction was stirred at room temperature for 6 h. It was diluted by 100 mL of EtOAc, and washed with H₂O (2×100 mL). It was dried over anhydrous Na₂SO₄, and then concentrated under vacuum. The residue was solidified upon adding 10 mL of EtOAc. To the mixture was further added 30 mL of MeOH. The solid material was collected by filtration, and then dissolved in 40 mL of THF/H₂O (4:1). To the solution was added tri-n-butylphosphine (554 mg, 2.74 mmol). The reaction was stirred at room temperature for 0.5 h. The solvent was removed under vacuum. The residue was dissolved in minimum amount of CH₂Cl₂ and the product was precipitated upon addition of hexanes. The solid was collected by filtration to give 630 mg (56% over three steps) of 6-tert-butyl-2-mercapto-5,6,7,8-tetrahydroquinoline-3-carboxylic acid methyl ester.

Step B:

2-Benzylsulfanyl-6-tert-butyl-5,6,7,8-tetrahydroquinoline-3-carboxylic acid methyl ester: To a solution of 6-tert-butyl-2-mercapto-5,6,7,8-tetrahydroquinoline-3-carboxylic acid methyl ester (630 mg, 2.26 mmol) in 7 mL of DMF, was added benzylbromide (425 mg, 2.48 mmol) followed by K₂CO₃ (312 mg, 2.26 mmol). The reaction was stirred at room temperature for 1 h. It was diluted with 80 mL of EtOAc/hexanes (7:1) and washed with H₂O. The organic phase was dried over anhydrous Na₂SO₄, and then concentrated under vacuum. To the residue was added 20 mL of ice cold acetonitrile, the solid thus formed was collected by filtration to give 590 mg (71%) of 2-benzylsulfanyl-6-tert-butyl-5,6,7,8-tetrahydroquinoline-3-carboxylic acid methyl ester.

Step C:

(2-Benzylsulfanyl-6-tert-butyl-5,6,7,8-tetrahydroquinolin-3-yl)methanol: To a solution of 2-benzylsulfanyl-6-tert-butyl-5,6,7,8-tetrahydroquinoline-3-carboxylic acid methyl ester (750 mg, 2.03 mmol) in 20 mL of THF stirred at −78° C., was added a solution of 1 M lithium triethylborohydride in THF (4.5 mL, 4.5 mmol). The reaction was stirred at −78° C. for 0.5 h when additional amount of 1 M lithium triethylborohydride in THF (2.0 mL, 2.0 mmol) was added. The reaction was stirred at −78° C. for additional 1 h and then gradually warmed to room temperature. It was cooled down to −78° C. when 2 mL of H₂O and 10 mL of saturated aqueous NH₄Cl were added. The mixture was extracted with CH₂Cl₂. The organic phase was dried over anhydrous Na₂SO₄ and then concentrated under vacuum to give 760 mg (109%) of crude (2-benzylsulfanyl-6-tert-butyl-5,6,7,8-tetrahydroquinolin-3-yl)methanol.

Step D:

(2-Benzylsulfanyl-6-tert-butyl-5,6,7,8-tetrahydroquinolin-3-yl)acetonitrile: A solution of (2-benzylsulfanyl-6-tert-butyl-5,6,7,8-tetrahydroquinolin-3-yl)methanol (370 mg, 1.08 mmol) in 5 mL of thionyl chloride was stirred at room temperature for 1 h. The solvent was removed under vacuum. The residue was diluted by 50 mL of CH₂Cl₂ and washed with 30 mL of saturated aqueous NaHCO₃. The organic layer was dried over anhydrous Na₂SO₄, and then concentrated under vacuum. The residue was dissolved in 1 mL of DMSO. The resulting solution was added to a solution of NaCN (106 mg, 2.16 mmol) in 1 mL of DMSO stirred at 85° C. The reaction was stirred at 85° C for 15 min. Upon cooling to room temperature, it was diluted by 50 mL of EtOAc/hexanes (1:1) and washed with H₂O (2×50 mL). The organic phase was dried over anhydrous Na₂SO₄ and then concentrated under vacuum. The residue was purified by flash chromatography eluting with 25% EtOAc/hexanes to give 300 mg (79%) of 2-benzylsulfanyl-6-tert-butyl-5,6,7,8-tetrahydroquinolin-3-yl)acetonitrile.

Step E:

6-tert-Butyl-5,6,7,8-tetrahydrothieno[2,3-b]quinolin-2-ylamine: To a solution of AlBr₃ (563 mg, 2.14 mmol) in 2 mL of benzene stirred under N₂, was added dropwise a solution of 2-benzylsulfanyl-6-tert-butyl-5,6,7,8-tetrahydroquinolin-3-yl)acetonitrile (300 mg, 0.857 mmol) in 0.7 mL of benzene. The reaction was stirred at room temperature under N₂ for 48 h. It was cooled to 0° C., and then slowly added 3 mL of H₂O. The mixture was diluted by 50 mL of CH₂Cl₂ and washed with 50 mL of H₂O. The organic phase was dried over anhydrous Na₂SO₄ and concentrated under vacuum. The residue was further purified by flash chromatography eluting with 20% EtOAc/hexanes to give 160 mg (72%) of crude 6-tert-butyl-5,6,7,8-tetrahydrothieno[2,3-b]quinolin-2-ylamine.

Step F:

N-(6-tert-Butyl-5,6,7,8-tetrahydrothieno[2,3-b]quinolin-2-yl)acetamide: To a solution of the crude 6-tert-butyl-5,6,7,8-tetrahydrothieno[2,3-b]quinolin-2-ylamine (26 mg, 0.10 mmol) in 1 mL of CH₂Cl₂, was added triethylamine (21 μL, 0.12 mmol) and acetyl chloride (8.5 μL, 0.15 mmol). The reaction was stirred at room temperature for 1 h. It was diluted with 20 mL of CH₂Cl₂, washed with 1 N aqueous HCl, and dried over anhydrous Na₂SO₄. The residue was further purified by flash chromatography eluting with 3% MeOH/CH₂Cl₂ to give 10 mg (33%) of N-(6-tert-butyl-5,6,7,8-tetrahydrothieno[2,3-b]quinolin-2-yl)acetamide. LCMS: MH⁺=303; mp (° C.)=260-300 (dec.).

### Example 125

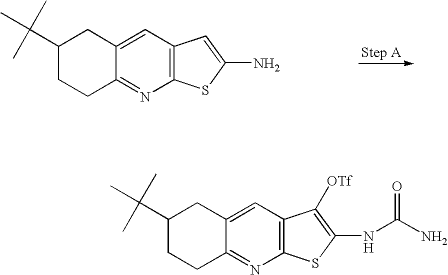 Step A:

(6-tert-Butyl-5,6,7,8-tetrahydrothieno[2,3-b]quinolin-2-yl)urea: To a solution of crude 6-tert-butyl-5,6,7,8-tetrahydrothieno[2,3-b]quinolin-2-ylamine (78 mg, 0.30 mmol) in 5 mL of CH₂Cl₂, was added trichloroacetyl isocyanate (113 mg, 0.60 mmol). The reaction was stirred at room temperature for 30 minutes before 10 mL of hexanes was added. The solid thus formed was collected by filtration and washed with hexanes to give 27 mg of crude material. This was added to a solution of 2 mL of MeOH/H₂O (10:1). To the resulting solution was added 1 mL of 2 M aqueous Na₂CO₃. The mixture was stirred at room temperature for 2 h. It was diluted with 20 mL of CH₂Cl₂, washed with H₂O and dried over anhydrous Na₂SO₄. The solvent was removed under vacuum. The residue was further purified by flash chromatography eluting with 15% MeOH/CH₂Cl₂ to give 13 mg (14%) of (6-tert-butyl-5,6,7,8-tetrahydrothieno[2,3-b]quinolin-2-yl)urea. LCMS: MH⁺=304; mp (° C.)=175-230 (dec.).

### Example 126

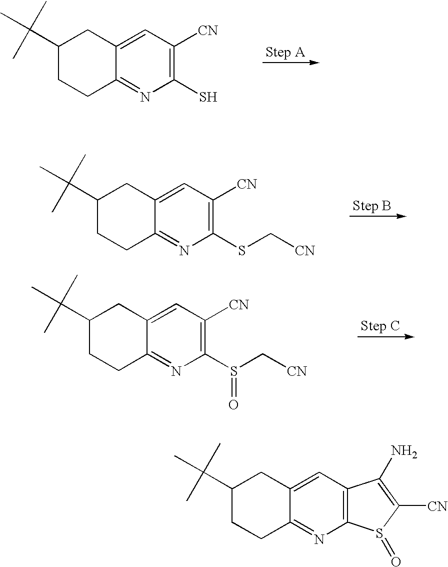 Step A:

6-tert-Butyl-2-cyanomethylsulfanyl-5,6,7,8-tetrahydroquinoline-3-carbonitrile: To a mixture of 6-tert-butyl-2-mercapto-5,6,7,8-tetrahydroquinoline-3-carbonitrile (526 mg, 2.14 mmol) in 20 mL of CH₂Cl₂ cooled at 0° C., was added triethylamine (216 mg, 2.14 mmol) followed by chloroacetonitrile (178 mg, 2.35 mmol). The reaction was stirred at 0° C. for 40 min. It was diluted with CH₂Cl₂ and H₂O. The organic phase was separated and washed with saturated aqueous NH₄Cl, H₂O and brine. It was then concentrated under vacuum, and the residue was purified by flash chromatography eluting with 18% EtOAc/hexanes to give 504 mg (83%) of 6-tert-butyl-2-cyanomethylsulfanyl-5,6,7,8-tetrahydroquinoline-3-carbonitrile.

Step B:

6-tert-Butyl-2-cyanomethanesulfinyl-5,6,7,8-tetrahydroquinoline-3-carbonitrile: To a solution of 6-tert-butyl-2-cyanomethylsulfanyl-5,6,7,8-tetrahydroquinoline-3-carbonitrile (100 mg, 0.351 mmol) in 2 mL of CH₂Cl₂, was added a solution of 3-chloroperoxybenzoic acid (127 mg, 0.737 mmol) in 2 mL of CH₂Cl₂. The reaction was stirred at room temperature for 45 min. It was diluted with 20 mL of CH₂Cl₂ and washed with a solution of 100 mg of sodium sulfite in 20 ml of saturated aqueous NaHCO₃, then with 20 mL of H₂O. It was dried over anhydrous Na₂SO₄ and then concentrated under vacuum. The residue was recrystallized from CH₂Cl₂/hexanes to give 70 mg (66%) of 6-tert-butyl-2-cyanomethanesulfinyl-5,6,7,8-tetrahydroquinoline-3-carbonitrile.

Step C:

3-Amino-6-tert-butyl-1-oxo-5,6,7,8-tetrahydro-1H-1λ⁴-thieno[2,3-b]quinoline-2-carbonitrile: To a solution of 6-tert-butyl-2-cyanomethanesulfinyl-5,6,7,8-tetrahydroquinoline-3-carbonitrile (39 mg, 0.13 mmol) in 2 mL of THF, was added NaH (4.7 mg, 0.19 mmol). The reaction was stirred at room temperature for 1 h. It was quenched by adding 10 drops of 2 N aqueous HCl, and diluted with 3 mL of H₂O. The content was concentrated under vacuum until solid material precipitated out from the solution. The solid was collected by filtration, washed with H₂O, and recrystallized from THF/hexanes to give 21 mg (54%) of 3-amino-6-tert-butyl-1-oxo-5,6,7,8-tetrahydro-1H-1λ⁴-thieno[2,3-b]quinoline-2-carbonitrile. LCMS: MH⁺=302; mp (° C.)=299-302 (dec.).

### Example 127

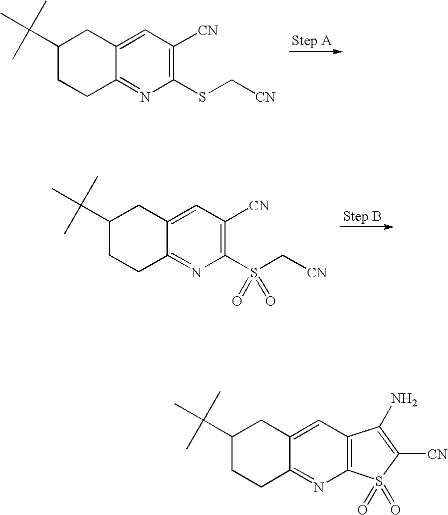 Step A:

6-tert-Butyl-2-cyanomethanesulfonyl-5,6,7,8-tetrahydroquinoline-3-carbonitrile: To a solution of 6-tert-butyl-2-cyanomethylsulfanyl-5,6,7,8-tetrahydroquinoline-3-carbonitrile (100 mg, 0.351 mmol) in 5 mL of CH₂Cl₂, was added 3-chloroperoxybenzoic acid (242 mg, 1.40 mmol). The reaction was stirred at room temperature for 16 h. It was diluted with 25 mL of CH₂Cl₂ and washed with a solution of 500 mg of sodium sulfite in 20 mL of saturated aqueous NaHCO₃, then with 20 mL of H₂O. It was dried over anhydrous Na₂SO₄ and then concentrated under vacuum. The residue was recrystallized from CH₂Cl₂/hexanes. The solid was collected by filtration to give 75 mg (68%) of 6-tert-butyl-2-cyanomethanesulfonyl-5,6,7,8-tetrahydroquinoline-3-carbonitrile.

Step B:

3-Amino-6-tert-butyl-1,1-dioxo-5,6,7,8-tetrahydro-1H-1λ⁶-thieno[2,3-b]quinoline-2-carbonitrile: To a solution of 6-tert-butyl-2-cyanomethanesulfonyl-5,6,7,8-tetrahydroquinoline-3-carbonitrile (30 mg, 0.095 mmol) in 2 mL of THF, was added NaH (3.4 mg, 0.14 mmol). The reaction was stirred at room temperature for 1 h. It was quenched by adding 10 drops of 2 N aqueous HCl, and diluted with 4 mL of H₂O. The content was concentrated under vacuum until solid material precipitated out from the solution. The solid was collected by filtration, washed with H₂O and CH₂Cl₂ to give 20 mg (67%) of 3-amino-6-tert-butyl-1,1-dioxo-5,6,7,8-tetrahydro-1H-1λ⁶-thieno[2,3-b]quinoline-2-carbonitrile. LCMS: MH⁺=318; mp (° C.)=>300.

### Example 128

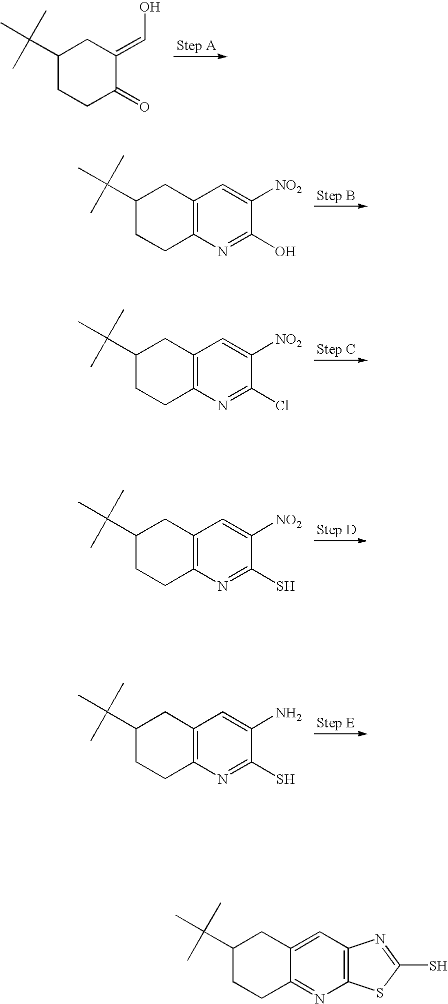 Step A:

6-tert-Butyl-3-nitro-5,6,7,8-tetrahydroquinolin-2-ol: To a solution of 5-tert-butyl-2-oxo-cyclohexanecarbaldehyde, sodium salt (6.3 g, 30.8 mmol) in 120 mL of H₂O, was added aqueous piperidinium acetate [4.72 mL, prepared from glacial acetic acid (42 mL), piperidine (72 mL) and H₂O (100 mL)]. The resulting solution was stirred at 100° C. for 5 min. when 2-nitro-acetamide (3.2 g, 30.8 mmol) was slowly added. The reaction mixture was stirred at reflux for 1.5 h. Upon cooling to room temperature, the solid was collected by filtration and washed with EtOAc to give 3.35 g (44%) of 6-tert-butyl-3-nitro-5,6,7,8-tetrahydroquinolin-2-ol.

Step B:

6-tert-Butyl-2-chloro-3-nitro-5,6,7,8-tetrahydroquinoline: To a mixture of 6-tert-butyl-3-nitro-5,6,7,8-tetrahydroquinolin-2-ol (1.50 g, 6.0 mmol) in POCl₃ (15.0 g, 98 mmol), was added diisopropylethylamine (810 mg, 6.3 mmol). The reaction mixture was stirred at 100° C. for 3 h. Upon cooling to room temperature, the content was poured into ice H₂O (250 mL) and neutralized by 2 N NaOH. The solid was collected by filtration, and re-dissolved in 150 mL of 30% EtOAc/hexanes. This was dried over anhydrous Na₂SO₄. The solvent was removed under vacuum to give 1.50 g (93%) of 6-tert-butyl-2-chloro-3-nitro-5,6,7,8-tetrahydroquinoline.

Step C:

6-tert-Butyl-3-nitro-5,6,7,8-tetrahydroquinoline-2-thiol: To a mixture of 6-tert-butyl-2-chloro-3-nitro-5,6,7,8-tetrahydroquinoline (50 mg, 0.19 mmol) and thiourea (182 mg, 2.4 mmol), was added 0.3 mL of ethanol. The reaction was heated at 100° C. when 0.2 mL of H₂O was added dropwise. The reaction was heated at 100° C. for 3 h. It was cooled to room temperature, and 5 mL of H₂O was added. The resulting solid was collected by filtration to give 26 mg of a yellow powder intermediate. The filtrate was heated at 100° C. for 1.5 h. It was cooled to room temperature. The solid was collected by filtration and washed with H₂O to give additional 16 mg of the yellow powder intermediate. The combined yellow intermediate (42 mg) was dissolved in 5 mL of THF/H₂O (1:1) solution. To this was added tributylphosphine (50 mg, 0.25 mmol). The reaction was stirred at room temperature for 5 min. It was concentrated under vacuum. The residue was precipitated from hexanes. The solid was collected by filtration and washed with 25% of CH₂Cl₂/hexanes to give 36 mg (73%) of 6-tert-butyl-3-nitro-5,6,7,8-tetrahydroquinoline-2-thiol.

Step D:

3-Amino-6-tert-butyl-5,6,7,8-tetrahydroquinoline-2-thiol: A mixture of 6-tert-butyl-3-nitro-5,6,7,8-tetrahydroquinoline-2-thiol (160 mg, 0.60 mmol), iron (240 mg, 4.3 mmol), and CaCl₂ (72 mg, 0.65 mmol) in 8 mL of absolute ethanol was refluxed for 2 h. It was cooled to room temperature and filtered through Celite. The filtrate was concentrated under vacuum. The residue was dissolved in 5 mL of MeOH. To this solution was added 40 mL of H₂O. The precipitate was collected by filtration and further recrystallized from CH₂Cl₂/hexanes to give 60 mg (42%) of 3-Amino-6-tert-butyl-5,6,7,8-tetrahydroquinoline-2-thiol. The mother liquor was concentrated under vacuum and further purified by flash chromatography eluting with 3% MeOH/CH₂Cl₂ to give additional 80 mg (56%) of 3-amino-6-tert-butyl-5,6,7,8-tetrahydroquinoline-2-thiol.

Step E:

7-tert-Butyl-5,6,7,8-tetrahydrothiazolo[5,4-b]quinoline-2-thiol: A mixture of 3-amino-6-tert-butyl-5,6,7,8-tetrahydroquinoline-2-thiol (115 mg, 0.487 mmol), and potassium ethylxanthate (156 mg, 0.975 mmol) in 1.5 mL of absolute ethanol was refluxed for 18 h. It was concentrated under vacuum. The residue was dissolved in 3 mL of H₂O. The pH of the solution was adjusted to 5 by adding AcOH. The solid was collected by filtration and washed with H₂O. This was recrystallized from MeOH to give 17 mg (12.5%) of 7-tert-butyl-5,6,7,8-tetrahydrothiazolo[5,4-b]quinoline-2-thiol. The mother liquor was concentrated and the residue was further purified by flash chromatography eluting with 10% EtOAc/CH₂Cl₂ to give additional 83 mg (61%) of 7-tert-butyl-5,6,7,8-tetrahydrothiazolo[5,4-b]quinoline-2-thiol. LCMS: MH⁺=279; mp (° C.)=259-270 (dec.).

### Example 129

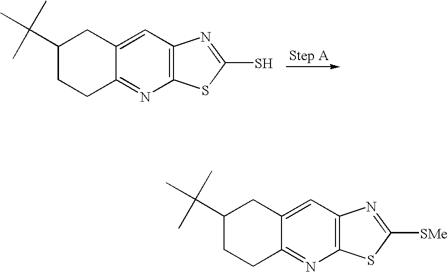 Step A:

7-tert-Butyl-2-methylsulfanyl-5,6,7,8-tetrahydrothiazolor5,4-b]quinoline: To a solution of 7-tert-butyl-5,6,7,8-tetrahydrothiazolo[5,4-b]quinoline-2-thiol (68 mg, 0.25 mmol) in 3 mL of DMF, was added K₂CO₃ (34 mg, 0.25 mmol) and iodomethane (42 mg, 0.29 mmol). The reaction was stirred at room temperature for 30 min. The mixture was diluted with 30 mL of H₂O and extracted with 30 mL of EtOAc. The organic phase was dried over anhydrous Na₂SO₄ and then concentrated under vacuum. The residue was purified by flash chromatography eluting with 25% EtOAc/hexanes to give 64 mg (90%) of 7-tert-butyl-2-methylsulfanyl-5,6,7,8-tetrahydrothiazolo[5,4-b]quinoline. ¹H NMR (CDCl₃, 400 MHz) δ 7.74 (s, 1H), 3.06-3.16 (m, 1H), 2.87-3.00 (m, 2H), 2.76 (s, 3H), 2.62-2.72 (m, 1H), 2.07-2.15 (m, 1H), 1.40-1.57 (m, 2H), 0.98 (s, 9H); LCMS: MH⁺=293.

### Example 130

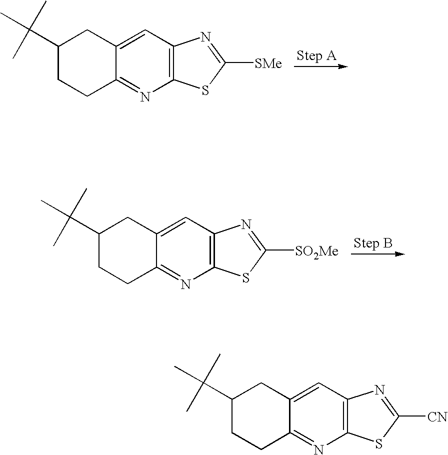 Step A:

7-tert-Butyl-2-methanesulfonyl-5,6,7,8-tetrahydrothiazolo[5,4-b]quinoline: To a solution of 7-tert-butyl-2-methylsulfanyl-5,6,7,8-tetrahydrothiazolo[5,4-b]quinoline (40 mg, 0.137 mmol) in 2.5 mL of AcOH, was added a solution of KMnO₄ (43 mg, 0.274 mmol in 1 mL of H₂O) dropwise. The reaction was stirred at room temperature for 0.5 h. This was quenched by adding an aqueous solution of Na₂SO₃ (1% wt. in H₂O) until the color of the reaction became clear. This was neutralized by 2 N of aqueous Na₂CO₃, and extracted with 20 mL EtOAc. The organic phase was dried over anhydrous Na₂SO₄. The solvent was removed under vacuum to give 37 mg (83%) of 7-tert-butyl-2-methanesulfonyl-5,6,7,8-tetrahydrothiazolo[5,4-b]quinoline.

Step B:

7-tert-Butyl-5,6,7,8-tetrahydrothiazolo[5,4-b]quinoline-2-carbonitrile: To a solution of 7-tert-butyl-2-methanesulfonyl-5,6,7,8-tetrahydrothiazolo[5,4-b]quinoline (37 mg, 0.11 mmol) in 1 mL of DMF, was added KCN (7.4 mg, 0.11 mmol). The reaction was stirred at room temperature for 3 h. It was diluted with 50 mL of EtOAc/hexanes (1:1), and washed with 50 mL of H₂O. The organic phase was dried over anhydrous Na₂SO₄ and concentrated under vacuum. The residue was purified by flash chromatography eluting with 30% EtOAc/hexanes to give 15 mg (48%) of 7-tert-butyl-5,6,7,8-tetrahydrothiazolo[5,4-b]quinoline-2-carbonitrile. LCMS: MH⁺=272; mp (° C.)=99-101.

### Example 131

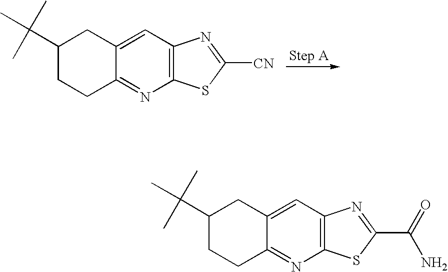 Step A:

7-tert-Butyl-5,6,7,8-tetrahydrothiazolo[5,4-b]quinoline-2-carboxylic acid amide: A mixture of 7-tert-butyl-5,6,7,8-tetrahydrothiazolo[5,4-b]quinoline-2-carbonitrile (15 mg, 0.055 mmol) and 1 gram of polyphosphoric acid was heated at 120° C. for 4 h. This was quenched by adding ice H₂O and neutralized with saturated aqueous Na₂CO₃. The resulting mixture was extracted with CH₂Cl₂. The organic phase was concentrated and further purified by flash chromatography eluting with 60% EtOAc/hexanes to give 15 mg (69%) of 7-tert-butyl-5,6,7,8-tetrahydrothiazolo[5,4-b]quinoline-2-carboxylic acid amide. LCMS: MH⁺=290; mp (° C.)=259-261.

### Example 131-A

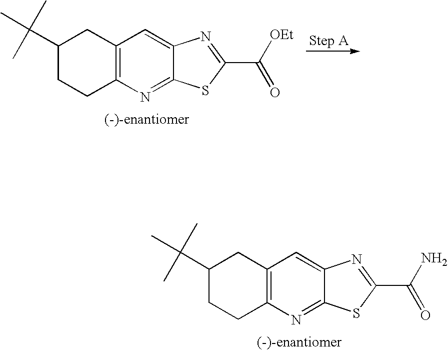

(−)-7-tert-Butyl-5,6,7,8-tetrahydro-thiazolo[5,4-b]quinoline-2-carboxylic acid amide: A sealed tube containing (−)-7-tert-butyl-5,6,7,8-tetrahydro-thiazolo[5,4-b]quinoline-2-carboxylic acid ethyl ester (51.7 mg, 0.162 mmol) (compound 107, see Examples 107-108) was added 4 ml of a solution of 7 N NH₃ in MeOH. The tube was heated at 120° C. for 12 h. The reaction was cooled to room temperature and concentrated in vacuo. Purification via silica gel chromatography (50% EtOAc/hexanes) provided 25.3 mg (54% yield) of (−)-7-tert-butyl-5,6,7,8-tetrahydro-thiazolo[5,4-b]quinoline-2-carboxylic acid amide as a white solid. [α]_{D}=−122.9 (MeOH, c=0.5), LCMS [M+1]⁺=290; mp (° C.)=247-249.

### Example 131-B

### (+)-7-tert-Butyl-5,6,7,8-tetrahydro-thiazolo[5,4-b]quinoline-2-carboxylic acid amide

Following a similar procedure set forth in previous paragraph (Example 131-A), only substituting with (+)-7-tert-butyl-5,6,7,8-tetrahydro-thiazolo[5,4-b]quinoline-2-carboxylic acid ethyl ester (32.3 mg, 0.101 mmol) (compound 108, see Examples 107-108) gave 14.1 mg (48% yield) of (+)-7-tert-butyl-5,6,7,8-tetrahydro-thiazolo[5,4-b]quinoline-2-carboxylic acid amide (compound 131-B) as a white solid. [α]_{D}=+122.8 (MeOH, c=0.5), LCMS [M+1]⁺=290; mp (° C.)=247-249.

### Example 132

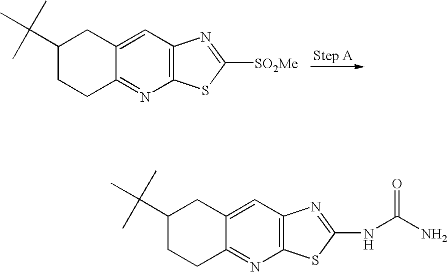 Step A:

(7-tert-Butyl-5,6,7,8-tetrahydrothiazolo[5,4-b]quinolin-2-yl)-urea: To a solution of urea (46 mg, 0.77 mmol) in 1 mL of DMSO, was added NaH (6.0 mg, 0.25 mmol). The reaction was stirred at room temperature for 30 min when a solution of 7-tert-butyl-2-methanesulfonyl-5,6,7,8-tetrahydrothiazolo[5,4-b]quinoline (33 mg, 0.10 mmol) in 0.8 mL of DMSO was added. The reaction was stirred at room temperature for 30 min. It was diluted with 30 mL of EtOAc, washed with 25 mL of 1 N HCl, and dried over anhydrous Na₂SO₄. The solvent was removed under vacuum. The residue was purified by flash chromatography eluting with 8% MeOH/CH₂Cl₂ to give 18.5 mg (60%) of (7-tert-butyl-5,6,7,8-tetrahydrothiazolo[5,4-b]quinolin-2-yl)-urea. LCMS: MH⁺=305; mp (° C.)=300 (dec.).

### Example 133

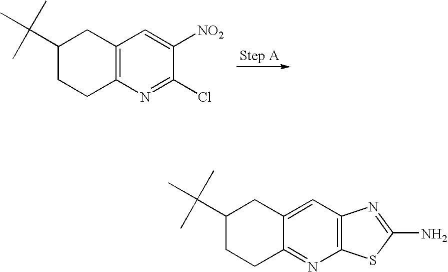 Step A:

7-tert-Butyl-5,6,7,8-tetrahydrothiazolo[5,4-b]quinolin-2-ylamine: A mixture of 6-tert-butyl-2-chloro-3-nitro-5,6,7,8-tetrahydroquinoline (100 mg, 0.372 mmol), KSCN (100 mg, 1.02 mmol) in 1.5 mL of AcOH was stirred at 75° C. for 4 h. The solvent was removed under vacuum. To the residue was added 10 mL of CH₂Cl₂. The resulting mixture was filtered. The filtrate was concentrated to give 109 mg of a light yellow solid, which was mixed with 300 mg of iron and 2 mL of AcOH. The mixture was stirred at 75° C. for 30 min. Upon cooling to room temperature, it was filtered through Celite and rinsed with 10 mL of AcOH. The filtrate was concentrated. The residue was purified by flash chromatography eluting with 80% EtOAc/hexanes to give 73 mg (75%) of 7-tert-butyl-5,6,7,8-tetrahydrothiazolo[5,4-b]quinolin-2-ylamine. LCMS: MH⁺=262; mp (° C.)=219-221.

### Example 134

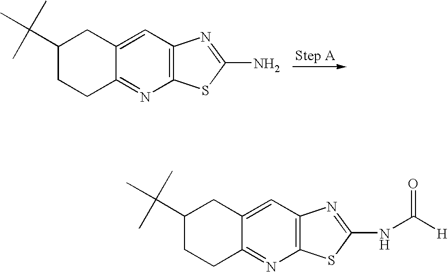 Step A:

N-(7-tert-Butyl-5,6,7,8-tetrahydrothiazolo[5,4-b]quinolin-2-yl)-formamide: A solution of 7-tert-butyl-5,6,7,8-tetrahydrothiazolo[5,4-b]quinolin-2-ylamine (20 mg, 0.077 mmol) in 2 mL of CH₂Cl₂ was added into a solution of acetic anhydride (46 mg, 0.46 mmol) and formic acid (21.2 mg, 0.46 mmol) in 1 mL of CH₂Cl₂. The reaction was stirred at room temperature for 24 h. It was diluted by 20 mL of CH₂Cl₂, washed with 20 mL of saturated aqueous NaHCO₃, and dried over anhydrous Na₂SO₄. The solvent was removed under vacuum and the residue was purified by flash chromatography eluting with 50% EtOAc/CH₂Cl₂ to give 73 mg (100%) of N-(7-tert-butyl-5,6,7,8-tetrahydrothiazolo[5,4-b]quinolin-2-yl)-formamide. LCMS: MH⁺=290; mp (° C.)=241-244 (dec.).

### Example 135

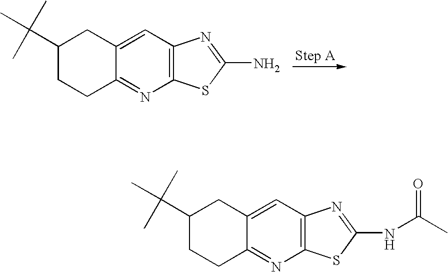 Step A:

N-(7-tert-Butyl-5,6,7,8-tetrahydrothiazolo[5,4-b]quinolin-2-yl)acetamide: To a solution of 7-tert-butyl-5,6,7,8-tetrahydrothiazolo[5,4-b]quinolin-2-ylamine (12.9 mg, 0.049 mmol) and triethylamine (7.4 mg, 0.074 mmol) in 1 mL of CH₂Cl₂, was added acetyl chloride (4.6 mg, 0.059 mmol). The reaction was stirred at room temperature for 30 min. Additional acetyl chloride (1.9 mg, 0.025 mmol) was added. The reaction was stirred at room temperature for additional 10 min. It was diluted by 20 mL of CH₂Cl₂, washed with 1 N HCl, and dried over anhydrous Na₂SO₄. The solvent was removed under vacuum and the residue was purified by flash chromatography eluting with 50% EtOAc/CH₂Cl₂ to give 14.5 mg (97%) of N-(7-tert-butyl-5,6,7,8-tetrahydrothiazolo[5,4-b]quinolin-2-yl)-acetamide. LCMS: MH⁺=304; mp (° C.)=273-75 (dec.).

### Example 144

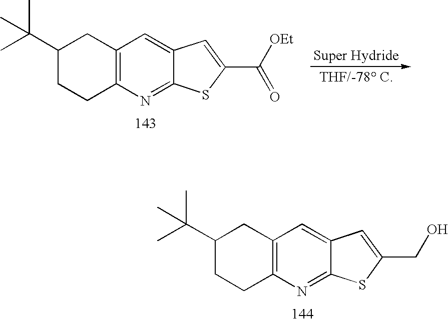

Super hydride (1M in THF; 2.9 mL) was added to a solution of the ester 143 (277 mg; 0.87 mmol) in THF at −78° C. and stirred for 30 minutes. The reaction was quenched with saturated NH₄Cl solution and warmed to RT. The organic product was extracted with EtOAc and washed with water and brine. Concentration to a crude product and FSGC (25% EtOAc in hexanes) gave the primary alcohol 144 (228 mg, 95%) as yellow foamy solid. mp: 52-54° C. LCMS (M+1=C₁₆H₂₂NOS): 276.

### Example 145

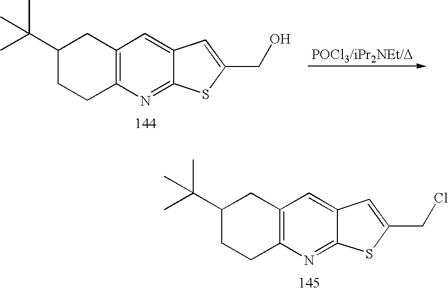

Diisopropylethyl amine (0.2 mL) was added to a solution of the alcohol 144 (287 mg; 1.04 mmol) in 1.7 mL of POCl₃ and the mixture was heated at 100° C. for 1.5 hr. The reaction mixture was cooled and poured over ice and neutralized with 2N NaOH solution. The organic product was extracted with CH₂Cl₂ and washed with water and brine. Concentration and FSGC (8% EtOAc in hexanes) furnished the chloride 145 (275 mg, 95%) as yellow solid. mp 48-50° C. LCMS (M+1=C₁₆H₂₀ClNS): 294.

### Example 146

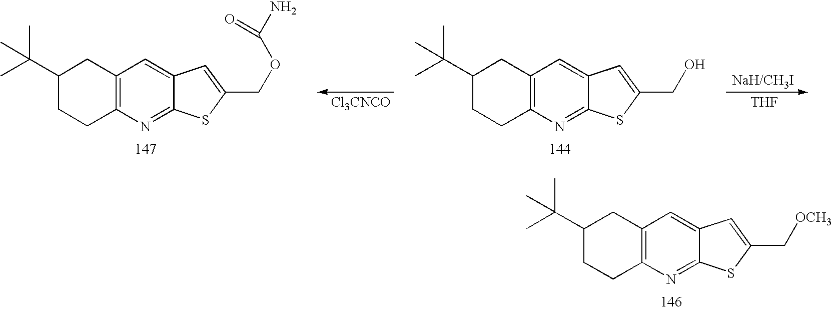

Sodium hydride (60% oil suspension, 10 mg) was added to a solution of the alcohol 144 (19 mg; 0.07 mmol) in THF, followed by iodomethane (10 μL). The reaction mixture was stirred at RT for 16 h. The reaction was quenched by the addition of water. Organic product was extracted into EtOAc and washed with water and brine. FSGC (10% EtOAc in hexane) gave 21 mg (100%) of 146 as yellow oil. LCMS (M+1; C₁₇H₂₄NOS)=290.

### Example 147

The primary alcohol 144 ( 20 mg; 0.072 mmol) in 1 mL of CH₂Cl₂ was added to trichloroacetyl isocyanate (27 mg; 0.144 mmol) and stirred at RT for 1 hr. Solvent was removed and the residue was re-dissolved in methanol-water (1:1, 1.4 mL). Na₂CO₃ (50 mg) was added and stirred at RT for 2 h. Diluted the reaction mixture with CH₂Cl₂ and washed with water and brine. FSGC of the crude product provided compound 147 (18 mg; 79%) as white solid. mp: 187° C. (dec). LCMS (M+1): 319.

### Example 148 (Sch-725558)

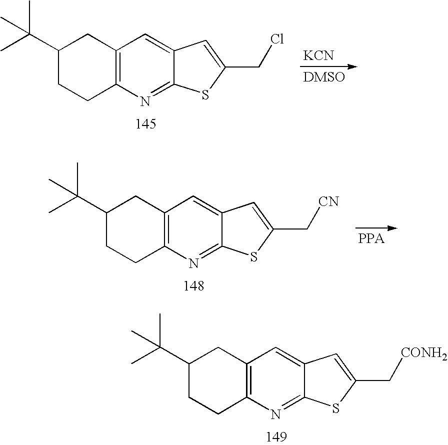

Potassium cyanide (68 mg; 1.04 mmol) was added to a solution of the chloride 145 (122 mg; 0.42 mmol) in 4.2 mL of DMSO. The resulting solution was stirred at RT for 5 h, diluted with EtOAc, washed with water and brine. Concentration to a crude residue and FSGC (10-25% EtOAc in hexanes) provided 46 mg (39%) of compound 148 as yellow solid. mp: 78-80° C. LCMS (M+1; C₁₇H₂₁N₂S): 285.

### Example 149

A solution of the cyanide 148 (28 mg; 0.1 mmol) in polyphosphoric acid (1 mL) was heated at 90° C. for 3 h, then cooled for 20 minutes and poured into crushed ice. Saturated NaHCO₃ was added to adjust the pH to ˜8. The organic product was extracted with CH₂Cl₂ and washed with water, brine and dried over Na₂SO₄. Concentration gave a yellow solid (28 mg) which was recrystallized from CH₂Cl₂-hexanes to obtain 149 as white solid (16 mg; 54%). mp: 191 (dec). LCMS (M+1): 303.

### Example 150

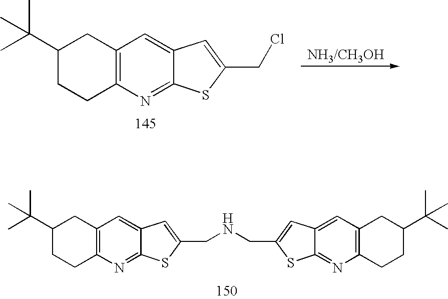

The chloride 145 (160 mg; 0.55 mmol) was dissolved in 5 mL of NH₃ in methanol was stirred at RT for 16 h. The solvent was evaporated and the residue was dissolved in CH₂Cl₂, washed with saturated NaHCO₃, water and brine. The residue from concentration of the organic extract was purified by FSGC (25-50% EtOAc in hexane) to obtain the dimeric amide 150 as yellow solid (45 mg; 16% of theory). mp: 160° C. (dec). LCMS (M+1): 532.

### Example 151

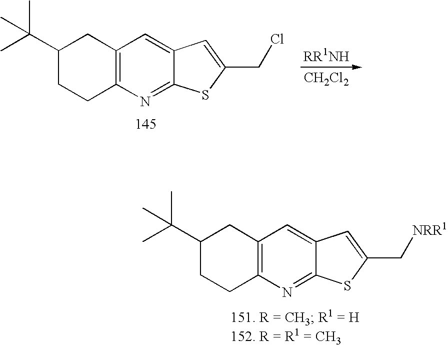

The chloride 145 (23 mg; 0.08 mmol) was dissolved in methyl amine (0.8 mL) and stirred overnight. Excess methyl amine was removed by evaporation and the residue was dissolved in CH₂Cl₂ and washed with saturated NaHCO₃ and brine. Concentration and FSGC (8% methanol in CH₂Cl₂) gave 13 mg (58%) of 151 as yellow solid. mp: 87-90° C. LCMS (M+1=C₁₇H₂₅N₂S): 289.

### Example 152

Prepared as described for 151 from 145 (32 mg; 0.011 mmol) and dimethyl amine (1 mL), stirred together for 24 h followed by standard work-up and chromatography. The dimethylamino derivative 152 was obtained as white solid (15 mg; 45%). mp: 95-97° C. LCMS (M+1): 303.

### Example 153

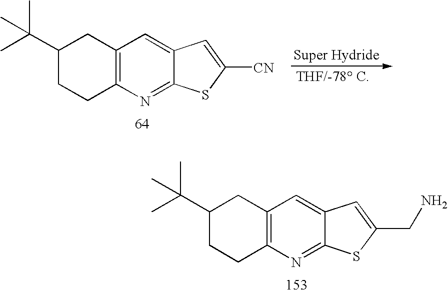

Super hydride (1M in THF; 0.32 mL) was added to a solution of the cyanide 64 (29 mg; 0.11 mmol) in THF (1 mL) at −78° C. and stirred for an hour. The reaction was warmed to RT and quenched with saturated NH₄Cl. Organic product was extracted with CH₂Cl₂, washed with water and brine. Concentration to yellow solid (50 mg) and FSGC (5% methanol in CH₂Cl₂) gave pale yellow solid 153 (15 mg; 51%). mp: 57-59° C. LCMS (M+1): 275.

### Example 154

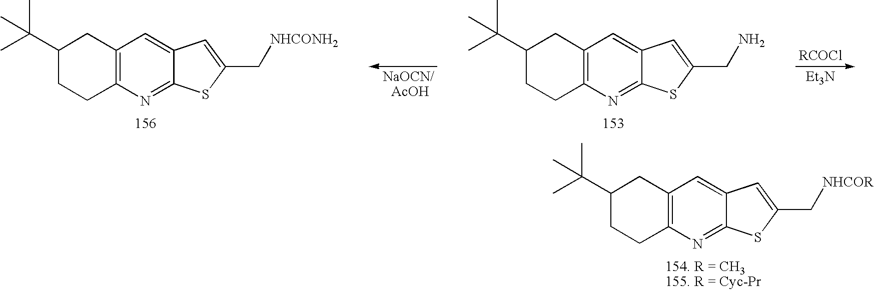

Acetyl chloride (5 μL) was added to a solution of the amine 153 (13 mg; 0.047 mmol) and Et₃N (20 μL) in 0.5 mL of CH₂Cl₂. After stirring for 40 min at RT, the reaction mixture was diluted with CH₂Cl₂, washed with 1N HCl, water, saturated NaHCO₃ and brine. Concentration to a crude residue followed by FSGC (2% methanol in CH₂Cl₂) fumished the acetamide 154 (10 mg; 68%) as yellow solid. mp: 94-96° C. LCMS (M+1): 317.

### Example 155

Prepared as described above for 154 from the amine 153 (16 mg; 0.06 mmol), Et₃N (20 μL) and cyclopropyl carbonyl chloride (7 μL) followed by standard work-up and purification. The cyclopropyl carboxamide 155 (15 mg; 75%) is a yellow solid. mp: 64-67° C. LCMS (M+1): 343.

### Example 156

Sodium cyanate (10 mg; 0.15 mmol) was added to a solution of the amine 153 (14 mg; 0.05 mmol) in 5 mL of glacial acetic acid. The reaction was stirred at RT for 3 h and then acetic acid was removed by evaporation. The residue was dissolved in CH₂Cl₂ and washed with water, saturated NaHCO₃ solution and brine. Concentration to crude product and FSGC (2% methanol in CH₂Cl₂) gave the urea 156 (7 mg; 44%) as yellow solid. LCMS (M+1; C₁₇H₂₄N₃OS)=318.

### Example 157

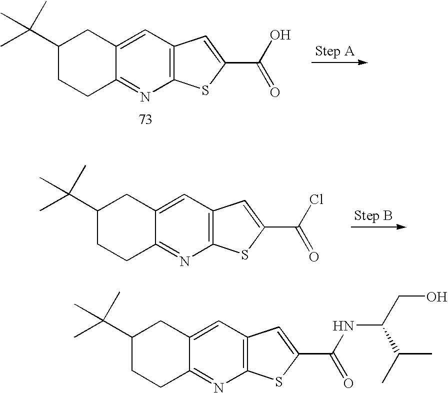 Step A:

6-tert-Butyl-5,6,7,8-tetrahydrothieno[2,3-b]quinoline-2-carbonyl chloride: To a solution of 6-tert-butyl-5,6,7,8-tetrahydrothieno[2,3-b]quinoline-2-carboxylic acid (1.00 g, 3.46 mmol.) in 15 mL of thionyl chloride and 15 mL of CH₂Cl₂ was added four drops of DMF. The reaction was stirred at 40° C. for 1.5 h. The solvent was removed under vacuum. To the residue was added 5 mL of CH₂Cl₂ and 5 mL of toluene. The resulting mixture was concentrated under vacuum to remove residual thionyl chloride. To the residue was added 5 mL of CH₂Cl₂ followed by 30 mL of hexane. The resulting solid was collected by filtration and dried under vacuum overnight to give 1.05 g (99%) of 6-tert-butyl-5,6,7,8-tetrahydrothieno[2,3-b]quinoline-2-carbonyl chloride.

Step B:

6-tert-Butyl-5,6,7,8-tetrahydrothieno[2,3-b]quinoline-2-carboxylic acid [(1S)-1-hydroxymethyl-2-methyl-propyl]amide: To a solution of (S)-2-amino-3-methyl-butan-1-ol (21 mg, 0.20 mmol) and diisopropylethylamine (52 mg, 0.40 mmol) in 2 mL of CH₂Cl₂, was added 6-tert-butyl-5,6,7,8-tetrahydrothieno[2,3-b]quinoline-2-carbonyl chloride (31 mg, 0.10 mmol). The reaction mixture was stirred at room temperature for 1 h. The content was concentrated under vacuum. The residue was purified by flash chromatography to give 36 mg (95%) of 6-tert-Butyl-5,6,7,8-tetrahydrothieno[2,3-b]quinoline-2-carboxylic acid [(1S)-1-hydroxymethyl-2-methyl-propyl]amide. LCMS: MH⁺=375; mp (° C.)=101-105.

### Examples 158-160

By essentially the same procedure set forth in Example 157, only substituting the aminoalcohol shown in Column 2 of Table 17 in Step B, the compounds in Column 3 were prepared:

TABLE 17

| Example | Column 2 | Column 3 | CMPD |
|---|---|---|---|
| 158 | 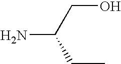 | 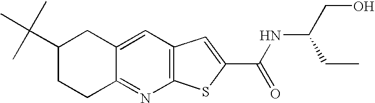 | LCMS: MH⁺ = 361; mp (° C.) =98-100 |
| 159 | 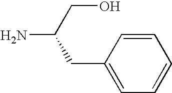 | 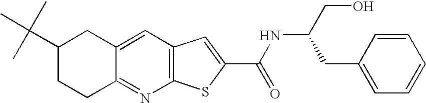 | LCMS: MH⁺ = 423; mp (° C.) =87-118 (dec.) |
| 160 | 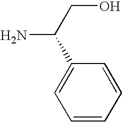 | 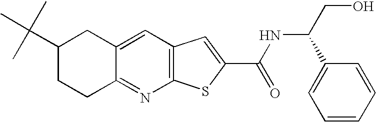 | LCMS: MH⁺ = 409; mp (° C.) =98-126 (dec.) |

### Example 161

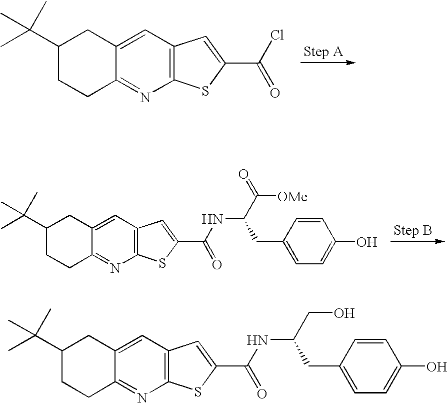 Step A:

(2S)-2-[(6-tert-Butyl-5,6,7,8-tetrahydrothieno[2,3-b]quinoline-2-carbonyl)amino]-3-(4-hydroxyphenyl)propionic acid methyl ester: To a solution of (L)-tyrosine methyl ester (64 mg, 0.33 mmol) and diisoprypylethylamine (84 mg, 0.65 mmol) in 2 mL of DMF, was added 6-tert-butyl-5,6,7,8-tetrahydrothieno[2,3-b]quinoline-2-carbonyl chloride (prepared as in Example 157 step A) (50 mg, 0.16 mmol). The reaction was stirred at room temperature for 1 h. The content was acidified by adding 0.5 mL of 2 N aqueous HCl. To the resulting solution was added 15 mL of water. The solid was collected by filtration and washed with water. It was dried under vacuum overnight to give 70 mg (92%) of (2S)-2-[(6-tert-butyl-5,6,7,8-tetrahydrothieno[2,3-b]quinoline-2-carbonyl)amino]-3-(4-hydroxyphenyl)propionic acid methyl ester.

Step B:

6-tert-Butyl-5,6,7,8-tetrahydrothieno[2,3-b]quinoline-2-carboxylic acid [2-hydroxy-(1S)-1-(4-hydroxybenzyl)ethyl]amide: To a solution of (2S)-2-[(6-tert-butyl-5,6,7,8-tetrahydrothieno[2,3-b]quinoline-2-carbonyl)amino]-3-(4-hydroxyphenyl)propionic acid methyl ester (35 mg, 0.075 mmol) in 1 mL of THF and 2 mL of EtOH, was added CaCl₂ (12.5 mg, 0.11 mmol) followed by NaBH₄ (5.7 mg, 0.15 mmol). The reaction mixture was stirred at room temperature for 2.5 h. It was quenched by adding 0.5 mL of 2 N aqueous HCl, followed by adding 10 mL of water. The content was concentrated under vacuum until white solid precipitated. The solid was collected by filtration, washed with water and dried under vacuum to give 20 mg (57%) of 6-tert-butyl-5,6,7,8-tetrahydrothieno[2,3-b]quinoline-2-carboxylic acid [2-hydroxy-(1S)-1-(4-hydroxybenzyl)ethyl]amide. LCMS: MH⁺=439; mp (° C.)=143-152 (dec.).

### Example 162

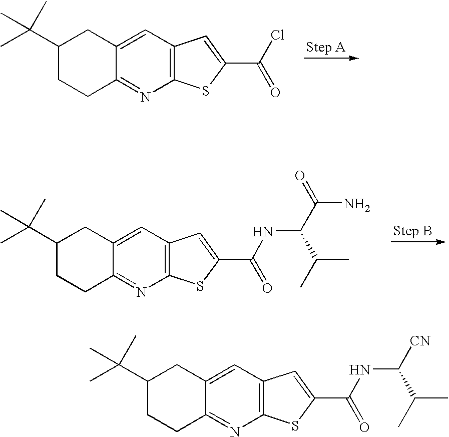 Step A:

6-tert-Butyl-5,6,7,8-tetrahydrothieno[2,3-b]quinoline-2-carboxylic acid [(1S)-1-carbamoyl-2-methylpropyl]amide: To a solution of (2S)-2-amino-3-methylbutyramide (199 mg, 1.30 mmol) and diisopropylethylamine (420 mg, 3.26 mmol) in 6 mL of DMF, was added 6-tert-butyl-5,6,7,8-tetrahydrothieno[2,3-b]quinoline-2-carbonyl chloride (prepared as in Example 157 step A) (250 mg, 0.814 mmol). The reaction was stirred at room temperature for 1 h. The content was acidified by adding 6 mL of 1 N aqueous HCl. To the resulting solution was added 150 mL of water. The solid was collected by filtration and washed with water. It was then dissolved in 60 mL of EtOAc and washed with 30 mL of dilute aqueous Na₂CO₃ and 30 mL of brine. The organic phase was concentrated to give 260 mg (75%) of 6-tert-butyl-5,6,7,8-tetrahydrothieno[2,3-b]quinoline-2-carboxylic acid [(1S)-1-carbamoyl-2-methylpropyl]amide.

Step B:

6-tert-Butyl-5,6,7,8-tetrahydrothieno[2,3-b]quinoline-2-carboxylic acid [(1S)-1-cyano-2-methylpropyl]amide: To a solution of 6-tert-butyl-5,6,7,8-tetrahydrothieno[2,3-b]quinoline-2-carboxylic acid [(1S)-1-carbamoyl-2-methylpropyl]amide (228 mg, 0.588 mmol) in 2 mL of pyridine stirred at −5° C., was added POCl₃ (100 mg, 0.654 mmol) dropwise. The reaction mixture was gradually warmed to room temperature over 0.5 h. It was diluted with 50 mL of EtOAc and washed with 1 N aqueous HCl. The organic phase was concentrated. The residue was purified by flash chromatography to give 90 mg (42%) of 6-tert-butyl-5,6,7,8-tetrahydrothieno[2,3-b]quinoline-2-carboxylic acid [(1S)-1-cyano-2-methylpropyl]amide. LCMS: MH⁺=370; mp (° C.)=189-191.

### Example 163

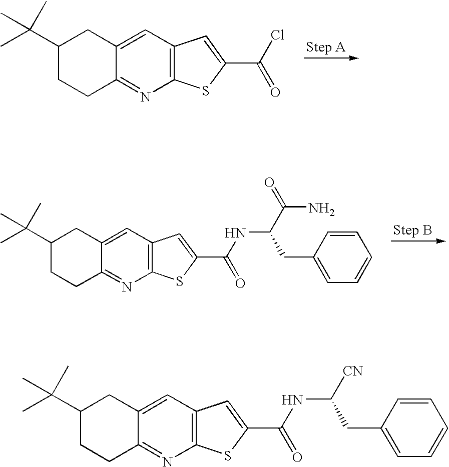 Step A:

6-tert-Butyl-5,6,7,8-tetrahydrothieno[2,3-b]quinoline-2-carboxylic acid [(1S)-1-carbamoyl-2-phenylethyl]amide: To a solution of (2S)-2-amino-3-phenylpropionamide HCl salt (261 mg, 1.30 mmol) and diisopropylethylamine (420 mg, 3.26 mmol) in 6 mL of DMF, was added 6-tert-butyl-5,6,7,8-tetrahydrothieno[2,3-b]quinoline-2-carbonyl chloride (prepared as in Example 157 step A) (250 mg, 0.814 mmol). The reaction was stirred at room temperature for 1 h. The content was acidified by adding 6 mL of 1 N aqueous HCl. To the resulting solution was added 150 ml of water. The solid was collected by filtration and washed with water. It was then dissolved in 60 mL of EtOAc and washed with 30 mL of dilute aqueous Na₂CO₃ and 30 mL of brine. The organic phase was concentrated to give 280 mg (73%) of 6-tert-butyl-5,6,7,8-tetrahydrothieno[2,3-b]quinoline-2-carboxylic acid [(1S)-1-carbamoyl-2-phenylethyl]amide.

Step B:

6-tert-Butyl-5,6,7,8-tetrahydrothieno[2,3-b]quinoline-2-carboxylic acid [(1S)-1-cyano-2-phenylethyl)amide: To a solution of 6-tert-butyl-5,6,7,8-tetrahydrothieno[2,3-b]quinoline-2-carboxylic acid [(1S)-1-carbamoyl-2-phenylethyl]amide (224 mg, 0.515 mmol) in 2 mL of THF, was added Burgess reagent (368 mg, 1.55 mmol) portionwise over 2 h. The reaction was stirred at room temperature for additional 15 min. The solvent was removed under vacuum. The residue was purified by flash chromatography to give 200 mg (93%) of 6-tert-butyl-5,6,7,8-tetrahydrothieno[2,3-b]quinoline-2-carboxylic acid [(1S)-1-cyano-2-phenylethyl)amide. LCMS: MH⁺=418; mp (° C.)=189-194 (dec).

### Example 164-165

By essentially the same procedure set forth in Example 163, only substituting the aminoamides shown in Column 2 of Table 18 in Step A, the compounds in Column 3 were prepared:

TABLE 18

| Example | Column 2 | Column 3 | CMPD |
|---|---|---|---|
| 164 | 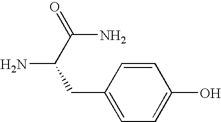 | 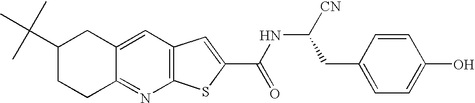 | LCMS: MH⁺ = 434; mp (° C.) =130-141 (dec.) |
| 165 | 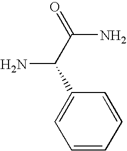 | 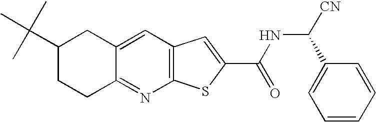 | LCMS: MH⁺ = 404; mp (° C.) =108-115 |

### Example 166

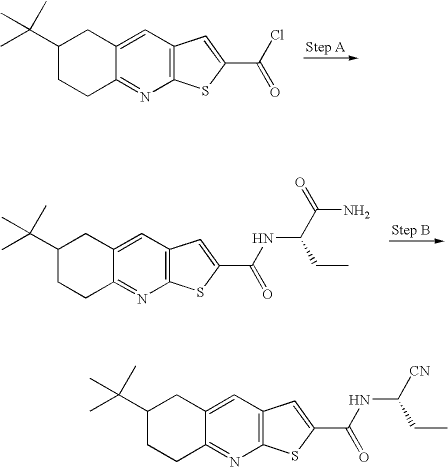 Step A:

6-tert-Butyl-5,6,7,8-tetrahydrothieno[2,3-b]quinoline-2-carboxylic acid [(1S)-1-carbamoylpropyl]amide: To a stirred solution of (2S)-2-aminobutyric acid (155 mg, 1.50 mmol) and diisopropylethylamine (387 mg, 3.00 mmol) in 3 mL of MeOH and 0.5 mL of water, was added a solution of 6-tert-butyl-5,6,7,8-tetrahydrothieno[2,3-b]quinoline-2-carbonyl chloride (prepared as in Example 157 step A) (230 mg, 0.748 mmol) in 4 mL THF/CH₂Cl₂ (1:1). The reaction was stirred at room temperature for 0.5 h. It was concentrated under vacuum. To the residue was added 10 mL of water and 1 mL of 1 N aqueous HCl. The resulting mixture was extracted by 15% MeOH/CH₂Cl₂. The organic phase was concentrated under vacuum. The residue was dissolved in 3 mL of DMF. To the resulting solution, was added K₂CO₃ (96.0 mg, 0.70 mmol) followed by iodomethane (109 mg, 0.765 mmol). The reaction mixture was stirred at room temperature for 4 h when it was acidified by 3 mL of 1 N aqueous HCl. The mixture was further diluted by 50 mL of water. The solid was collected by filtration and further purified by flash chromatography to give 225 mg of a methyl ester intermediate. This was dissolved in 10 mL of 7 N NH₃/MeOH. The reaction was stirred at 40° C. in a sealed container for 72 h. The solvent was then removed under vacuum. The residue was purified by flash chromatography to give 190 mg (68%) of 6-tert-butyl-5,6,7,8-tetrahydrothieno[2,3-b]quinoline-2-carboxylic acid [(1S)-1-carbamoylpropyl]amide.

Step B:

6-tert-Butyl-5,6,7,8-tetrahydrothieno[2,3-b]quinoline-2-carboxylic acid [(1S)-1-cyanopropyl)amide: By essentially the same procedure set forth in Example 163 step B, only replacing 6-tert-butyl-5,6,7,8-tetrahydrothieno[2,3-b]quinoline-2-carboxylic acid [(1S)-1-carbamoyl-2-phenylethyl]amide with 6-tert-butyl-5,6,7,8-tetrahydrothieno[2,3-b]quinoline-2-carboxylic acid [(1S)-1-carbamoylpropyl]amide, 6-tert-butyl-5,6,7,8-tetrahydrothieno[2,3-b]quinoline-2-carboxylic acid [(1S)-1-cyanopropyl)amide was obtained. LCMS: MH⁺=356; mp (° C.)=209-211.

### Example 167

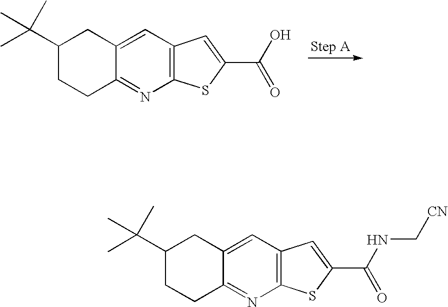

6-tert-Butyl-5,6,7,8-tetrahydrothieno[2,3-b]quinoline-2-carboxylic acid cyanomethyl-amide: To a mixture of tert-butyl-5,6,7,8-tetrahydrothieno[2,3-b]quinoline-2-carboxylic acid (250 mg, 0.865 mmol), aminoacetonitrile bisulfate (470 mg, 3.05 mmol) and HATU (525 mg, 1.38 mmol) in 4 mL of DMF, was added N-methyl morpholine (442 mg, 4.37 mmol). The reaction mixture was stirred at room temperature for 24 h. It was diluted with 40 mL of 0.5 N aqueous HCl. The resulting mixture was extracted by 50 mL of 90% EtOAc/hexanes. The organic was concentrated and the residue was purified by flash chromatography to give 260 mg (92%) of 6-tert-butyl-5,6,7,8-tetrahydrothieno[2,3-b]quinoline-2-carboxylic acid cyanomethyl-amide. LCMS: MH⁺=328; mp (° C.)=215-216.

### Example 168

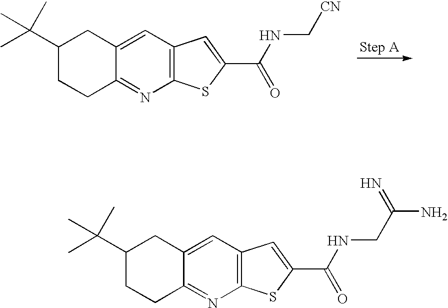 Step A:

6-tert-Butyl-5,6,7,8-tetrahydrothieno[2,3-b]quinoline-2-carboxylic acid carbamimidoylmethylamide: A mixture of 6-tert-butyl-5,6,7,8-tetrahydrothieno[2,3-b]quinoline-2-carboxylic acid cyanomethyl-amide (50 mg, 0.15 mmol) in 2 mL of EtOH cooled at 0° C., was saturated with HCl gas. The reaction container was sealed and placed in a 5° C. refrigerator for 24 h. To the reaction mixture was added 2 mL of ether. The solid was collected by filtration and dried under vacuum. 30 mg of this solid was dissolved in 2 mL of 7 N NH₃/MeOH. The reaction was stirred at room temperature for 3 h. The solvent was removed under vacuum. The residue was recrystallized from MeOH/CH₂Cl₂/hexanes to give 22 mg of 6-tert-butyl-5,6,7,8-tetrahydrothieno[2,3-b]quinoline-2-carboxylic acid carbamimidoylmethylamide as its HCl salt form. LCMS: MH⁺=345; mp (° C.)=178-199.

### Example 169-174

By essentially the same procedure set forth in Example 168, only substituting the cyano compounds shown in Column 2 of Table 19, the compounds in Column 3 were prepared:

TABLE 19

| Example | Column 2 | Column 3 | CMPD |
|---|---|---|---|
| 169 | 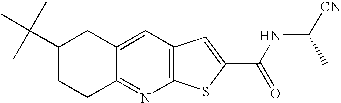 | 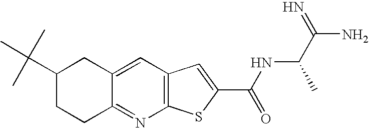 | LCMS: MH⁺ = 359; mp (° C.) =186-214 (dec.) |
| 170 | 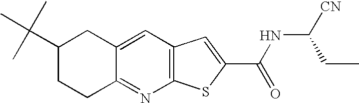 | 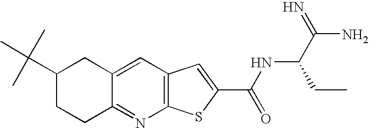 | LCMS: MH⁺ = 373; mp (° C.) =187-204 (dec.) |
| 171 | 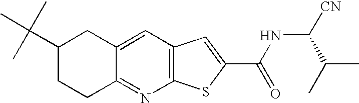 | 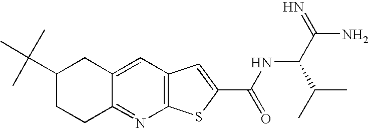 | LCMS: MH⁺ = 387; mp (° C.) =191-210 (dec.) |
| 172 | 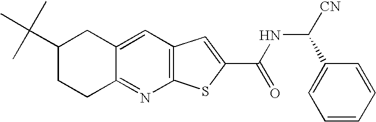 | 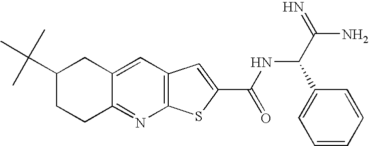 | LCMS: MH⁺ = 421; mp (° C.) =172-208 (dec.) |
| 173 | 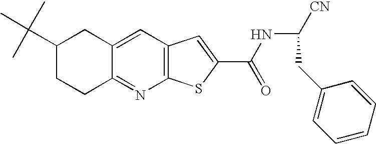 | 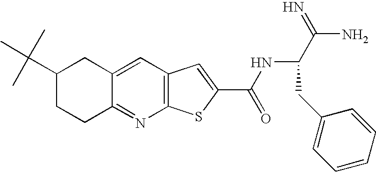 | LCMS: MH⁺ = 435; mp (° C.) =185-202 (dec.) |
| 174 | 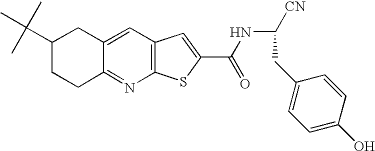 | 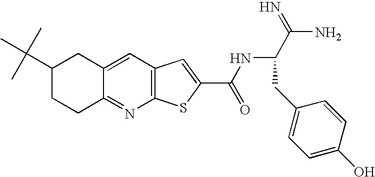 | LCMS: MH⁺ = 451; mp (° C.) =182-219 (dec.) |

### Example 175

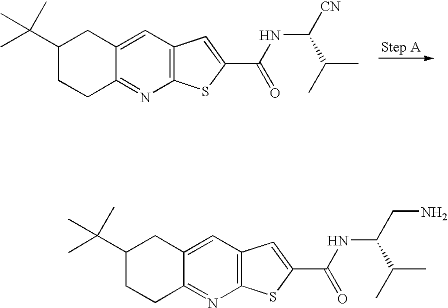

6-tert-Butyl-5,6,7,8-tetrahydrothieno[2,3-b]quinoline-2-carboxylic acid [(1S)-1-aminomethyl-2-methyl]propyl)amide: To a stirred solution of 6-tert-butyl-5,6,7,8-tetrahydrothieno[2,3-b]quinoline-2-carboxylic acid [(1S)-1-cyano-2-methylpropyl]amide (32 mg, 0.077 mmol) and CoCl₂.6H₂O (37 mg, 0.15 mmol) in 2 mL of THF/MeOH (1:3) cooled at −5° C., was added NaBH₄. The reaction was stirred at −5° C. for 0.5 h and then warmed to room temperature. It was quenched by adding 3 mL of 2 N aqueous HCl. The resulting mixture was stirred at room temperature for 0.5 h. The content was filtered. The filtrate was concentrated under vacuum to remove MEOH and THF. To the aqueous residue was added 5 mL of aqueous NH₄OH. The mixture was extracted with CH₂Cl₂. The organic layer was concentrated and the residue was further purified by flash chromatography to give 20 mg (62%) of 6-tert-butyl-5,6,7,8-tetrahydrothieno[2,3-b]quinoline-2-carboxylic acid [(1S)-1-aminomethyl-2-methylpropyl)amide. LCMS: MH⁺=374; mp (° C.)=76-88 (dec.).

### Example 176-180

By essentially the same procedure set forth in Example 175, only substituting th e cyano compounds shown in Column 2 of Table 20, the compounds in Column 3 were prepared:

TABLE 20

| Example | Column 2 | Column 3 | CMPD |
|---|---|---|---|
| 176 | 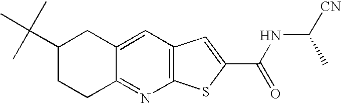 | 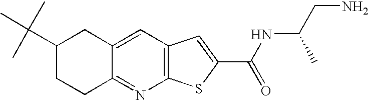 | LCMS: MH⁺ = 346; mp (° C.) =96 (dec.) |
| 177 | 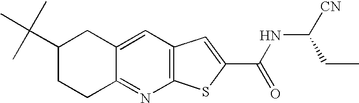 | 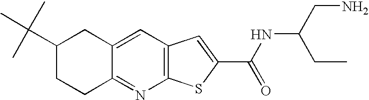 | LCMS: MH⁺ = 360; mp (° C.) =71-84 (dec.) |
| 178 | 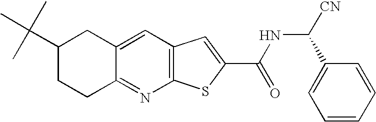 | 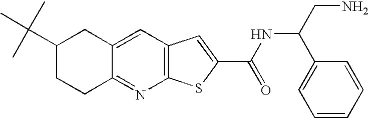 | LCMS: MH⁺ = 408; mp (° C.) =99-130 (dec.) |
| 179 | 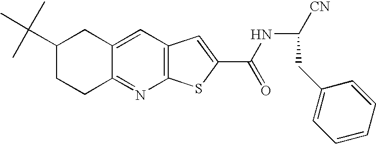 | 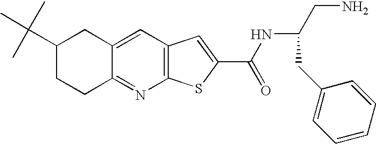 | LCMS: MH⁺ = 422; mp (° C.) =68-78 (dec.) |
| 180 | 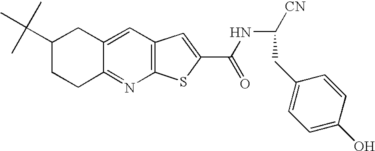 | 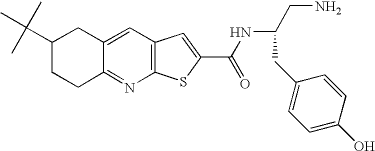 | LCMS: MH⁺ = 438; mp (° C.) =128-174 (dec.) |

### Example 181

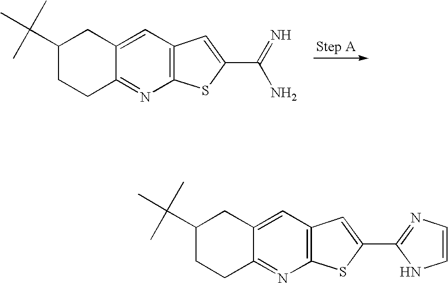 Step A:

6-tert-Butyl-2-(1H-imidazol-2-yl)-5,6,7,8-tetrahydrothieno[2,3-b]quinoline: To a solution of 6-tert-butyl-5,6,7,8-tetrahydrothieno[2,3-b]quinoline-2-carboxamidine (33.0 mg, 0.115 mmol) in 1 mL of THF, was added chloroacetaldehyde (260 mg, 3.31 mmol) followed by 5 drops of saturated NaHCO₃ aqueous solution. The reaction was stirred at room temperature for 60 h. It was diluted by 60 mL of CH₂Cl₂ and washed with 10 mL of water. The organic phase was dried over anhydrous Na₂SO₄ and then concentrated. The residue was further purified by flash chromatography to give 19.5 mg (55%) of 6-tert-butyl-2-(1H-imidazol-2-yl)-5,6,7,8-tetrahydrothieno[2,3-b]quinoline. LCMS: MH⁺=312; mp (° C.)=142-190 (dec.).

### Example 182

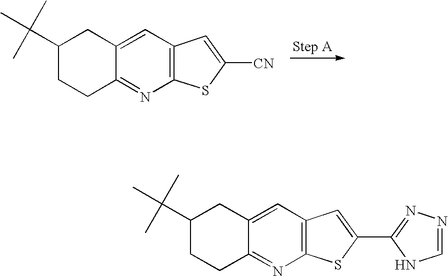 Step A:

6-tert-Butyl-2-(4H-[1,2,4]triazol-3-yl)-5,6,7,8-tetrahydrothieno[2,3-b]quinoline: A mixture of 6-tert-butyl-5,6,7,8-tetrahydrothieno[2,3-b]quinoline-2-carbonitrile (200 mg, 0.741 mmol), hydrazine monohydrate (370 mg, 7.40 mmol) in 5 ml of DMSO was stirred at room temperature for 48 h. Additional hydrazine monohydrate (185 mg, 3.70 mmol) was added at this time and the reaction was stirred at room temperature for additional 16 h. To the reaction solution was added 50 mL of water. The resulting solid (170 mg) was collected by filtration, washed with ether and dried under vacuum. A portion of the solid (33 mg) was mixed with 0.5 ml of triethyl orthoformate and the resulting mixture was stirred at 140° C. for 3 h. The solvent was removed under vacuum. The residue was purified by flash chromatography to provide 21 mg of a UV-active material which was then dissolved in 2 mL of 12 N aqueous HCl and stirred at room temperature for 1 h. This was neutralized by 2 N aqueous NaOH. The resulting mixture was extracted with CH₂Cl₂. The organic phase was dried over anhydrous Na₂SO₄ and then concentrated to give 14.5 mg of 6-tert-butyl-2-(4H-[1,2,4]triazol-3-yl)-5,6,7,8-tetrahydrothieno[2,3-b]quinoline. LCMS: MH⁺=313; mp (° C.)=102-125 (dec.).

### Example 183

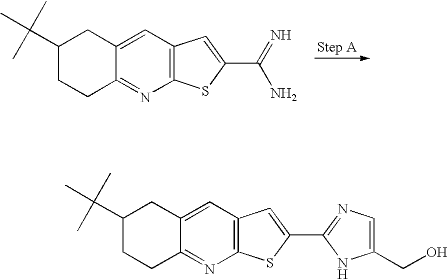 Step A:

[2-(6-tert-Butyl-5,6,7,8-tetrahydrothieno[2,3-b]quinolin-2-yl)-3H-imidazol-4-yl]methanol: A mixture of 6-tert-butyl-5,6,7,8-tetrahydrothieno[2,3-b]quinoline-2-carboxamidine (118 mg, 0.41 mmol), 1,3-dihydroxyacetone (75 mg, 0.84 mmol) and NH₄Cl (90 mg, 1.7 mmol) in 1.5 mL of 7 N NH₃/MeOH was sealed in a reaction vial and stirred at 80° C. for 1 h. After it was cooled to room temperature, 15 mL of water was added. The solid was collected by filtration and further purified by recrystallization from MeOH/CH₂Cl₂ to give 70 mg (50%) of [2-(6-tert-butyl-5,6,7,8-tetrahydrothieno[2,3-b]quinolin-2-yl)-3H-imidazol-4-yl]methanol. LCMS: MH⁺=342; mp (° C.)=228-237 (dec.).

### Example 184

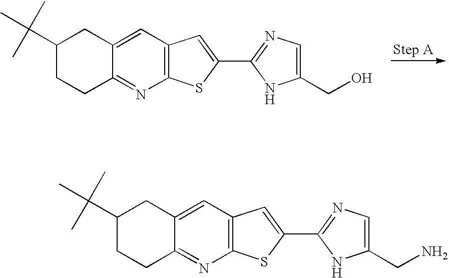 Step A:

[2-(6-tert-Butyl-5,6,7,8-tetrahydrothieno[2,3-b]quinolin-2-yl)-3H-imidazol-4-yl]methylamine: A solution of [2-(6-tert-butyl-5,6,7,8-tetrahydrothieno[2,3-b]quinolin-2-yl)-3H-imidazol-4-yl]methanol (38 mg, 0.11 mmol) in 1.5 mL of thionyl chloride was stirred at 80° C. for 15 min. The solvent was removed under vacuum. To the residue was added NaN₃ (36 mg, 0.56 mmol) followed by 1.5 mL of DMF. The reaction was stirred at room temperature for 5 h. It was diluted with 10 mL of water. The resulting solid was collected by filtration and dissolved in 5 mL of MeOH. To the solution was added 10% Pd/C (36 mg). The resulting mixture was stirred under 1 atm of hydrogen gas for 3 h. The mixture was filtered through celite. The filtrate was concentrated. The residue was by flash chromatography to give 18 mg (47%) of [2-(6-tert-butyl-5,6,7,8-tetrahydrothieno[2,3-b]quinolin-2-yl)-3H-imidazol-4-yl]methylamine. LCMS: MH⁺=341; mp (° C.)=185-220 (dec.).

### Example 185

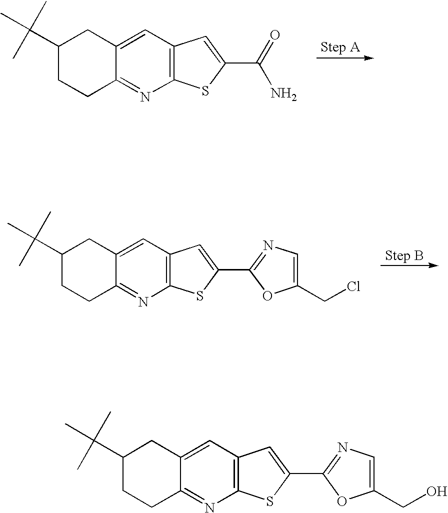 Step A:

6-tert-Butyl-2-(5-chloromethyl-oxazol-2-yl)-5,6,7,8-tetrahydrothieno[2,3-b]quinoline: A mixture of 6-tert-butyl-5,6,7,8-tetrahydrothieno[2,3-b]quinoline-2-carboxylic acid amide (200 mg, 0.694 mmol) and 1,3-dichloroacetone (448 mg, 3.47 mmol) was stirred at 130 oC. for 1 h. The resulting dark mixture was cooled to room temperature. It was diluted with 20 mL of CH₂Cl₂ and washed with 10 mL of water. The organic phase was dried over anhydrous Na₂SO₄ and then concentrated. The residue was further purified by flash chromatography to give 60 mg (24%) of 6-tert-butyl-2-(5-chloromethyl-oxazol-2-yl)-5,6,7,8-tetrahydrothieno[2,3-b]quinoline.

Step B:

[2-(6-tert-Butyl-5,6,7,8-tetrahydrothieno[2,3-b]quinolin-2-yl)oxazol-5-yl]methanol: A mixture of 6-tert-butyl-2-(5-chloromethyl-oxazol-2-yl)-5,6,7,8-tetrahydrothieno[2,3-b]quinoline (45 mg, 0.13 mmol) and NaHCO₃ (105 mg, 1.3 mmol) in 1 mL of DMSO was heated at 130° C. under N₂ for 1 h. It was cooled to room temperature and diluted with 60 mL of water. The mixture was extracted by 60% EtOAc/hexane. The organic phase was dried over anhydrous Na₂SO₄ and then concentrated. The residue was dissolved in 3 mL of MeOH/CH₂Cl₂ (1:1). To this was added NaBH₄ (7 mg, 0.19 mmol). The reaction was stirred at room temperature for 1 h. The solvent was removed under vacuum. The residue was further purified by flash chromatography to give 20 mg (47%) of [2-(6-tert-butyl-5,6,7,8-tetrahydrothieno[2,3-b]quinolin-2-yl)oxazol-5-yl]methanol. LCMS: MH⁺=343; mp (° C.)=93-97 (dec.).

### Example 186

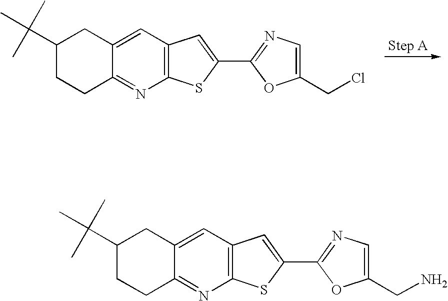 Step A:

[2-(6-tert-Butyl-5,6,7,8-tetrahydrothieno[2,3-b]quinolin-2-yl)oxazol-5-yl]methylamine: To a solution of 6-tert-butyl-2-(5-chloromethyl-oxazol-2-yl)-5,6,7,8-tetrahydrothieno[2,3-b]quinoline (30 mg, 0.083 mmol) in 1 mL of DMF was added NaN₃ (16 mg, 0.25 mmol). The reaction was stirred at room temperature for 3 h. To the solution was added 20 mL of water. The mixture was extracted by 40% EtOAc/hexane. The organic phase was concentrated to give a residue, which was dissolved in 2 mL of THF/H₂O (4:1). To the solution was added triphenylphosphine (33 mg, 0.13 mmol) and triethylamine (13 mg, 0.13 mmol). The reaction was stirred at room temperature for 24 h. The solvent was removed under vacuum. The residue was purified by flash chromatography to give 13 mg (46%) of [2-(6-tert-butyl-5,6,7,8-tetrahydrothieno[2,3-b]quinolin-2-yl)oxazol-5-yl]methylamine. LCMS: MH⁺=342; mp (° C.)=142-178 (dec.).

### Example 187

### Ethyl 3-Amino-5,6,7,8-Tetrahydro-6-(Trimethylsilyl)thieno[2,3-b]quinoline-2-Carboxylate

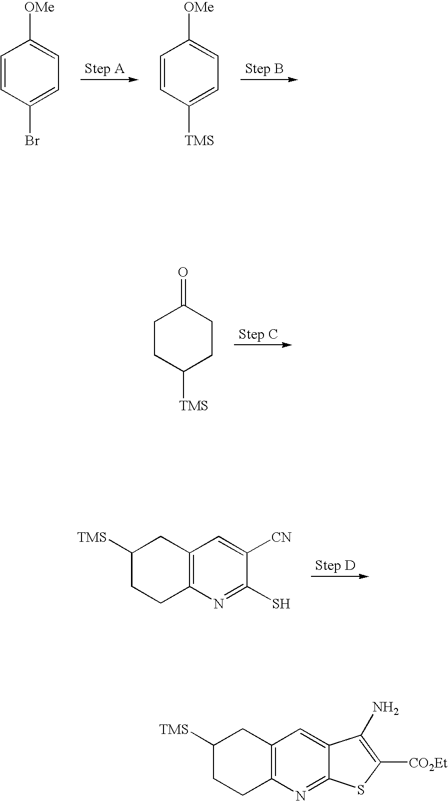

STEP A: To a suspension of magnesium turnings (8.7 g, 0.36 mol) in tetrahydrofuran (300 ml) at room temperature under nitrogen, 4-bromoanisole (37.5 ml, 0.30 mol) was added in small portions in which the reaction mixture was kept in gentle reflux. After the addition of 4-bromoanisole, the mixture was heated at 70° C. for another 3 hr. The reaction mixture was cooled at 0° C. and trimethylsilyl chloride (16.5 ml, 0.36 mol) in tetrahydrofuran (50 ml) was added dropwise. The mixture was stirred at 0° C. for another hour before it was quenched with saturated ammonium chloride solution. Water and ethyl acetate were added. Layers were separated and the separated aqueous layer was extracted with ethyl acetate (×2). The combined organic layers were dried (MgSO₄) and filtered. Removal of solvents in vacuo followed by high vacuum distillation gave a colorless oil (35 g, 65%).

STEP B: To a solution of trimethylsilylanisole (6.0 g, 0.033 mol) in a mixture of liquid ammonia (50 ml), ethanol (30 ml) and ether (40 ml) at −30° C., sodium was added in small pieces. After the addition of sodium, the mixture was stirred at −30° C. until the color of the mixture was turned from blue to colorless. The cooling bath was then removed and the mixture was warmed to room temperature slowly. The mixture was stirred at room temperature until all ammonia was evaporated to give a white solid. Water was added to dissolve the solid and the mixture was extracted with ether (×2). The combined organic layers were dried (MgSO₄) and filtered. Solvents were removed from the filtrate to give a colorless oil. The colorless oil was then dissolved in a mixture of ethanol and water. Oxalic acid hydrate (840 mg, 6.66 mmol) was added and the mixture was stirred at room temperature for 3 hrs. Water and ether were added to the mixture and layers were separated. The separated aqueous layer was extracted with ether (×2), dried (MgSO₄) and filtered. Solvents in the filtrate were removed to give a ketone (4.5 g, 79%) as colorless oil.

STEP C: To a solution of ketone (4.5 g, 0.026 mol) and methyl formate (3.2 ml, 0.040 mol) in ether (100 ml) at room temperature, a solution of sodium ethoxide (14 ml, 0.040 mol, 21 wt % in ethanol) was added. The mixture was stirred at room temperature for 3 hrs. Water and ether were added. Layers were separated and the organic layer was extracted with water. All aqueous layers were combined and a solution of piperidine/acetic acid and cyanothioacetamide were added. The mixture was then heated at 100° C. for 1 hr. After being cooled at room temperature, water and ethyl acetate were added. Layers were separated and the aqueous layer was extracted with ethyl acetate (×2). The combined organic layers were dried (MgSO₄) and filtered. Removal of solvents in vacuum gave a yellow solid. The yellow solid was extensively washed with ether and then dried to give thio (3.9 g, 57%) yellow solid. Electrospray LCMS [M+1]⁺=263.

STEP D: To a suspension of thio (1.0 g, 3.81 mmol) in acetone (100 ml) at room temperature, potassium bicarbonate (1.58 g, 11.4 mmol) was added followed by ethyl chloroacetate (0.7 g, 5.72 mmol). The mixture was stirred at room temperature overnight and solvents were removed in vacuum. Ethanol was added and the mixture was heated to reflux for 1 hr. Solvents were removed in vacuum. Water and ethyl acetate were added. Layers were allowed to separate and the separated aqueous layer was extracted with ethyl acetate (×2). The combined organic layers were dried (MgSO₄) and filtered. Removal of solvents in vacuum gave a yellow solid. The yellow solid was washed with ether to give ethyl 3-amino-5,6,7,8-tetrahydro-6-(trimethylsilyl)thieno[2,3-b]quinoline-2-carboxylate (969 mg, 73%) as yellow solid. Electrospray LCMS [M+1]⁺=349.

### Example 188

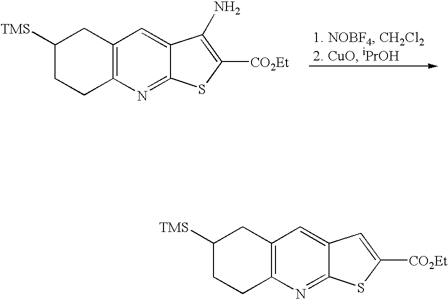

### Ethyl 5,6,7,8-Tetrahydro-6-(Trimethylsilyl)thieno[2,3b]quinoline-2-Carboxylate

To a solution of aminoester (450 mg, 1.29 mmol) in dichloromethane (10 ml) at room temperature, nitrosonium tetrafuoroborate (226 mg, 1.94 mmol) was added in small portions. The mixture was stirred at room temperature for 1 hr. and copper oxide (185 mg, 1.29 mmol) and isopropanol (10 ml) were added. The red suspension was stirred at room temperature for an additional hour and solid was filtered through Celite. Solvents were removed in vacuum to give a red oil. Column purification [hexanes/ethyl acetate, 5:1 (v/v)] gave ethyl 5,6,7,8-tetrahydro-6-(trimethylsilyl)thieno[2,3-b]quinoline-2-carboxylate (353 mg, 82%) as a yellow solid. Chiral HPLC separation using Chiralpak OD (9:1v/v=hexanes-isopropanol) gave first the less polar enantiomer A as white solid. The more polar enantiomer B was also obtained as white solid. Electrospray LCMS [M+1]⁺=334.

### Example 189

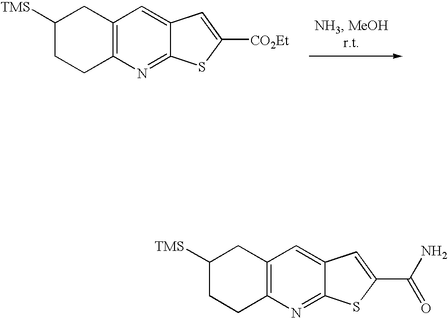

5,6,8-Tetrahydro-6-(Trimethylsilyl)thieno[2,3-b]quinoline-2-Carboxamide: To a solution of ethyl 5,6,7,8-tetrahydro-6-(trimethylsilyl)thieno[2,3-b]quinoline-2-carboxylate (105 mg, 0.32 mmol, enantiomer B) in methanol (5 ml) at 0° C., ammonia was bubble through the solution for 20 min. The mixture was stirred in a sealed-tube for 2 days. Removal of solvents in vacuum gave a white solid. The solid was washed extensively with ether to give 5,6,7,8-tetrahydro-6-(trimethylsilyl)thieno[2,3-b]quinoline-2-carboxamide (85 mg, 89%) as a white solid. Electrospray LCMS [M+1]⁺=305.

### For Examples 190-191

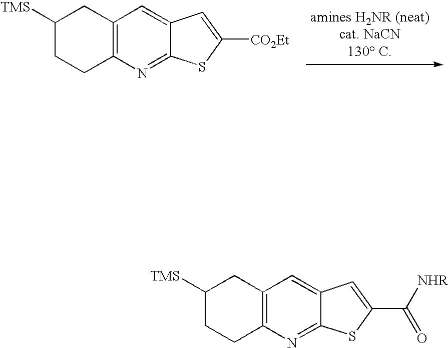

A mixture of ethyl 5,6,7,8-tetrahydro-6-(trimethylsilyl)thieno[2,3-b]quinoline-2-carboxylate and catalytic sodium cyanide was heated in the corresponding neat amines at 130° C. overnight. After being cooled to room temperature, water and ethyl acetate were added. Layers were separated and the organic layer was washed with water (×2). The organic layer was dried (MgSO₄) and filtered. Solvents were removed in vacuum and ether was added to induce crystallization of the product carboxamides. The carboxamides were then washed extensively with ether to give pure amides.

### Example 190

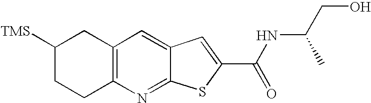

5,6,7,8-Tetrahydro-N-(2-hydroxy-1(S)-methylethyl)-6-(Trimethylsilyl)thieno[2,3-b]quinoline-2-Carboxamide: The title compound (24 mg, 50%) was obtained as white solid. Electrospray LCMS [M+1]⁺=363.

### Example 191

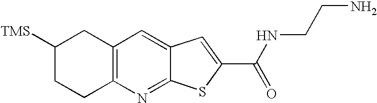

N-(2-Aminoethyl) 5,6,7,8-Tetrahydro-6-(Trimethylsilyl)thieno[2,3-b]quinoline-2-Carboxamide: The title compound (11 mg, 48%) was obtained as white solid. Electrospray LCMS [M+1]⁺=348.

### For Examples 192-199

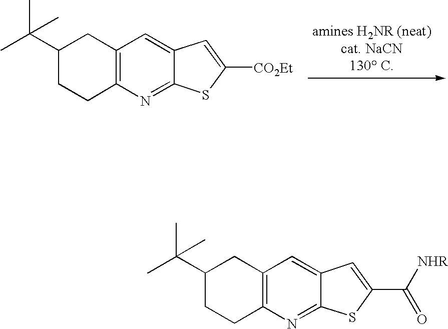

A mixture of ester and catalytic sodium cyanide was heated in the corresponding neat amines at 130° C. overnight. After being cooled to room temperature, water and ethyl acetate were added. Layers were separated and the organic layer was washed with water (×2). The organic layer was dried (MgSO₄) and filtered. Solvents were removed in vacuum and ether was added to induce crystallization of the product carboxamides. The carboxamides were washed extensively with ether to give pure carboxamides.

### Example 192

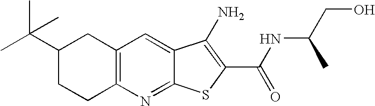

3-Amino-6-(1,1-Dimethylethyl)-5,6,7,8-Tetrahydro-N-(2-Hydroxy-1(S)-Methylethyl)thieno[

2,3-b]quinoline-2-Carboxamide: The title compound (15 mg, 55%) was obtained as white solid. Electrospray LCMS [M+1]⁺=363.

### Example 193

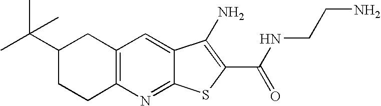

3-Amino-N-(2-Aminoethyl)-6-(1,1-Dimethylethyl)-5,6,7,8-Tetrahydrothieno[2,3-b]quinoline-2-Carboxamide: The title compound (12 mg, 52%) was obtained as white solid. Electrospray LCMS [M+1]⁺=347.

### Example 194

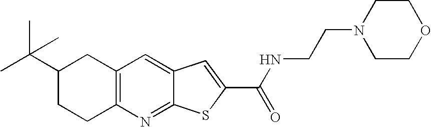

6-(1,1-Dimethylethyl)-5,7,8-Tetrahydro-N-[2-(4-Morpholinyl)ethyl]thieno[2,3-b]quinoline-2-Carboxamide: The title compound (91 mg, 48%) was obtained as white solid. Electrospray LCMS [M+1]⁺=402.

### Example 195

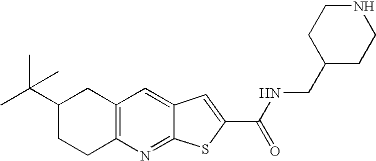

6-(1,1-Dimethylethyl)-5,6,7,8-Tetrahydro-N-(4-Piperidinylmethyl)thieno[2,3-b]quinoline-2-Carboxamide: The title compound (72 mg, 40%) was obtained as white solid. Electrospray LCMS [M+1]⁺=386.

### Example 196

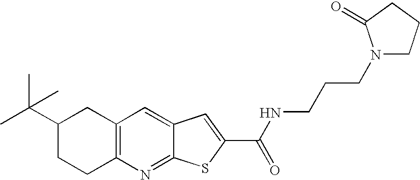

6-(1,1-Dimethylethyl)-5,6,7,8-Tetrahydro-N-[3-(2-oxo-1-Pyrrolidinyl)propyl]thieno[2,3-b]quinoline-2-Carboxamide: The title compound (86 mg, 44%) was obtained as white solid. Electrospray LCMS [M+1]⁺=414.

### Example 197

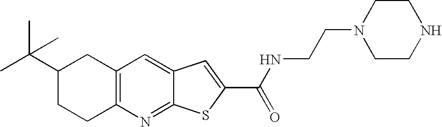

6-(1,1-Dimethylethyl)-5,6,7,8-Tetrahydro-N-[2-(1-Piperazinyl)ethyl]thieno[2,3-b]quinoline-2-Carboxamide: The title compound (94 mg, 50%) was obtained as white solid. Electrospray LCMS [M+1]⁺=401.

### Example 198

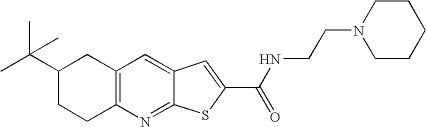

6-(1,1-Dimethylethyl)-5,6,7,8-Tetrahydro-N-[2-(1-Piperidinyl)ethyl]thieno[2,3-b]quinoline-2-Carboxamide: The title compound (98 mg, 52%) was obtained as white solid. Electrospray LCMS [M+1]⁺=400.

### Example 199

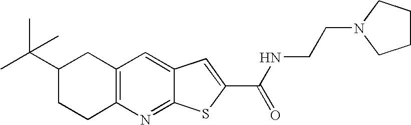

6-(1,1-Dimethylethyl)-5,6,7,8-tetrahydro-N-[2-(1-Pyrrolidinyl)ethyl]thieno[2,3-b]quinoline-2-Carboxamide: The title compound (100 mg, 55%) was obtained as white solid. Electrospray LCMS [M+1]⁺=386.

### Example 200

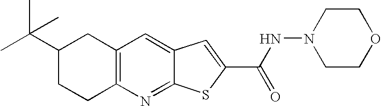

6-(1,1-Dimethylethyl)-5,6,7,8-Tetrahydro-N-(4-Morpholinyl)thieno[2,3-b]quinoline-2-Carboxamide: The title compound (35 mg, 20%) was obtained as white solid. Electrospray LCMS [M+1]⁺=374.

### Example 201

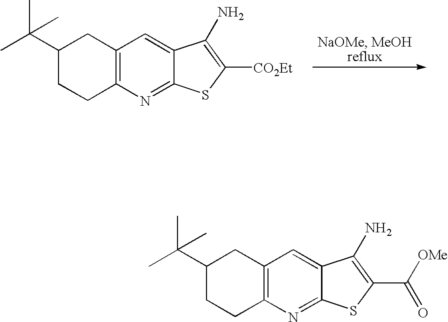

Methyl 3-Amino-6-(1,1-Dimethylethyl)-5,6,7,8-Tetrahydrothieno[2,3-b]quinoline-2-Carboxylate: To a solution of ethyl 3-amino-6-(1,1-dimethylethyl)-5,6,7,8-[2,3-b]quinoline-2-carboxylate (100 mg, 0.30 mmol) in methanol (2 ml), a catalytic amount of sodium methoxide was added. The mixture was heated to reflux overnight. After being cooled at room temperature, water and ethyl acetate were added. Layers were separated and the organic layer was washed with water, dried (MgSO4) and filtered. Removal of solvents in vacuum gave yellow solid which was washed extensively with ether to give methyl 3-amino-6-(1,1-dimethylehtly)-5,6,7,8-tetrahydrothieno[2,3-b]quinoline-2-carboxylate as pale yellow solid (86 mg, 90%). Electrospray LCMS [M+1]⁺=319.

### Example 202

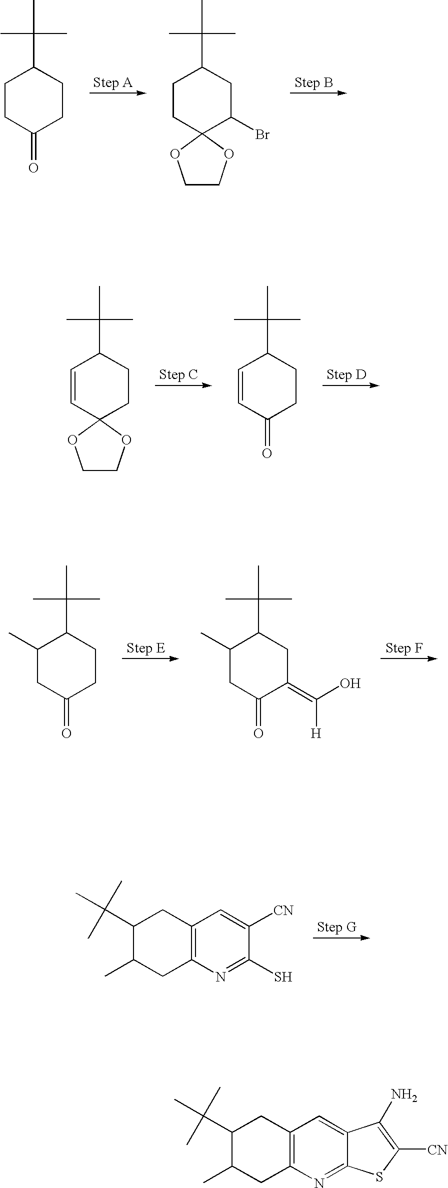 Step A:

6-Bromo-8-tert-butyl-1,4-dioxa-spiro[4,5]decane. To a solution of 4-tert-butylcyclohexanone (10.0 g, 64.8 mmol) in ethylene glycol (130 mL) at 0° C. was added bromine (3.3 mL, 64.8 mmol). The reaction was allowed to warm to room temperature and stir for 12 h. The reaction was diluted with pentane and quenched at 0° C. by the addition of solid Na₂CO₃. The reaction was stirred for 20 minutes, water was added and the layers were separated. The pentane layer was washed with 10% aqueous sodium thiosulfate solution, dried over MgSO₄, and concentrated in vacuo to give 6-bromo-8-tert-butyl-1,4-dioxa-spiro[4,5]decane as a colorless liquid (17.5 g, 97% yield).

Step B:

8-tert-Butyl-1,4-dioxa-spiro[4,5]dec-6-ene. To a flask containing 6-bromo-8-tert-butyl-1,4-dioxa-spiro[4,5]decane (17.5 g, 63.2 mmol) in DMSO (73.5 mL) was added NaOMe (13.7 g, 253.6 mmol). The mixture was heated at 55° C. for 12 h. The reaction was cooled to room temperature and water was added. The aqueous layer was extracted with pentane. The organic phase was dried over MgSO₄, and concentrated in vacuo to give 8-tert-butyl-1,4-dioxa-spiro[4,5]dec-6-ene a colorless liquid that was taken on to step C.

Step C:

4-tert-Butylcyclohex-2-enone. A solution of 8-tert-butyl-1,4-dioxa-spiro[4,5]dec-6-ene (11 g, 56 mmol) in 1,4-dioxane (33 mL) was treated with 1 N H₂SO₄ solution (40 mL). The reaction was stirred at room temperature for 16 h. The aqueous layer was extracted with ether. The combined organic layer was washed with saturated NaHCO₃, brine, dried over MgSO₄, and concentrated in vacuo. Purification via silica gel chromatograghy (20% EtOAc/hexanes) provided 4-tert-butylcyclohex-2-enone as a colorless liquid (6.98 g, 82% yield, 2 steps).

Step D:

4-tert-Butyl-3-methyl-cyclohexenone. A flask was charged with copper bromide-dimethylsulfide complex (12.5 g, 61.0 mmol) in Et₂O (61 mL). The mixture was cooled to −40° C. and a solution of MeLi (52 mL, 1.5 M in Et₂O, 77.9 mmol) was slowly added. The reaction was stirred at −40° C. for 20 minutes, then cooled to −78° C. A solution of 4-tert-butylcyclohex-2-enone (6.98 g, 45.8 mmol) in Et₂O was added slowly to the reaction flask. The yellow reaction was continued to stir under a N₂ atmosphere at −78° C. for 3 h. The reaction was allowed to slowly warm to room temperature and stir for an additional 12 h. The reaction was diluted with ether and quenched by the slow addition of saturated NH₄Cl. The aqueous layer was extracted with ether. The combined organic phase was washed with saturated NH₄Cl, dried over MgSO₄, and concentrated in vacuo. Purification via silica gel chromatography (10% -20% EtOAc/hexanes) provided 4-tert-butyl-3-methyl-cyclohexenone as a yellow oil (2.01 g, 26% yield).

Step E:

5-tert-Butyl-4-methyl 2-oxo-cyclohanecarbaldehyde. Following a similar procedure set forth in Example 1, Step A, only substituting the ketone shown in Example 1 with 4-tert-butyl-3-methyl-cyclohexenone (2.01 g, 11.94 mmol) gave 2.33 g (99% yield) of 5-tert-butyl-4-methyl-2-oxo-cyclohanecarbaldehyde as a yellow oil.

Step F:

6-tert-Butyl-2-mercapto-7-methyl-5,6,7,8-tetrahydro-quinoline-3-carbonitrile. Following a similar procedure set forth in Example 1, Step B, only substituting the α-formyl ketone shown in Example 1 with 5-tert-butyl-4-methyl-2-oxo-cyclohexanecarbaldehyde (2.33 g, 11.87 mmol) gave 2.00 g (65% yield) of 6-tert-butyl-2-mercapto-7-methyl-5,6,7,8-tetrahydro-quinoline-3-carbonitrile as a yellow solid that was used without further purification.

Step G:

3-Amino-6-tert-butyl-7-methyl-5,6,7,8-tetrahydrothieno[2,3-b]quinoline-2-carbonitrile. Following a similar procedure set forth in Example 1, Step C, only substituting the mercapto-nitrile shown in Example 1 with 6-tert-butyl-2-mercapto-7-methyl-5,6,7,8-tetrahydro-quinoline-3-carbonitrile (1.90 g, 7.31 mmol) gave 1.345 g (61% yield) of 3-amino-6-tert-butyl-7-methyl-5,6,7,8-tetrahydrothieno[2,3-b]quinoline-2-carbonitrile as an orange solid. LCMS [M+1]⁺=300; mp (° C.)=181-197.

### Example 203

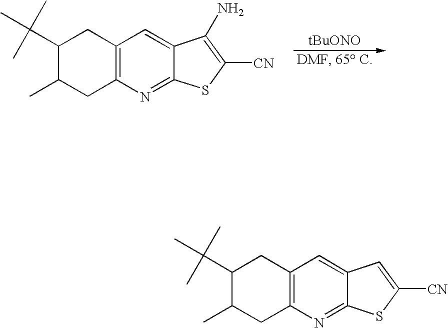

6-tert-Butyl-7-methyl-5,6,7,8-tetrahydro-thieno[2,3,-b]quinoline-2-carbonitrile. Following a similar procedure set forth in Example 58, Step A, only substituting the amino-nitrile shown in Example 58 with 6-tert-butyl-2-mercapto-7-methyl-5,6,7,8-tetrahydro-quinoline-3-carbonitrile (1.35 g, 4.49 mmol) gave 0.4804 g (38% yield) of 6-tert-butyl-7-methyl-5,6,7,8-tetrahydro-thieno[2,3,-b]quinoline-2-carbonitrile as an orange solid. LCMS [M+1]⁺=285; mp (° C.)=107-110.

### Example 204

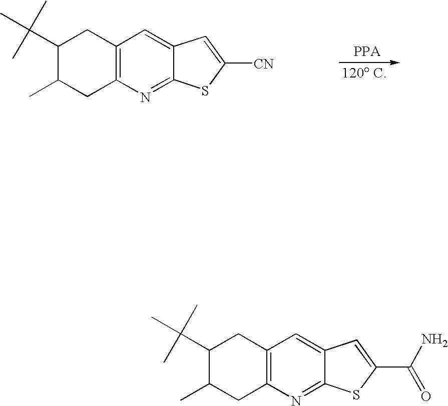

6-tert-Butyl-7-methyl-5,6,7,8-tetrahydro-thieno[2,3,-b]quinoline-2-carboxylic acid amide. Following a similar procedure set forth in Example 64, Step A, only substituting the carbonitrile shown in Example 64 with 6-tert-butyl-7-methyl-5,6,7,8-tetrahydro-thieno[2,3,-b]quinoline-2-carbonitrile (0.335 g, 1.18 mmol) gave 0.3327 g (93% yield) of 6-tert-butyl-7-methyl-5,6,7,8-tetrahydro-thieno[2,3,-b]quinoline-2-carboxylic acid amide as a cream-colored solid. LCMS [M+1]⁺=303; mp (° C.)=145-154 (dec).

### Example 205

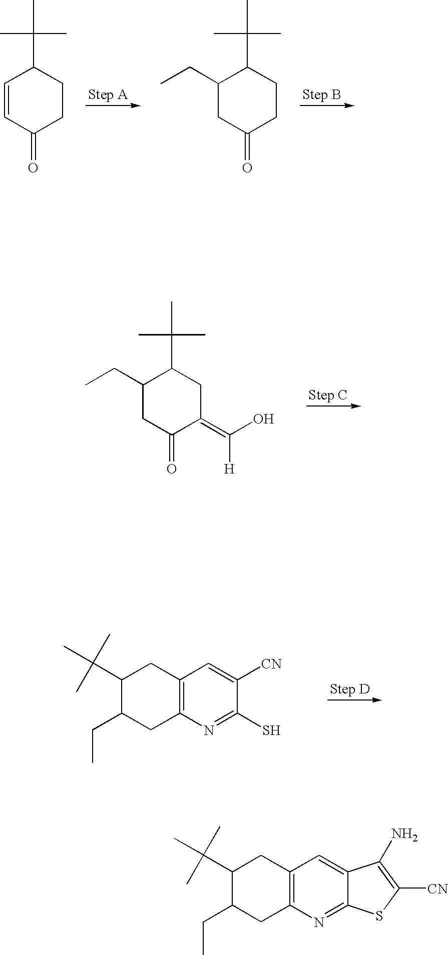 Step A:

4-tert-Butyl-3-ethyl-cyclohexenone. Following the same procedure set forth in Example 202, Step D, only substituting MeLi shown in example 202 with ethyl magnesium bromide (1.7 eq, 3.0 M in Et₂O) gave 4-tert-butyl-3-ethyl cyclohexanone (31% yield) as a yellow oil.

Step B:

5-tert-Butyl-4-ethyl 2-oxo-cyclohanecarbaldehyde. Following a similar procedure set forth in Example 1, Step A, only substituting the ketone shown in Example 1 with 4-tert-butyl-3-ethyl-cyclohexenone (2.258 g, 12.39 mmol) gave 1.521 g (58% yield) of 5-tert-butyl4-ethyl 2-oxo-cyclohanecarbaldehyde as a yellow oil.

Step C:

6-tert-Butyl-2-mercapto-7-ethyl-5,6,7,8-tetrahydroquinoline-3-carbonitrile. Following a similar procedure set forth in Example 1, Step B, only substituting the α-formyl ketone shown in Example 1 with 5-tert-butyl4-ethyl-2-oxo-cyclohanecarbaldehyde (1.521 g, 7.233 mmol) gave 1.518 g (76% yield) of 6-tert-butyl-2-mercapto-7-ethyl-5,6,7,8-tetrahydro-quinoline-3-carbonitrile as a red-orange solid that was used without further purification.

Step D:

3-Amino-6-tert-butyl-7-ethyl-5,6,7,8-tetrahydrothieno[2,3-b]quinoline-2-carbonitrile. Following a similar procedure set forth in Example 1, Step C, only substituting the mercapto-nitrile shown in Example 1 with 6-tert-butyl-2-mercapto-7-ethyl-5,6,7,8-tetrahydro-quinoline-3-carbonitrile (1.291 g, 4.706 mmol) gave 1.111 g (75% yield) of 3-amino-6-tert-butyl-7-ethyl-5,6,7,8-tetrahydrothieno[2,3-b]quinoline-2-carbonitrile as an orange solid. LCMS [M+1]⁺=314; mp (° C.)=171-186 (dec).

### Example 206

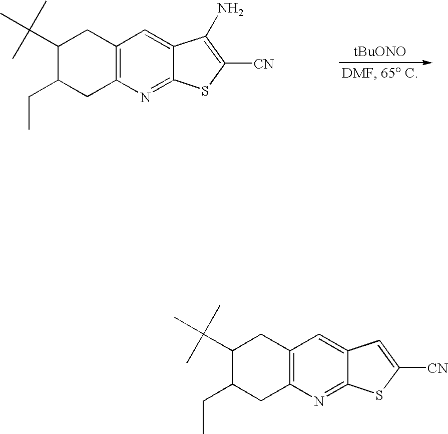

6-tert-Butyl-7-ethyl-5,6,7,8-tetrahydro-thieno[2,3,-b]quinoline-2-carbonitrile. Following a similar procedure set forth in Example 58, Step A, only substituting the amino-nitrile shown in Example 58 with 6-tert-butyl-2-mercapto-7-ethyl-5,6,7,8-tetrahydro-quinoline-3-carbonitrile (1.07 g, 3.40 mmol) gave 0.808 g (80% yield) of 6-tert-butyl-7-ethyl-5,6,7,8-tetrahydro-thieno[2,3,-b]quinoline-2-carbonitrile as a yellow solid. LCMS [M+1]⁺=299; mp (° C.)=162-184.

### Example 207

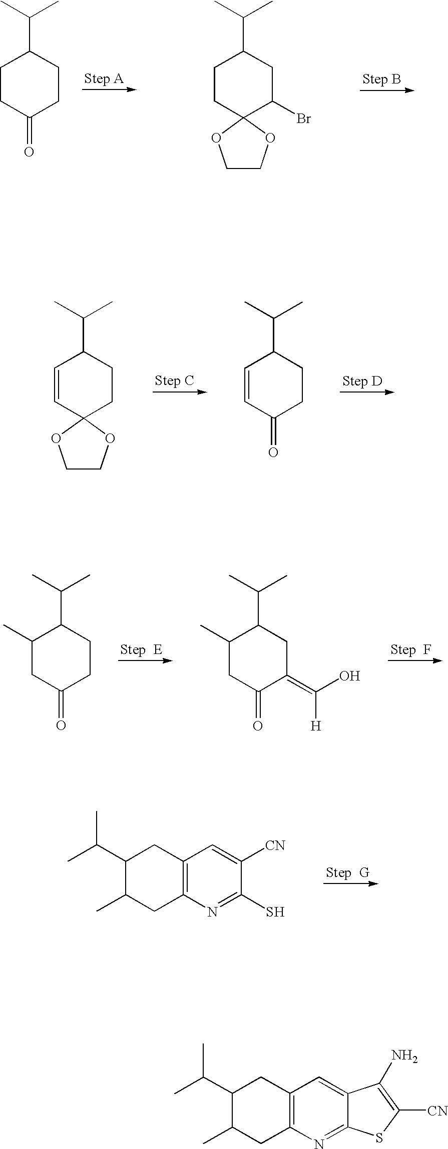

STEP A: 6-Bromo-8-isopropyl-1,4-dioxa-spiro[4,5]decane. Following a similar procedure set forth in Example 202, Step A, only substituting the ketone shown in Example 202 with 4-iso-propylcyclohexenone (10.10 g, 72.02 mmol) gave 17.88 g (94% yield) of 6-bromo-8-isopropyl-1,4-dioxa-spiro[4,5]decane as a pale yellow oil.

STEP B: 8-Isopropyl-1,4-dioxa-spiro[4,5]dec-6-ene. Following a similar procedure set forth in Example 202, Step B, only substituting the ketal shown in Example 202 with 6-bromo-8-isopropyl-1,4-dioxa-spiro[4,5]decane (17.88 g, 67.93 mmol) gave 11.81 g (95% yield) of 8-isopropyl-1,4-dioxa-spiro[4,5]dec-6-ene as a pale yellow oil.

STEP C: 4-Isopropyl-cyclohex-2-enone. Following a similar procedure set forth in Example 202, Step C, only substituting the ketal shown in Example 202 with 8-isopropyl-1,4-dioxa-spiro[4,5]dec-6-ene (11.81 g, 64.78 mmol) gave 5.61 g (63% yield) of 4-isopropyl-cyclohex-2-enone as a pale yellow oil.

STEP D: 5-Isopropyl-3-methyl-cyclohexanone. Following a similar procedure set forth in Example 202, Step D, only substituting the enone shown in Example 202 with 4-isopropyl-cyclohex-2-enone (2.65 g, 19.14 mmol) gave 1.46 g (49% yield) of a mixture of diastereomers of 5-isopropyl-3-methyl-cyclohexanone as a pale yellow liquid.

STEP E: 5-Isopropyl-4-methyl-2-oxo-cyclohexanecarbaldehyde. Following a similar procedure set forth in Example 1, Step A, only substituting the ketone shown in Example 1 with 5-isopropyl-3-methyl-cyclohexanone (1.46 g, 9.468 mmol) gave 0.6280 g (36% yield) of 5-isopropyl-4-methyl-2-oxo-cyclohexanecarbaldehyde as a yellow liquid.

STEP F: 6-Isopropyl-2-mercapto-7-methyl-5,6,7,8-tetrahydro-quinoline-3-carbonitrile. Following a similar procedure set forth in Example 1, Step B, only substituting the ketone shown in Example 1 with 5-isopropyl-4-methyl-2-oxo-cyclohexanecarbaldehyde (0.6280 g, 3.445 mmol) gave 0.7436 g (88% yield) of 6-isopropyl-2-mercapto-7-methyl-5,6,7,8-tetrahydro-quinoline-3-carbonitrile as a 1:1 ratio of diasteromers.

STEP G: 3-Amino-6-isopropyl-7-methyl-5,6,7,8-tetrahydro-thieno[2,3-b]quinoline-2-carbonitrile. Following a similar procedure set forth in Example 1, Step C, only substituting the mercapto-nitrile shown in Example 1 with 6-isopropyl-2-mercapto-7-methyl-5,6,7,8-tetrahydro-quinoline-3-carbonitrile (0.4560 g, 1.851 mmol) gave 0.2395 g (45% yield) of 3-amino-6-isopropyl-7-methyl-5,6,7,8-tetrahydrothieno[2,3-b]quinoline-2-carbonitrile as a green solid. LCMS [M+1]⁺=286; mp (° C.)=195-206 (dec).

### Example 208

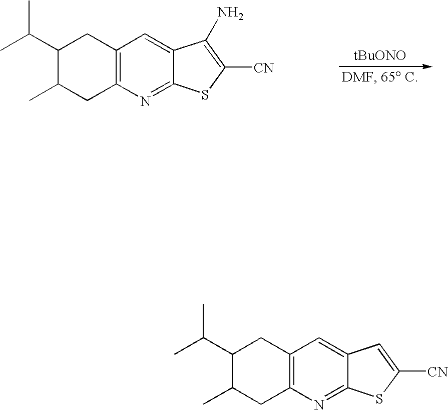

6-isopropyl-7-methyl-5,6,7,8-tetrahydro-thieno[2,3,-b]quinoline-2-carbonitrile. Following a similar procedure set forth in Example 58, Step A, only substituting the amino-nitrile shown in Example 58 with 6-isopropyl-2-mercapto-7-methyl-5,6,7,8-tetrahydro-quinoline-3-carbonitrile (0.1258 g, 0.4408 mmol) gave 0.0450 g (38% yield) of 6-isopropyl-7-methyl-5,6,7,8-tetrahydro-thieno[2,3,-b]quinoline-2-carbonitrile, as a mixture of diastereomers. Waxy orange solid.; LCMS [M+1]⁺=271; mp (° C.)=76-80.

### Example 209

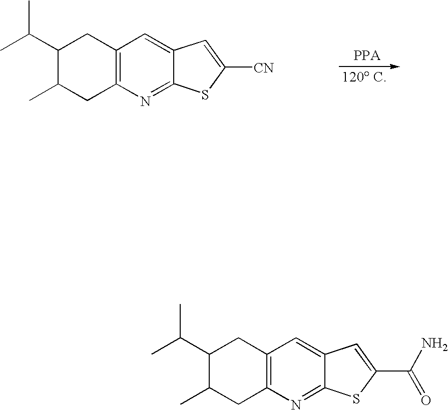

6-isopropyl-7-methyl-5,6,7,8-tetrahydro-thieno[2,3,-b]quinoline-2-carboxylic acid amide. Following a similar procedure set forth in Example 64, Step A, only substituting the carbonitrile shown in Example 64 with 6-isopropyl-7-methyl-5,6,7,8-tetrahydro-thieno[2,3,-b]quinoline-2-carbonitrile (0.0233 g, 0862 mmol) gave 0.0194 g (78% yield) a diastereomeric mixture of 6-isopropyl-7-methyl-5,6,7,8-tetrahydro-thieno[2,3,-b]quinoline-2-carboxylic acid amide as an orange foam. LCMS [M+1]⁺=289.

### Example 210

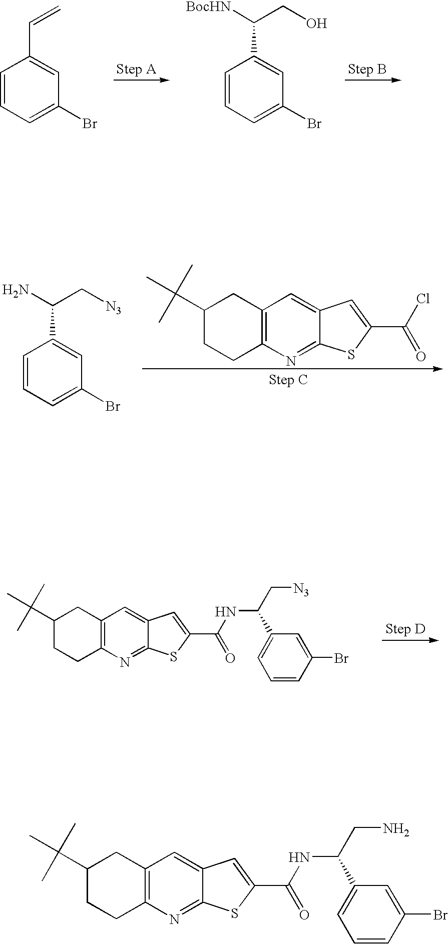 Step A:

(1S)-N-(tert-Butyloxycarbonyl)-1-(3-bromophenyl)-2-hydroxyethylamine. A solution of tert-butyl carbamate (0.73 g, 6.21 mmol) in n-PrOH (8 mL) was treated with a solution of NaOH (0.24 g in 15 mL H₂O) followed by t-BuOCl (0.66 g). After stirring at room temperature for 5 min, the solution was cooled to 0° C. A solution of (DHQ)₂PHAL (96 mg, 0.12 mmol) in n-PrOH (8 mL) was added. 3-Bromostyrene (366 mg, 2.0 mmol) in 14 mL n-PrOH was added to the reaction flask followed by K₂OsO₂(OH)₄ (29.6 mg, 0.08 mmol). The reaction was stirred at 0° C. for 1 h. The reaction was quenched by the addition of 20 mL of saturated aqueous Na₂SO₃ solution. The aqueous phase was extracted with EtOAc (3×25 mL). The combined organic phase was washed with brine (1×25 mL), dried over MgSO₄, filtered, and concentrated in vacuo. Purification via silica gel chromatography (20% EtOAc/hexane) gave 0.42 g (67% yield) of (1S)-N-(tert-butyloxycarbonyl)-1-(3-bromophenyl)-2-hydroxyethylamine as a white solid. The regioisomer was also isolated as a white solid (0.14 g, 22%).

Step B:

(1S)-2-azido-1-(3-bromo-phenyl)-ethylamine. A solution of (1S)-N-(tert-butyloxycarbonyl)-1-(3-bromophenyl)-2-hydroxyethylamine (0.586 g, 1.85 mmol) in dichloromethane (4 mL) at 0° C. was treated with triethylamine (0.39 mL, 2.78 mmol) followed by methanesulfonyl chloride (170 μL, 2.22 mmol). The reaction was stirred at 0° C. for 1 h. The reaction was quenched by the addition of 1 N HCl (aq) solution. The aqueous phase was extracted with CH₂CI₂. The combined organic phase was dried over Na₂SO₄, filtered, and concentrated in vacuo.

The crude mesylate (0.73 g, 1.85 mmol) was taken up in DMF (12 mL) and sodium azide (0.36 g, 5.56 mmol) was added. The reaction was heated at 75° C. for 10 h. Upon cooling, EtOAc and hexane were added. The layers were separated and the aqueous layer was extracted with 70% EtOAc/hexane. The combined organic phase was washed with water, brine, dried over Na₂SO₄, filtered, and concentrated in vacuo to give (1S)-[2-azido-1-(3-bromo-phenyl)-ethyl]-carbamic acid tert-butyl ester a yellow oil.

The crude azide (0.632 g, 1.85 mmol) in 1:3 TFA/CH₂Cl₂ (12 mL) was stirred at room temperature for 1 h. The reaction was diluted with dichloromethane and made basic with dilute aqueous NaOH solution. The organic layer was dried over Na₂SO₄, filtered, and concentrated in vacuo to give (1S)-2-azido-1-(3-bromo-phenyl)-ethylamine.

Step C:

6-tert-Butyl-5,6,7,8-tetrahydro-thieno[2,3-b]quinoline-2-carboxylic acid [(1S)-2-azido-1-(3-bromophenyl)ethyl]-amide. A solution of 6-tert-butyl-5,6,7,8-tetrahydro-thieno[2,3-b]quinoline-2-carbonyl chloride (0.38 g, 1.23 mmol) in dichloromethane (6 mL) at 0° C. was treated with a solution of (1S)-2-azido-1-(3-bromo-phenyl)-ethylamine (0.45 g, 1.85 mmol) and diisopropylethylamine (0.97 mL, 5.55 mmol) in dichloromethane (6 mL). The reaction was stirred at 0° C. for 1 h. The reaction was quenched by the addition of 1 N HCl (aq) solution. The aqueous phase was extracted with CH₂Cl₂. The combined organic phase was dried over Na₂SO₄, filtered, and concentrated in vacuo. Purification via silica gel chromatography (10% EtOAc/CH₂Cl₂) gave 0.48 g (77% yield) of 6-tert-butyl-5,6,7,8-tetrahydro-thieno[2,3-b]quinoline-2-carboxylic acid [(1S)-2-azido-1-(3-bromophenyl)ethyl]-amide as a white solid.

Step D:

6-tert-Butyl-5,6,7,8-tetrahydro-thieno[2,3-b]quinoline-2-carboxylic acid [(1S)-2-amino-1-3-bromo-phenyl)ethyl]-amide. A solution of 6-tert-butyl-5,6,7,8-tetrahydro-thieno[2,3-b]quinoline-2-carboxylic acid [(1S)-2-azido-1-(3-bromo-phenyl)ethyl]-amide (0.063 g, 0.123 mmol) in 4:1 THF/H₂O (12 mL) was treated with triethylamine (69 μL, 0.492 mmol) followed by triphenylphosphine (0.065 g, 0.246 mmol). The reaction was stirred at room temperature for 20 h. The solvent was concentrated in vacuo and purified via silica gel chromatography (5% MeOH/CH₂Cl₂) to give 49.8 mg (83%) of 6-tert-butyl-5,6,7,8-tetrahydro-thieno[2,3-b]quinoline-2-carboxylic acid [(1S)-2-amino-1-(3-bromo-phenyl)-ethyl]-amide as a white solid. LCMS: MH⁺=488; mp (° C.)=95-104.

### Example 211

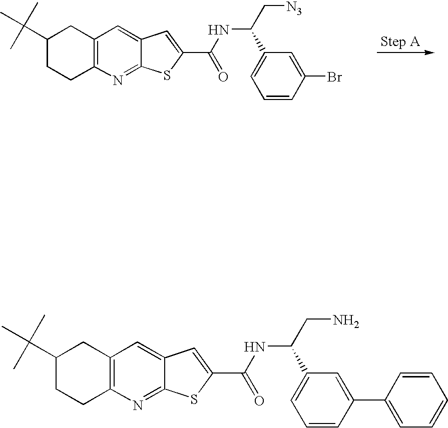 Step A:

6-tert-Butyl-5,6,7,8-tetrahydro-thieno[2,3-b]quinoline-2-carboxylic acid [(1S)-2-amino-1-biphenyl-3-yl-ethyl)-amide. 6-tert-Butyl-5,6,7,8-tetrahydro-thieno[2,3-b]quinoline-2-carboxylic acid [(1S)-2-azido-1-(3-bromo-phenyl)-ethyl]-amide (18.0 mg, 0.035 mmol), phenylboronic acid (4.7 mg, 0.039 mmol), Pd(Ph₃P)₄ (4.1 mg, 10 mol %), Ph₃P (9.2 mg, 0.035 mmol), 2 M aqueous Na₂CO₃ solution (0.10 mL) in DME (1 mL) were placed into a microwave reactor vial and heated with microwave irradiation at 140° C. for 20 min. The mixture was filtered through Celite and concentrated in vacuo. Purification on silica gel (5% MeOH/CH₂Cl₂) gave 6-tert-butyl-5,6,7,8-tetrahydro-thieno[2,3-b]quinoline-2-carboxylic acid [(1S)-2-amino-1-biphenyl-3-yl-ethyl)-amide 10.2 mg (60%) as a white solid. LCMS: MH⁺=484; mp (° C.)=101-108.

### Example 212

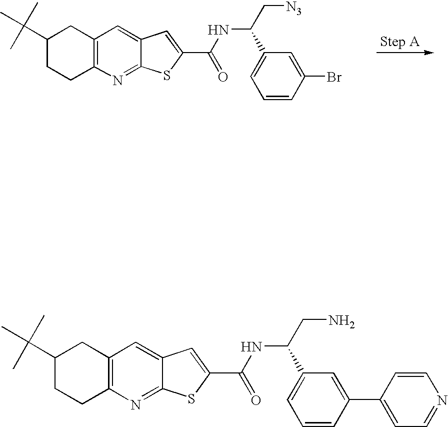 Step A:

6-tert-Butyl-5,6,7,8-tetrahydro-thieno[2,3-b]quinoline-2-carboxylic acid [(1S)-2-amino-1-3-pyridin-4-yl-phenyl)-ethyl]-amide. 6-tert-Butyl-5,6,7,8-tetrahydro-thieno[2,3-b]quinoline-2-carboxylic acid-[(1S)-2-azido-1-(3-bromo-phenyl)-ethyl]-amide (13.8 mg, 0.027 mmol), pyridine 4-boronic acid (4.0 mg, 0.033 mmol), Pd(Ph₃P)₄ (3.1 mg, 10 mol %), Ph₃P (7.1 mg, 0.027 mmol), 2 M aqueous Na₂CO₃ solution (0.10 mL) in DME (1 mL) were placed into a microwave reactor vial and heated with microwave irradiation at 140° C. for 20 min. The mixture was filtered through Celite and concentrated in vacuo. Purification on silica gel (5% MeOH/CH₂Cl₂) gave 6-tert-butyl-5,6,7,8-tetrahydro-thieno[2,3-b]quinoline-2-carboxylic acid [(1S)-2-amino-1-(3-pyridin-4-yl-phenyl)-ethyl]-amide 3.3 mg (25%) as a white solid. LCMS: MH⁺=485; mp (° C.)=127-138 (dec.).

### Example 213

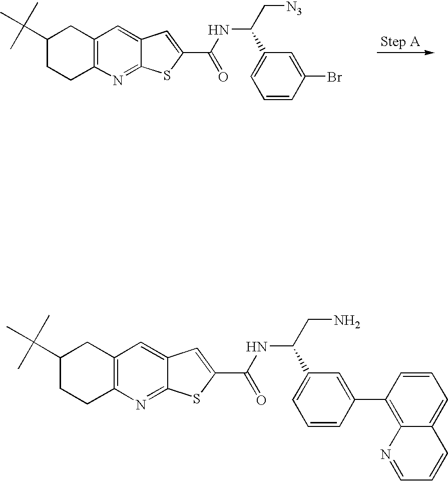 Step A:

6-tert-Butyl-5,6,7,8-tetrahydro-thieno[2,3-b]quinoline-2-carboxylic acid [(1S)-2-amino-1-(3-quinolin-8-yl-phenyl)-ethyl]-amide. 6-tert-Butyl-5,6,7,8-tetrahydro-thieno[2,3-b]quinoline-2-carboxylic acid [(1S)-2-azido-1-(3-bromo-phenyl)ethyl]-amide (12.9 mg, 0.025 mmol), 8-quinoline boronic acid (4.8 mg, 0.028 mmol), Pd(Ph₃P)₄ (2.9 mg, 10 mol %), Ph₃P (6.6 mg, 0.025 mmol), 2 M aqueous Na₂CO₃ solution (0.10 mL) in DME (1 mL) were placed into a microwave reactor vial and heated with microwave irradiation at 140° C. for 20 min. The mixture was filtered through Celite and concentrated in vacuo. Purification on silica gel (5% MeOH/CH₂Cl₂) gave 6-tert-butyl-5,6,7,8-tetrahydro-thieno[2,3-b]quinoline-2-carboxylic acid [(1S)-2-amino-1-(3-quinolin-8-yl-phenyl)-ethyl]-amide 11.2 mg (83%) as a white solid. LCMS: MH⁺=535; mp (° C.)=128-134.

### Examples 214-221

Through essentially the same procedure set forth in Example 210, except in the case of example 217-221 where azide reductions were carried out using the conditions set forth in step B of example 222, by substituting the styrene shown in Column 2 of Table 21 in Step A, the compounds in Column 3 were prepared:

TABLE 21

| Example | Column 2 | Column 3 | CMPD |
|---|---|---|---|
| 214 | 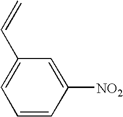 | 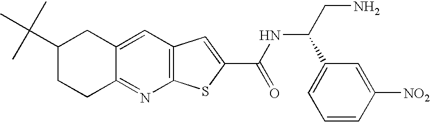 | MS: MH⁺ = 453 mp (° C.) = 175(dec.) |
| 215 | 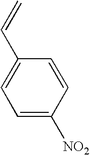 | 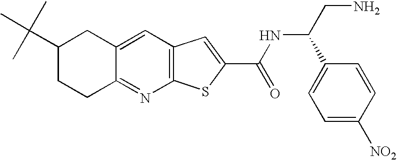 | MS: MH⁺ = 453; mp (° C.) = 185(dec.) |
| 216 | 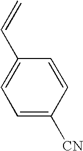 | 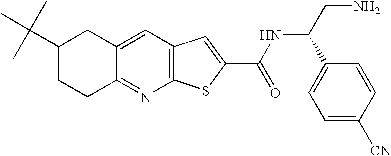 | MS: MH⁺ = 433; mp (° C.) = 192(dec.) |
| 217 | 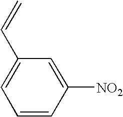 | 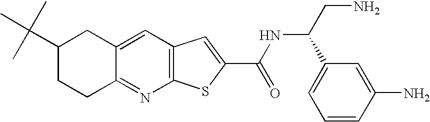 | MS: MH⁺ = 423; mp (° C.) = 213(dec.) |
| 218 | 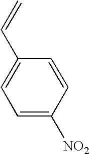 | 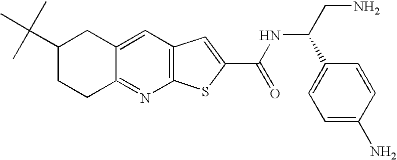 | MS: MH⁺ = 423; mp (° C.) = 210(dec.) |
| 219 | 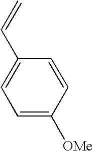 | 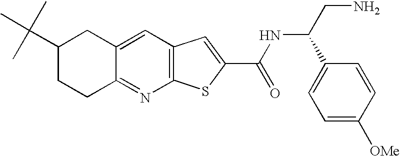 | MS: MH⁺ = 438; mp (° C.) = 181(dec.) |
| 220 | 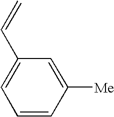 | 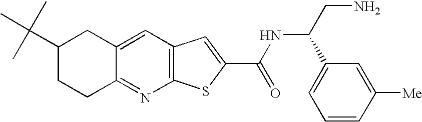 | MS: MH⁺ = 422; mp (° C.) = 194(dec.) |
| 221 | 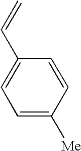 | 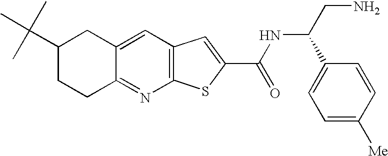 | MS: MH⁺ = 422; mp (° C.) = 191(dec.) |

### Example 222

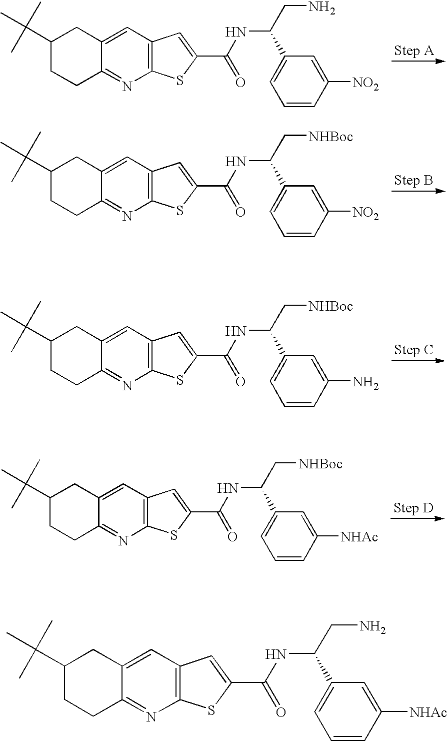 Step A:

[2-[(6-tert-Butyl-5,6,7,8-tetrahydrothieno[2,3-b]quinoline-2-carbonyl)amino]-2-(3-nitrophenyl)ethyl]carbamic acid tert-butyl ester: To a solution of 6-tert-butyl-5,6,7,8-tetrahydrothieno[2,3-b]quinoline-2-carboxylic acid [2-amino-1-(3-nitrophenyl)ethyl]amide (548 mg, 1.21 mmol) in 5 mL of CH₂Cl₂, was added triethylamine (243 mg, 2.40 mmol) and di-tert-butyl dicarbonate (343 mg, 1.57 mmol). The reaction was stirred ar room temperature for 2 h. The solvent was removed under vacuum. The residue was purified by flash chromatography eluting with 60% EtOAc/hexanes to give 573 mg (85%) of [2-[(6-tert-butyl-5,6,7,8-tetrahydrothieno[2,3-b]quinoline-2-carbonyl)amino]-2-(3-nitrophenyl)ethyl]carbamic acid tert-butyl ester.

Step B:

{2-(3-Aminophenyl)-2-[(6-tert-butyl-5,6,7,8-tetrahydrothieno[2,3-b]quinoline-2-carbonyl)-amino]-ethyl}carbamic acid tert-butyl ester: To a solution of [2-[(6-tert-butyl-5,6,7,8-tetrahydrothieno[2,3-b]quinoline-2-carbonyl)amino]-2-(3-nitrophenyl)ethyl]carbamic acid tert-butyl ester (573 mg, 1.04 mmol) in 40 mL of MeOH, was added 10% wt. Pd/C (220 mg). The reaction was stirred at room temperature under an atmosphere of H₂ for 4 h. It was filtered through celite. The celite layer was further rinsed with 80 mL of CH₂Cl₂/MeOH (1:1). The solvent was removed under vacuum to give 540 mg (100%) of {2-(3-aminophenyl)-2-[(6-tert-butyl-5,6,7,8-tetrahydrothieno[2,3-b]quinoline-2-carbonyl)-amino]-ethyl}carbamic acid tert-butyl ester.

Step C:

{2-(3-Acetylaminophenyl)-2-[(6-tert-butyl-5,6,7,8-tetrahydrothieno[2,3-b]quinoline-2-carbonyl)-amino]ethyl]carbamic acid tert-butyl ester: To a solution of {2-(3-aminophenyl)-2-[(6-tert-butyl-5,6,7,8-tetrahydrothieno[2,3-b]quinoline-2-carbonyl)-amino]-ethyl}carbamic acid tert-butyl ester (20 mg, 0.038 mmol) in 1 mL of CH₂Cl₂, was added triethylamine (5.8 mg, 0.058 mmol) and acetylchloride (3.6 mg, 0.046 mmol). The reaction was stirred at room temperature for 0.5 h. It was diluted with 20 mL of CH₂Cl₂, washed with 1 N aqueous HCl. The organic was concentrated under vacuum. The residue was purified by flash chromatography eluting with 8% MeOH/CH₂Cl₂ to give 21 mg (97%) of {2-(3-acetylaminophenyl)-2-[(6-tert-butyl-5,6,7,8-tetrahydrothieno[2,3-b]quinoline-2-carbonyl)amino]ethyl}carbamic acid tert-butyl ester.

Step D:

6-tert-Butyl-5,6,7,8-tetrahydrothieno[2,3-b]quinoline-2-carboxylic acid [1-(3-acetylaminophenyl)-2-aminoethyl]amide: To a solution of {2-(3-acetylaminophenyl)-2-[(6-tert-butyl-5,6,7,8-tetrahydrothieno[2,3-b]quinoline-2-carbonyl)amino]ethyl}carbamic acid tert-butyl ester (21 mg, 0.037 mmol) in 1 mL of CH₂Cl₂, was added 1 mL of TFA/CH₂Cl₂ (1:2). The reaction was stirred at room temperature for 2 h. The solvent was removed under vacuum. The residue was partitioned between 20 mL of 20% MeOH/CH₂Cl₂ and 10 mL of dilute aqueous NaOH. The organic was concentrated. The residue was purified by flash chromatography eluting with 20% MeOH/CH₂Cl₂ to give 17 mg (98%) of 6-tert-butyl-5,6,7,8-tetrahydrothieno[2,3-b]quinoline-2-carboxylic acid [1-(3-acetylaminophenyl)-2-aminoethyl]amide. LCMS: MH⁺=465; mp (° C.)=142 (dec.).

### Example 223

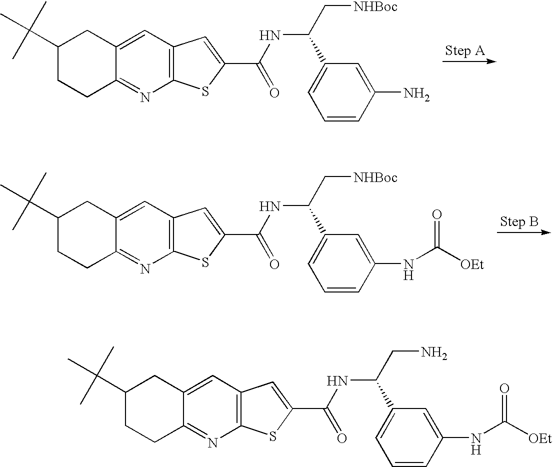 Step A:

(3-{2-tert-Butoxycarbonylamino-1-[(6-tert-butyl-5,6,7,8-tetrahydrothieno[2,3-b]quinoline-2-carbonyl)amino]ethyl}phenyl)carbamic acid ethyl ester: To a solution of {2-(3-aminophenyl)-2-[(6-tert-butyl-5,6,7,8-tetrahydrothieno[2,3-b]quinoline-2-carbonyl)-amino]-ethyl}carbamic acid tert-butyl ester (20 mg, 0.038 mmol) in 1 mL of CH₂Cl₂, was added triethylamine (7.7 mg, 0.076 mmol) and ethyl chloroformate (5.0 mg, 0.046 mmol). The reaction was stirred at room temperature for 0.5 h. Additional ethyl chloroformate (12 mg, 0.11 mmol) was added. The content was concentrated under vacuum. The residue was purified by flash chromatography eluting with 35% EtOAc/CH₂Cl₂ to give 12 mg (53%) of (3-{2-tert-butoxycarbonylamino-1-[(6-tert-butyl-5,6,7,8-tetrahydrothieno[2,3-b]quinoline-2-carbonyl)amino]ethyl}phenyl)carbamic acid ethyl ester.

Step B:

(3-{2-Amino-1-[(6-tert-butyl-5,6,7,8-tetrahydrothieno[2,3-b]quinoline-2-carbonyl)amino]ethyl}phenyl)carbamic acid ethyl ester: To a solution of {3-(2-acetylaminophenyl)-2-[(6-tert-butyl-5,6,7,8-tetrahydrothieno[2,3-b]quinoline-2-carbonyl)amino]ethyl}carbamic acid tert-butyl ester (21 mg, 0.037 mmol) in 1 mL of CH₂Cl₂, was added 1 mL of TFA/CH₂Cl₂ (1:2). The reaction was stirred at room temperature for 2 h. The solvent was removed under vacuum. The residue was dissolved in 1 mL of MeOH. To the resulting solution was added 6 drops of 2 N aqueous Na₂CO₃ followed by 20 mL of 20% MeOH/CH₂Cl₂ and anhydrous Na₂SO₄. It was then filtered and the organic was concentrated. The residue was purified by flash chromatography eluting with 15% MeOH/CH₂Cl₂ to give 18 mg (90%) of (3-{2-amino-1-[(6-tert-butyl-5,6,7,8-tetrahydrothieno[2,3-b]quinoline-2-carbonyl)amino]ethyl}phenyl)carbamic acid ethyl ester. LCMS: MH⁺=495; mp (° C.)=108-130 (dec.).

### Example 224

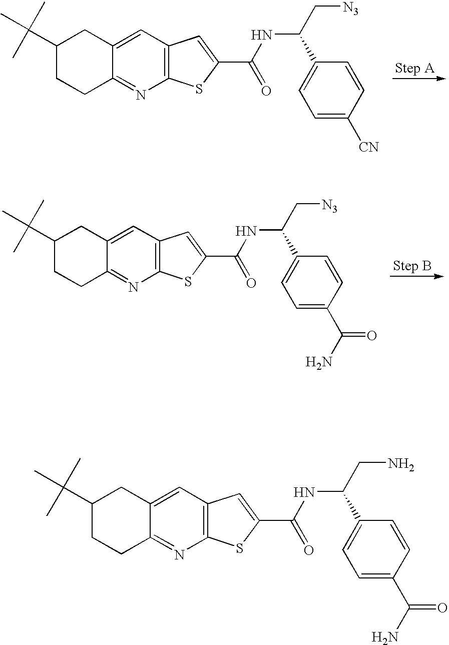 Step A:

6-tert-Butyl-5,6,7,8-tetrahydrothieno[2,3-b]quinoline-2-carboxylic acid [2-azido-1-(4-carbamoylphenyl)ethyl]amide: To a solution of 6-tert-butyl-5,6,7,8-tetrahydro-thieno[2,3-b]quinoline-2-carboxylic acid [2-azido-1-(4-cyanophenyl)ethyl]amide (65 mg, 0.14 mmol) in 1 mL of DMSO, was added K₂CO₃ (60 mg, 0.44 mmol) and 0.1 mL of H₂O₂ (50% wt.). The reaction was stirred at room temperature for 1 h. It was diluted with 15 mL of water and then acidified by 2 N aqueous HCl. The resulting solid was collected by filtration, washed with water and dried under vacuum to give 67 mg (99%) of 6-tert-butyl-5,6,7,8-tetrahydrothieno[2,3-b]quinoline-2-carboxylic acid [2-azido-1-(4-carbamoylphenyl)ethyl]amide.

Step B:

6-tert-Butyl-5,6,7,8-tetrahydrothieno[2,3-b]quinoline-2-carboxylic acid [2-amino-1-(4-carbamoylphenyl)ethyl]amide: To a solution of 6-tert-butyl-5,6,7,8-tetrahydro-thieno[2,3-b]quinoline-2-carboxylic acid [2-azido-1-(4-carbamoylphenyl)ethyl]amide (67 mg, 0.14 mmol) in 8 mL of THF/H₂O (4:1), was added triethylamine (57 mg, 0.56 mmol) and triphenylphosphine (74 mg, 0.28 mmol). The reaction was stirred at room temperature overnight. The solvent was removed under vacuum. The residue was purified by flash chromatography eluting with 20% MeOH/CH₂Cl₂ to give 53 mg (96%) of 6-tert-butyl-5,6,7,8-tetrahydrothieno[2,3-b]quinoline-2-carboxylic acid [2-amino-1-(4-carbamoylphenyl)ethyl]amide. LCMS: MH⁺=451; mp (° C.)=219 (dec.).

### Example 225

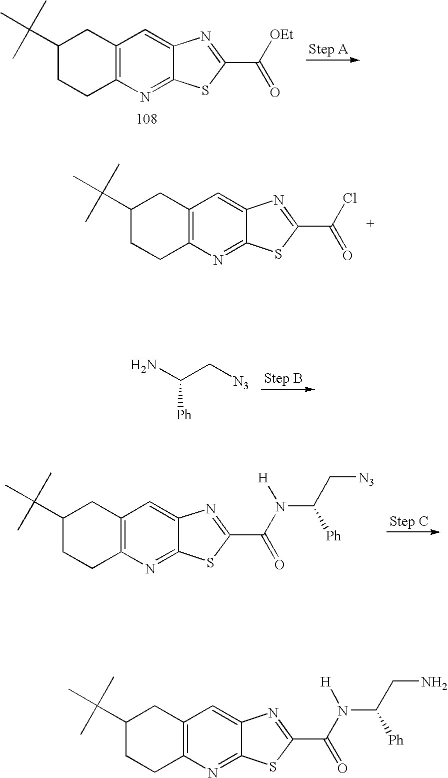

6-tert-Butyl-5,6,7,8-tetrahydro-thiazolo[5,4-b]quinoline-2-carboxylic acid [2(S)-amino-1-phenyl-ethyl]-amide. Following the same procedure set forth in steps A and B of Example 210, the compound (1S)-2-azido-1-phenyl-ethylamine was prepared. Thereafter, (+)-7-tert-butyl-5,6,7,8-tetrahydro-thiazolo[5,4-b]quinoline-2-carboxylic acid chloride prepared from the corresponding ethyl ester (compound 108; 45 mg, 0.104 mmol of compound 108 was used) was reacted with (1S)-2-azido-1-phenyl-ethylamine as shown in step B (analogous in procedure to step C of Example 210) to give the azide, 6-tert-Butyl-5,6,7,8-tetrahydro-thiazolo[5,4-b]quinoline-2-carboxylic acid [(1S)-2-azido-1-phenyl-ethyl]-amide. The azide was then converted as shown in step C (anlagous in procedure to step D of Example 210) to give 24.3 mg (57%) of 6-tert-butyl-5,6,7,8-tetrahydro-thiazolo[5,4-b]quinoline-2-carboxylic acid [2(S)-amino-1-phenyl-ethyl]-amide (compound 225) as a white solid. The HCl salt was prepared by adding 59 μL of 1 N HCl in ether to a solution of 6-tert-butyl-5,6,7,8-tetrahydro-thiazolo[5,4-b]quinoline-2-carboxylic acid [2(S)-amino-1-phenyl-ethyl]-amide in minimal THF (0.5 mL). LCMS: MH⁺=409; mp (° C.)=225-236 (dec).

### Example 226

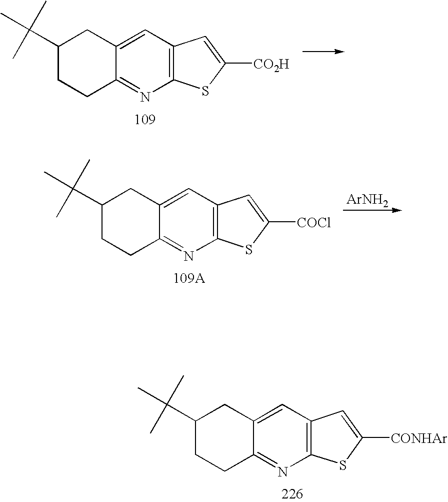

Acid Chloride (109A): To a solution of the tricyclic acid 109 (0.87 g; 3 mmol) in dichloromethane (DCM; 15 mL) was added thionyl chloride (15 ml) and 5 drops of DMF. The reaction mixture was heated to 40° C. for 1.5 hr. The solvent and unreacted thionyl chloride were removed on the rotary evaporator and the residue was dissolved in 3 mL of DCM. Hexane was added to obtain a precipitate, which was filtered. The filter cake was washed with more hexane to leave a yellow solid (0.95 g; 100%).

Method-A: 2-(1-Amino-4-Hydroxyphenyl) carboxamido-6-tert-butyl-5,6,7,8-tetrahydrothieno [2,3-b]quinoline: The tricyclic acid chloride (0.95 g; 3 mmol) was added to a solution of 4-aminophenol (0.68 g; 6.2 mmol) and pyridine (0.75 mL; 9.23 mmol) in 30 mL of THF. The reaction mixture was stirred at room temperature for 2 hrs. The supernatant reaction mixture was filtered from the sticky brown precipitate, which is unreacted 4-amino phenol. The solid free reaction mixture was carefully quenched with water and 1 N.HCl solution. This resulted in the formation of a brown precipitate, which was collected by filtration. Washing with solvent (3× with 5 mL of 2:1 DCM-Methanol) produced the desired aryl carboxamide as a white solid 226A (0.65 g; 56%).

Method-B: 3-Aminopyridine (0.055 g; 0.58 mmol) was added to a solution of the tricyclic acid chloride (0.045 g; 0.145 mmol) in 2 mL of DCM. The reaction mixture was stirred at room temperature for 2 hrs and then diluted with DCM (10 mL). The DCM extract was washed with 1 N sodium hydroxide solution, 1 N HCl solution, and brine and dried over Na₂SO₄. Concentration produced a yellow solid, which was stirred with 2 mL of DCM and filtered. The filter cake was washed with 5 mL of DCM to leave a white solid 226B (0.028 g; 53%). Table 22 below sets forth the various compounds of the general structure 226, their method of production and their characterization data.

TABLE 22

| | |
|---|---|
| | 226 |
| 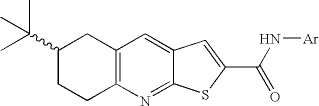 | |

| | Method | | mp (° C.)/ |
|---|---|---|---|
| Comp # | (% Yield) | Ar in Structure 226 | MH⁺ (LCMS) |
| 226A | A (56) | 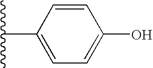 | 188(dec)/381 |
| 226B | B (53) | 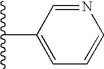 | 240(dec)/366 |
| 226C | B (50) | 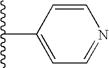 | 219(dec)/366 |
| 226D | B (45) | 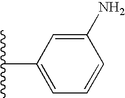 | 222(dec)/380 |
| 226E | B (30) | 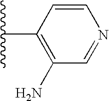 | 226(dec)/381 |
| 226F | B (48) | 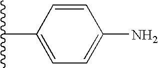 | 242/380 |
| 226G | B (44) | 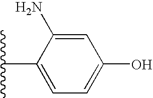 | 140-144/396 |
| 226H | B (49) | 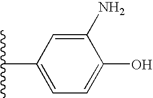 | 186(dec)/396 |
| 226J | B (37) | 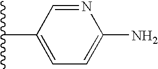 | 276(dec)/381 |

### Example 227

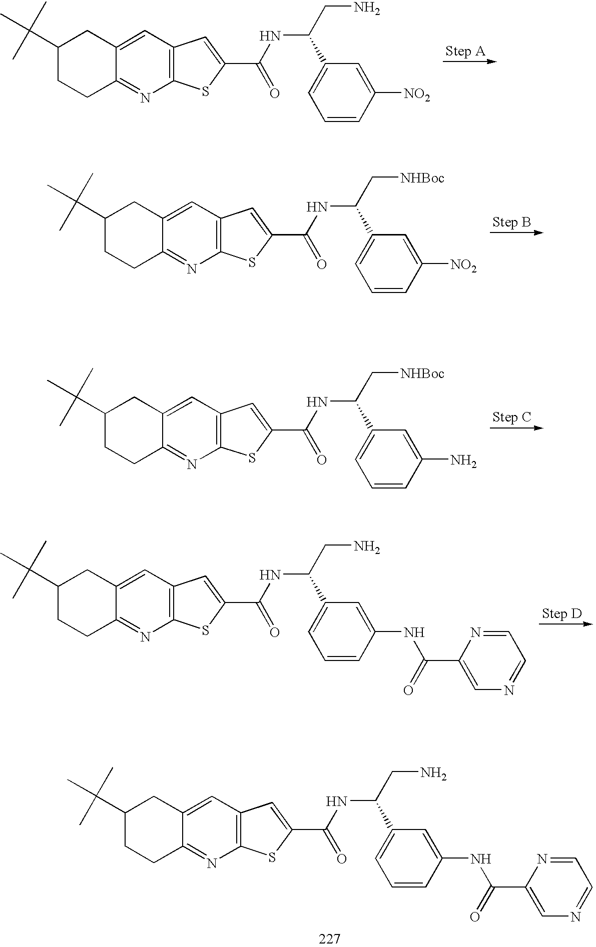

6-tert-Butyl-5,6,7,8-tetrahydrothieno[2,3-b]quinoline-2-carboxylic acid (2-amino-1-{3-[(pyrazine-2-carbonyl)amino]phenyl}ethyl)amide (227): This compound was prepared: by essentially thee same procedure-set forth in Preparative Example 222, only substituting acetylchloride with pyrazine-2-carbonyl chloride in step C. LCMS: MH⁺=529; mp (° C.)=212 (dec.).

### Examples 228-230

Through essentially the same procedure set forth in Example 227 by substituting the acid chloride in Column 2 of Table 22 in Step C, the compounds in Column 3 were prepared:

TABLE 22

| Ex- | | | |
|---|---|---|---|
| ample | Column 2 | Column 3 | CMPD |
| 228 | 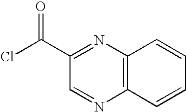 | 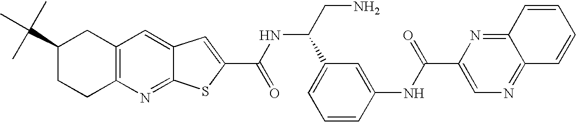 | MS: MH⁺ =579.2; mp (° C.) =147-166 (dec) free amine |
| 229 | 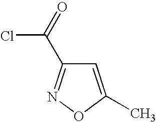 | 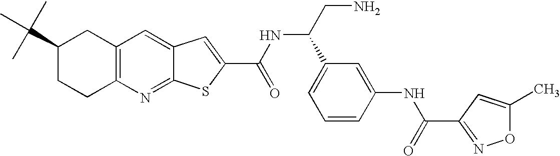 | MS: MH⁺ =532.3; mp (° C.) =104-107 free amine |
| 230 | 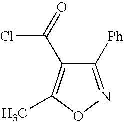 | 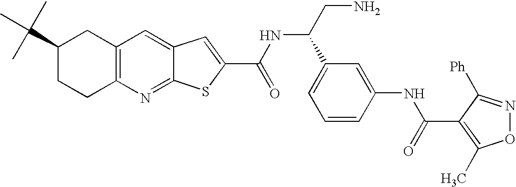 | MS: MH⁺ =608.3; mp =232° C. (dec) free amine |

### Example 231

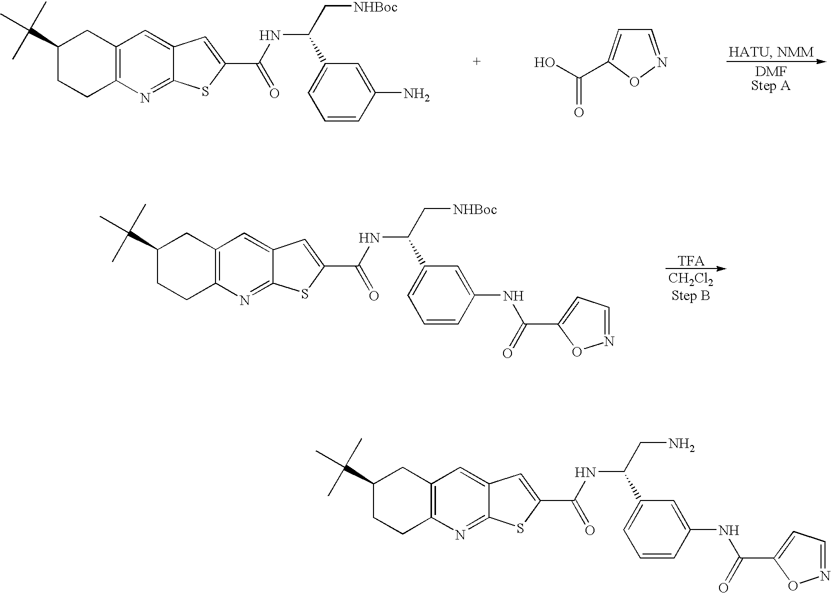 Step A:

(2-[(6(R)-tert-Butyl-5,6,7,8-tetrahydro-thieno[2,3-b]quinoline-2-carbonyl)-amino]-2(S)-{3-[(isoxazole-5-carbonyl)-amino]-phenyl}-ethyl)-carbamic acid tert-butyl ester. To a solution of {2(S)-(3-amino-phenyl)-2-[(6(R)-tert-butyl-5,6,7,8-tetrahydro-thieno[2,3-b]quinoline-2-carbonyl)-amino]-ethyl-carbamic acid tert-butyl ester (19.7 mg, 0.04 mmol) in DMF (0.5 mL) was added isoxazole-5-carboxylic acid (12.8 mg, 0.11 mmol), NMM (20.7 μL, 0.19 mmol), followed by HATU (43 mg, 0.11 mmol). The reaction mixture was stirred at rt for overnight. The reaction was diluted with H₂O (10 mL), the solid was collected by filtration (washed with H₂O), and dried under vacuum to give 23 mg of (2-[(6(R)-tert-butyl-5,6,7,8-tetrahydro-thieno[2,3-b]quinoline-2-carbonyl)-amino]-2(S)-{3-[(isoxazole-5-carbonyl)-amino]-phenyl}-ethyl)-carbamic acid tert-butyl ester that was used directly in step B.

Step B:

6(R)-tert-Butyl-5,6,7,8-tetrahydro-thieno[2,3-b]quinoline-2-carboxylic acid (2-amino-1(S)-{3-[(isoxazole-5-carbonyl)-amino]-phenyl}-ethyl)-amide. To a solution of (2-[(6(R)-tert-butyl-5,6,7,8-tetrahydro-thieno[2,3-b]quinoline-2-carbonyl)-amino]-2(S)-{3-[(isoxazole-5-carbonyl)-amino]-phenyl}-ethyl)-carbamic acid tert-butyl ester (23 mg, 0.038 mmol) in 0.2 mL/0.6 mL (TFA/CH₂Cl₂) was stirred at rt for 1.5 hr. The solvent was removed in vacuo. The residue was treated with MeOH (1 mL) and saturated Na₂CO₃ solution. The organic phase was diluted with CH₂Cl₂, dried (anhydrous Na₂SO₄), filtered and concentrated. The product was purified by preparative TLC (10% MeOH/CH₂Cl₂ containing 1% NH₄OH) to yield 10.8 mg (55%, 2 steps) of 6(R)-tert-butyl-5,6,7,8-tetrahydro-thieno[2,3-b]quinoline-2-carboxylic acid (2-amino-1(S)-{3-[(isoxazole-5-carbonyl)-amino]-phenyl}-ethyl)-amide. LC-MS: MH⁺=518.3; mp=98-102° C.

General Procedure for Preparing the HCl Salt: The product was dissolved in the minimal amount of CH₂Cl₂ and/or MeOH, one equivalent of HCl (1 M in Et₂O) was added while vigorously stirring the solution. Et₂O was added to the suspension yielding a precipitate. The precipitate was collected by filtration, (the filter cake was washed with Et₂O) and dried under vacuum.

### Examples 232-261

Through essentially the same procedure set forth in Example 231, by substituting the acid in Column 2 of Table 23 in Step 1, the compounds in Column 3 were prepared:

TABLE 23

| Ex- | | | |
|---|---|---|---|
| ample | Column 2 | Column 3 | CMPD |
| 232 | 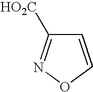 | 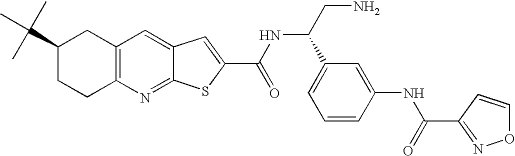 | MS: MH⁺ =518.3; mp (° C.) =137-145 (dec) free amine |
| 233 | 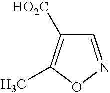 | 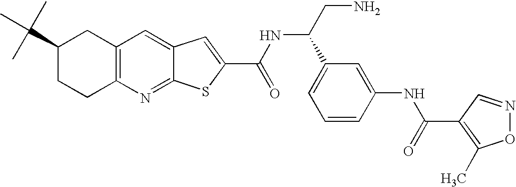 | MS: MH⁺ =532.3 free amine |
| 234 | 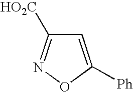 | 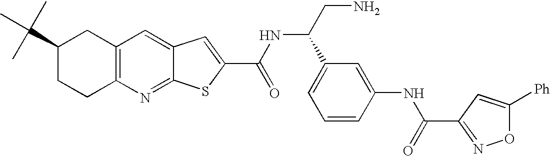 | MS: MH⁺ =594.3; mp (° C.) =126-135 free amine |
| 235 | 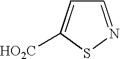 | 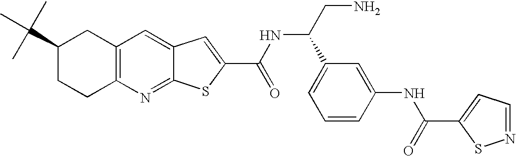 | MS: MH⁺ =534.2 |
| 236 | 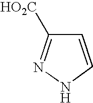 | 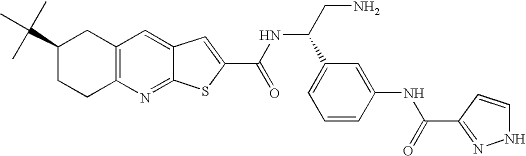 | MS: MH⁺ =517.3 free amine |
| 237 | 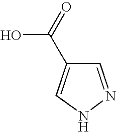 | 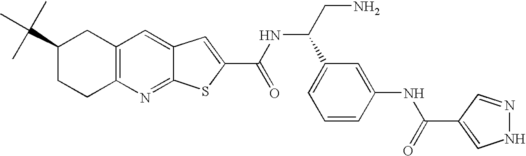 | MS: MH⁺ =517.3; mp (° C.) =176-221 (dec) |
| 238 | 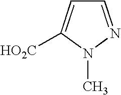 | 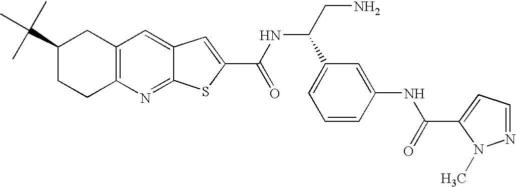 | MS: MH⁺ =531.2; mp (° C.) =151-157 free amine |
| 239 | 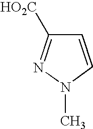 | 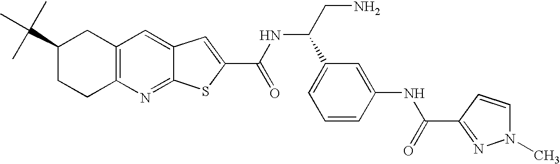 | MS: MH⁺ =531.2; mp (° C.) =139-156 free amine |
| 240 | 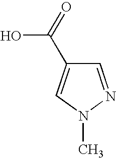 | 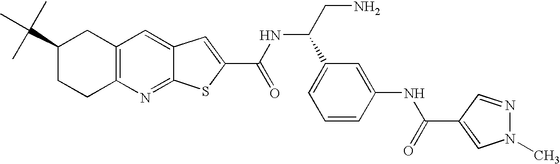 | MS: MH⁺ =531.3 free amine |
| 241 | 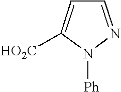 | 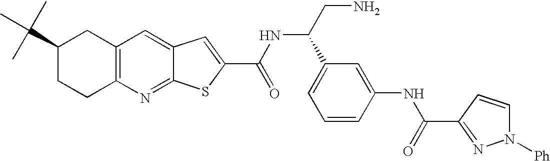 | MS: MH⁺ =593.3; mp (° C.) =147-159 (dec) free amine |
| 242 | 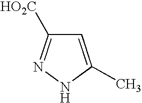 | 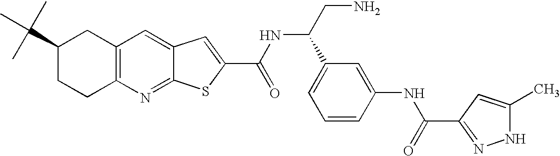 | MS: MH⁺ =531.2; mp (° C.) =183-189 free amine |
| 243 | 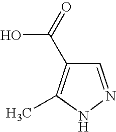 | 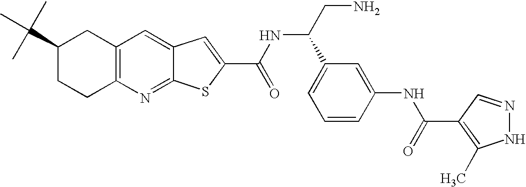 | MS: MH⁺ =531.2; mp (° C.) =150-209 (dec) free amine |
| 244 | 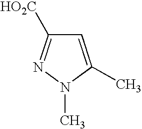 | 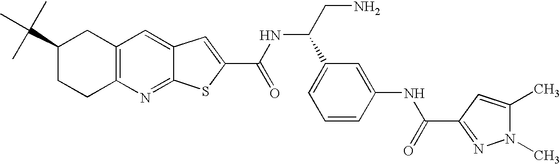 | MS: MH⁺ =545.2 free amine |
| 245 | 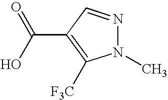 | 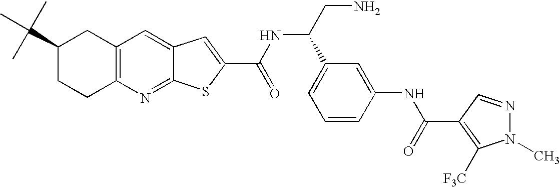 | MS: MH⁺ =599.2; mp (° C.) =183-189 free amine |
| 246 | 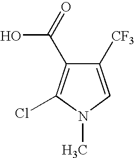 | 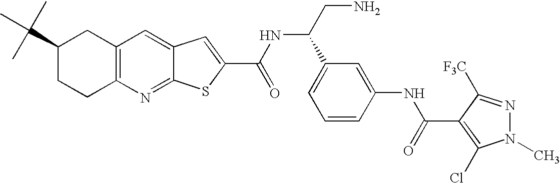 | MS: MH⁺ =633.3; mp (° C.) =152-154 free amine |
| 247 | 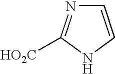 | 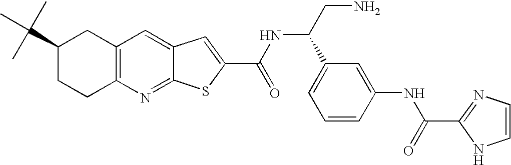 | MS: MH⁺ =517.3; mp (° C.) =176-189 (dec) free amine |
| 248 | 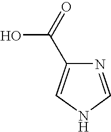 | 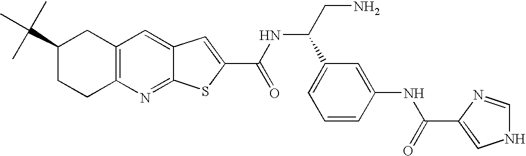 | MS: MH⁺ =517.3; mp (° C.) =174-182 (dec) free amine |
| 249 | 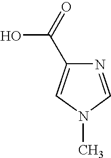 | 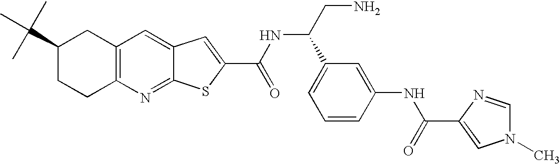 | MS: MH⁺ =531.2; mp (° C.) =148-157 (dec) free amine |
| 250 | 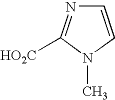 | 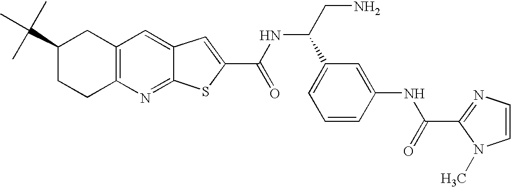 | MS: MH⁺ =531.3; mp (° C.) =208-215 (dec) free amine |
| 251 | 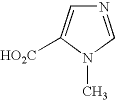 | 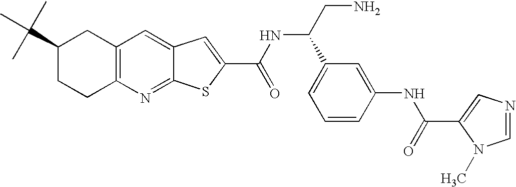 | MS: MH⁺ =531.3; mp (° C.) 160->250 (dec) free amine |
| 252 | 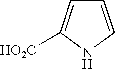 | 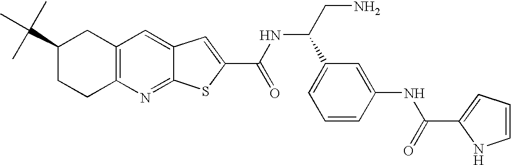 | MS: MH⁺ =516.8 |
| 253 | 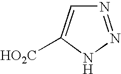 | 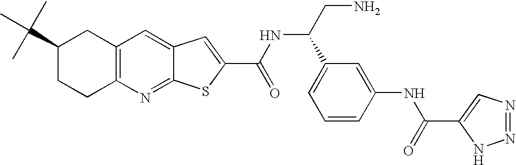 | MS: [(MH⁺)- NH₃] =501.3; mp (° C.) =116-124 (dec) free amine |
| 253A | 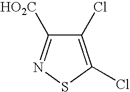 | 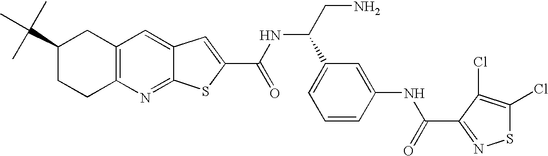 | MS: MH⁺ =602.1; mp (° C.) =146-154 (dec) free amine |
| 254 | 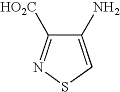 | 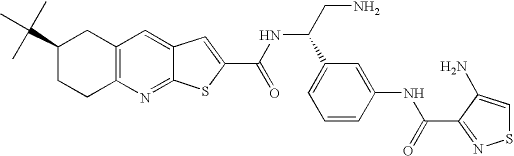 | MS: MH⁺ =549.3; mp (° C.) =109-112 free amine |
| 255 | 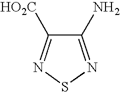 | 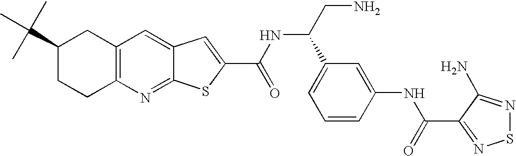 | MS: MH⁺ =550.3; mp (° C.) =148-157 free amine |
| 256 | 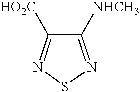 | 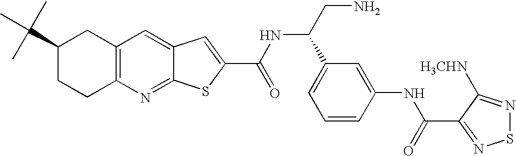 | MS: MH⁺ =564.2; mp (° C.) =128-140 free amine |
| 257 | 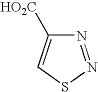 | 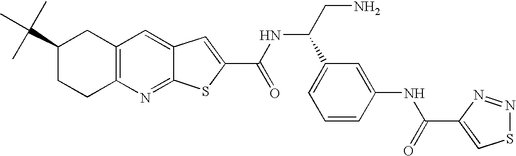 | MS: MH⁺ =535.2; mp (° C.) =205-227 (dec) free amine |
| 258 | 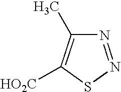 | 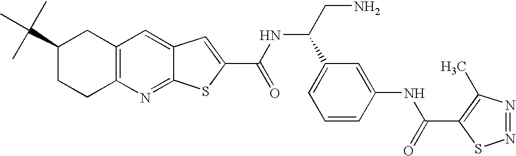 | MS: MH⁺ =549.2; mp (° C.) =187-203 (dec) free amine |
| 259 | 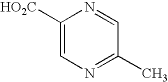 | 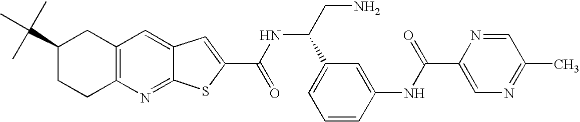 | MS: MH⁺ =543.3; mp (° C.) =89-97 free amine |
| 260 | 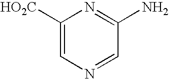 | 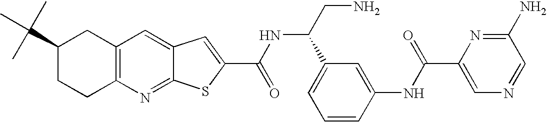 | MS: MH⁺ =544.3; mp (° C.) =135-142 free amine |
| 261 | 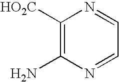 | 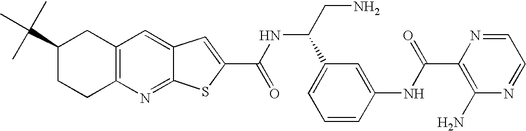 | MS: MH⁺ =544.3; mp (° C.) =143-146 free amine |

### Example 262

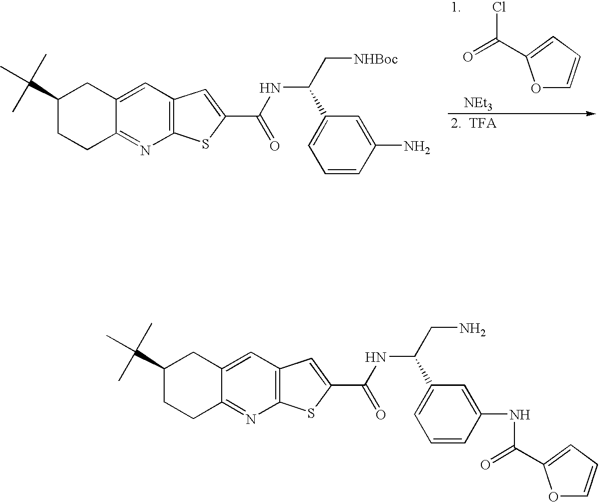

6-tert-Butyl-5,6,7,8-tetrahydrothieno[2,3-b]quinoline-2-carboxylic acid (2-amino-1-{3-[(furan-2-carbonyl)amino]phenyl}ethyl)amide: This compound was prepared by essentially the same procedure set forth in step C and D in Preparative Example 222, only substituting acetylchloride with furan-2-carbonyl chloride in step C. LCMS: MH⁺=517; mp (° C.)=199 (dec.).

### Example 263

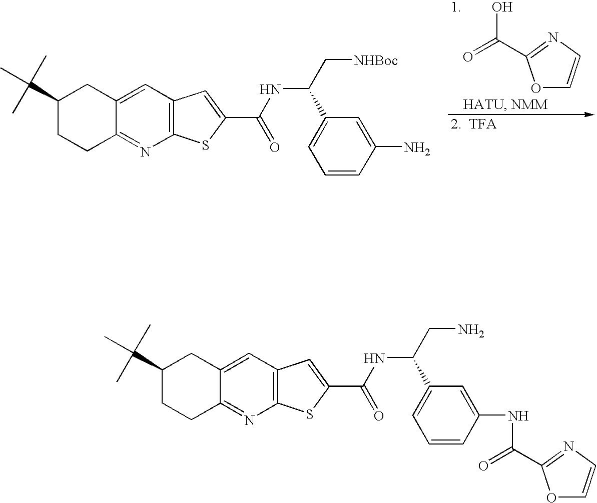

6-tert-Butyl-5,6,7,8-tetrahydrothieno[2,3-b]quinoline-2-carboxylic acid (2-amino-1-{3-](oxazole-2-carbonyl)amino]phenyl}ethyl)amide: To a solution of {2-(3-aminophenyl)-2-[(6-tert-butyl-5,6,7,8-tetrahydrothieno[2,3-b]quinoline-2-carbonyl)amino]ethyl}carbamic acid tert-butyl ester (60 mg, 0.115 mmol) in 1.5 mL of DMF, was added oxazole-2-carboxylic acid (26 mg, 0.23 mmol), 4-methylmorpholine (58 mg, 0.58 mmol) and O-(7-azabenotriazol-1-yl)-N,N,N′N′-tetramethyluronium PF₆ (87 mg, 0.23 mmol). The reaction was stirred at room temperature for 16 h. It was diluted with 15 mL of water. The solid was collected by filtration, washed with water, and dried under vacuum. It was then dissolved in 2 mL of CH₂Cl₂/TFA (3:1). The reaction solution was stirred at room temperature for 1.5 h. The solvent was removed under vacuum. The residue was dissolved in 3 mL of MeOH. It was basified by 1 N aqueous NaOH. The mixture was extracted by 20 mL of CH₂Cl₂. The organic was washed with brine (10 mL) and then concentrated. The residue was purified by flash chromatography eluting with 14% MeOH/CH₂Cl₂ to give 55 mg (93%) of 6-tert-butyl-5,6,7,8-tetrahydrothieno[2,3-b]quinoline-2-carboxylic acid (2-amino-1-{3-[(oxazole-2-carbonyl)amino]phenyl}ethyl)amide. LCMS: MH⁺=518; mp (° C.)=209 (dec.).

### Example 264

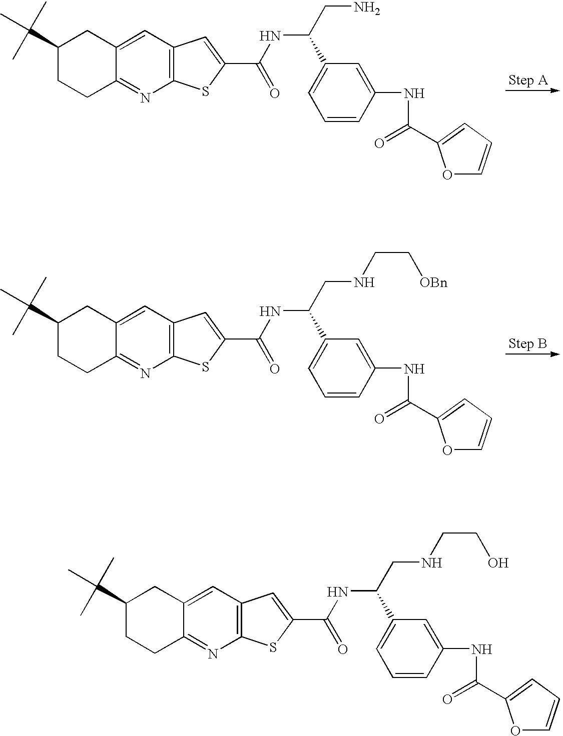 Step A:

6-tert-Butyl-5,6,7,8-tetrahydrothieno[2,3-b]quinoline-2-carboxylic acid (2-(2-benzyloxy-ethylamino)-1-{3-[(furan-2-carbonyl)amino]phenyl}ethyl)amide: To a solution of 6-tert-butyl-5,6,7,8-tetrahydrothieno[2,3-b]quinoline-2-carboxylic acid (2-amino-1-{3-[(furan-2-carbonyl)amino]phenyl}ethyl)amide (60 mg, 0.12 mmol) in 4 mL of CH₂Cl₂, was added 0.02 mL of NEt₃ and 320 mg of anhydrous Na₂SO₄. The mixture was stirred at room temperature for 2 h. It was cooled to 0° C., and 3.2 mL of MeOH was added. To the resulting mixture, was added NaBH₄ (4.4 mg, 0.12 mmol). The reaction was stirred at 0° C. for 5 min. It was quenched by adding 2 mL of 2 N aqueous HCl. The mixture was stirred at room temperature for 1 h. It was basified by 1 N aqueous NaOH, and extracted by 30 mL of CH₂Cl₂. The organic was concentrated under vacuum. The residue was further purified by flash chromatography eluting with 6% MeOH/CH₂Cl₂ to give 62 mg of crude 6-tert-butyl-5,6,7,8-tetrahydrothieno[2,3-b]quinoline-2-carboxylic acid (2-(2-benzyloxyethylamino)-1-{3-[(furan-2-carbonyl)amino]phenyl}ethyl)-amide.

Step B:

6-tert-Butyl-5,6,7,8-tetrahydrothieno[2,3-b]quinoline-2-carboxylic acid [1-{3-[(furan-2-carbonyl)amino]phenyl}-2-(2-hydroxyethylamino)ethyl]amide: A solution of the crude 6-tert-butyl-5,6,7,8-tetrahydrothieno[2,3-b]quinoline-2-carboxylic acid (2-(2-benzyloxyethylamino)-1-{3-[(furan-2-carbonyl)amino]phenyl}ethyl)amide (62 mg) in 1.5 ml of CHCl₃ and 0.75 mL of CH₃SO₃H was stirred at room temperature for 2 h. It was added to 20 mL of ice water. It was washed with 20 ml of ether. The aqueous portion was basified by 1 N NaOH, and extracted by 9:1 CH₂Cl₂/MeOH (20 mL×2). The organic was concentrated and further purified by flash chromatography eluting with 15% MeOH/CH₂Cl₂ to give 25 mg of 6-tert-butyl-5,6,7,8-tetrahydrothieno[2,3-b]quinoline-2-carboxylic acid [1-{3-[(furan-2-carbonyl)amino]phenyl}-2-(2-hydroxyethylamino)ethyl]amide. LCMS: MH⁺=561; mp (° C.)=183 (dec.).

### Example 265

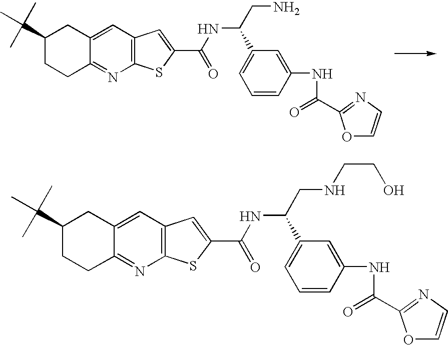

6-tert-Butyl-5,6,7,8-tetrahydrothieno[2,3-b]quinoline-2-carboxylic acid (2-(2-hydroxy-ethylamino)-1-{3-[(oxazole-2-carbonyl)amino]phenyl}ethyl)amide: This compound was prepared by essentially the same procedure set forth in Preparative Example 229, only substituting 6-tert-butyl-5,6,7,8-tetrahydrothieno[2,3-b]quinoline-2-carboxylic acid (2-amino-1-{3-[(furan-2-carbonyl)amino]phenyl}ethyl)amide with 6-tert-butyl-5,6,7,8-tetrahydrothieno[2,3-b]quinoline-2-carboxylic acid (2-amino-1-{3-[(oxazole-2-carbonyl)amino]phenyl}-ethyl)amide in step A. LCMS: MH⁺=562; mp (° C.)=179 (dec.).

### Example 266

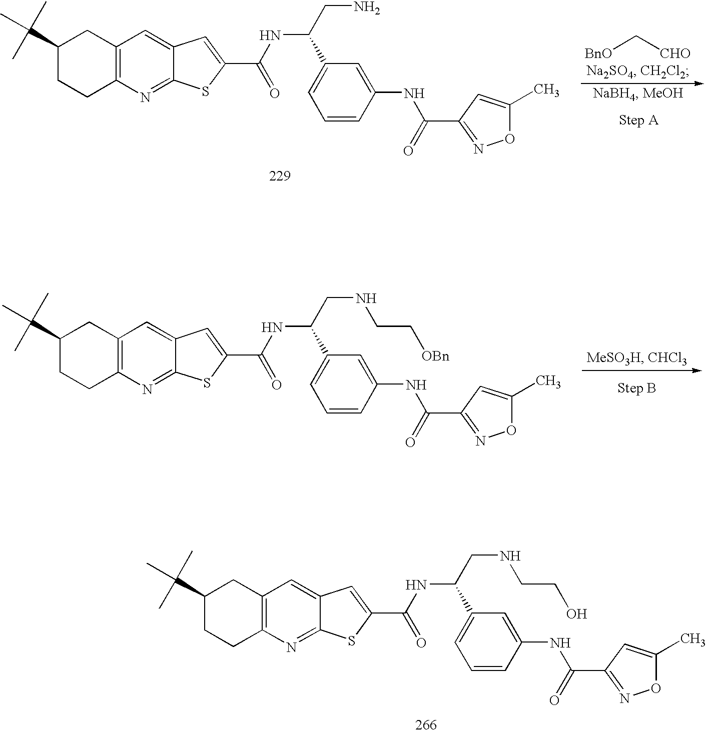 Step A:

6(R)-tert-Butyl-5,6,7,8-tetrahydro-thieno[2,3-b]quinoline-2-carboxylic acid (2-(2-benzyloxy-ethylamino)-1(S)-{3-[(5-methyl-isoxazole-3-carbonyl)-amino]-phenyl}-ethyl)-amide. To a solution of 6(R)-tert-butyl-5,6,7,8-tetrahydro-thieno[2,3-b]quinoline-2-carboxylic acid (2-amino-1(S)-3-[(5-methyl-isoxazole-3-carbonyl)-amino]-phenyl)ethyl)-amide (229) (31 mg, 0.06 mmol) in CH₂Cl₂ (1 mL), was added Et₃N (10 μL, 0.07 mmol), anhydrous Na₂SO₄ (120 mg), and benzyloxyacetaldehyde (9.0 μL, 0.06 mmol). The mixture was stirred at rt for 2 hr. The reaction was cooled to 0° C., MeOH (1.6 mL) was added, followed by NaBH₄ (2.8 mg, 0.07 mmol). The reaction was allowed to proceed for 15 min. The reaction was treated with CH₂Cl₂ (4×3 mL), dried over Na₂SO₄, filtered and concentrated. The product was purified by preparative TLC (7% MeOH/CH₂Cl₂) to yield 24.8 mg of product that was used directly in step B.

Step B:

6(R)-tert-Butyl-5,6,7,8-tetrahydro-thieno[2,3-b]quinoline-2-carboxylic acid (2-(2-hydroxy-ethylamino)-1(S)-{3-[(5-methyl-isoxazole-3-carbonyl)-amino]-phenyl}-ethyl)-amide. To a solution of 6(R)-tert-butyl-5,6,7,8-tetrahydro-thieno[2,3-b]quinoline-2-carboxylic acid (2-(2-benzyloxy-ethylamino)-1(S)-{3-[(5-methyl-isoxazole-3-carbonyl)-amino]-phenyl}-ethyl)amide (24.8 mg, 0.04 mmol) in CHCl₃ (1 mL), was added methanesulfonic acid (94 μL, 1.4 mmol), let it stir under a N₂ atmosphere at rt for 3 hr. The reaction was diluted with MeOH and CH₂Cl₂. The reaction solution was treated with 1 N NaOH (aqueous) until basic pH was reached. The aqueous layer was extracted with CH₂Cl₂ (3×), dried over Na₂SO₄, filtered and concentrated in vacuo to give a peach oil. The product was purified by preparative TLC (10% MeOH/CH₂Cl₂) to give 6(R)-tert-butyl-5,6,7,8-tetrahydro-thieno[2,3-b]quinoline-2-carboxylic acid (2-(2-hydroxy-ethylamino)-1(S)-{3-[(5-methyl-isoxazole-3-carbonyl)-amino]-phenyl}-ethyl)-amide as a pale yellow solid (5.5 mg, 26% yield). MS: MH⁺=576.3.

### Examples 267-268

Through essentially the same procedure set forth in Example 266, by substituting the amines 229 with the amines in Column 2 of Table 24 in Step 1, the compounds in Column 3 were prepared:

TABLE 24

| Example | Column 2 | Column 3 | CMPD |
|---|---|---|---|
| 267 | 237 | 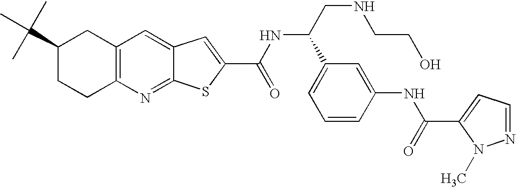 | MS: MH⁺ =575.3 free amine |
| 268 | 238 | 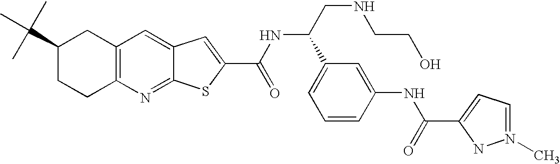 | MS: MH⁺ =575.3 free amine |

### Example 269

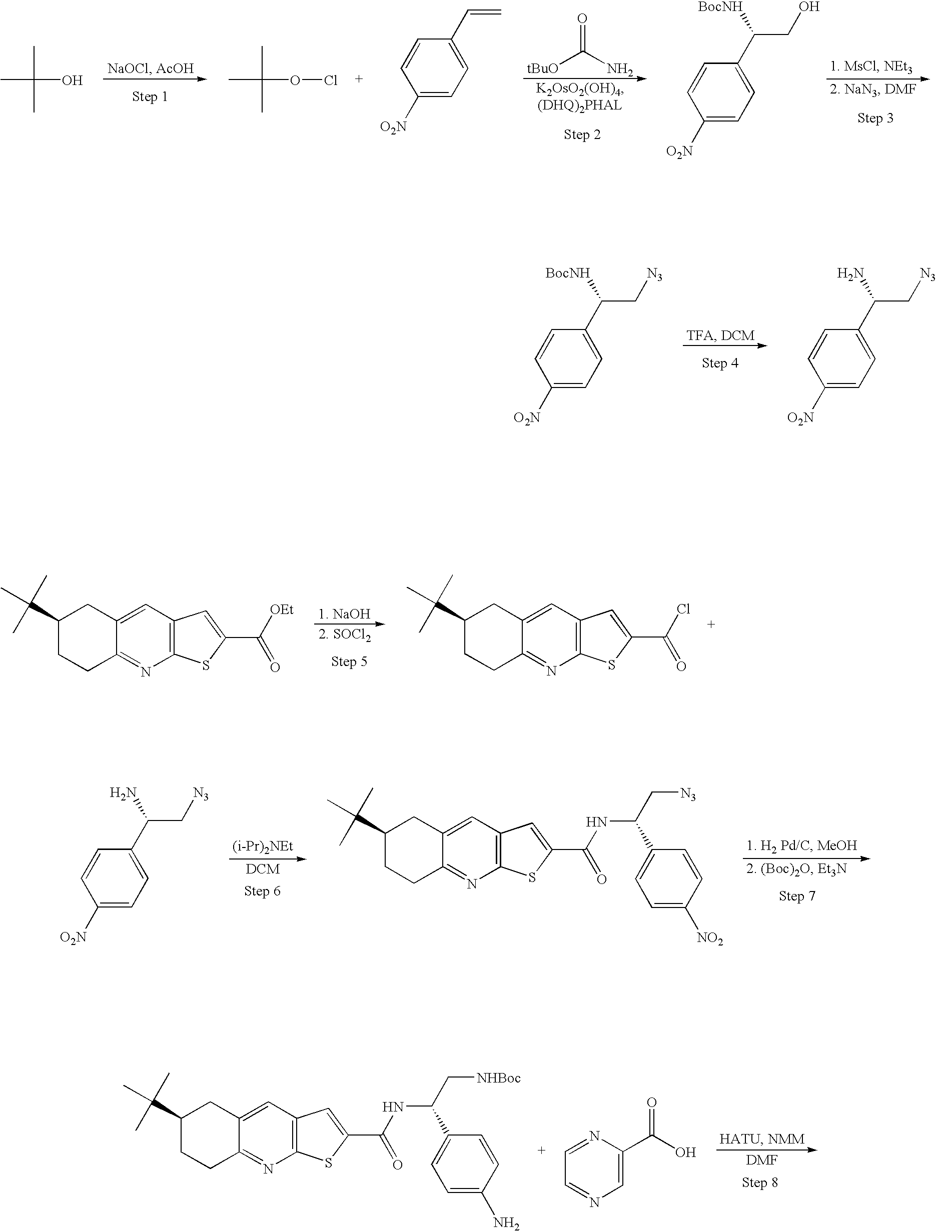 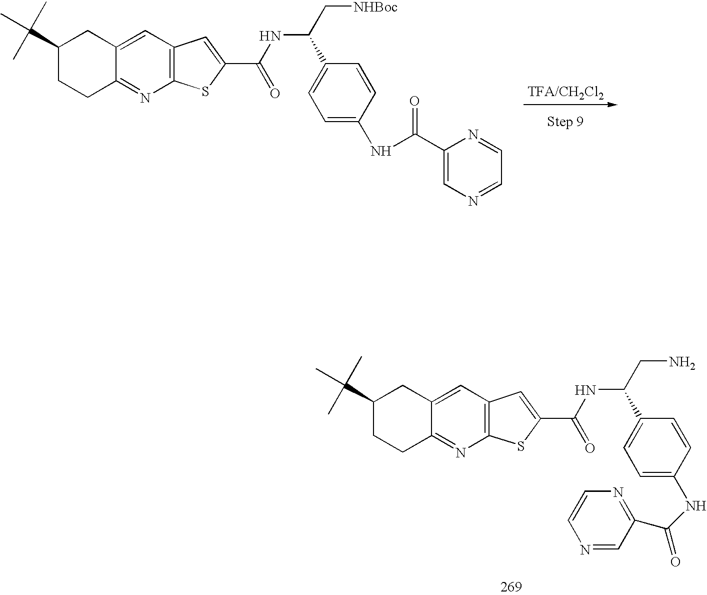 Step 1:

t-Butyl hypochlorite. 5 L of Clorox was stirred at 5° C. under dimmed light. To this was added 2-methyl-propan-2-ol (370 mL) and acetic acid (245 mL). The reaction was stirred at this temperature for 4 min. The top orange layer was separated and washed with 500 mL of cooled 10% Na₂CO₃ solution and water (500 mL). It was dried over anhydrous CaCl₂ and filtered. The freshly prepared t-butyl hypochlorite (˜300 g) was then stored in a freezer with 2 g of CaCl₂.

Step 2:

(1S)-N-(tert-Butyloxycarbonyl)-1-(4-nitrophenyl)-2-hydroxyethylamine. A solution of tert-butyl carbamate (7.18 g, 61 mmol) in n-PrOH (80 ml) was sequentially treated with a freshly prepared solution of NaOH (2.46 g in 150 ml of H₂O, save 16 ml for later use), followed by t-BuOCl (7 mL, 61 mmol). After stirring at room temperature for 5 min, the solution was cooled to 0° C. A solution of (DHQ)₂PHAL (0.94 g, 1.2 mmol) in n-PrOH (80 ml) was added, then 140 ml of n-PrOH was added, followed by K₂OsO₂(OH)₄ solution (prepared by 300 mg, 0.8 mmol of K₂OsO₂(OH)₄ in 16 ml NaOH solution mentioned above), and the 4-nitrostyrene (4 g, 26.8 mmol) was added portionwise (if melt to liquid, was added dropwise) to avoid the polymerize. The reaction was stirred at 0° C. for 1 hr. Quenched with Sat.Na₂S₂O₃ (200 mL), extracted with EtOAc (500 mL). It was dried over anhydrous Na2SO4 and then concentrated. The residue was purified by flash chromatography eluting with 33% EtOAc/hexanes to give 2.53 g of pure one produce, and the mixture of two isomers 3.0 g, the mixture was further purified by chromatography eluting with 25% EtOAc/hexanes to give 1.51 g of pure product, combined the two products, give 4.04 g (53% yield) of (1S)-N-(tert-butyloxycarbonyl)-1-(4-nitrophenyl)-2-hydroxyethylamine as a white foam.

Step 3:

(1S)-[2-azido-1-(4-nitrophenyl)ethyl]carbamic acid tert-butyl ester. A solution of (1S)-N-(tert-butyloxycarbonyl)-1-(4-nitrophenyl)-2-hydroxyethylamine (3.92 g, 13.9 mmol) and Et₃N (2.1 g, 2.9 ml, 20.85 mmol) in dichloromethane (65 ml) was treated with methanesulfonyl chloride (1.9 g, 1.29 ml, 16.68 mmol) at 0° C. The reaction was stirred at 0° C. for 1 h. It was diluted with dichloromethane (65 mL), washed with 1N HCl (20 mL). The organic layer was dried over anhydrous Na₂SO₄ and was concentrated under vacuum to give 5.6 g of crude mesylate as a solid

The above mentioned solid was taken up in DMF (65 mL), and sodium azide (2.7 g, 41.7 mmol) was then added. The reaction was heated at 70° C. under N₂ for 4 h. Low the temperate to R/T, quenched with 500 ml of H₂O, filter, washed the filter cake with H2O, collect the yellow solid from the filter cake to give 1.7 g of yellow solid. Then extracted the residue solution with 70% EtOAc/hexanes, the organic layer was dried over dry Na₂SO₄ and then concentrated to give crude yellow solid 2.1 g. Combined the two solid together (3.8 g), purified by flash chromatography eluting with 14% EtOAc/hexanes to give 1 g of mixture of pure product & impurity and 2.4 g of pure (1S)[2-azido-1-(4-nitrophenyl)ethyl]carbamic acid tert-butyl ester. (>56% yield)

Step 4:

(1S)-2-Azido-1-(4-nitrophenyl)ethylamine. The azide (1.64 g, 5.3 mmol) in 1:3 TFA/CH₂Cl₂ (52 mL) was stirred at room temperature for 2.5 h. The reaction was concentrated under vacuum. The residue was dissolved in dichloromethane (30 mL), basified with 1N NaOH to PH=9. The organic layer was extracted with CH₂Cl₂ many times until no product at the water layer. Dried over Na₂SO₄, filtered, and concentrated under vacuum to give 1.1 g (100% yield) of (1S)-2-azido-1-(4-nitrophenyl)ethylamine, which was used without further purification in the coupling reaction with the corresponding acid chloride.

Step 5:

6(R)-tert-Butyl-5,6,7,8-tetrahydro-thieno[2,3-b]quinoline-2-carbonyl chloride. To a solution of 6(R)-tert-butyl-5,6,7,8-tetrahydrothieno[2,3-b]quinoline-2-carboxylic acid ethyl ester (5.6 g, 17.7 mmol) in THF/MeOH (120 mL/60 mL), was added 1 N aqueous NaOH (26 mL). The reaction mixture was stirred at room temperature for 3 h. The solvent was removed under vacuum. The residue was dissolved in H₂O (20 mL), acidified with 2 N HCl. The solid was collected by filtration, washed with H₂O, and dried under vacuum to give the Acid. To this acid was added dichloromethane (80 mL), SOCl₂ (100 mL), 8 drops of DMF. The reaction was stirred at 43° C. for 2 h. The homogenous solution was concentrated under vacuum to remove the remaining SOCl₂. Dry dichloromethane (15 mL) was then added, followed by hexanes (300 mL). The solid was collected by filtration, and washed with hexanes to give 6-tert-butyl-5,6,7,8-tetrahydro-thieno[2,3-b]quinoline-2-carbonyl chloride 5.3 g.

Step 6:

6(R)-tert-Butyl-5,6,7,8-tetrahydrothieno[2,3-b]quinoline-2-carboxylic acid [(1S)-2-azido-1-(4-nitrophenyl)ethyl]amide. The above mentioned (1S)-2-azido-1-(4-nitro-phenyl)-ethylamine was dissolved in dry dichloromethane (50 ml). To this solution was added diisopropylethylamine (2.07 g, 2.79 mL, 16 mmol). It was cooled to 0° C. and 6(R)tert-butyl-5,6,7,8-tetrahydro-thieno[2,3-b]quinoline-2-carbonyl chloride (1.97 g, 6.4 mmol) was added, the reaction mixture was stirred at 0° C. for 10 min, then warm to R/T, stirred at R/T for half an hour, check mass, still has S.M, so more carbonyl chloride (300 mg) was added, continue to stirred at R/T for 5 min, it was diluted with dichloromethane (100 ml), washed with 0.5N HCl (50 mL), brine (30 mL), and back extracted the water layer with CH₂Cl₂, combined the organic layer, then dried over Na₂SO₄ and concentrated under vacuum to give 3.6 g of crude one. The crude one was purified by flash chromatography eluting with 7% MeOH/CH₂Cl₂ to give white foam 2.39 g (94% yield) of 6(R)-tert-butyl-5,6,7,8-tetrahydrothieno[2,3-b]quinoline-2-carboxylic acid [(1S)-2-azido-1-(4-nitrophenyl)ethyl]amide.

Step 7:

{2(S)-4-Aminophenyl)-2-[(6(R)-tert-butyl-5,6,7,8-tetrahydrothieno[2,3-b]quinoline-2-carbonyl)amino]ethylcarbamic acid tert-butyl ester. A mixture of 6(R)-tert-butyl-5,6,7,8-tetrahydrothieno[2,3-b]quinoline-2-carboxylic acid, [(1S)-2-azido-1-(4-nitrophenyl)ethyl]amide (433.8 mg, 0.91 mmol), 10% Pd/C (340 mg) in MeOH (30 mL) was stirred under a balloon of H₂ overnight. It was filtered through celite, washed the filter cake with 50% MeOH/CH₂Cl₂. The organic layer was concentrated to give yellow solid, further purified by flash chromatography eluting with MeOH/CH₂Cl₂/NH₄OH (100:10:1) to give white foam 322 mg (86% yield) of free amine. This was dissolved in dichloromethane (7.6 mL), followed by the addition of Et₃N (154 mg, 1.53 mmol). It was cooled to 0° C., and (Boc)₂O (158 mg, 0.72 mmol) was then added in one portion. The reaction was stirred from 0° C. to R/T for O/N. It was diluted with CH₂Cl₂ (10 mL), washed with H₂O, brine, dried over Na₂SO₄. The organic layer was concentrated. The residue was purified by silica gel chromatograph with 66% EtOAc/hexanes to give 347.1 mg of {2(S)-(4-Amino-phenyl)-2-[(6(R)-tert-butyl-5,6,7,8-tetrahydrothieno[2,3-b]quinoline-2-carbonyl)amino]ethylcarbamic acid tert-butyl ester (87% yield).

Step 8:

### (2-[(6(R)-tert-Butyl-5,6,7,8-tetrahydro-thieno[2,3-b]quinoline-2-carbonyl)-amino]-2(S)-{4-[(pyrazine-2-carbonyl)-amino]-phenyl}-ethyl)-carbamic acid tert-butyl ester

To a solution of {2(S)-(4-Amino-phenyl)-2-[(6(R)-tert-butyl-5,6,7,8-tetrahydrothieno[2,3-b]quinoline-2-carbonyl)amino]ethylcarbamic acid tert-butyl ester (35.9 mg, 0.069 mmol) and pyrazine-2-carboxylic acid (17 mg, 0.14 mmol) in 1 ml of DMF was added NMM (38 μL, 0.34 mmol), HATU (52.3 mg, 0.14 mmol). The reaction mixture was stirred at R/T for O/N. Dilute with H₂O, filtered, washed the filter cake with H₂O. Collect the white solid. Purified with EtOAc/CH₂Cl₂ (1:1) to elute with 34.5 mg white solid of (2-[(6(R)-tert-Butyl-5,6,7,8-tetrahydro-thieno[2,3-b]quinoline-2-carbonyl)-amino]-2(S)-{4-[(pyrazine-2-carbonyl)-amino]-phenyl}-ethyl)-carbamic acid tert-butyl ester. (80% yield)

Step 9:

6(R)-tert-Butyl-5,6,7,8-tetrahydro-thieno[2,3-b]quinoline-2-carboxylic acid (2-amino-1(S)-{4-[(pyrazine-2-carbonyl)-amino]-phenyl}-ethyl)-amide. A solution of (2-[(6(R)-tert-Butyl-5,6,7,8-tetrahydro-thieno[2,3-b]quinoline-2-carbonyl)-amino]-2(S)-{4-[(pyrazine-2-carbonyl)-amino]-phenyl}-ethyl)-carbamic acid tert-butyl ester (34.5 mg, 0.055 mmol) in THF/CH₂Cl₂ (0.1 mL/0.3 mL) was stirred at R/T for 1.5 hr. Evaporate most solvent, re-dissolved in 0.3 ml MeOH, basified with 1N NaOH, extracted with CH₂Cl₂ many times until no product at the water layer. The organic layer was dried over Na₂SO₄, concentrated in vacuum to give 61 mg of white solid. Purified by silica gel chromatograph with MeOH/CH2Cl2 (1:1) to elute 26 mg white solid of 6(R)-tert-Butyl-5,6,7,8-tetrahydro-thieno[2,3-b]quinoline-2-carboxylic acid (2-amino-1(S)-{4-[(pyrazine-2-carbonyl)-amino]-phenyl}-ethyl)-amide. (90% yield)

General Procedure for Making HCl Salt:

Dissolved the pure product in minimum MeOH, then 1 eq. of HCl (1M in Et₂O) was added, more Et₂O was added to give the participation. Then filter, washed the filter cake with Et₂O, collect the solid filter cake.

### Examples 270-272

Through essentially the same procedure set forth in Example 231, by substituting the acid in Column 2 of Table 25 in Step 8, the compounds in Column 3 were prepared:

TABLE 25

| Example | Column 2 | Column 3 | CMPD |
|---|---|---|---|
| 269 | 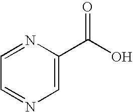 | 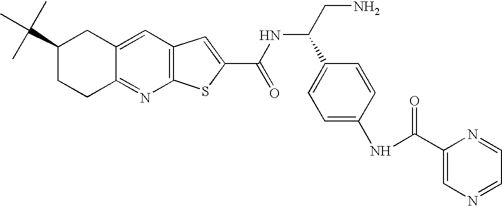 | MS: MH⁺ =529.2; mp (° C.) =149-152 |
| 270 | 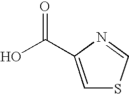 | 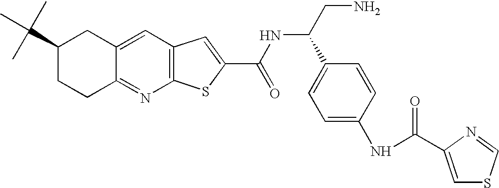 | MS: MH⁺ =534.2; mp (° C.) =164 (dec.) |
| 271 | 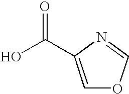 | 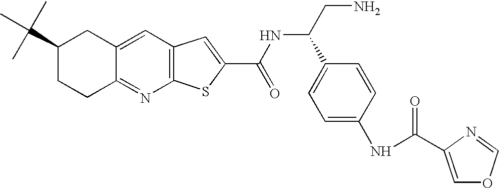 | MS: MH⁺ =518.2; mp (° C.) =182 (dec.) |
| 272 | 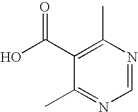 | 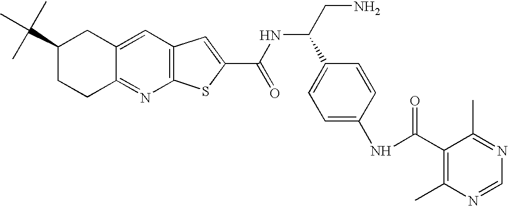 | MS: MH⁺ =557.3; mp (° C.) =225 (dec.) |

### Example 273

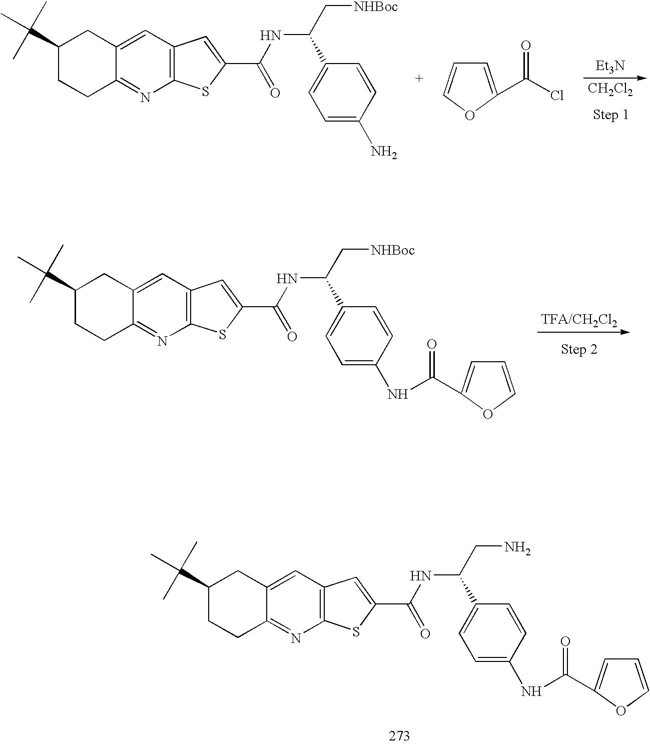 Step 1:

(2-[(6(R)-tert-Butyl-5,6,7,8-tetrahydro-thieno[2,3-b]quinoline-2-carbonyl)-amino]-2(S){4-[(furan-2-carbonyl)-amino]-phenyl}-ethyl)-carbamic acid tert-butyl ester. To a solution of {2(S)-(4-Amino-phenyl)-2-[(6(R)-tert-butyl-5,6,7,8-tetrahydrothieno[2,3-b]quinoline-2-carbonyl)amino]ethylcarbamic acid tert-butyl ester (37.2 mg, 0.07 mmol) in 1 mL of DCM, was added Et₃N (20 μL, 0.14 mmol), followed by 2-furoic acid chloride (8.4 μl, 0.086 mmol). The reaction mixture was stirred at R/T for 1 hr. Then more MeOH was added (1 mL). Stirred for 1 hr, diluted with 6 ml of CH₂Cl₂, washed with 0.5N HCl (3 mL). Back extracted the water layer with CH₂Cl₂ many times until no product at the water layer. dried over Na₂SO₄, concentrated in vacuum to give 59 mg. Purified by silica gel chromatograph with CH₂Cl₂/EtOAc (2:1) to elute white solid 35.6 mg (2-[(6(R)-tert-Butyl-5,6,7,8-tetrahydro-thieno[2,3-b]quinoline-2-carbonyl)amino]-2(S)-{4-[(furan-2-carbonyl)-amino]-phenyl}-ethyl)-carbamic acid tert-butyl ester. (81% yield)

Step 2:

6(R)-tert-Butyl-5,6,7,8-tetrahydro-thieno[2,3-b]quinoline-2-carboxylic acid (2-amino-1(S)-4-[(furan-2-carbonyl)-amino]-phenyl}-ethyl)-amide. A solution of (2-[(6(R)-tert-Butyl-5,6,7,8-tetrahydro-thieno[2,3-b]quinoline-2-carbonyl)-amino]-2(S)-{4-[(furan-2-carbonyl)amino]-phenyl}-ethyl)-carbamic acid tert-butyl ester (35.6 mg, 0.058 mmol) in TFA/DCM (0.15 mL/0.45 mL) was stirred at R/T for 1.5 hr. Evaporate most solvent. Re-dissolved in 0.1 ml of MeOH, basified with 1N NaOH to PH=10, more H₂O was added, white solid participate out. Filtered and washed the solid with more H₂O, collect the white solid to give 29.2 mg, purified by silica gel chromatograph with CH₂Cl₂/MeOH (10:1) to elute white solid 22.1 mg of 6(R)-tert-Butyl-5,6,7,8-tetrahydro-thieno[2,3-b]quinoline-2-carboxylic acid (2-amino-1(S)-{4-[(furan-2-carbonyl)-amino]-phenyl}-ethyl)-amide. (74% yield)

General Procedure for Making HCl Salt:

Dissolved the pure product in minimum MeOH, then 1 eq. of HCl (1M in Et₂O) was added, more Et₂O was added: to give the participation. Then filter, washed the filter cake with Et₂O, collect the solid from the filter cake.

### Examples 273-274

Through essentially the same procedure set forth in Example 227, by substituting the acid chloride in Column 2 of Table 27 in Step 1, the compounds in Column 3 were prepared:

TABLE 27

| Example | Column 2 | Column 3 | CMPD |
|---|---|---|---|
| 273 | 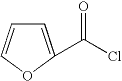 | 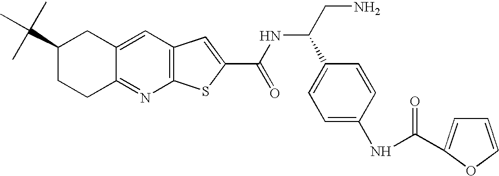 | MS: MH⁺ =517.2; mp (° C.) =209 (dec.) |
| 274 | 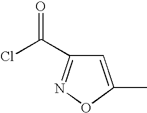 | 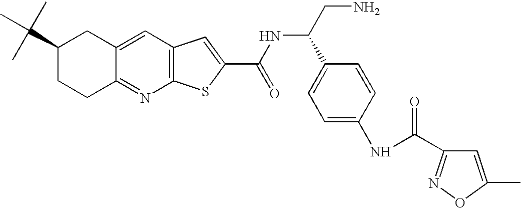 | MS: MH⁺ =532.2; mp (° C.) =205 (dec.) |

### Example 275

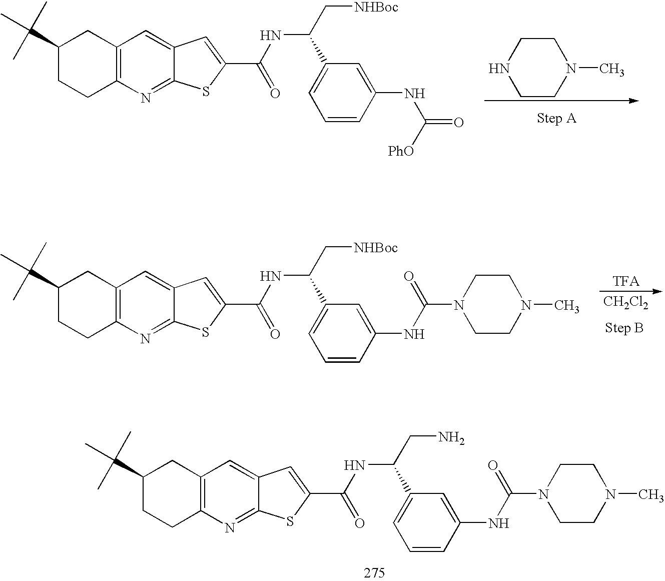 Step A:

(2-[(6(R)-tert-Butyl-5,6,7,8-tetrahydro-thieno[2,3-b]quinoline-2-carbonyl)-amino]-2(S)-{3-[(4-methyl-piperazine-1-carbonyl)-amino]-phenyl}-ethyl)-carbamic acid tert-butyl ester. To a solution of 1-methyl-piperazine (8.5 mg, 0.08 mmol) in DMSO (1 mL) was added (3(S)-{2-tert-butoxycarbonylamino-1-[(6(R)-tert-butyl-5,6,7,8-tetrahydro-thieno[2,3-b]quinoline-2-carbonyl)amino]-ethyl}-phenyl)-carbamic acid phenyl ester (18 mg, 0.03 mmol). The reaction mixture was stirred at rt for 1 hr. The reaction was diluted with H₂O (5 mL), and 3 drops of 2 N HCl (aqueous) was added. A white solid precipitated out of the solution. The reaction mixture was stirred for a few minutes, the white solid was filtered and washed with H₂O. The solid was diluted with CH₂Cl₂, dried over Na₂SO₄, filtered, and concentrated to give pale yellow oil that was used directly in step B.

Step B:

6(R)-tert-Butyl-5,6,7,8-tetrahydro-thieno[2,3-b]quinoline-2-carboxylic acid (2-amino-1(S)-{3-[(4-methyl-piperazine-1-carbonyl)-amino]-phenyl}-ethyl)-amide. To a flask containing (2-[(6(R)-tert-butyl-5,6,7,8-tetrahydro-thieno[2,3-b]quinoline-2-carbonyl)-amino]-2(S)-{3-[(4-methyl-piperazine-1-carbonyl)-amino]-phenyl}-ethyl)-carbamic acid tert-butyl ester (18.2 mg, 0.03 mmol) was added 2.0 mL of 1:3 TFA/CH₂Cl₂ solution. The reaction was allowed to stir under a N₂ atmosphere for 1-2 hr. The solvent was removed in vacuo, and the residue was treated with 2 mL of MeOH, followed by 10 drops of saturated Na₂CO₃ solution. Dichloromethane (10 mL) and Na₂SO₄ (anhydrous) were added, the reaction mixture was filtered and concentrated. The product was purified via preparative TLC (20% MeOH/CH₂Cl₂, eluted 2×) to give 6.0 mg (39% yield) of 6(R)-tert-butyl-5,6,7,8-tetrahydro-thieno[2,3-b]quinoline-2-carboxylic acid (2-amino-1(S)-{3-[(4-methyl-piperazine-1-carbonyl)-amino]-phenyl}-ethyl)-amide.

General Procedure for making HCl Salt: The product was dissolved in a minimal amount of CH₂Cl₂, and one equivalent of HCl solution (1 M in Et₂O) was added to the solution while rapidly stirring. Et₂O was added and the product salt precipitated from the solution. The solid was collected by filtration, washed with Et₂O, and dried under vacuum. LCMS: MH⁺=549; mp (° C.)=198 (dec).

### Example 276-280

Through essentially the same procedure set forth in Example 275, by substituting the amine in Column 2 of Table 28 in Step 1, the compounds in Column 3 were prepared:

TABLE 28

| Example | Column 2 | Column 3 | CMPD |
|---|---|---|---|
| 276 | 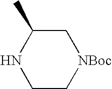 | 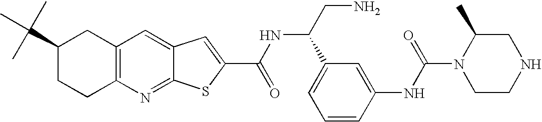 | MS: MH⁺ =549.2; mp (° C.) =238 (dec.) |
| 277 | 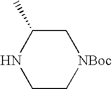 | 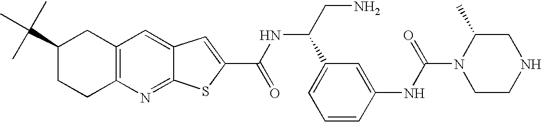 | MS: MH⁺ =549.2; mp (° C.) =223 (dec.) |
| 278 | 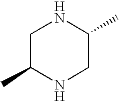 | 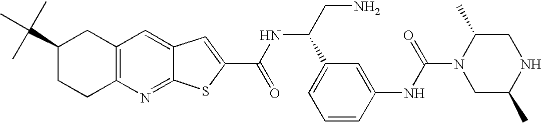 | MS: MH⁺ =563.2; mp (° C.) =246 (dec.) |
| 279 | 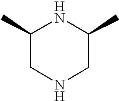 | 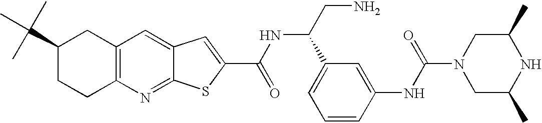 | MS: MH⁺ =563.2; mp (° C.) =250 (dec.) free amine |
| 280 | 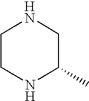 | 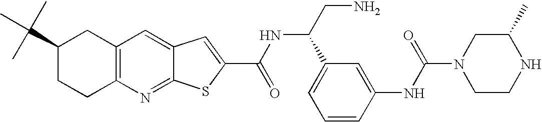 | MS: MH⁺ =548.2 free amine |

### Example 281

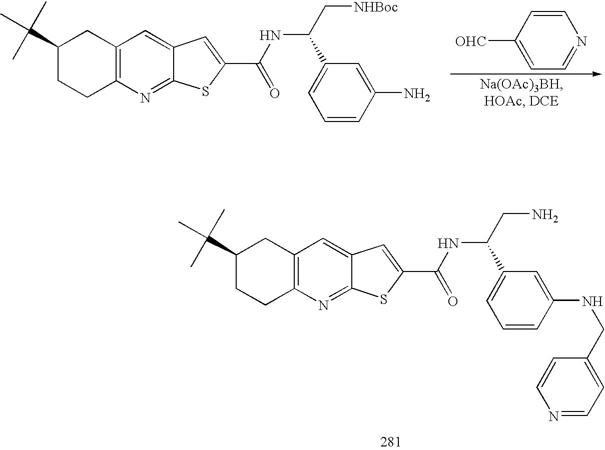

6(R)-tert-Butyl-5,6,7,8,-tetrahydro-thieno[2,3-b]quinoline-2-carboxylic acid (2(S)-amino-1-{3-[(pyridin-4-ylmethyl)-amino]-phenyl}-ethyl)-amide. To a solution of {2(S)-(3-amino-phenyl)-2-[(6(R)-tert-butyl-5,6,7,8-tetrahydro-thieno[2,3-b]quinoline-2-carbonyl)-amino]-ethyl}-carbamic acid tert-butyl ester (34 mg, 0.07 mmol) in 1,2-dichloroethane (1.0 mL), was added 4-pyridinecarboxaldehyde (14 mg, 0.13 mmol), Na(OAc)₃BH (42 mg, 0.20 mmol), and HOAc (19 μL) . The reaction mixture was stirred at rt for 18 hr. The reaction was diluted with CH₂Cl₂ and quenched by the addition of saturated NaHCO₃ solution. The aqueous layer was extracted with CH₂Cl₂. The organic phase was dried over anhydrous Na₂SO₄, filtered and concentrated. The product was purified by preparative TLC (15% MeOH/CH₂Cl₂ containing 1% NH₄OH) to give 29.3 mg (87% yield) of 6(R)-tert-butyl-5,6,7,8-tetrahydro-thieno[2,3-b]quinoline-2-carboxylic acid (2(S)-amino-1-3-[(pyridin-4-ylmethyl)-amino]-phenyl}-ethyl)-amide as a yellow solid. LCMS: MH⁺=514.3; mp (° C.)=113-117.

### Examples 282-283

Through essentially the same procedure set forth in Example 280, by substituting the aldehydes in Column 2 of Table 29, the compounds in Column 3 were prepared:

TABLE 29

| Example | Column 2 | Column 3 | CMPD |
|---|---|---|---|
| 282 | 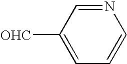 | 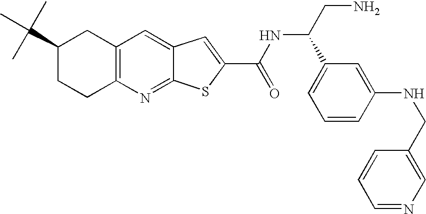 | LC-MS: MH⁺ =514.3; mp (° C.) =120-122 (dec) free amine |
| 283 | 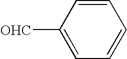 | 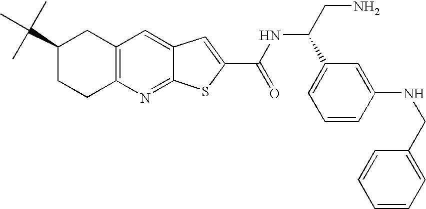 | LC-MS: MH⁺ =513.3; mp (° C.) =220-242 (dec) free amine |

### Example 284

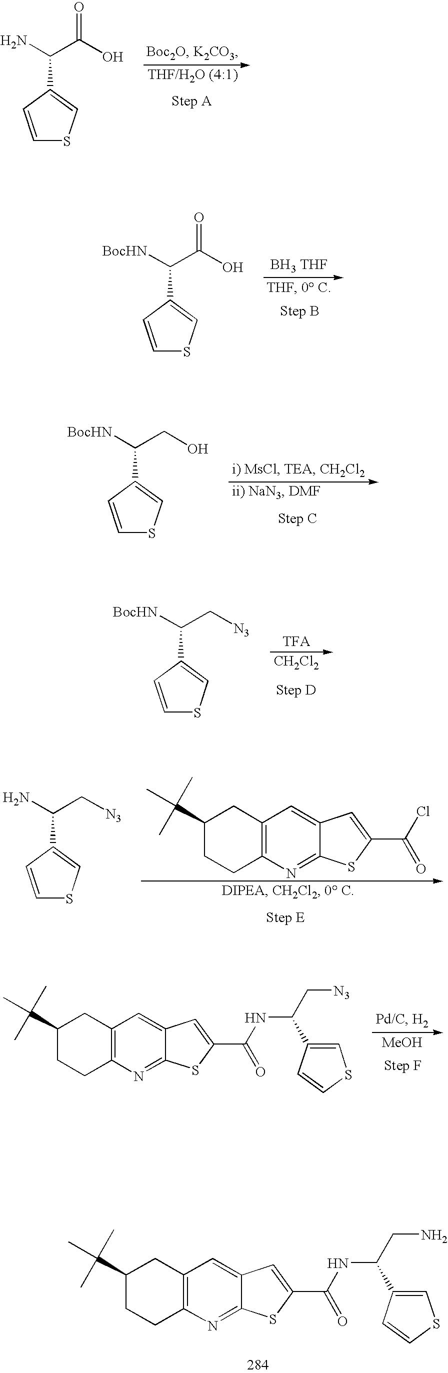 Step A:

tert-Butoxycarbonylamino-(S)-thiophen-3-yl-acetic acid. To a solution of amino-(S)-thiophen-3-yl-acetic acid (500 mg, 3.18 mmol) in THF/H₂O (24 mL/6 mL), was added K₂CO₃ (650 mg, 4.77 mmol) and Boc₂O (763 mg, 3.5 mmol). The reaction mixture was stirred at rt for 12 hr. The reaction was diluted with EtOAc and H₂O, The aqueous layer was extracted with EtOAc. The aqueous phase was made acidic (pH ˜5-6) with 2 N HCl (aqueous). The acidic aqueous layer was extracted with EtOAc. The combined organic layers were dried over anhydrous Na₂SO₄, filtered and concentrated in vacuo to give a white solid 510 mg (62% yield). The product was used directly in step B without further purification.

Step B:

(2-Hydroxy-1(S)-thiophen-3-yl-ethyl)-carbamic acid tert-butyl ester. To a solution of tert-butoxycarbonylamino-(S)-thiophen-3-yl-acetic acid (510 mg, 1.98 mmol) in THF (20 mL), at 0° C. was added slowly a solution of BH₃. THF (4 mL, 3.96 mmol) complex via syringe. The reaction was stirred at 0° C. for 2 hr. The reaction was cooled to 0° C. and quenched by the slow addition of H₂O. Ethyl acetate was added to the reaction mixture and stirring was continued at rt for 1 hr. The aqueous phase was extracted with EtOAc. The combined organic phase was washed with brine, dried over MgSO₄, filtered and concentrated. The product was purified by preparative TLC (5% MeOH/CH₂Cl₂) to isolate 88.3 mg (18% yield) of (2-hydroxy-1(S)-thiophen-3-yl-ethyl)carbamic acid tert-butyl ester as a white solid.

Step C:

(2-Azido-1(S)-thiophen-3-yl-ethyl)-carbamic acid tert-butyl ester. To a solution of (2-hydroxy-1(S)-thiophen-3-yl-ethyl)carbamic acid tert-butyl ester (88 mg, 0.36 mmol) in CH₂Cl₂ (4 mL), at 0° C. was added Et₃N (76 μL, 0.54 mmol), followed by methanesulfonyl chloride (34 μL, 0.43 mmol). The reaction was stirred at 0° C. under a N₂ atmosphere for 2.5 hr. The reaction was quenched by the addition of CH₂Cl₂ and 1 N HCl (aqueous). The organic layer was dried (anhydrous Na₂SO₄), filter and concentrated to give a pale yellow solid. The yellow solid was dissolved in DMF (0.8 mL), and NaN₃ (70.6 mg, 1.09 mmol) was added. The reaction mixture was heated at 65° C. for 20 hr. The reaction was cooled to rt. A solid precipitated from the solution upon addition of H₂O. The solid was collect by filtration and washed with H₂O. The product was dried under vacuum to give 2-azido-1(S)-thiophen-3-yl-ethyl)-carbamic acid tert-butyl ester as a white solid 72.1 mg (74% yield).

Step D:

2-Azido-1(S)-thiophen-3-yl-ethylamine. A solution of (2-azido-1(S)-thiophen-3-yl-ethyl)-carbamic acid tert-butyl ester (72.1 mg, 0.27 mmol) in TFA/CH₂Cl₂ (0.5 mL/1.5 mL) was stirred at rt for 1.5 hr. The reaction was diluted with CH₂Cl₂ and quenched with 1 N NaOH (aqueous). The aqueous layer was extracted with 10% MeOH/CH₂Cl₂, dried (anhydrous Na₂SO₄), filter, and concentrated to give 41.6 mg (92% yield) of 2-azido-1(S)-thiophen-3-yl-ethylamine.

Step E:

6-tert-Butyl-5,6,7,8-tetrahydro-thieno[2,3-b]quinoline-2-carboxylic acid (2-azido-1(S)-thiophen-3-yl-ethyl)-amide. Following the same procedure of Example 225, except substituting 2-azido-1(S)-thiophen-3-yl-ethylamine (42 mg, 0.25 mmol) in place of 2-azido-1-phenyl-ethylamine provided 6-tert-butyl-5,6,7,8-tetrahydro-thieno[2,3-b]quinoline-2-carboxylic acid (2-azido-1(S)-thiophen-3-yl-ethyl)-amide.

Step F:

6-tert-Butyl-5,6,7,8-tetrahydro-thieno[2,3-b]quinoline-2-carboxylic acid (2-amino-1(S)-thiophen-3-yl-ethyl)-amide. To a solution of 6-tert-butyl-5,6,7,8-tetrahydro-thieno[2,3-b]quinoline-2-carboxylic acid (2-azido-1(S)-thiophen-3-yl-ethyl)-amide (118 mg, 0.27 mmol) in MeOH (3 mL) was added 10% Pd/C (50 mg). The reaction was vigorously stirred at rt under a H₂ atmosphere (1 atm) for 3 hr. The reaction was filtered through a pad of Celite (eluent/washed with MeOH/CH₂Cl₂). The product was purified by preparative TLC (10% MeOH/CH₂Cl₂) to give 25.2 mg (23% yield, 2 steps) of 6-tert-butyl-5,6,7,8-tetrahydro-thieno[2,3-b]quinoline-2-carboxylic acid (2-amino-1(S)-thiophen-3-yl-ethyl)-amide as a white solid. LCMS: MH⁺=414.2, mp (° C.)=117-121.

### Example 285

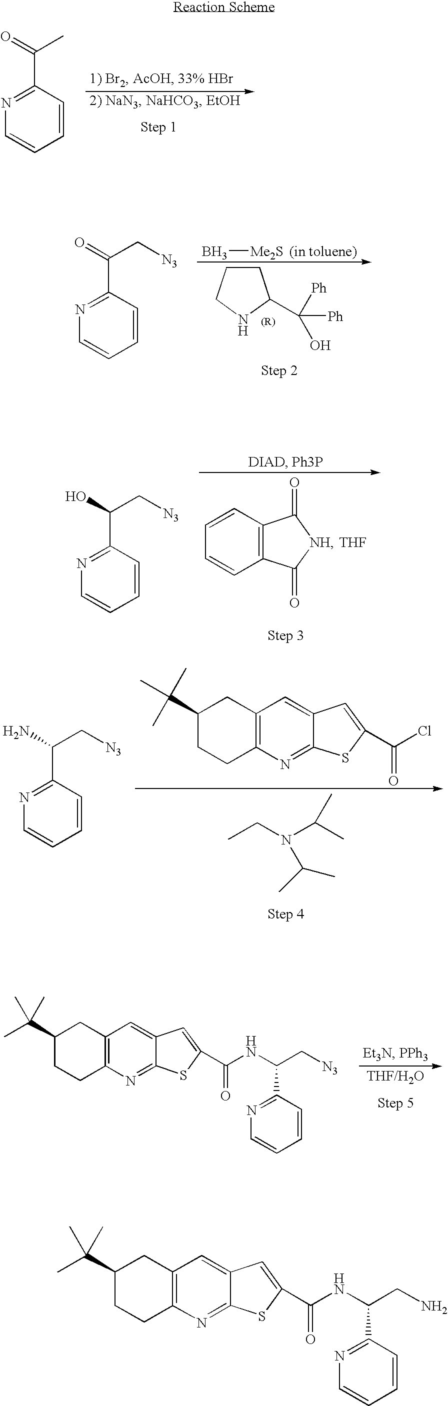 Step 1:

2-Azido-1-pyridin-2-yl-ethanone. To a solution of 1-Pyridin-2-yl-propan-1-one (3 g, 24.8 mmol) in acetic acid (28 mL) was dropwise added a bromine solution (4 g) in 33% HBr at 0° C. The reaction mixture was stirred warmed to 40° C. and stirred for 1.5 hr, followed by stirring at 75° C. for 1 hr. The mixture was cooled to room temperature and diluted with ether (100 mL), filtered and washed with ether and concentrated to give 0.25 g of the bromo-product. Taken in EtOH (4 mL), added NaHCO₃ (75 mg, 0.89 mmol, 1 eq), 2 eq of sodium azide (116 mg, 1.78 mmol, 2 eq) and the reaction mixture was stirred at room temperature for 4 hr. Poured into 200 ml of EtOAc, washed with H₂O (1×100 mL), the organic layer was dried over Na₂SO₄, filtered and concentrated, purified via Biotage using 15% EtOAc/Hexane to give 88 mg of the desired product.

Step 2:

2-Azido-1(S)-pyridin-2-yl-ethanol. To a solution of (R)-Methyl-CBS-oxazilidinone (1.85 mL, 1.85 mmol, 3 eq) in 200 mL RBF, was added 2M solution of BH₃.Me₂S in toluene (3.1 mL, 6.2 mmol, 1 eq) and stirred at room temperature for 10 min, then a solution of 2-Azido-1-pyridin-2-yl-ethanone (1 g, 6.2 mmol, 1 eq) in toluene (10 mL) was added via syringe over 1 hr. The reaction mixture was stirred at room temperature for 30 min. Then the reaction mixture was cooled to 0° C., quenched carefully with MeOH and concentrated. Purified on Biotage using 35% EtOAC/Hexanes to give 0.64 g of the product.

Step 3:

2-Azido-1(S)-pyridin-2-yl-ethylamine. To a solution of Ph₃P (2.05 g, 7.08 mmol, 2 eq) in THF (50 mL), at 0° C. was added DIAD (1.51 mL, 7.8 mmol, 2 eq), stirred for 20 min, then the 2-Azido-1(S)-pyridin-2-yl-ethanol (0.64 g, 3.9 mmol, 1 eq) in THF (20 mL) was added, followed by Phthalimide (1.15 g, 7.8 mmol, 2 eq) in small portions. Stirred at room temperature for 10 hr. Concentrated in vacuum and purified via Biotage using 35% EtOAc/Hexanes to give white solid product. Dissolved the white solid in THF (20 mL), H₂O (20 mL), hydrazine (0.62 mL) and MeOH (minimum amount to make solution homogeneous), stirred homogeneous solution at room temperature for 10 hr. Poured into EtOAc (200 mL), washed with Sat. NaHCO₃ (1×100 mL) and the aqueous layer was washed with EtOAc (100 mL). Combined the organic layers, dried over Na₂SO₄, filtered and concentrated. Purified via Biotage using 3% MeOH (NH₃)/CH₂Cl₂ to 5% MeOH(NH₃)/CH₂Cl₂ to give 0.5 g of product.

Step 4:

6(R)-tert-Butyl-5,6,7,8-tetrahydro-thieno[2,3-b]quinoline-2-carboxylic acid (2-azido-1(S)-pyridin-2-yl-ethyl)-amide. To a solution of 2-Azido-1(S)-pyridin-2-yl-ethylamine (200 mg, 1.23, 1.5 eq) in CH₂Cl₂ (10 mL) was added i-Pr₂EtN (0.64 mL, 3.68 mmol, 4.5 eq), at −78° C. was added the acid chloride (0.25 g, 0.82 mmol, 1 eq). The reaction mixture was warmed to room temperature and stirred for 18 hr. Poured into 200 mL and washed with Sat. NaHCO₃ (1×100 mL). The organic layer was dried over Na₂SO₄, filtered and concentrated. Purified via Biotage using 35% EtOAc/Hexanes to give 350 mg product.

Step 5:

6(R)-tert-Butyl-5,6,7,8-tetrahydro-thieno[2,3-b]quinoline-2-carboxylic acid (2-amino-1(S)-pyridin-2-yl-ethyl)-amide. A solution of 6(R)-tert-Butyl-5,6,7,8-tetrahydro-thieno[2,3-b]quinoline-2-carboxylic acid (2-azido-1(S)-pyridin-2-yl-ethyl)-amide (0.15 g, 0.35 mmol, 1 eq), Ph₃P (181 mg, 0.69 mmom, 2 eq), Et₃N (0.195 ml, 1.4 mmol, 4 eq) in 20 mL of THF/H₂O (4:1) was stirred room temperature for 18 hr. Concentrated and purified via Biotage using 4% MeOH(NH₃)/CH₂Cl₂ to give 90 mg of product.

### Examples 286-288

Through essentially the same procedure set forth in Example 286, by substituting the acid in Column 2 of Table 30 in Step 9, the compounds in Column 3 were prepared:

TABLE 30

| Example | Column 2 | Column 3 | CMPD |
|---|---|---|---|
| 286 | 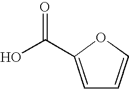 | 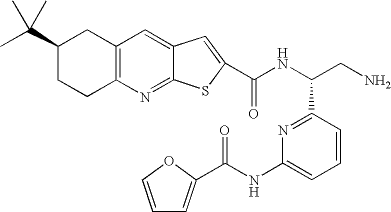 | MS: MH⁺ =518.2; |
| 287 | 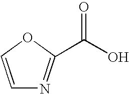 | 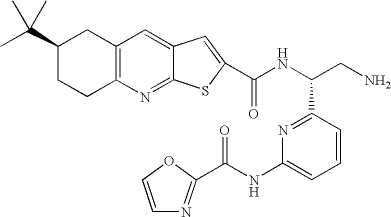 | MS: MH⁺ =519.2; |
| 288 | 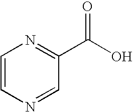 | 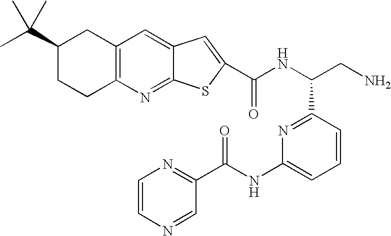 | MS: MH⁺ =530.2; |

### Example 286

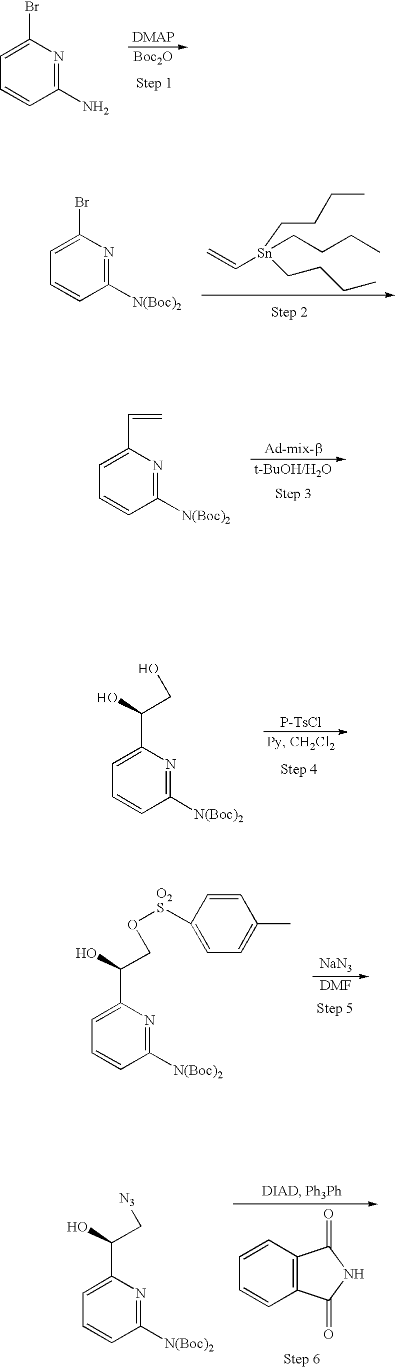 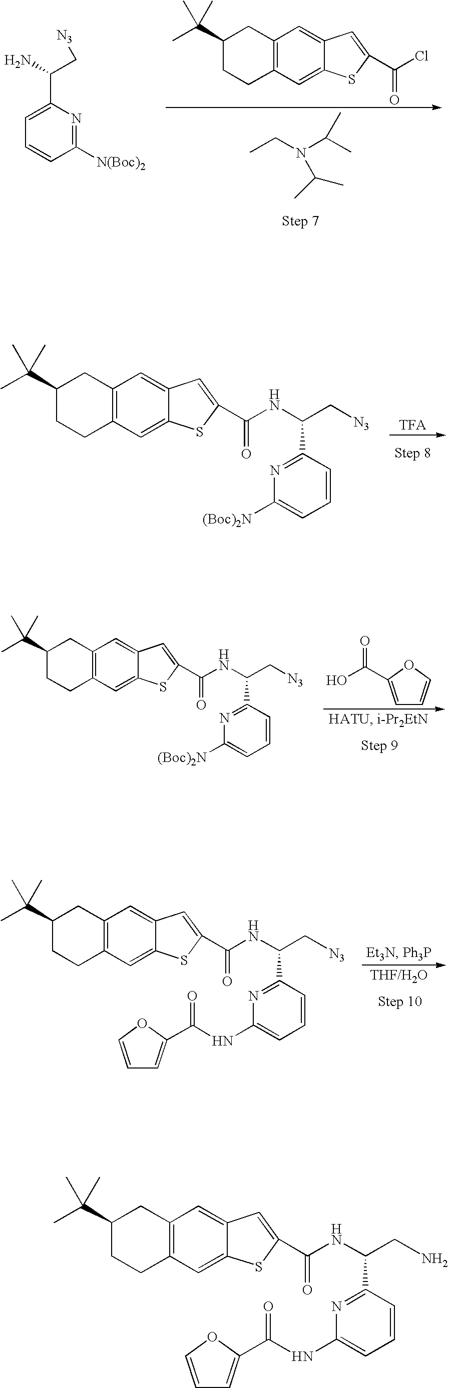 Step 1:

(6-Bromo-pyridin-2-yl)-carbamic acid di-tert-butyl ester. To a solution of 6-Bromo-pyridin-2-ylamine (5 g, 28.9 mmol, 1 eq) in 50 mL of CH₂Cl₂ was added Boc₂O (9.5 g, 43.4 mmol, 1.5 eq), DMAP (0.35 g, 2.89 mmol, 0.1 eq), the reaction mixture was stirred at room temperature for 72 hr. Concentrated and ether was added, filtered to give 5 g of solid product.

Step 2:

(6-Vinyl-pyridin-2-yl)-carbamic acid di-tert-butyl ester. To s solution of (6-Bromo-pyridin-2-yl)-carbamic acid di-tert-butyl ester (0.5 g, 1.34 mmol, 1 eq) in DMF (5 ml), was added tributyl-vinyl-stannane (1.6 mL, 5.36 mmol, 4 eq) and Pd(Ph₃P)₄ (155 mg, 0.134 mmol, 0.1 eq). The reaction mixture was heated at 100° C. for 18 hr. Cooled and poured into EtOAc (100 mL), washed with Sat. NaHCO₃ (1×100 mL), H₂O and brine. The organic layer was dried over Na₂SO₄, filtered and concentrated. Purified via Biotage using 9/1 Hexanes/EtOAc to give 0.4 g of the product.

Step 3:

[6-(1(R),2-Dihydroxy-ethyl)-pyridin-2-yl]-carbamic acid di-tert-butyl ester. To a solution of (6-Vinyl-pyridin-2-yl)-carbamic acid di-tert-butyl ester (0.28 g, 0.84 mmol, 1 eq) in 20 mL of a mixture of t-BuOH/H₂O (1:1), at 0° C. was added AD-mix-β (2.52 g, 1.67 mmol, 2 eq). The reaction mixture was stirred at 0° C. for 6 hr, then warm to room temperature and stirred for 18 hr. Na₂SO₃ (3 g) was added and continue stirring at room temperature for 30 min. Poured into EtOAc (100 mL), washed with Sat. NaHCO₃ and dried over Na₂SO₄, filtered and concentrated. Purified via Biotage using EtOAc to give 0.27 g of product.

Step 4:

Toluene-4-sulfonic acid 2-(6-di-tert-butoxycarbonylamino-pyridin-2-yl)-2(R)-hydroxy-ethyl ester. To a solution of [6-(1,2-Dihydroxy-ethyl)-pyridin-2-yl]-carbamic acid di-tert-butyl ester (0.27 g, 0.76 mmol, 1 eq) in Pyridine (5 ml), at 0° C. was added P-TsCl (59 mg, 0.84 mmol, 1.1 eq). The reaction mixture was stirred at room temperature for 18 hr. Concentrated and poured into EtOAc (100 mL), washed with H₂O (110 mL) and Sat. NaHCO₃ (100 mL). The organic layer was dried over Na₂SO₄, filtered and concentrated. Purified via Biotage using 1:1 EtOAc/Hexanes to give 170 mg of the product.

Step 5:

[6-(2-Azido-1(R)-hydroxy-ethyl)-pyridin-2-yl]-carbamic acid di-tert-butyl ester. To a solution of toluene-4-sulfonic acid 2-(6-di-tert-butoxycarbonylamino-pyridin-2-yl)-2-hydroxy-ethyl ester (170 mg, 0.334 mmol, 1 eq) in DMF (3 mL) was added NaN₃(44 mg, 0.67 mmol, 2 eq). The reaction mixture was heated at 85° C. for 2 hr. Then cooled and poured into EtOAc (200 mL) and washed with H₂O (2×100 mL), sat.NaHCO₃ (1×100 mL). The organic layer was dried, filtered and concentrated to give 140 mg of product which directly used in the next step without further purification.

Step 6:

[6-(1(S)-Amino-2-azido-ethyl)-pyridin-2-yl]-carbamic acid di-tert-butyl ester. The first step is the same as above Example 285, step 3. Taken 170 mg from step 1 and was added 5 mL of THF & 5 ml of H₂O and minimum amount of MeOH to make solution homogeneous, 58 μl of hydrazine was added and stirred at room temperature for 18 hr, then heated at 40° C. for 2 hr and continue to stir at room temperature for 18 hr, heated at 40° C. for 2 hr and continue to stir at room temperature for 48 hr. Poured into 200 mL of EtOAc and washed with Sat.NaHCO₃ (2×100 mL). The organic layer was dried over Na₂SO₄ and filtered and concentrated. Purified via Biotage using 1:1 EtOAc/Hexanes to give 100 mg of product.

Step 7:

(6-{2-Azido-1-[(6(R)-tert-butyl-5,6,7,8-tetrahydro-thieno[2,3-b]quinoline-2-carbonyl)-amino]-ethyl}-1-(S)-pyridin-2-yl)-carbamic acid di-tert-butyl ester. Following the same procedure set forth in Example 285 step 4.

Step 8

6(R)-tert-Butyl-5,6,7,8-tetrahydro-thieno[2,3-b]quinoline-2-carboxylic acid [1(S)-(6-amino-pyridin-2-yl)-2-azido-ethyl]-amide. Following the same procedure set forth in Example 231 step B

Step 9:

6(R)-tert-Butyl-5,6,7,8-tetrahydro-thieno[2,3-b]quinoline-2-carboxylic acid (2-azido-1(S)-{6-[(furan-2-carbonyl)-amino]-pyridin-2-yl}-ethyl)-amide. Following the same procedure set forth in Example 231 step A

Step 10:

6(R)-tert-Butyl-5,6,7,8-tetrahydro-thieno[2,3-b]quinoline-2-carboxylic acid (2-amino-1(S)-{6-[(furan-2-carbonyl)-amino]-pyridin-2-yl}-ethyl)-amide. Folowing the same procedure set forth in Example XX step 5

### Examples 289-291

Through essentially the same procedure set forth in Example 289, by substituting the acid in Column 2 of Table 31 in Step 8, the compounds in Column 3 were prepared:

TABLE 31

| Example | Column 2 | Column 3 | CMPD |
|---|---|---|---|
| 289 | 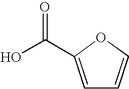 | 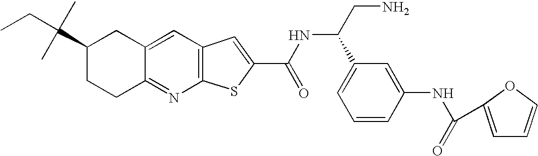 | MS: MH⁺ =531.2; |
| 290 | 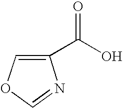 | 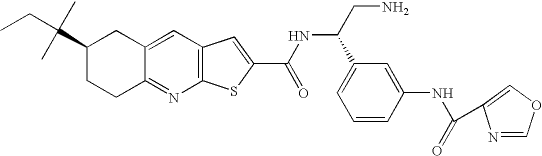 | MS: MH⁺ =532.2; |
| 291 | 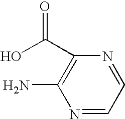 | 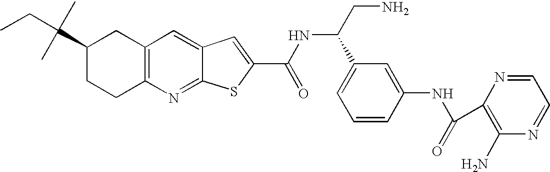 | MS: MH⁺ =558.2; |

### Example 289

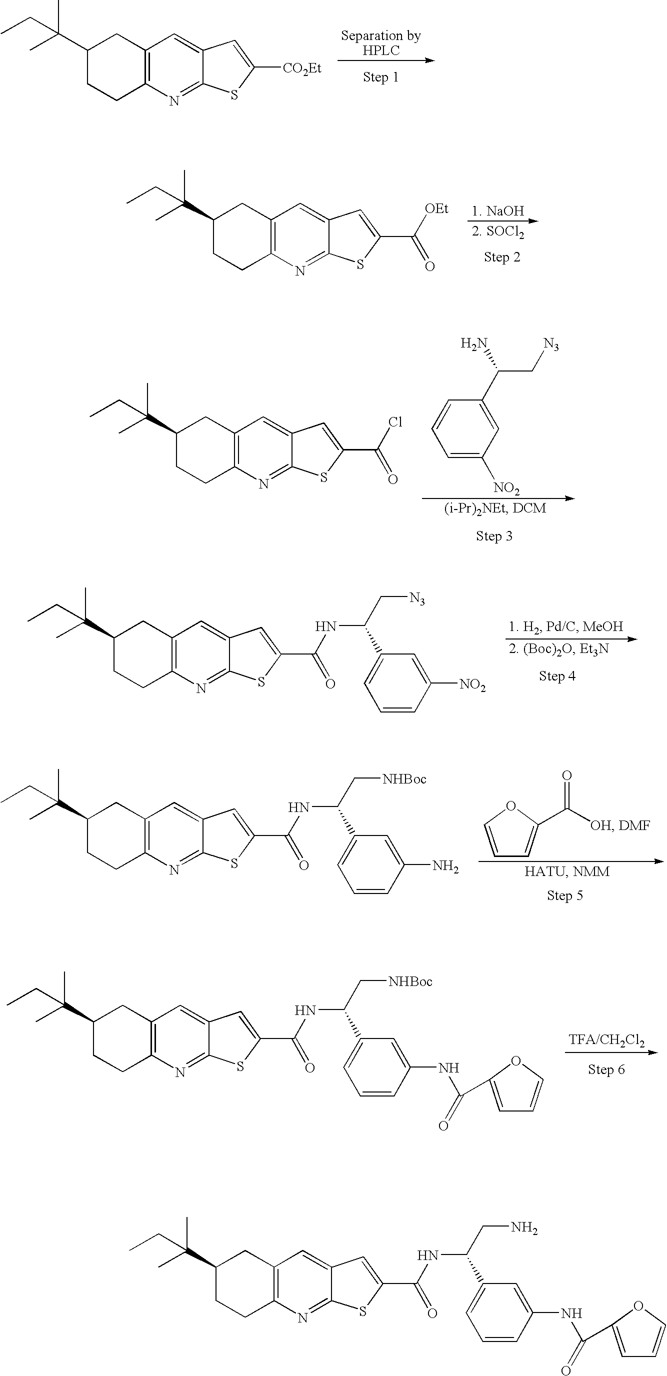

Compound 6(R,S)-(1,1-Dimethyl-propyl)-5,6,7,8-tetrahydro-thieno[2,3-b]quinoline-2-carboxylic acid ethyl ester was prepared from 4-(1,1-Dimethyl-propyl)-cyclohexanone by following similar procedure as described for example 110. Compound 6(R,S)-(1,1-Dimethyl-propyl)-5,6,7,8-tetrahydro-thieno[2,3-b]quinoline-2-carboxylic acid ethyl ester was separated on ChiralPak AS column eluting with 0.5 IPA/95.5% Hexane. The Peak A is R isomer, and the Peak B is S isomer.

Step 2 to step 6: Follow the procedure set forth in Example 269 step 5 to step 9.

### Examples 292-294

Through essentially the same procedure set forth in Example 291, by substituting the acid in Column 2 of Table 32 in Step 4, the compounds in Column 3 were prepared:

TABLE 32

| Example | Column 2 | Column 3 | CMPD |
|---|---|---|---|
| 292 | 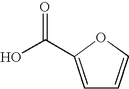 | 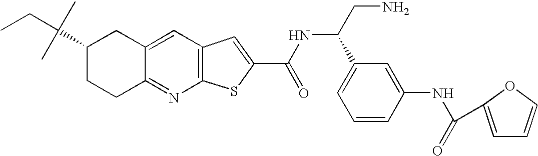 | MS: MH⁺ =531.2; |
| 293 | 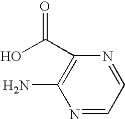 | 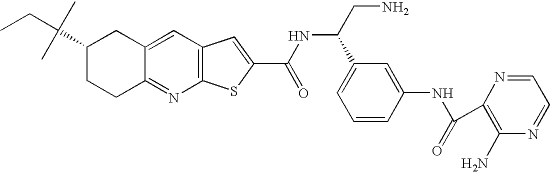 | MS: MH⁺ =558.3; |
| 294 | 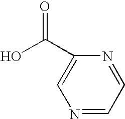 | 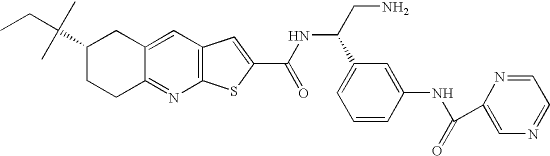 | MS: MH⁺ =543.3; |

### Example 291

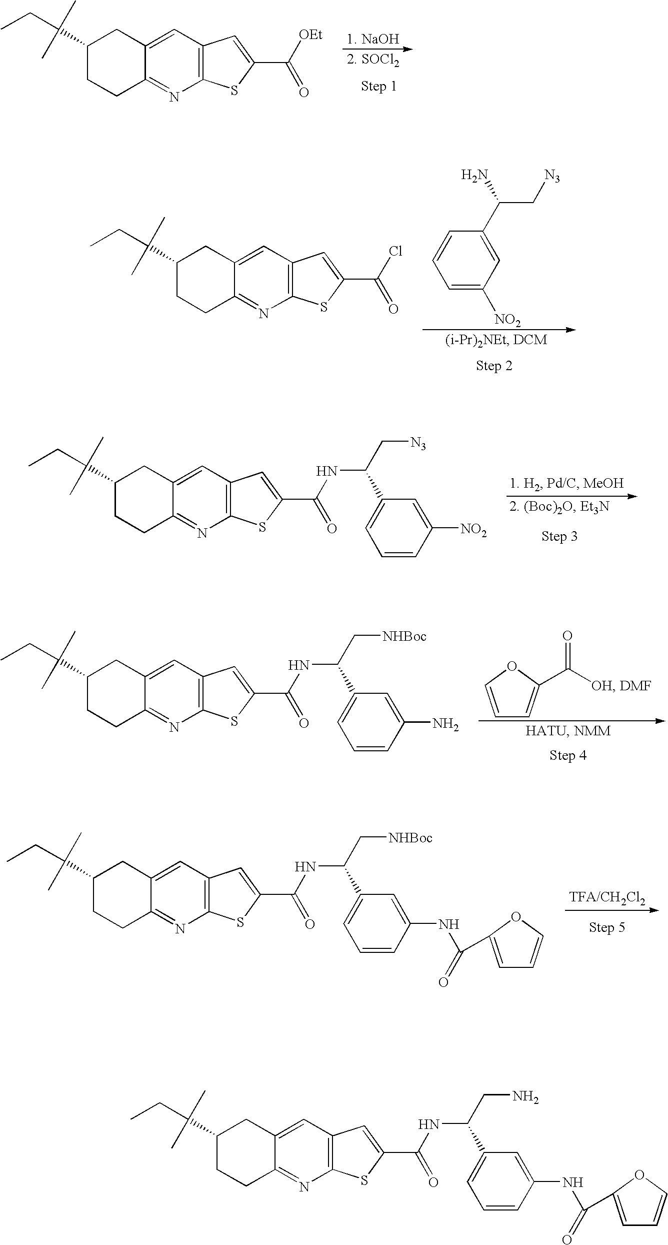

Step 1 to step 5: Follow the same procedure set forth in Example 269 step 5 to step 9

KSP Assays:

Endpoint Assay:

Serial dilutions of the compounds were prepared in a low binding, 96-well microtiter plate (Costar #3600) using 40% DMSO (Fisher BP231). The diluted compounds were added to a 384-well microtiter plate (Fisher 12-565-506). The following was then added to each well of the 384 microtiter plate: 55 μg/mL purified microtubules (Cytoskeleton TL238), 2.5-10 nM KSP motor domain (made according to Hopkins et al, Biochemistry, (2000) 39, 2805-2814), 20 mM ACES pH 7.0 (Sigma A-7949), 1 mM EGTA (Sigma E-3889), 1 mM MgCl₂ (Sigma M-2670),25 mM KCl (Sigma P-9333), 10 μM paclitaxel (Cytoskeleton TXD01), and 1 mM DTT (Sigma D5545) (final concentration). Following a 10 minute incubation, ATP (Sigma A-3377) (final concentration of ATP: 100 μM) was added to start the reaction. The final reaction volume was 25 μL. Final test compound concentration ranged from 50 μM to 5 nM and in another embodiment from 0.128 nM to 10 μM from. The reaction was incubated for 1 hour at room temperature. The reaction was stopped by the addition of 50 μL Biomol green reagent (Biomol AK111) per well, and was allowed to incubate for 20 minutes at room temperature. The 384-well microtiter plate was then transferred to an absorbance reader (Molecular Devices SpectraMax plus) and a single measurement was taken at 620 nm.

Kinetic Assay:

Compound dilutions were prepared as described previously. 25A25 buffer consisted of the following: 25 mM ACES pH 6.9, 2 mM MgOAc (Sigma M-9147), 2 mM EGTA, 0.1 mM EDTA (Gibco 144475-038), 25 mM KCl, 1 mM 2-mercaptoethanol (Biorad 161-0710), 10 μM paclitaxel, and 0.5 mM DTT. Solution 1 consisted of the following: 3.75 mM (final concentration) phosphoenol pyruvic acid (PEP, 2.5×) (Sigma P-7127), 0.75 mM MgATP (2.5×) (Sigma A-9187) in 1×25A25 buffer. Solution 2 consisted of the following: 100-500 nM KSP motor domain (2×), 6 U/mL pyruvate kinase/lactate dehydrogenase (2×) (Sigma P-0294), 110 μg/mL purified microtubules (2×), 1.6 μM β-nicotinamide adenine di-nucleotide, reduced form (NADH, 2×) (Sigma N-8129) in 1×25A25 buffer. Compound dilutions (8) were added to a 96-well microtiter plate (Costar 9018), and 40 μL of solution 1 was added to each well. The reaction was started by adding 50 μL of solution 2 to each well. The respective final assay concentrations were: 1.5 mM PEP, 0.3 mM MgATP, 50-250 nM KSP motor domain, 3 U/mL pyruvate kinase/lactate dehydrogenase, 55 μg/mL purified microtubules, 0.8 μM NADH (final concentrate). The microtiter plate was then transferred to an absorbance reader and multiple readings were taken for each well in a kinetic mode at 340 nm (25 measurements for each well approximately every 12 seconds, spread approximately over about 5 minutes time span). For each reaction, a rate of change was determined.

Calculations:

For both endpoint and kinetic assays, the percent activity for each concentration is calculated using the following equation:
Y=((X−background)/(positive control−background))*100
Y is the % activity and X is the measured reading (OD620 or rate)

For an IC₅₀ determination, the % activity was fit by the following equation using a nonlinear curve-fitting program for sigmoidal dose-responses (variable slopes) (GraphPad Prizm).
Y=Bottom+(Top-Bottom)/(1+10ˆ((LogEC50-X)*HillSlope))
X is the logarithm of concentration. Y is the response.
Y starts at Bottom and goes to Top with a sigmoid shape.

KSP inhibitory activities (based on end-point assay) for representative compounds are shown in Table 1 below. IC₅₀ values greater than 10000 nM (10 μM) are descignated as D class. IC₅₀ values between 1000 nM (1 μM) and 10000 nM (10 μM) are designated as C class. IC₅₀ values between 100 nM (0.1 μM) and 1000 nM (1 μM) are designated as B class. IC₅₀ values less than 100 nM (0.1 μM) are designated as A class.

TABLE 1

| | IC₅₀ | | IC₅₀ | | IC₅₀ |
|---|---|---|---|---|---|
| COMD | (nM) | COMD | (nM) | COMD | (nM) |
| 1 | C | 3 | D | 4 | D |
| 5 | C | 6 | B | 7 | D |
| 8 | D | 9 | C | 10 | B |
| 11 | D | 12 | A | 13 | D |
| 14 | C | 15 | C | 16 | D |
| 17 | C | 18 | D | 19 | D |
| 20 | D | 21 | C | 22 | D |
| 23 | D | 24 | C | 25 | B |
| 26 | B | 27 | C | 28 | B |
| 29 | D | 30 | B | 31 | D |
| 32 | D | — | — | — | — |
| 35 | D | 36 | D | 37 | D |
| 38 | C | 39-1* | — | 39-2 | — |
| 40 | A | 41 | C | 42 | D |
| 43 | B | 44 | D | 45 | D |
| 46 | D | 47 | D | 48 | D |
| 49 | D | 50 | D | 51 | D |
| 52 | D | 53 | D | 54 | D |
| 55 | D | 56 | D | 57 | D |
| 58 | B | 59 | A | 60 | A |
| 61 | C | 62 | B | 63 | A |
| 64 | A | 65 | A | 66 | C |
| 67 | B | 68 | A | 69 | B/C |
| | | | | | (1000 nM) |
| 70 | B | 71 | C | 72 | C |
| 73 | D | 74 | A | 75 | C |
| 76 | C | 77 | D | 78 | D |
| 79 | C | 80 | D | 81 | A/B |
| | | | | | (100 nM) |
| 82 | C | 83 | B | 84 | C |
| 85 | C | 86 | C | 87 | D |
| 88 | D | 89 | D | 90 | D |
| 91 | D | 92 | D | 93 | D |
| 94 | B | 95 | D | 96 | B |
| 97 | D | 98 | C | 99 | A |
| 100 | B | 101 | B | 102 | B |
| 103 | C | 104 | A | 105 | C |
| 111 | D | 112 | D | 113 | C |
| 114 | A | 115 | C | 116 | D |
| 118 | C | 119 | C | 120 | B |
| 121 | D | 122 | C | 123 | B |
| 124 | C | 125 | B/C | 126 | C |
| | | | (1000 nM) | | |
| 127 | C | 128 | B | 129 | D |
| 130 | C | 131 | B | 132 | C |
| 133 | D | 134 | B/C | 135 | B |
| | | | (1000 nM) | | |
| 144 | A | 145 | B | 146 | C |
| 147 | C | 148 | C | 149 | C |
| 150 | C | 151 | C | 152 | C |
| 153 | C | 154 | C | 155 | C |
| 156 | B | 157 | D | 158 | D |
| 159 | D | 160 | C | 161 | D |
| 162 | D | 163 | D | 164 | B |
| 165 | D | 166 | D | 167 | B |
| 168 | A | 169 | A | 170 | B |
| 171 | C | 172 | B | 173 | C |
| 174 | C | 175 | C | 176 | A |
| 177 | A | 178 | A | 179 | B |
| 180 | B | 181 | B | 182 | B |
| 183 | C | 184 | B | 185 | C |
| 186 | C | 187 | D | 188 | D |
| 189 | A | 190 | B/C | 191 | A |
| | | | (1000 nM) | | |
| 192 | C | 193 | C | 194 | C |
| 195 | C | 196 | C | 197 | B |
| 198 | C | 199 | C | 200 | C |
| 201 | — | 202 | B | 203 | B |
| 204 | B | 205 | C | 206 | C |
| 207 | D | 208 | B | 209 | B |
| 210 | B | 211 | B | 212 | B |
| 213 | B | 214 | A | 215 | A |
| 216 | A | 217 | A | 218 | A |
| 219 | A | 220 | A | 221 | A |
| 222 | A | 223 | A | 224 | A |
| 225 | A | 226A | B | 226B | B |
| 226C | B | 226D | B | 226E | B |
| 226F | B | 226G | B | 226H | B |
| 226J | B | 227 | A | 228 | A |
| 229 | A | 230 | B | 231 | A |
| 232 | A | 233 | A | 234 | A |
| 235 | A | 236 | A | 237 | A |
| 238 | A | 239 | A | 240 | A |
| 241 | A | 242 | A | 243 | A |
| 244 | A | 245 | A | 246 | A |
| 247 | A | 248 | A | 249 | A |
| 250 | A | 251 | A | 252 | A |
| 253 | B | 253A | B | 254 | B |
| 255 | A | 256 | A | 257 | A |
| 258 | A | 259 | A | 260 | A |
| 261 | A | 262 | A | 263 | A |
| 264 | A | 265 | A | 266 | A |
| 267 | B | 268 | A | 269 | A |
| 270 | A | 271 | A | 272 | A |
| 273 | A | 274 | A | 275 | A |
| 276 | A | 277 | A | 278 | B |
| 279 | A | 280 | B | 281 | A |
| 282 | A | 283 | A | 284 | A |

*Compounds 39-1 and 39-2 were an inseparable mixture and were not tested

Exact IC₅₀ values for some of the representative compounds in Table 1 are shown in Table 2 below:

TABLE 2

| | IC₅₀ | | IC₅₀ | | IC₅₀ |
|---|---|---|---|---|---|
| COMD | (nM) | COMD | (nM) | COMD | (nM) |
| 168 | 8 | 191 | 10 | 68 | 20 |
| 99 | 22 | 178 | 25 | 63 | 40 |
| 189 | 50 | 59 | 55 | 144 | 60 |
| 40 | 60 | 65 | 62 | 177 | 65 |
| 227 | 21 | 237 | 10 | 259 | 17 |
| 273 | 17 | 277 | 10 | 284 | 8 |
| 250 | 19 | 235 | 23 | 257 | 23 |

### REFERENCES

KSP/Kinesin as Target

1) Blangy, A et al. (1995) Cell 83, 1159-1169 (cloning of human KSP, function in mitosis).
2) Sawin, K. and Mitchison, T. J. (1995) Proc. Natl. Acad. Sci. 92, 4289-4293 (Xenopus Egd5, conserved motor domain, function).
3) Huang, T.-G. and Hackney, D. D. (1994) J. Biol. Chem. 269,16493-16501 (Drosphila kinesin minimal motor domain definition, expression and purification from E. coli).
4) Kaiser A. et al. (1999) J. Biol. Chem. 274, 18925-18931 (overexpression of KSP motor domain, function in mitosis, inhibition of growth by targeting KSP).
5) Kapoor T. M and Mitchison, T. J. (1999) Proc. Natl. Acad. Sci. 96, 9106-9111 (use of KSP motor domain, inhibitors thereof).
6) Mayer, T. U. (1999) Science 286, 971-974 (KSP inhibitors as anticancer drugs).
   KSP Assays (Endpoint and Kinetics)
7) Wohlke, G. et al. (1997) Cell 90, 207-216 (expression and purification of kinesin motor domain, kinetics assay, endpoint assay).
8) Geladeopoulos, T. P. et al. (1991) Anal. Biochem. 192, 112-116 (basis for endpoint assay).
9) Sakowicz, R. et al. (1998) Science 280, 292-295 (kinetics assay).
10) Hopkins, S. C. et al. (2000) Biochemistry 39, 2805-2814 (endpoint and kinetics assay).
11) Maliga, Z. et al. (2002) Chem. & Biol. 9, 989-996 (kinetics assay).

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

Therefore, we claim:

1. A compound represented by the structural Formula I: 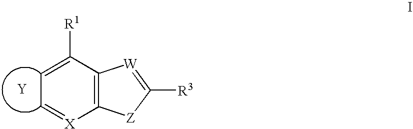
or a pharmaceutically acceptable salt, solvate or ester thereof, wherein:
ring Y is a 5- to 7-membered ring selected from the group consisting of cycloalkyl, cycloalkenyl, heterocyclyl or heterocyclenyl fused as shown in Formula I, wherein in each of said 5- to 7-membered ring, each substitutable ring carbon is independently substituted with 1-2 R² moieties and each substitutable ring heteroatom is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;
each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;
or two R²s on the same carbon atom are optionally taken together with the carbon atom to which they are attached to form a C═O, a C═S or an ethylenedioxy group;
R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)N R⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(═NR⁷)NR⁴R⁵, —C(O)N(R⁷)(CR⁴⁰R⁴¹)₁₋₅—NR⁴R⁵, —C(O)N(R⁷)(CR⁴⁰R⁴¹)₁₋₅—C(O)—NR⁴R⁵, —C(O)N(R⁷)(CR⁴⁰R⁴¹)₁₋₅—OR⁷, —C(S)NR⁷(CH₂)₁₋₅NR⁴R⁵, and —C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;
each of R⁴ and R⁵ is independently selected from the group consisting of H, alkyl, cycloalkyl, cycloalkenyl, heterocyclyl, heterocyclenyl, aryl, heteroaryl, —OR⁷, —C(O)R⁷, and —C(O)OR⁷, wherein each of said alkyl, cycloalkyl, cycloalkenyl, heterocyclyl, heterocyclenyl, aryl, and heteroaryl, is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, cycloalkenyl, heterocyclyl, heterocyclenyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring;
or two R⁸ groups, when attached to the same carbon, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; wherein each of said R¹¹ alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
R⁴⁰ and R⁴¹ can be the same or different, each being independently selected from the group consisting of H, alkyl, aryl, heteroaryl, heterocyclyl, heterocyclenyl, cycloalkyl and cycloalkenyl;
each R⁴² is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —N(R¹⁰)C(O)R¹¹, and —NR¹⁰C(O)OR¹¹;
with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹, —C(O)OR¹¹, —C(O)N(R¹⁰)(R¹¹), or —N(R¹⁰)C(O)R¹¹;
with the further proviso that the compound of Formula (I) is other than any of the following: 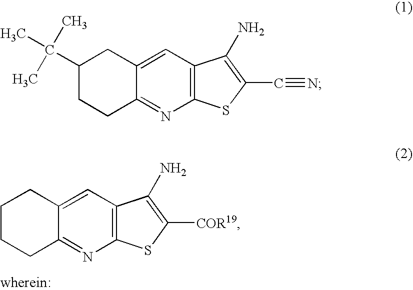
R¹⁹ is —NHOH, —OMe, —OEt, —O-n-propyl, or —O-i-propyl; 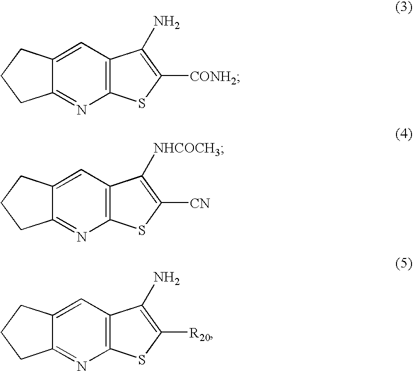
wherein:
R²⁰ is —CN, —C(O)C₆H₅, —CO₂C₂H₅, —CO₂H, or —C(O)NH₂; 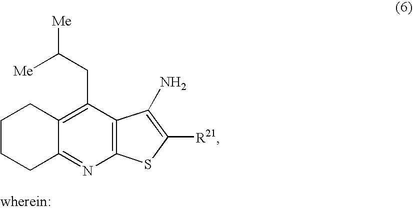
R²¹ is 4-ClC₆H₄C(O)— or 4-PhC₆H₄C(O)—; 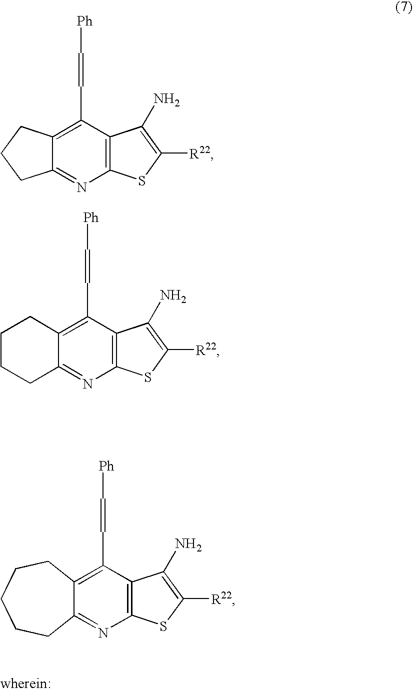
R²² is —CN, —C(O)CH₃ or —CO₂C₂H₅; 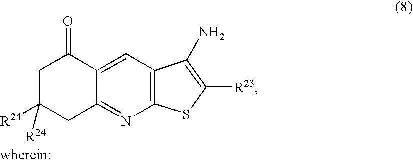
R²³ is —C(O)NH₂, —C(O)NHPh, or benzoyl and R²⁴ is H or methyl;

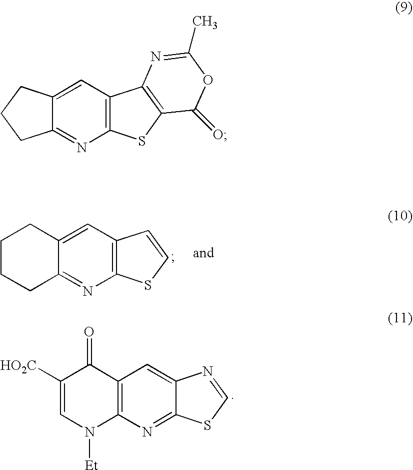

2. The compound of claim 1 represented by Formula II:

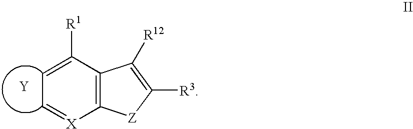

3. The compound of claim 1 represented by Formula III

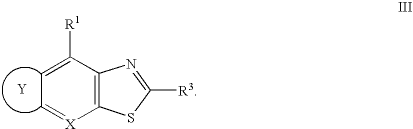

4. The compound of claim 1, wherein X is N.

5. The compound of claim 1, wherein X is N-oxide.

6. The compound of claim 1, wherein Z is S.

7. The compound of claim 1, wherein Z is S(═O).

8. The compound of claim 1, wherein Z is S(═O)₂.

9. The compound of claim 1, wherein ring Y is a 5- to 7-membered cycloalkyl, wherein each substitutable ring carbon is independently substituted with 1-2 R² moieties.

10. The compound of claim 1, wherein ring Y is a 5- to 7-membered cycloalkenyl, wherein each substitutable ring carbon is independently substituted with 1-2 R² moieties.

11. The compound of claim 9, wherein ring Y is a 6-membered cycloalkyl ring, wherein each substitutable ring carbon is independently substituted with 1-2 R² moieties.

12. The compound of claim 10, wherein ring Y is a 6-membered cycloalkenyl, wherein each substitutable ring carbon is independently substituted with 1-2 R² moieties.

13. The compound of claim 2, wherein ring Y is a 5- to 7-membered heterocyclyl, wherein in said ring Y, each substitutable ring carbon is independently substituted with 1-2 R² moieties and each substitutable ring heteroatom, when nitrogen, is independently substituted with R⁶.

14. The compound of claim 2, wherein ring Y is a 5- to 7-membered heterocyclenyl, wherein in said ring Y, each substitutable ring carbon is independently substituted with 1-2 R² moieties and each substitutable ring heteroatom, when nitrogen, is independently substituted with R⁶.

15. The compound of claim 9, wherein R² is H, alkyl, aryl, aralkyl, cycloalkyl, cycloalkylalkyl, —CF₃, alkylsilyl, alkoxy or —NR⁴R⁵; or two R²s attached to the same ring carbon atom are taken together with the carbon atom to which they are attached to form a C═O, a C═S or an ethylenedioxy group.

16. The compound of claim 13, wherein R⁶ is selected from the group consisting of H, alkyl, cycloalkyl, cycloalkylalkyl, aryl, aralkyl, heteroaryl, —(CH₂)₁₋₆CF₃, and —C(O)OR⁷ wherein R⁷ is alkyl.

17. The compound of claim 1, wherein R¹² is H, halo, —NR⁴R⁵ or —OR⁷.

18. The compound of claim 1, wherein R³ is H, alkyl, cycloalkyl, cycloalkenyl, heterocyclyl, heterocyclenyl, heteroaryl, —C(O)OR⁷, —C(O)NR⁴R⁵, —C(S)NR⁴R⁵, —C(O)NR⁴OR⁷, —NR⁴R⁵, —NR⁴C(O)R⁵, —NR⁴C(O)NR⁴R⁵, —(CR¹⁰R¹¹)₀₋₆SR⁷, S(O₂)R⁷, —S(O₂)NR⁴R⁵, —CN, or —C(═NR⁷)NR⁴R⁵ wherein said alkyl, heterocyclyl or heteroaryl is optionally substituted with 1-3 R⁹ moieties.

19. The compound of any one of claim 1, wherein R¹ is H, halo, —S-alkyl, alkoxy or hydroxy.

20. The compound of claim 19, wherein R¹ is H, Cl, OH or —SCH₃.

21. The compound of claim 2 wherein:
Y is a 5- to 7-membered cycloalkyl ring, wherein each substitutable ring carbon atom is independently substituted with 1-2 R² moieties;
X is N; and
Z is S.

22. The compound of claim 21, wherein:
R¹ is selected from the group consisting H, hydroxy, halo, and
—S(O)ₘ-alkyl, wherein m is 0;
each R² independently is selected from the group consisting of H, alkyl, alkenyl, aryl, alkylsilyl, cycloalkyl, and —CF₃; wherein said alkyl or alkenyl is either unsubstituted or optionally substituted with aryl or cycloalkyl;
or two R²s on the same carbon atom are optionally taken together with the carbon atom to which they are attached to form a C═O, a C═S or an ethylenedioxy group;
R³ is selected from the group consisting of H, alkyl, cycloalkyl, cycloalkenyl, heterocyclyl, heterocyclenyl, heteroaryl, —C(O)OR⁷, —C(O)NR⁴R⁵, —C(S)NR⁴R⁵, —C(O)NR⁴OR⁷, —NR⁴R⁵, —NR⁴C(O)R⁵, —NR⁴C(O)NR⁴R⁵, —(CR¹⁰R¹¹)₀₋₆SR⁷, S(O₂)R⁷, —S(O₂)NR⁴R⁵, —CN, or —C(═NR⁷)NR⁴R⁵ wherein said alkyl, cycloalkyl, cycloalkenyl, heterocyclyl, heterocyclenyl, or heteroaryl is optionally substituted with 1-3 R⁹ moieties; and
R¹² is H, halo, —NR⁴R⁵, or —OR⁷.

23. The compound of claim 21, represented by Formula IIa:

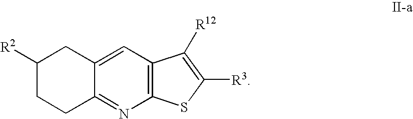

24. The compound of claim 23, wherein R³ is —CN.

25. The compound of claim 23, wherein R³ is —C(O)NR⁴R⁵ wherein:
each of R⁴ and R⁵ is independently selcted from the group consisting of H, alkyl, cycloalkyl, aryl, heterocyclyl, and heteroaryl; wherein each of said alkyl, cycloalkyl, aryl, heterocyclyl and heteroaryl is unsubstituted or optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S.

26. The compound of claim 25, wherein:
each of said R⁴ and R⁵ alkyl is unsubstituted or optionally substituted with 1-3 R⁸ moieties independently selected from the group consisting of —OR¹⁰, —C(O)NR¹⁰R¹¹, —C(O)OR¹⁰, —NR¹⁰R¹¹, —CN, —C(═NR¹⁰)NR¹⁰R¹¹, heterocyclyl, aryl, and heteroaryl; wherein each of said R⁸ heterocyclyl, aryl, and heteroaryl moieties is unsubstituted or optionally substituted with 1-3 R⁴² moieties selected from the group consisting of halo, alkyl, aryl, heteroaryl, —NO₂, —CN, —NR¹⁰R¹¹, —OR¹⁰, —N(R¹⁰)C(O)R¹¹, —N(R¹⁰)C(O)OR¹¹, —C(O)NR¹⁰R¹¹, and —C(O)OR¹⁰; wherein when each of said R⁴² aryl and heteroaryl contains two radicals on adjacent carbon atoms anywhere within said aryl or heteroaryl, such radicals may optionally and independently in each occurrence, be taken together with the carbon atoms to which they are attached, to form a five to six membered carbocyclic or heterocyclic ring;
each of said R⁴ and R⁵ cycloalkyl is unsubstituted or is optionally substituted with 1-3 R⁸ moieties independently selected from the group consisting of halo, hydroxy, and alkyl;
each of said R⁴ and R⁵ heterocyclyl is unsubstituted or is optionally substituted with 1-3 R⁸ moieties independently selected from the group consisting of halo, hydroxy, —C(O)OH, and —C(O)O-alkyl;
each of said R⁴ and R⁵ aryl is unsubstituted or optionally substituted with 1-3 R⁸ moieties independently selected from the group consisting of —OR¹⁰, —NR¹⁰R¹¹, halo, and alkyl;
each of said R⁴ and R⁵ heteroaryl is unsubstituted or is optionally substituted with 1-3 R⁸ moieties independently selected from the group consisting of —OR¹⁰, —NR¹⁰R¹¹, halo, and alkyl;
said 3-6 membered heterocyclic ring formed by R⁴, R⁵, and the nitrogen atom to which R⁴ and R⁵ are attached, is unsubstituted or is optionally substituted with 1-3 substitutents selected from the group consisting of hydroxy,halo, alkyl —C(O)OH, and —C(O)O-alkyl.

27. The compound of claim 25, wherein:
each of R⁴ and R⁵ is independently selcted from the group consisting of H and alkyl; wherein said alkyl is optionally substituted with 1-4 R⁸ moieties;
R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities;
each R¹⁰ is independently H or alkyl;
each R¹¹ is independently H, alkyl, heterocyclyl, aryl, or heteroaryl; wherein each of said R¹¹ alkyl, heterocyclyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and
each R⁴² is independently selected from the group consisting of halo, alkyl, heterocyclyl, aryl, heteroaryl, —NO₂, —NR¹⁰R¹¹, —OR¹⁰, —CN, —C(O)NR¹⁰R¹¹, —CF₃, —OCF₃, —N(R¹⁰)C(O)R¹¹, and —NR¹⁰C(O)OR¹¹.

28. The compound of claim 27, wherein said R⁸ aryl is phenyl, and said R⁸ heteroaryl is selected from the group consisting of pyridyl and thiophenyl.

29. The compound of claim 28, wherein R⁴² is —N(R¹⁰)C(O)R¹¹, wherein R¹⁰ of said is is —N(R¹⁰)C(O)R¹¹ is H, and R¹¹ of said —N(R¹⁰)C(O)R¹¹ is selected from the group consisting of heterocyclyl and heteroaryl, each of which is optionally substituted.

30. The compound of claim 29, wherein said R¹¹ heterocyclyl of said —N(R¹⁰)C(O)R¹¹ is selected from the group consisting of pyrrolidinyl, piperidinyl, piperizinyl, and morpholinyl, each of which is optionally substituted.

31. The compound of claim 29, wherein said R¹¹ heteroaryl of said
—N(R¹⁰)C(O)R¹¹ is selected from the group consisting of benzopyrazinyl, pyrazinyl, oxazolyl, isoxazolyl, thiazolyl, isothizolyl, pyrazolyl, imidazolyl, pyrrolyl, triazolyl, 1,2,3-triazolyl, thiadiazolyl, tetrazolyl, furanyl, thiophenyl, pyrrolyl, and pyrimidyl, each of which is optionally substituted.

32. The compound of claim 23, wherein R³ is alkyl, wherein said alkyl is unsubstituted or optionally substituted with 1-3 R⁹ moieties independently selected from the group consisting of —OH, —CN, halo, alkoxy, —OC(O)NR⁴R⁵, —C(O)NR⁴R⁵, —(CR¹⁰R¹¹)₀₋₄NR⁴R⁵, —NR⁴C(O)R⁵ and —NR⁴C(O)NR⁴R⁵.

33. The compound of claim 3 represented by Formula III-a:

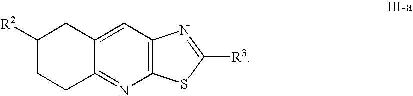

34. The compound of claim 33, wherein:
R² is alkyl; and
R³ is selected from the group consisting of —(CR¹⁰R¹¹)₀₋₆R⁷, —CN,
—C(O)NR⁴R⁵, —NR⁴C(O)NR⁴R⁵, —NR⁴R⁵, and —NR⁴C(O)R⁵.

35. The compound of claim 33, wherein R³ is —C(O)NR⁴R⁵ wherein:
each of R⁴ and R⁵ is independently selcted from the group consisting of H, alkyl, cycloalkyl, aryl, heterocyclyl, and heteroaryl; wherein each of said alkyl, cycloalkyl, aryl, heterocyclyl and heteroaryl is unsubstituted or optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S.

36. The compound of claim 35, wherein:
each of said R⁴ and R⁵ alkyl is unsubstituted or optionally substituted with 1-3 R⁸ moieties independently selected from the group consisting of —OR¹⁰, —C(O)NR¹⁰R¹¹, —C(O)OR¹⁰, —NR¹⁰R¹¹, —CN, —C(═NR¹⁰)NR¹⁰R¹¹, heterocyclyl, aryl, and heteroaryl; wherein each of said R⁸ heterocyclyl, aryl, and heteroaryl moieties is unsubstituted or optionally substituted with 1-3 R⁴² moieties selected from the group consisting of halo, alkyl, aryl, heteroaryl, —NO₂, —CN, —NR¹⁰R¹¹, —OR¹⁰, —N(R¹⁰)C(O)R¹¹, —N(R¹⁰)C(O)OR¹¹, —C(O)NR¹⁰R¹¹, and —C(O)OR¹⁰; wherein when each of said R⁴² aryl and heteroaryl contains two radicals on adjacent carbon atoms anywhere within said aryl or heteroaryl, such radicals may optionally and independently in each occurrence, be taken together with the carbon atoms to which they are attached, to form a five to six membered carbocyclic or heterocyclic ring;
each of said R⁴ and R⁵ cycloalkyl is unsubstituted or is optionally substituted with 1-3 R⁸ moieties independently selected from the group consisting of halo, hydroxy, and alkyl;
each of said R⁴ and R⁵ heterocyclyl is unsubstituted or is optionally substituted with 1-3 R⁸ moieties independently selected from the group consisting of halo, hydroxy, —C(O)OH, and —C(O)O-alkyl;
each of said R⁴ and R⁵ aryl is unsubstituted or optionally substituted with 1-3 R⁸ moieties independently selected from the group consisting of —OR¹⁰, —NR¹⁰R¹¹, halo, and alkyl;
each of said R⁴ and R⁵ heteroaryl is unsubstituted or is optionally substituted with 1-3 R⁸ moieties independently selected from the group consisting of —OR¹⁰, —NR¹⁰R¹¹, halo, and alkyl;
said 3-6 membered heterocyclic ring formed by R⁴, R⁵, and the nitrogen atom to which R⁴ and R⁵ are attached, is unsubstituted or is optionally substituted with 1-3 substitutents selected from the group consisting of hydroxy,halo, alkyl —C(O)OH, and —C(O)O-alkyl.

37. The compound of claim 35, wherein:
each of R⁴ and R⁵ is independently selcted from the group consisting of H and alkyl; wherein said alkyl is optionally substituted with 1-4 R⁸ moieties;
R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities;
each R¹⁰ is independently H or alkyl;
each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and
each R⁴² is independently selected from the group consisting of halo, alkyl, heterocyclyl, aryl, heteroaryl, —NO₂, —NR¹⁰R¹¹, —OR¹⁰, —CN, —C(O)NR¹⁰R¹¹, —CF₃, —OCF₃, —N(R¹⁰)C(O)R¹¹, and —NR¹⁰C(O)OR¹¹.

38. The compound of claim 37, wherein said R⁸ aryl is phenyl, and said R⁸ heteroaryl is selected from the group consisting of pyridyl and thiophenyl.

39. The compound of claim 38, wherein R⁴² is —N(R¹⁰)C(O)R¹¹, wherein R¹⁰ in said —N(R¹⁰)C(O)R¹¹ is H and R¹¹ in said —N(R¹⁰)C(O)R¹¹ is selected from the group consisting of heterocyclyl and heteroaryl, each of which is optionally substituted.

40. The compound of claim 39, wherein said R¹¹ heterocyclyl is selected from the group consisting of pyrrolidinyl, piperidinyl, piperizinyl, and morpholinyl, each of which is optionally substituted.

41. The compound of claim 39, wherein said R¹¹ heteroaryl is selected from the group consisting of benzopyrazinyl, pyrazinyl, oxazolyl, isoxazolyl, thiazolyl, isothizolyl, pyrazolyl, imidazolyl, pyrrolyl, triazolyl, 1,2,3-triazolyl, thiadiazolyl, tetrazolyl, furanyl, thiophenyl, pyrrolyl, and pyrimidyl, each of which is optionally substituted.

42. The compound of claim 13, represented by formula IV:

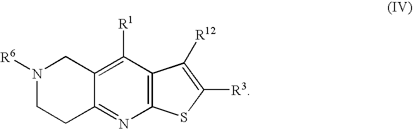

43. The compound of claim 30, wherein:
R¹ is H;
R³is —CN;
R⁶ is selected from the group consisting of H, alkyl, cycloalkylalkyl, aralkyl, —(CH₂)₁₋₆CF₃, and —C(O)OR⁷ wherein R⁷is alkyl; and
R¹² is —NR⁴R⁵, wherein both R⁴ and R⁵ are H.

44. The compound of claim 42, wherein R³ is —C(O)NR⁴R⁵ wherein:
each of R⁴ and R⁵ is independently selcted from the group consisting of H, alkyl, cycloalkyl, aryl, heterocyclyl, and heteroaryl; wherein each of said alkyl, cycloalkyl, aryl, heterocyclyl and heteroaryl is unsubstituted or optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S.

45. The compound of claim 44, wherein:
each of said R⁴ and R⁵ alkyl is unsubstituted or optionally substituted with 1-3 R⁸ moieties independently selected from the group consisting of —OR¹⁰, —C(O)NR¹⁰R¹¹, —C(O)OR¹⁰, —NR¹⁰R¹¹, —CN, —C(═NR¹⁰)NR¹⁰R¹¹, heterocyclyl, aryl, and heteroaryl; wherein each of said R⁸ heterocyclyl, aryl, and heteroaryl moieties is unsubstituted or optionally substituted with 1-3 R⁴² moieties selected from the group consisting of halo, alkyl, aryl, heteroaryl, —NO₂, —CN, —NR¹⁰R¹¹, —OR¹⁰, —N(R¹⁰)C(O)R¹¹, —N(R¹⁰)C(O)OR¹¹, —C(O)NR¹⁰R¹¹, and —C(O)OR¹⁰; wherein when each of said R⁴² aryl and heteroaryl contains two radicals on adjacent carbon atoms anywhere within said aryl or heteroaryl, such radicals may optionally and independently in each occurrence, be taken together with the carbon atoms to which they are attached, to form a five to six membered carbocyclic or heterocyclic ring;
each of said R⁴ and R⁵ cycloalkyl is unsubstituted or is optionally substituted with 1-3 R⁸ moieties independently selected from the group consisting of halo, hydroxy, and alkyl;
each of said R⁴ and R⁵ heterocyclyl is unsubstituted or is optionally substituted with 1-3 R⁸ moieties independently selected from the group consisting of halo, hydroxy, —C(O)OH, and —C(O)O-alkyl;
each of said R⁴ and R⁵ aryl is unsubstituted or optionally substituted with 1-3 R⁸ moieties independently selected from the group consisting of —OR¹⁰, —NR¹⁰R¹¹, halo, and alkyl;
each of said R⁴ and R⁵ heteroaryl is unsubstituted or is optionally substituted with 1-3 R⁸ moieties independently selected from the group consisting of —OR¹⁰, —NR¹⁰R¹¹, halo, and alkyl;
said 3-6 membered heterocyclic ring formed by R⁴, R⁵, and the nitrogen atom to which R⁴ and R⁵ are attached, is unsubstituted or is optionally substituted with 1-3 substitutents selected from the group consisting of hydroxy, halo, alkyl —C(O)OH, and —C(O)O-alkyl.

46. The compound of claim 44, wherein:
each of R⁴ and R⁵ is independently selcted from the group consisting of H and alkyl; wherein said alkyl is optionally substituted with 1-4 R⁸ moieties;
R⁸ is selected from the group consisting of —NR¹⁰R¹¹, —CN, —C(═NR¹⁰)NR¹⁰R¹¹, —C(O)NR¹⁰R¹¹, —C(O)OR¹⁰, —OR¹⁰, heterocyclyl, aryl, and heteroaryl; wherein each of said R⁸ alkyl, heterocyclyl, aryl, and heteroaryl is optionally substituted with 1-4 R⁴² moities;
each R¹⁰ is independently H or alkyl;
each R¹¹ is independently H, alkyl, heterocyclyl, aryl, or heteroaryl; wherein each of said R¹¹ alkyl, aryl, and heteroaryl is independently optionally substituted with 1-3 moieties independently selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, heterocyclyl, and heteroaryl; and
each R⁴² is independently selected from the group consisting of halo, alkyl, heterocyclyl, aryl, heteroaryl, —NO₂, —NR¹⁰R¹¹, —OR¹⁰, —CN, —C(O)NR¹⁰R¹¹, —CF₃, —OCF₃, —N(R¹⁰)C(O)R¹¹, and —NR¹⁰C(O)OR¹¹.

47. The compound of claim 46, wherein said R⁸ aryl is phenyl; and said R⁸ heteroaryl is selected from the group consisting of pyridyl and thiophenyl.

48. The compound of claim 47, wherein R⁴² is —N(R¹⁰)C(O)R¹¹, wherein R¹⁰ in said —N(R¹⁰)C(O)R¹¹ is H, and R¹¹ in said —N(R¹⁰)C(O)R¹¹ is selected from the group consisting of heterocyclyl and heteroaryl, each of which is optionally substituted.

49. The compound of claim 48, wherein said R¹¹ heterocyclyl in said —N(R¹⁰)C(O)R¹¹ is selected from the group consisting of pyrrolidinyl, piperidinyl, piperizinyl, and morpholinyl, each of which is optionally substituted.

50. The compound of claim 49, wherein said R¹¹ heteroaryl in said —N(R¹⁰)C(O)R¹¹ is selected from the group consisting of benzopyrazinyl, pyrazinyl, oxazolyl, isoxazolyl, thiazolyl, isothizolyl, pyrazolyl, imidazolyl, pyrrolyl, triazolyl, 1,2,3-triazolyl, thiadiazolyl, tetrazolyl, furanyl, thiophenyl, pyrrolyl, and pyrimidyl, each of which is optionally substituted.

51. The compound of claim 1, wherein the compound is selected from the group consisting of: 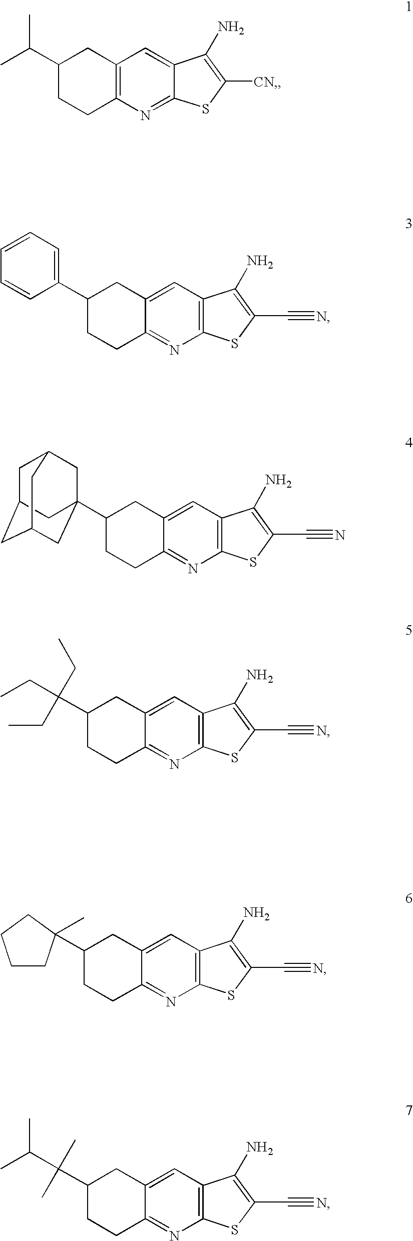 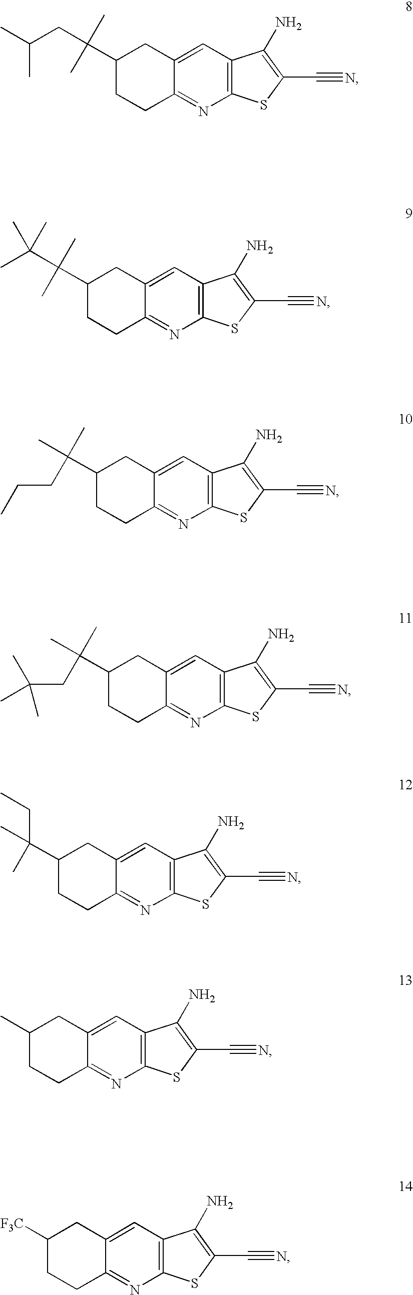 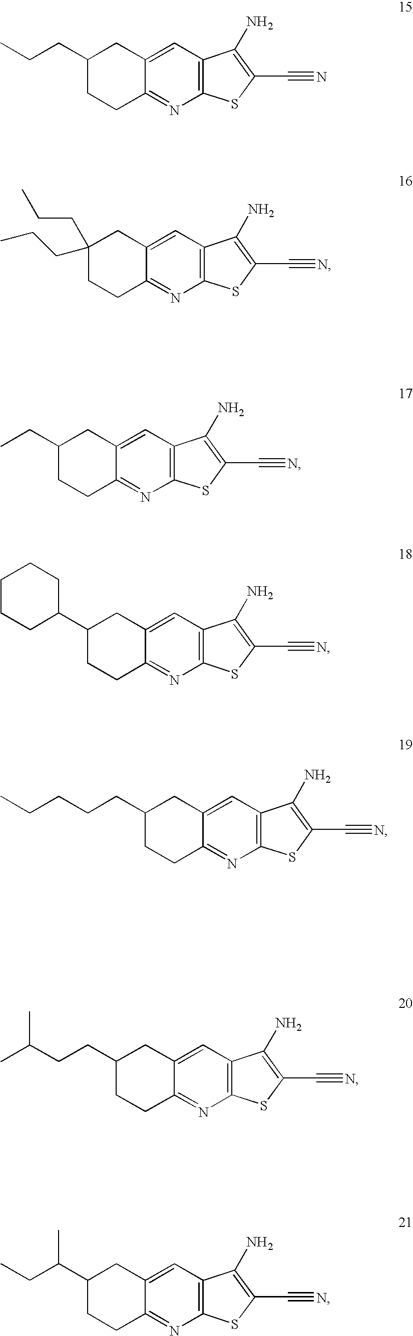 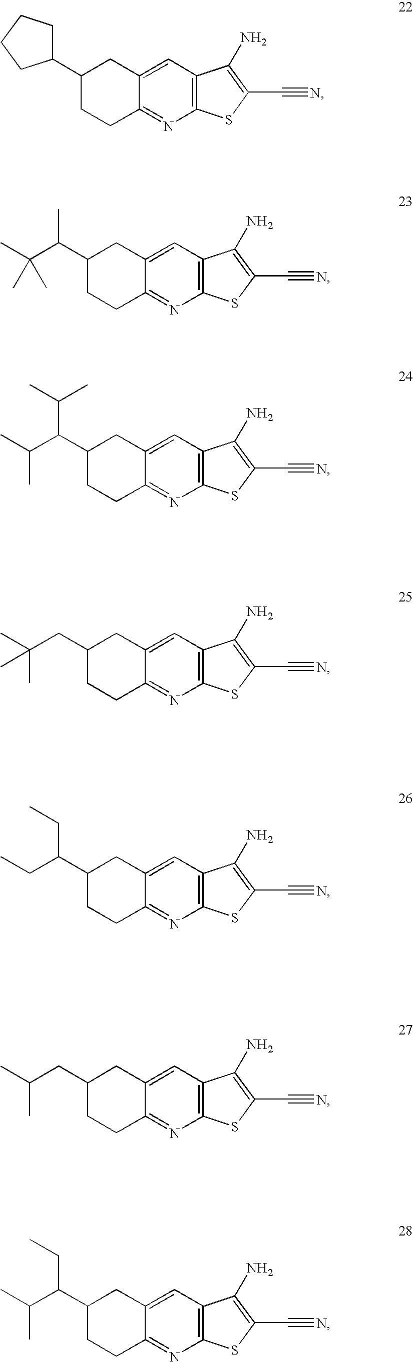 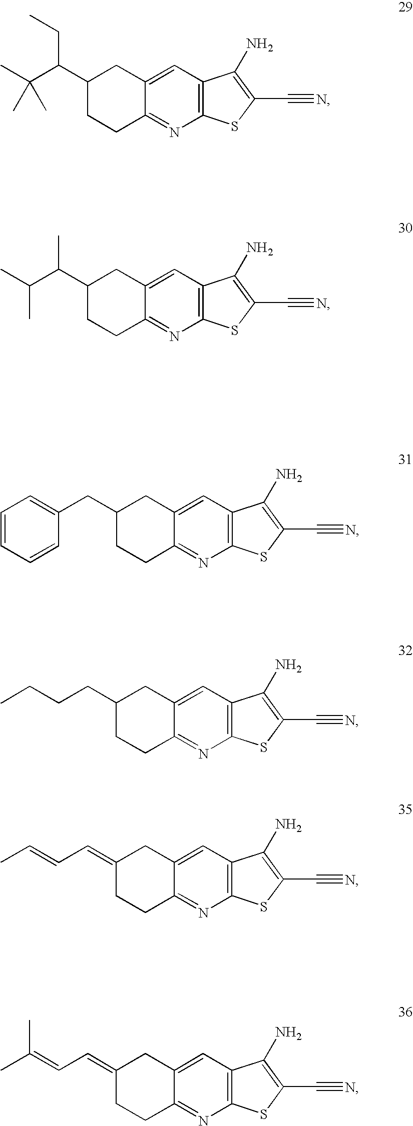 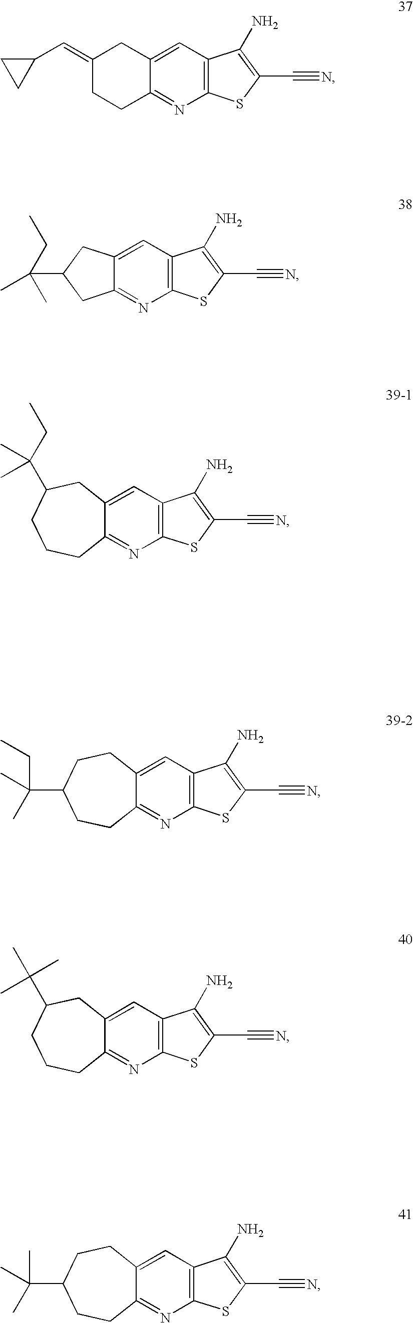 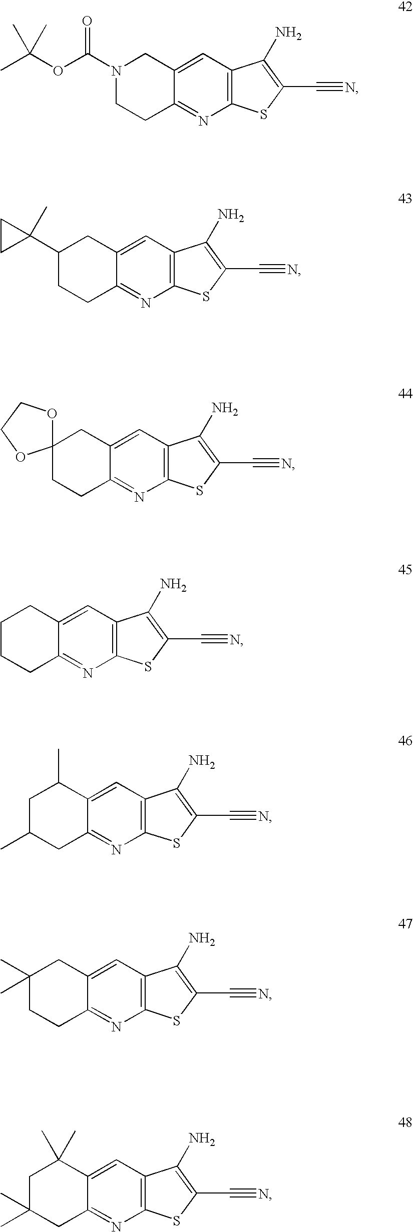 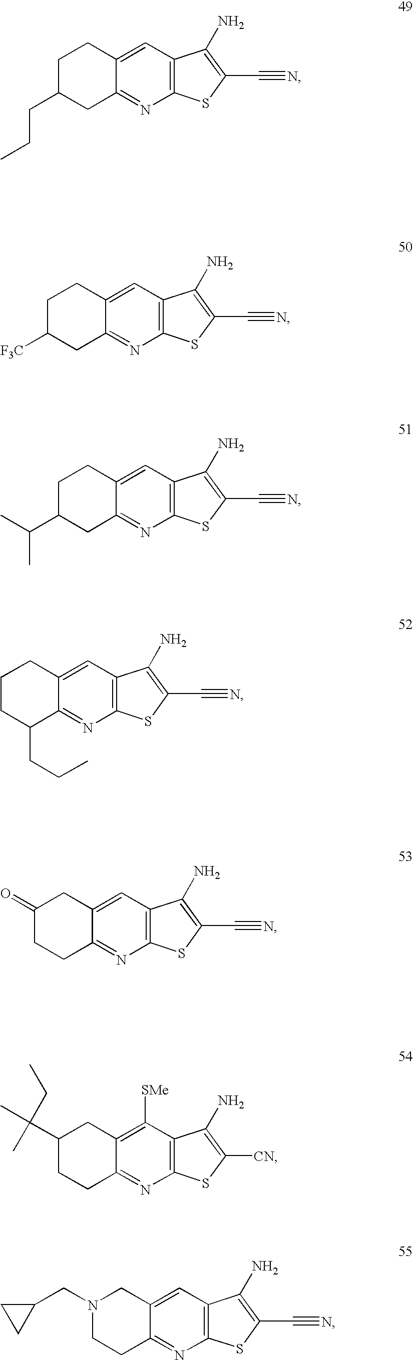 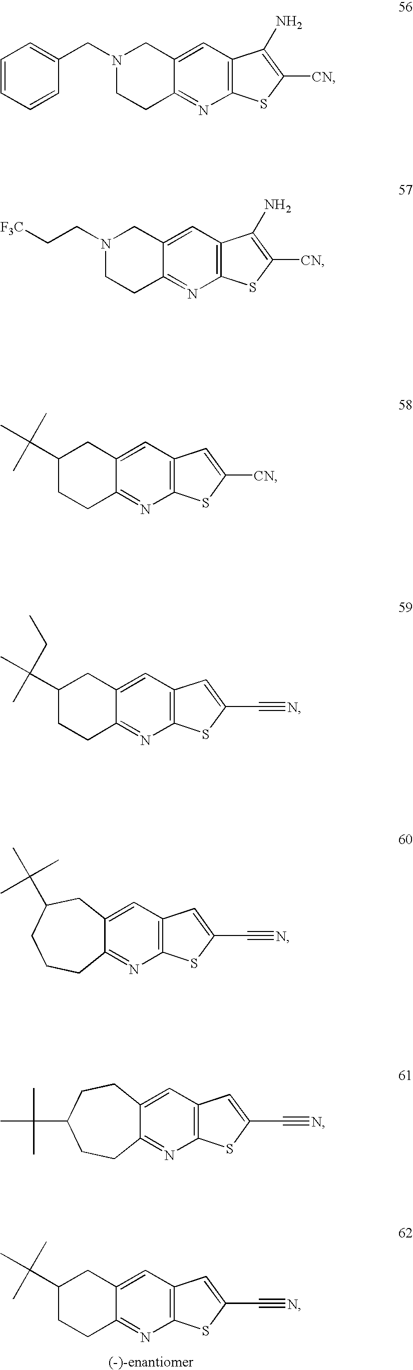 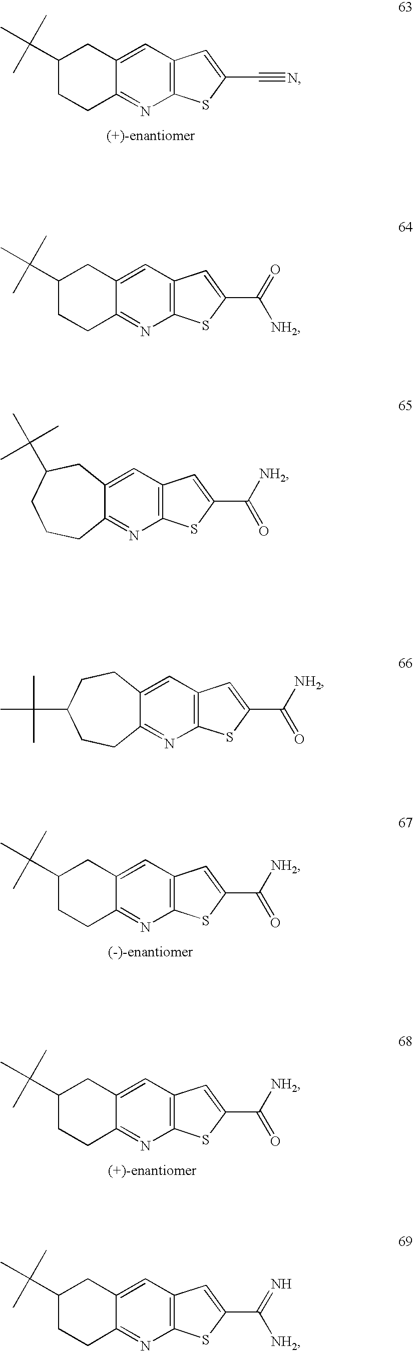 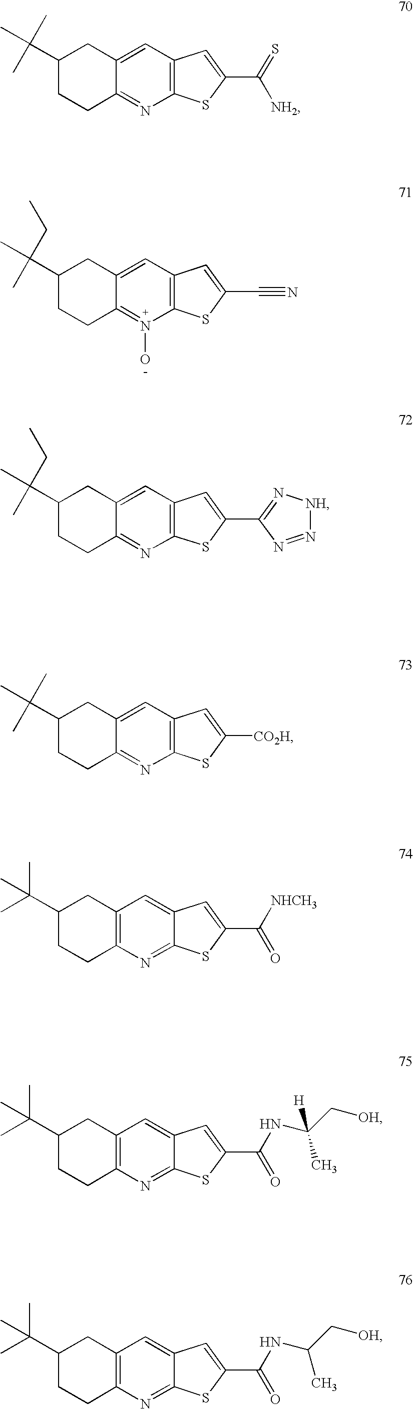 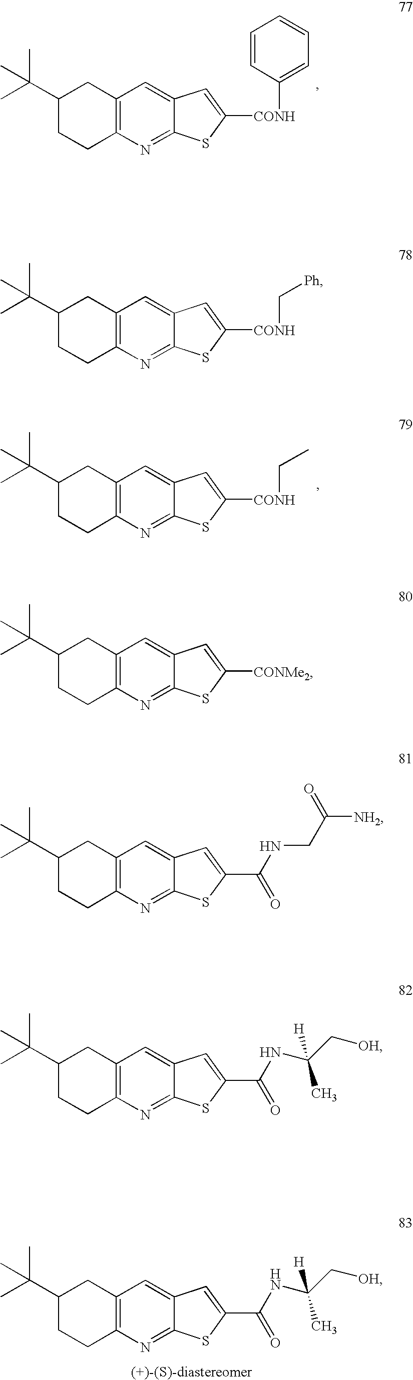 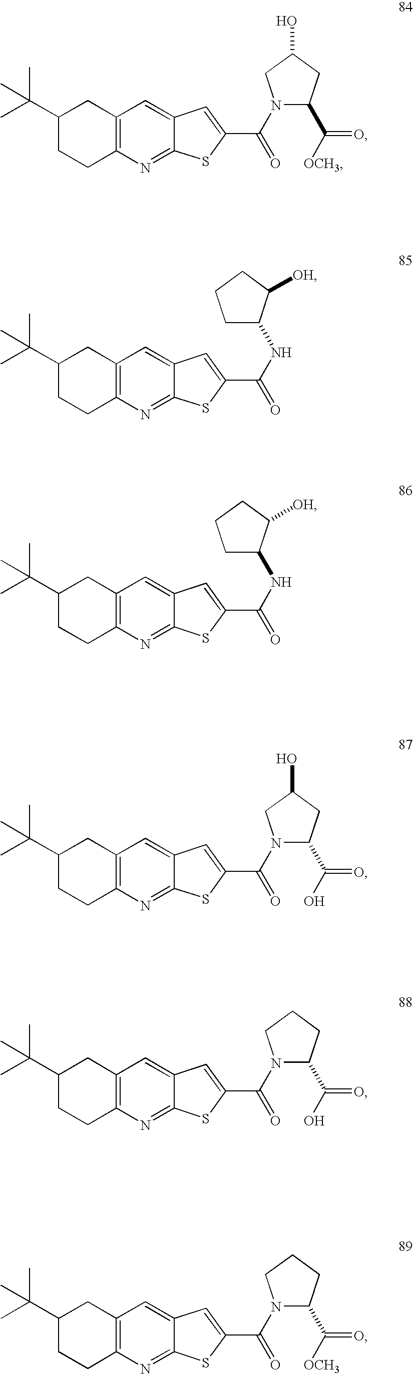 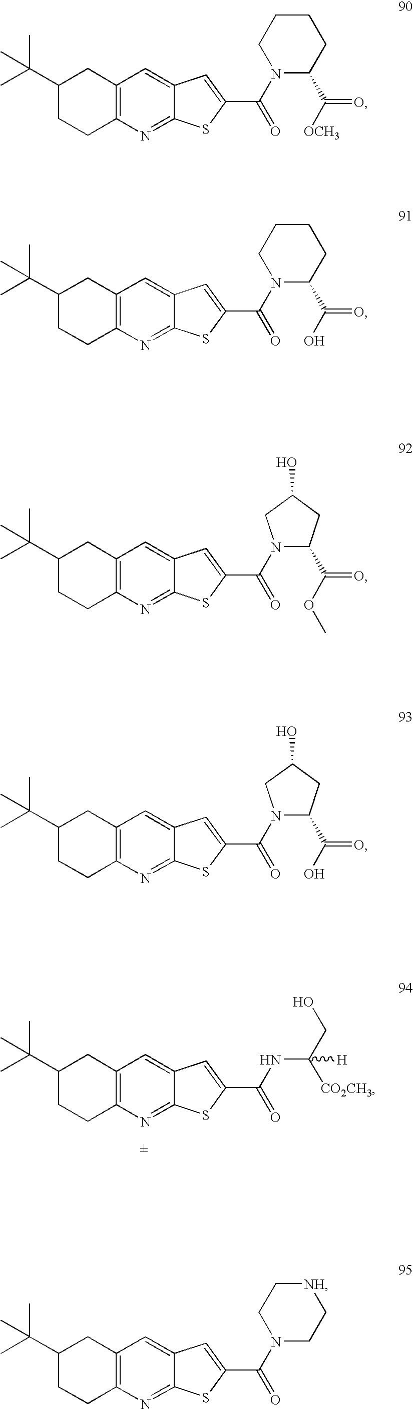 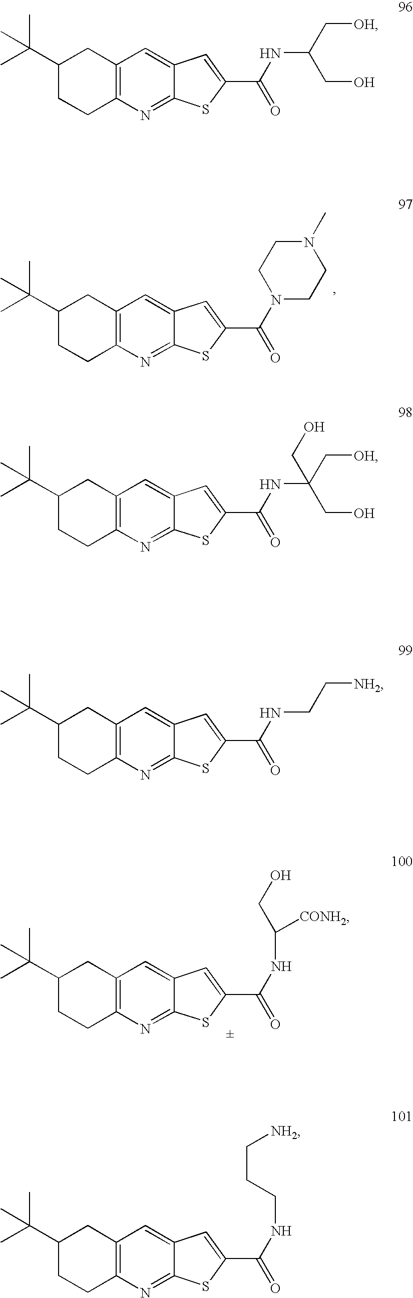 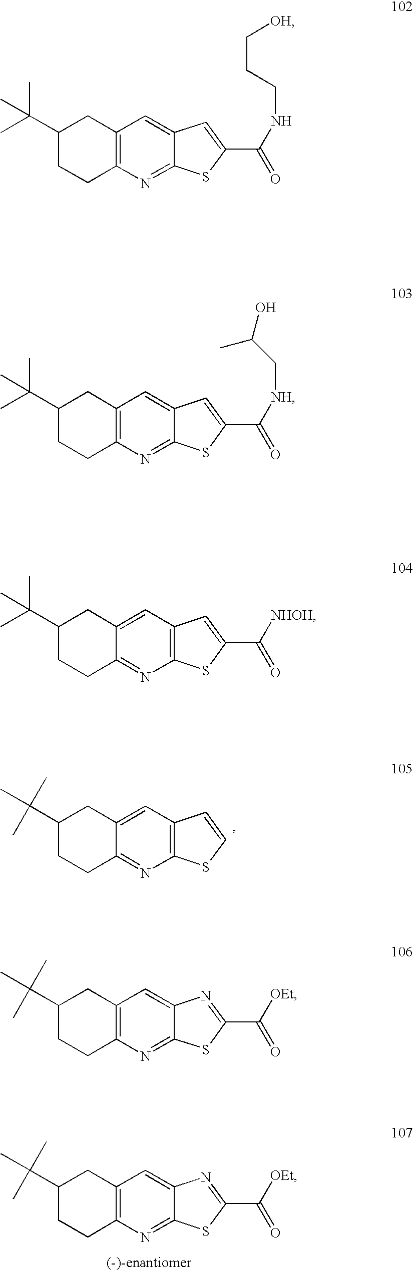 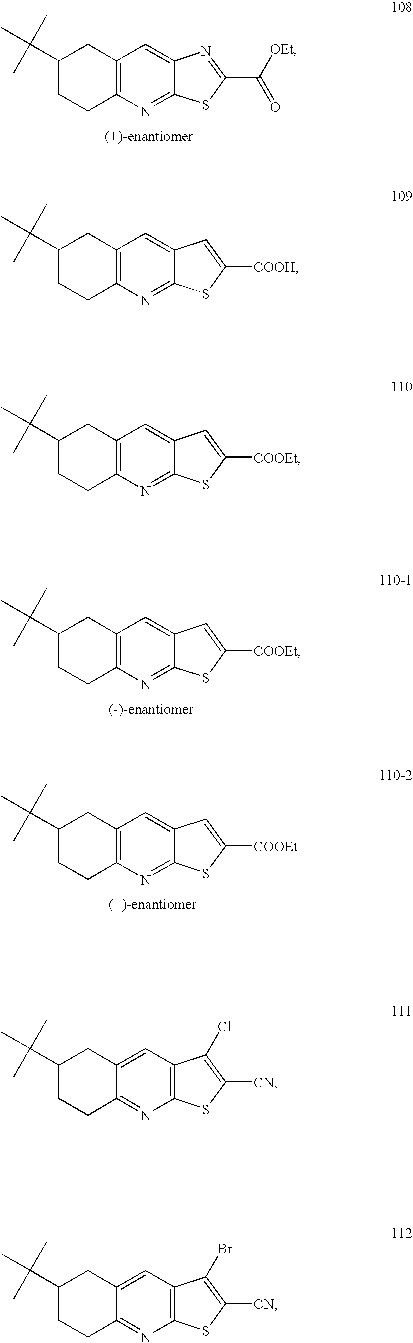 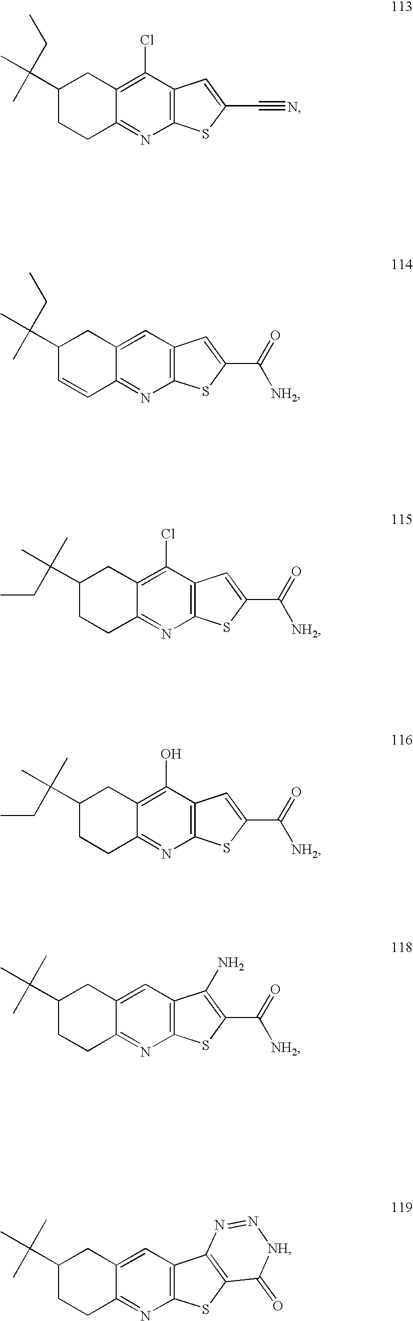 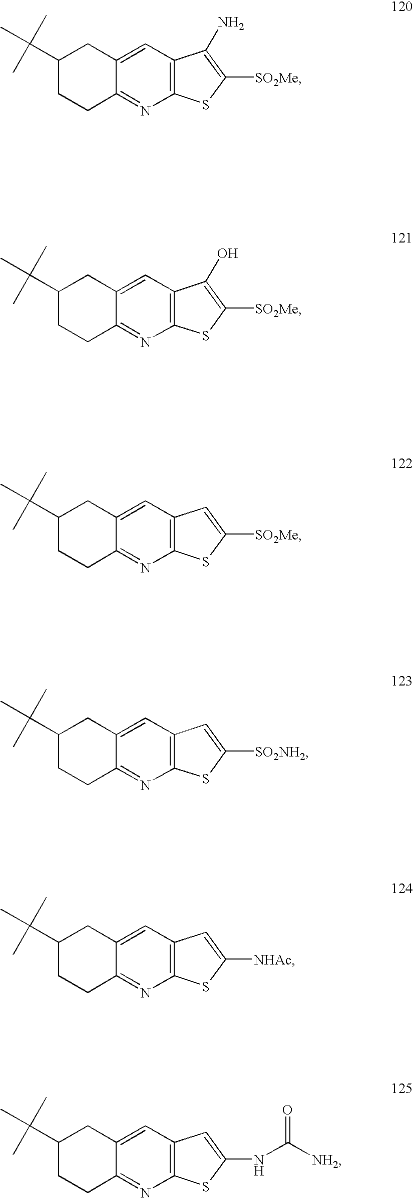 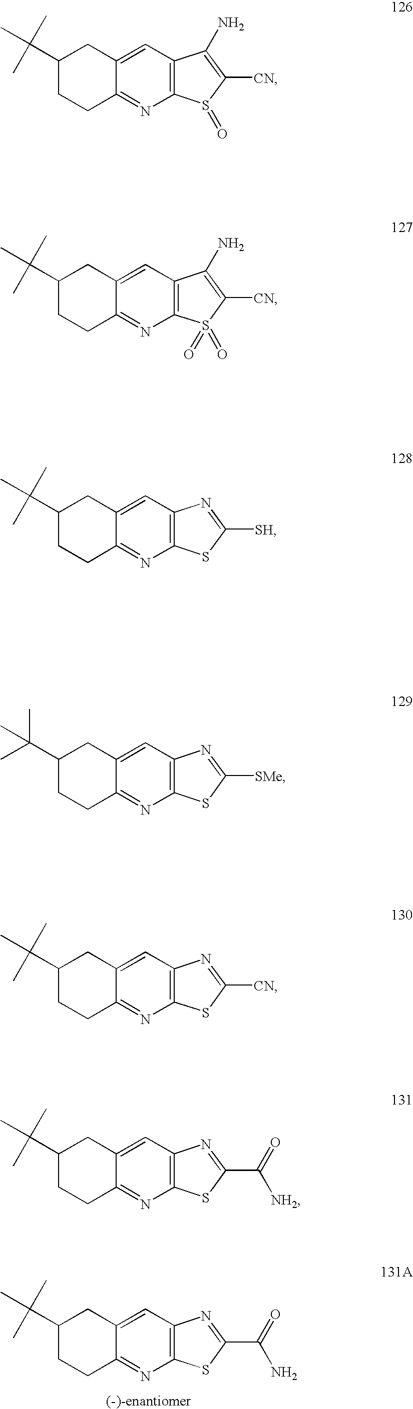 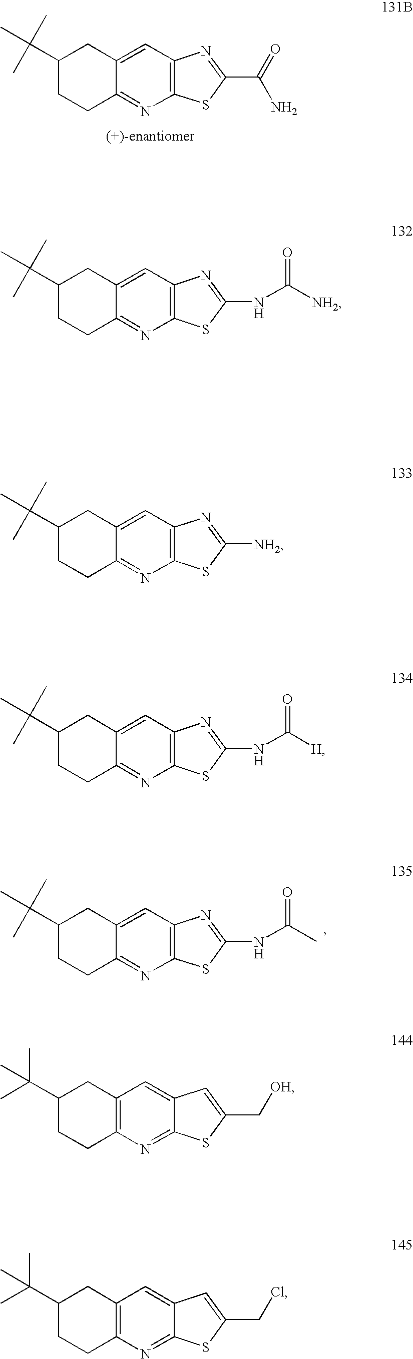 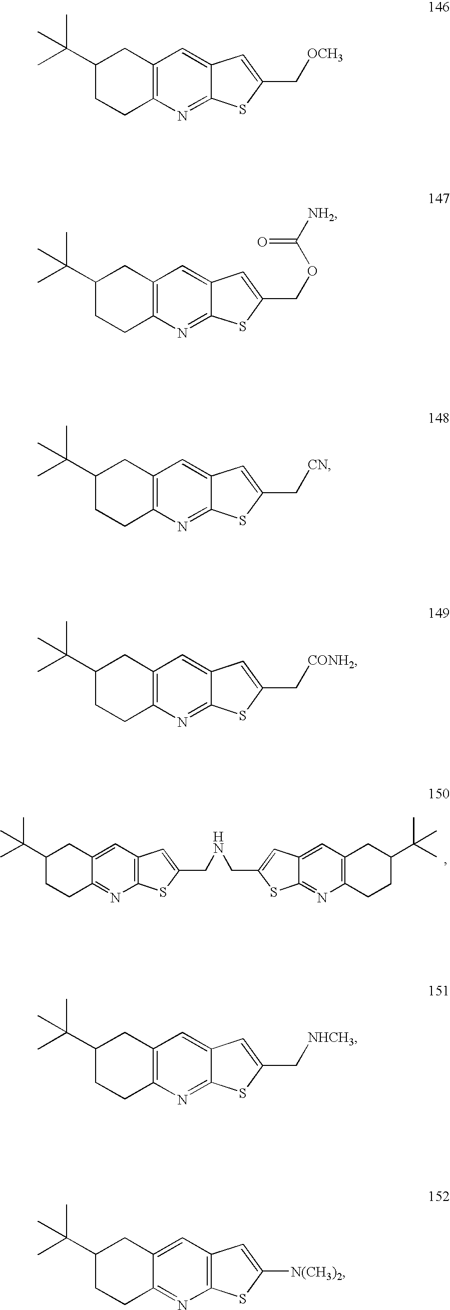 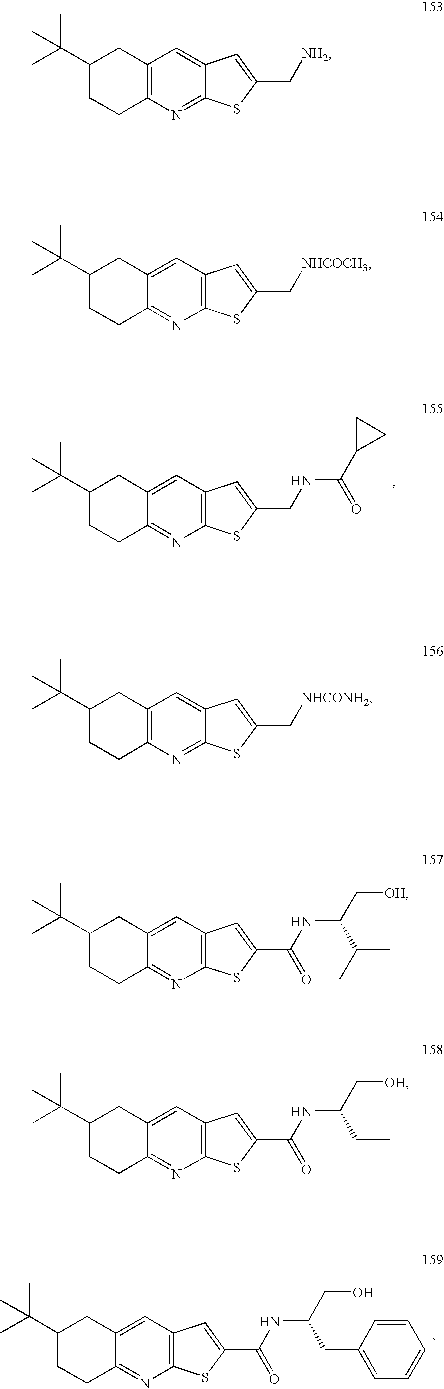 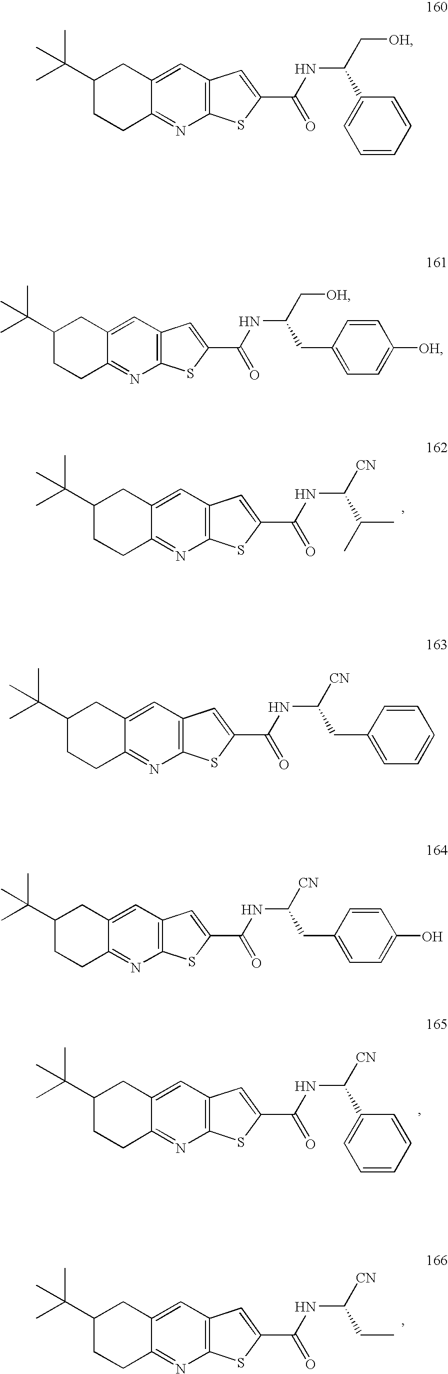 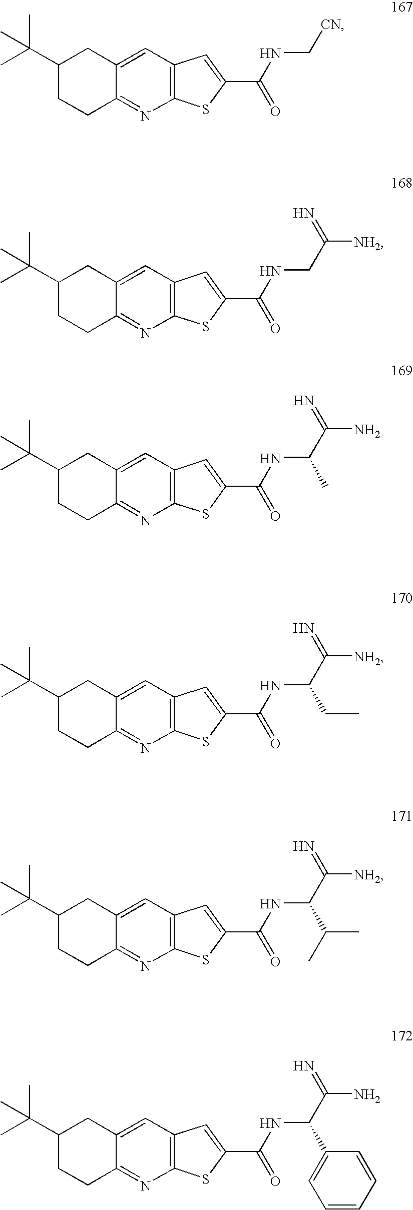 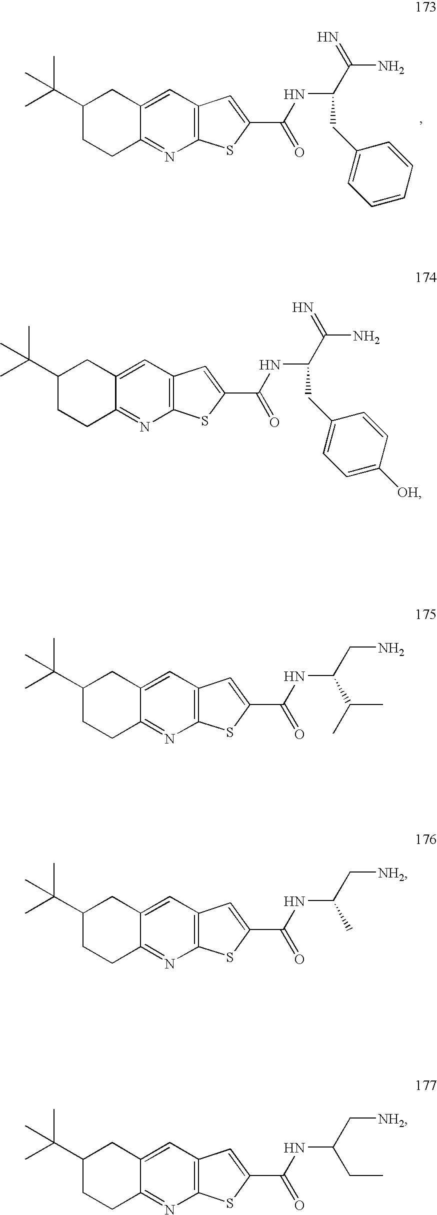 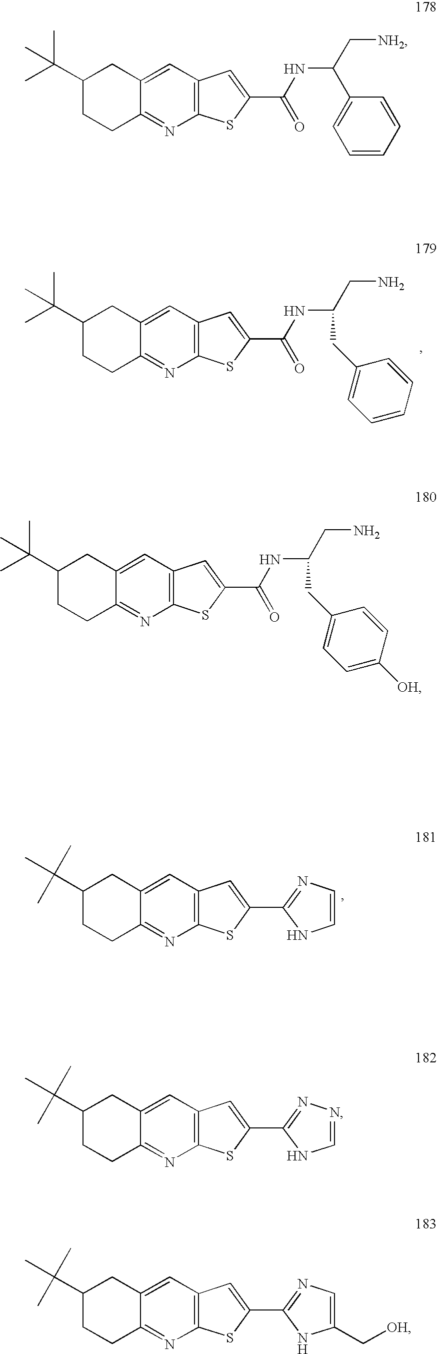 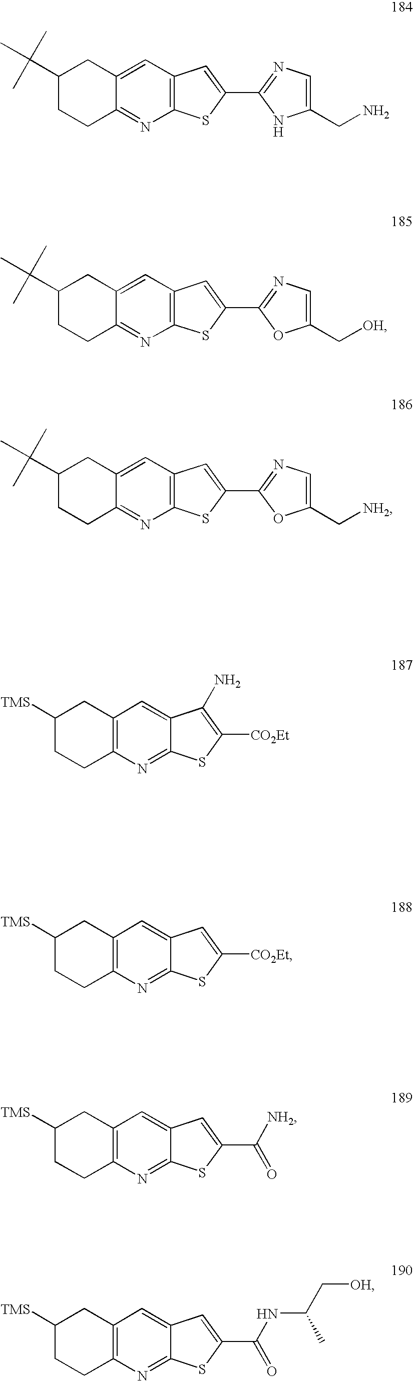 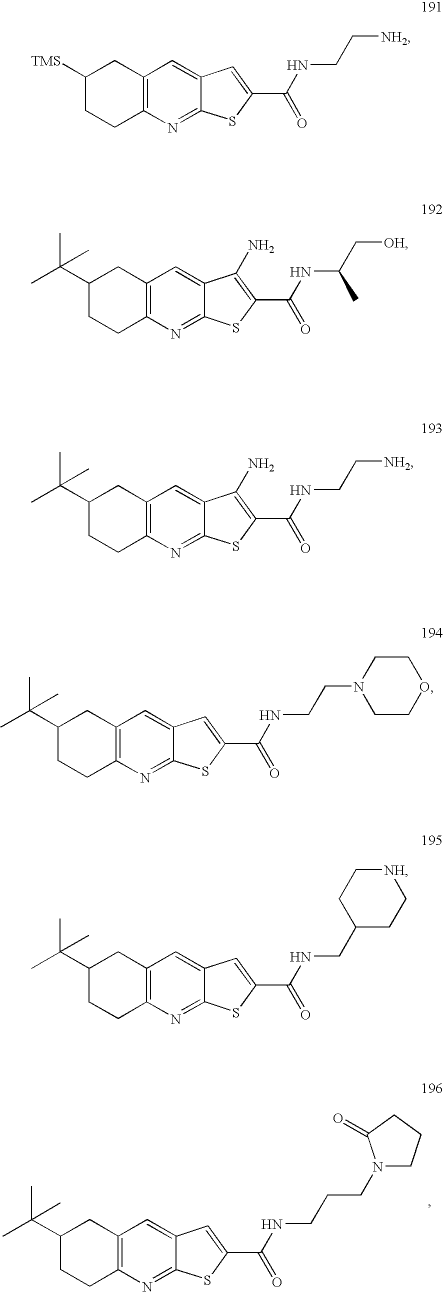 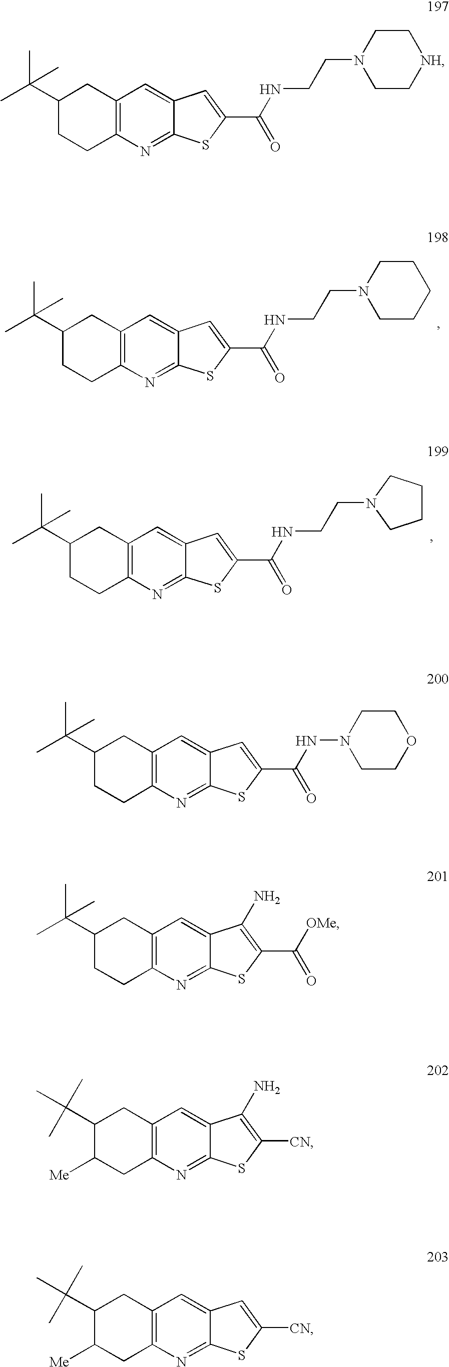 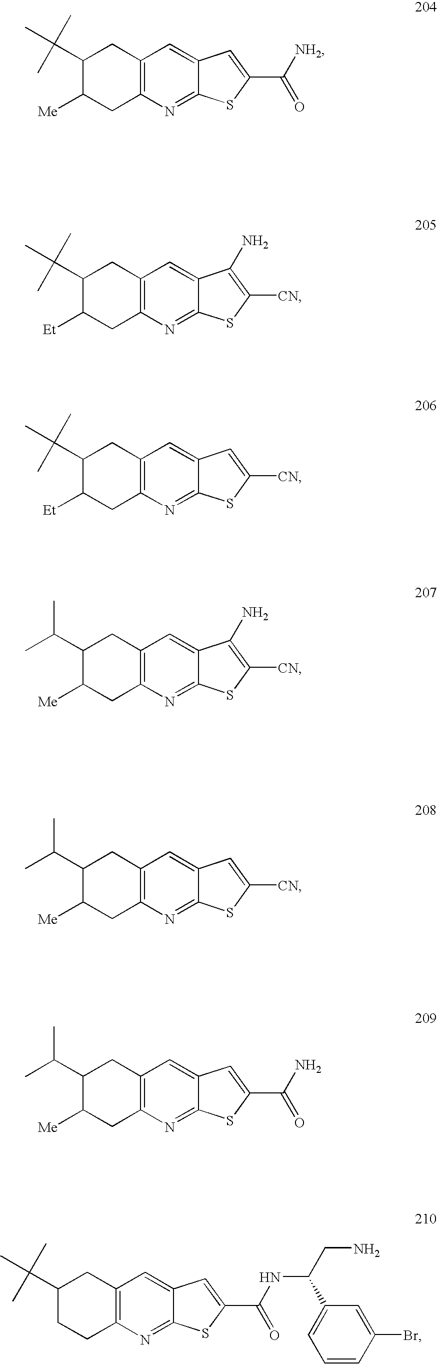 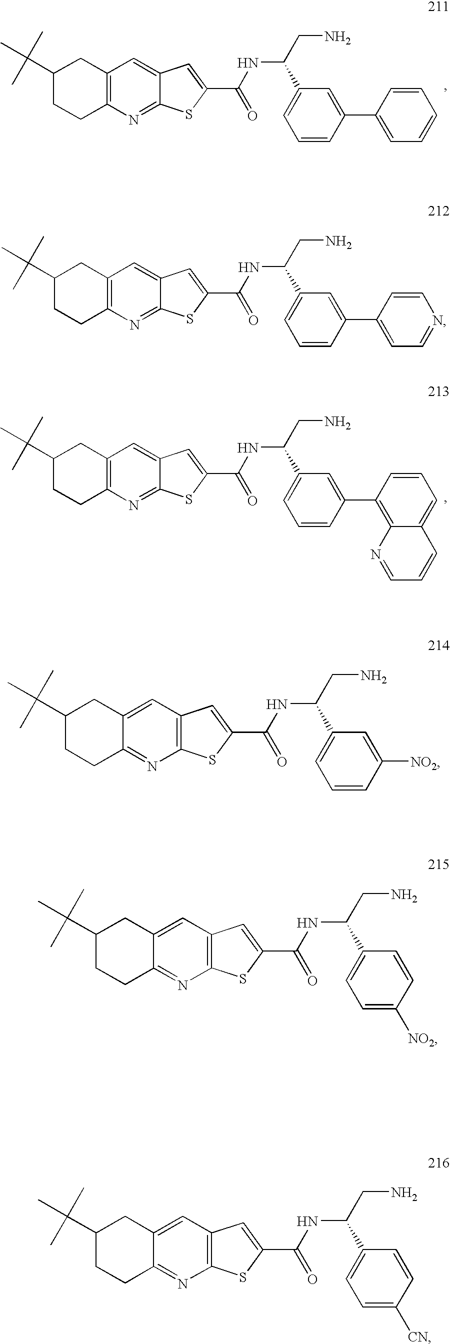 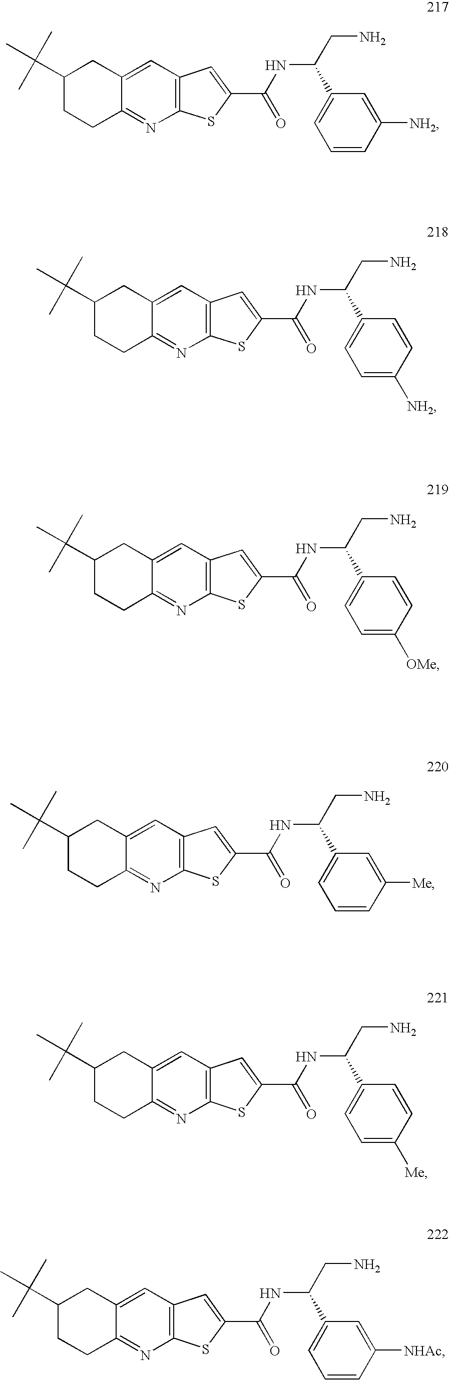 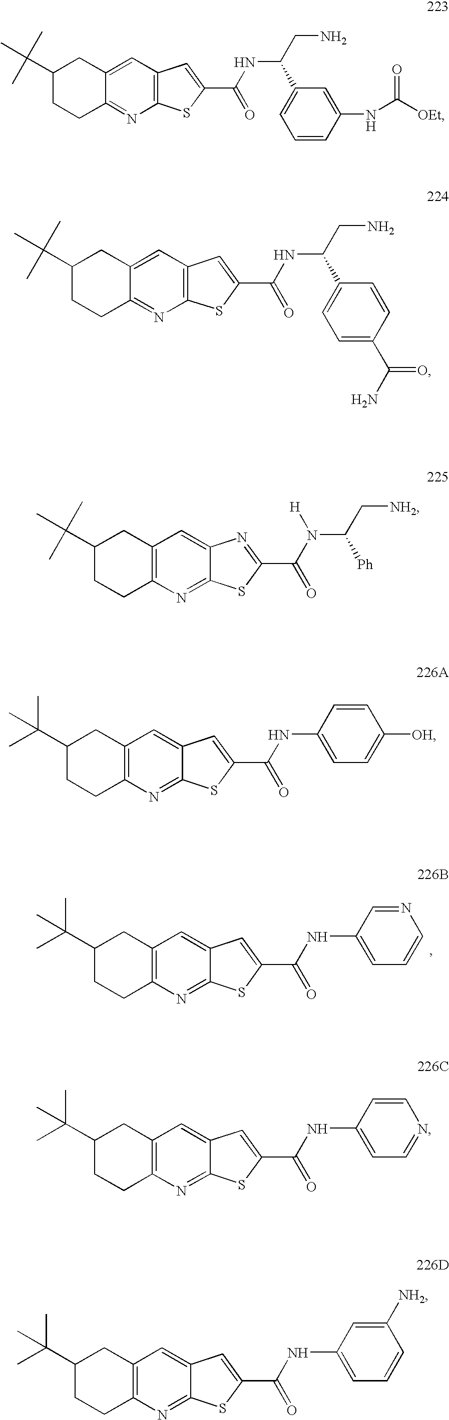 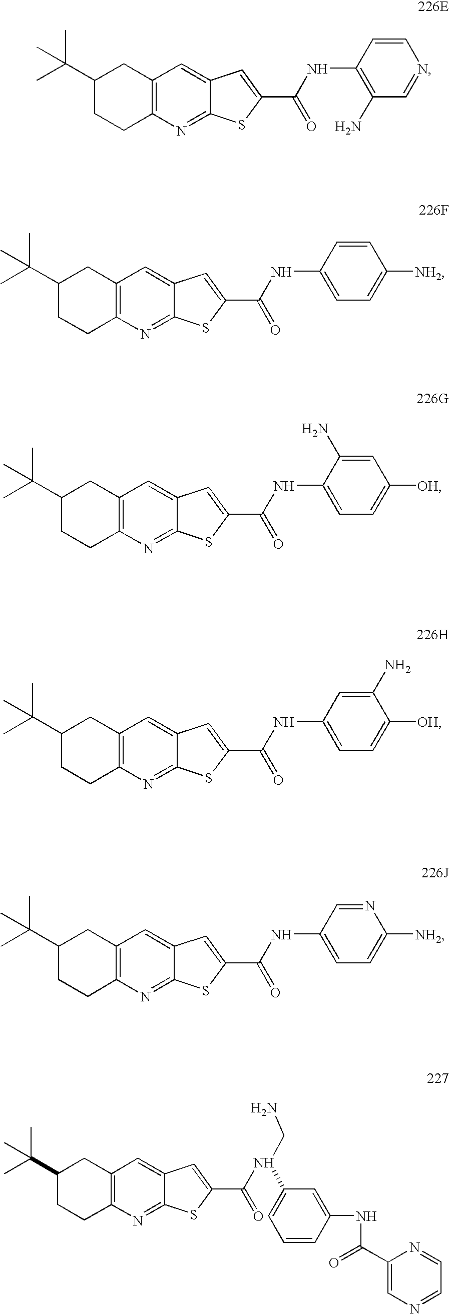 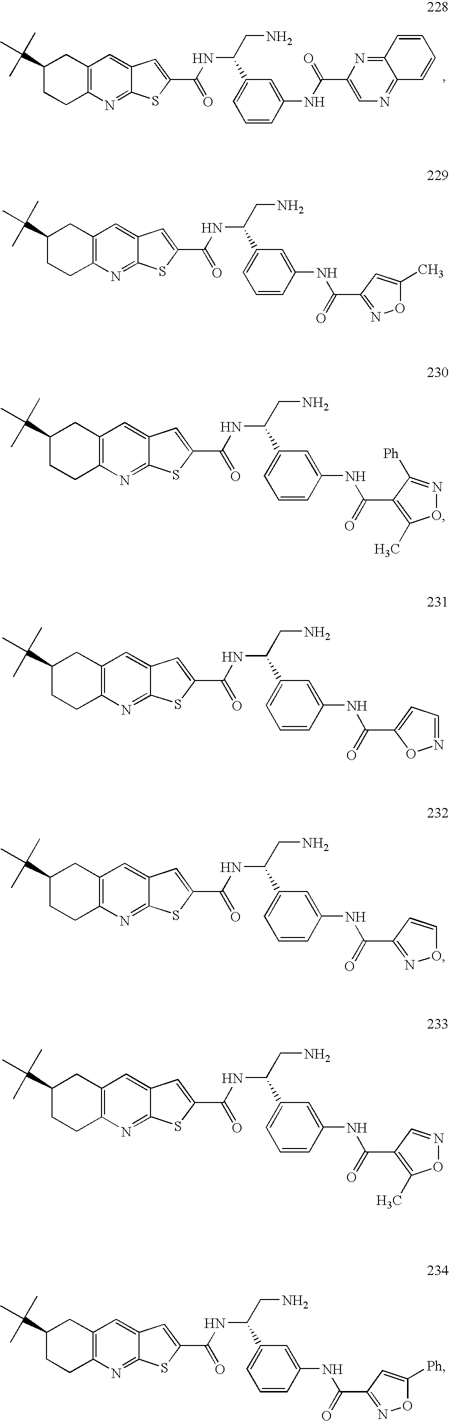 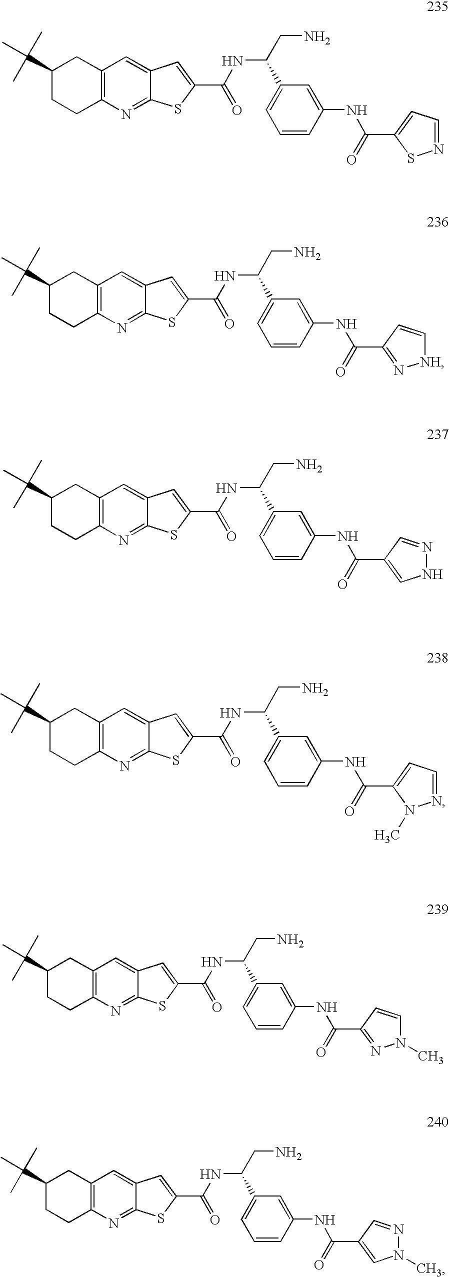 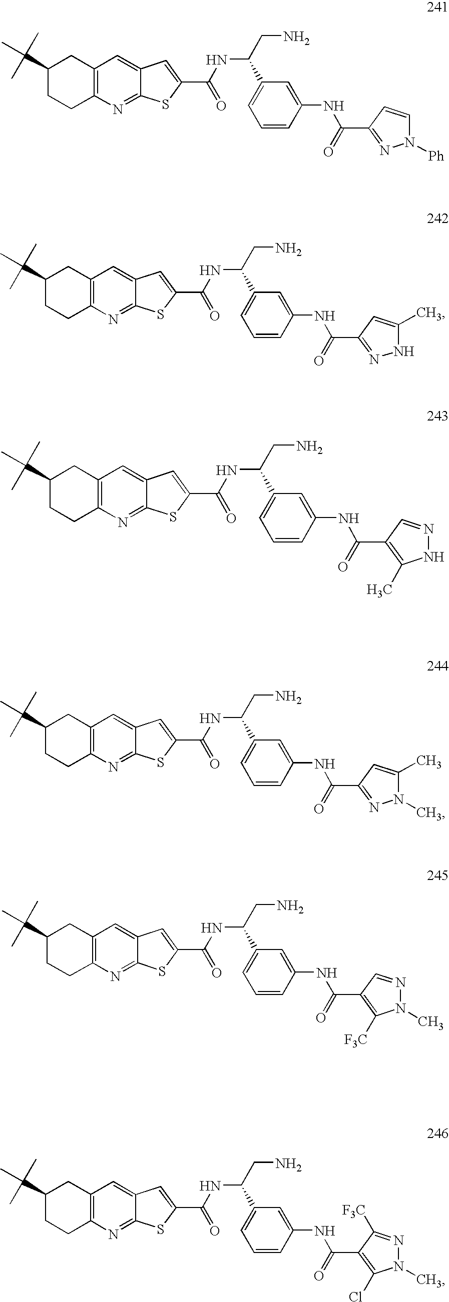 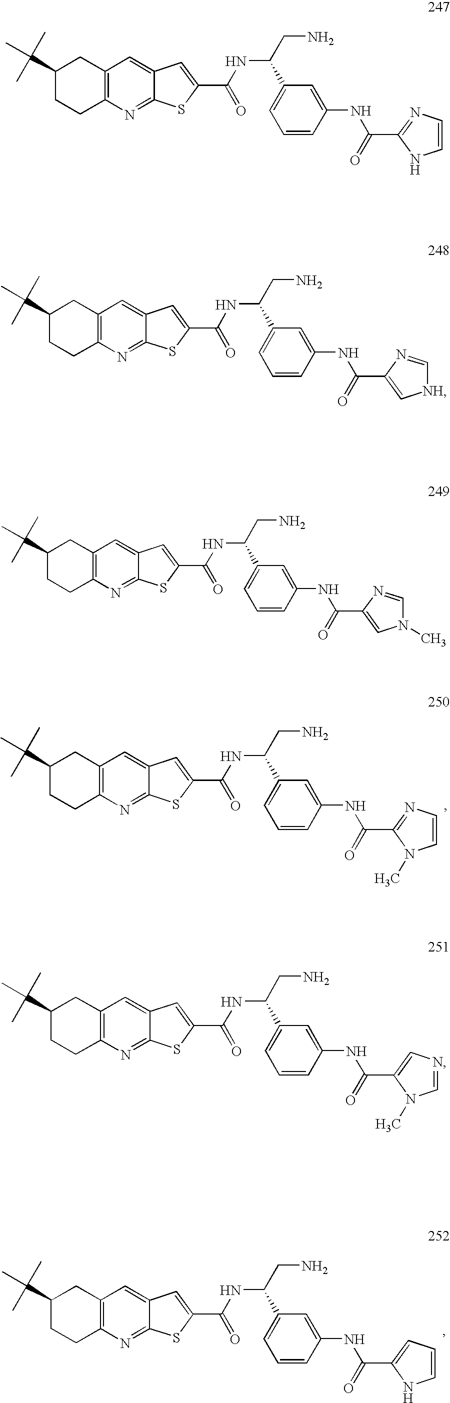 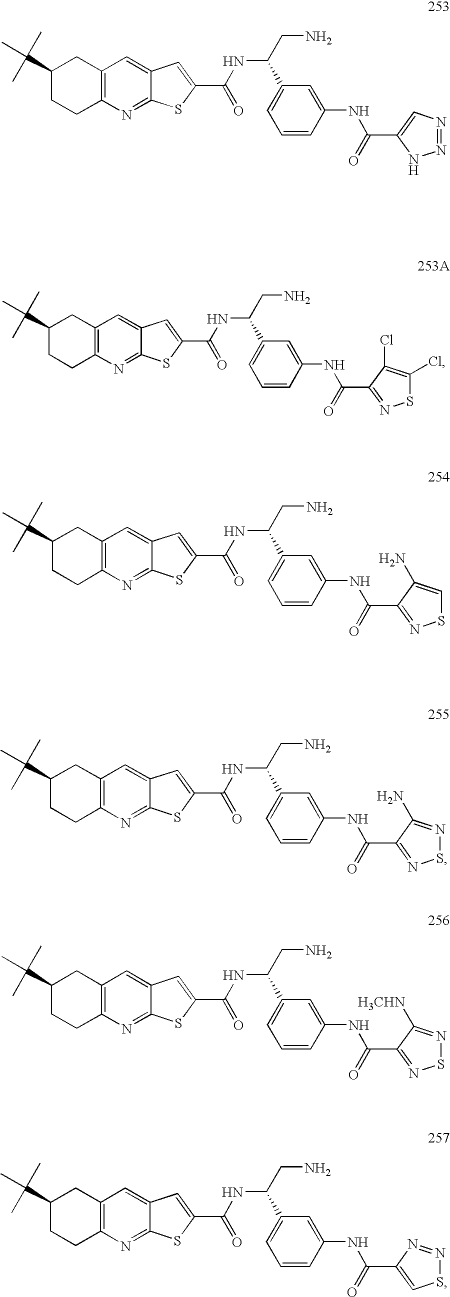 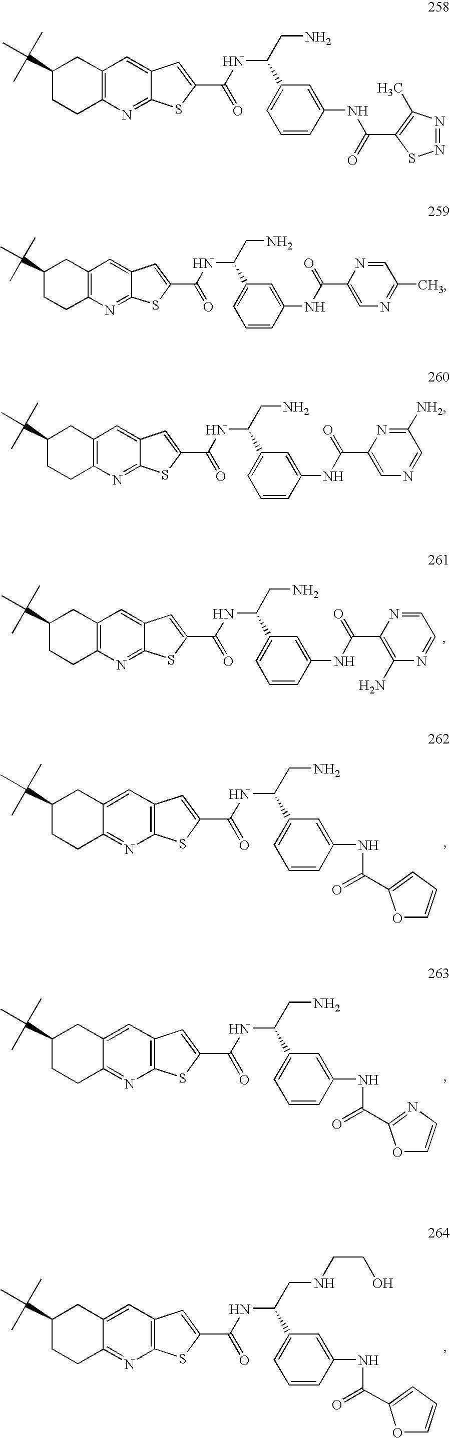 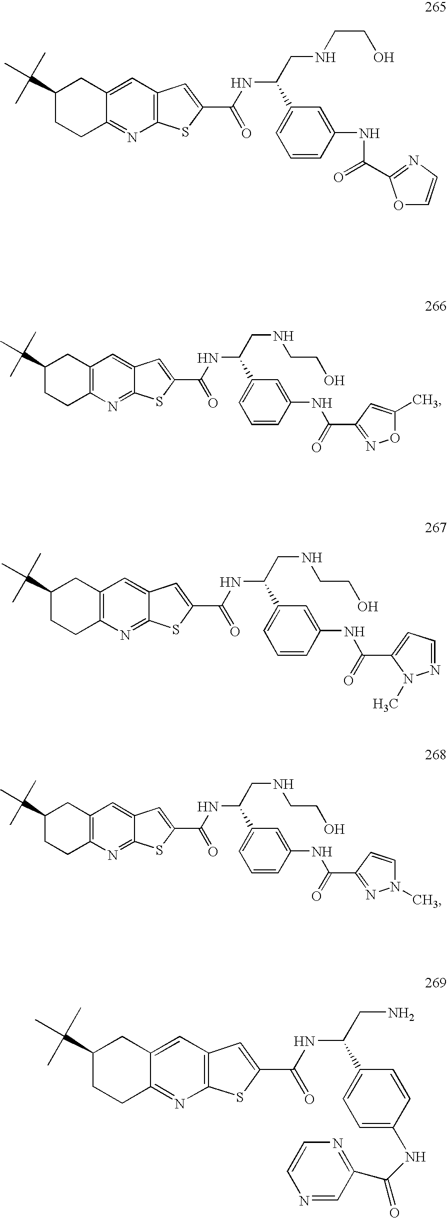 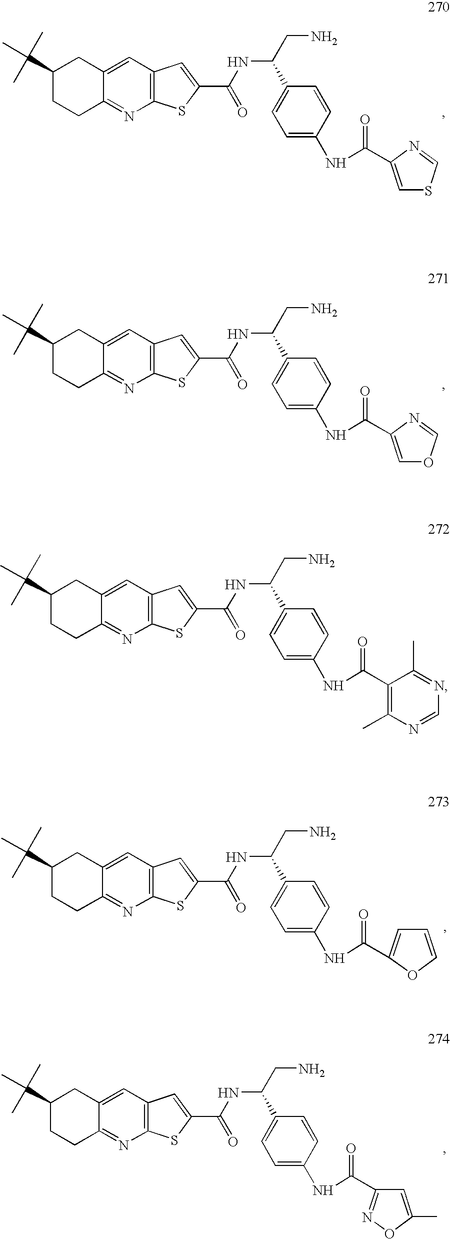 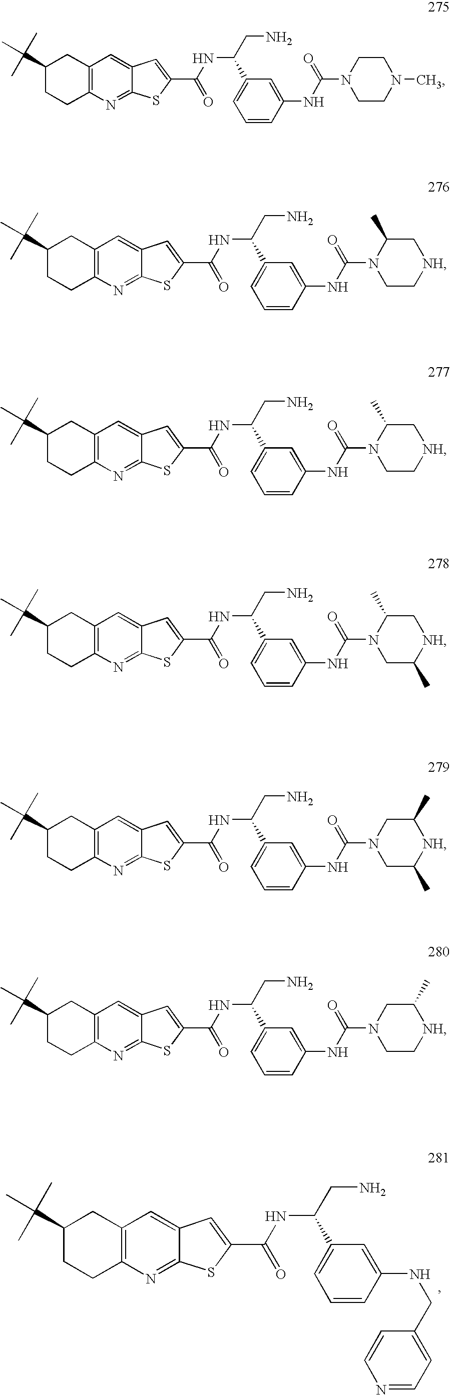 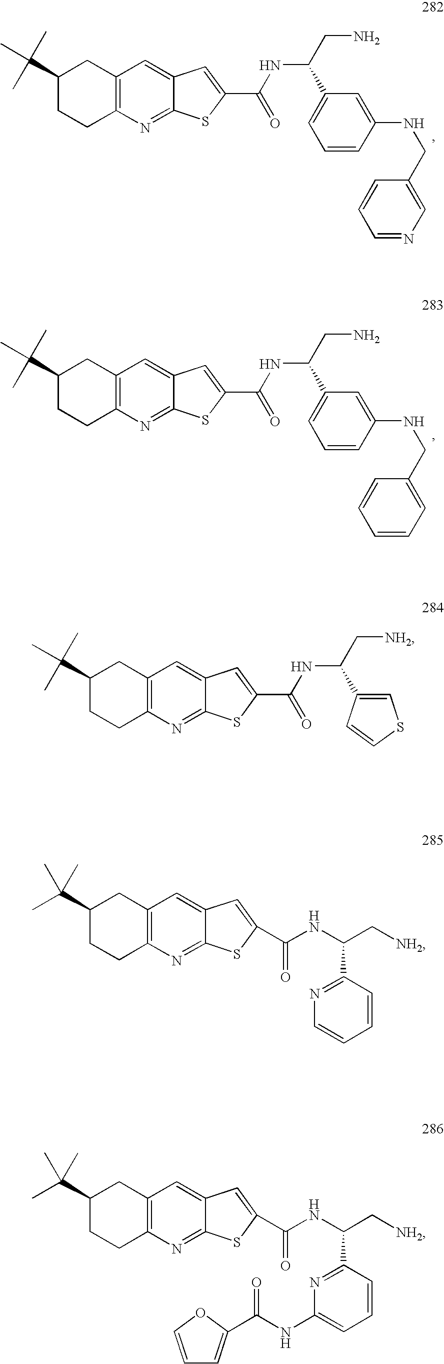 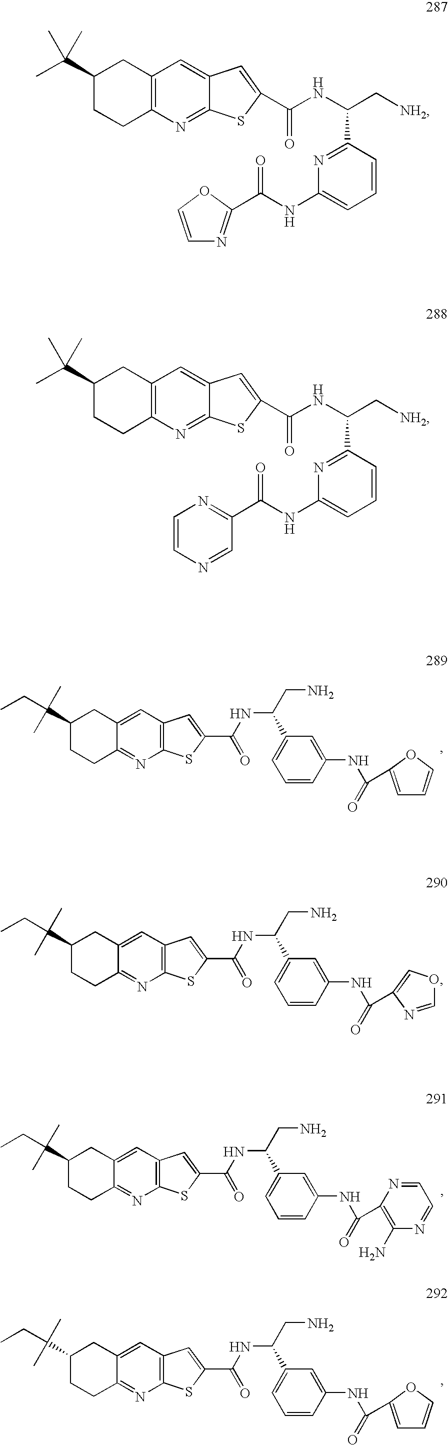 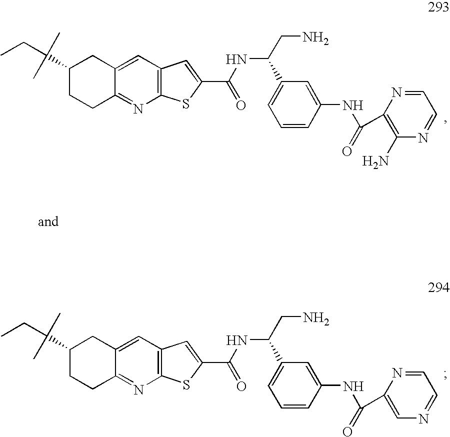
or pharmaceutically acceptable salt or solvate thereof.

52. The compound of claim 51, wherein the compound is selected from the group consisting of compound #s 6, 10, 12, 25, 26, 28, 30, 40, 43, 58, 59, 62, 63, 64, 65, 67, 68, 74, 75, 79, 83, 85, 86, 99,104, 123,131, 131A, 131B, 144, 157, 158, 160, 167, 168, 169, 170, 177, 178, 179, 180, 181, 183, 184, 189, 191, 210, 211, 212, 217, 218, 222, 223, 224, 225, 226A, 226B, 226C, 226D, 226E, 226F, 226J, and 227, and 228-284; or a pharmaceutically acceptable salt or solvate thereof.

53. The compound of claim 52, wherein the compound is selected from the group consisting of compound #s 40, 59, 63, 64, 65, 67, 68, 99, 144, 168, 177, 178, 189, 191, 210, 211, 212, 217, 218, 222, 223, 224, 225, 226A, 226B, 226C, 226D, 226E, 226F, 226J, and 227, and 228-284; or a pharmaceutically acceptable salt or solvate thereof.

54. An isolated or purified form of a compound of claim 1.

55. A pharmaceutical composition comprising a therapeutically effective amount of a compound claim 1 or a pharmaceutically acceptable salt or ester thereof, in combination with a pharmaceutically acceptable carrier.

56. The pharmaceutical composition of claim 55, further comprising one or more compounds selected from the group consisting of an anti-cancer agent, a PPAR-γ agonist, a PPAR-δ agonist, an inhibitor of inherent multidrug resistance, an anti-emetic agent, and an immunologic-enhancing drug.

57. The pharmaceutical composition of claim 56, wherein the anti-cancer agent is selected from the group consisting of an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, an inhibitor of cell proliferation and survival signaling, an agent that interferes with a cell cycle checkpoint, and an apoptosis inducing agent.

58. The pharmaceutical composition of claim 57, further comprising one or more anti-cancer agents selected from the group consisting of cytostatic agent, cytotoxic agent, taxane, topoisomerase II inhibitor, topoisomerase I inhibitor, tubulin interacting agent, hormonal agent, thymidilate synthase inhibitor, anti-metabolite, alkylating agent, farnesyl protein transferase inhibitor, signal transduction inhibitor, EGFR kinase inhibitor, antibody to EGFR, C-abl kinase inhibitor, hormonal therapy combination, and aromatase combination.

59. The pharmaceutical composition of claim 58, further comprising one or more agents selected from the group consisting of Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Torernifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin, cyclophosphamide, gemcitabine, interferons, pegylated interferons, Erbitux and mixtures thereof.

60. A method of inhibiting KSP activity in a subject in need thereof comprising administering to said subject an effective amount of at least one compound of claim 1 or a pharmaceutically acceptable salt, solvate or ester thereof.

61. A method of treating a cellular proliferative disease in a subject comprising administering to said subject in need of such treatment an effective amount of at least one compound of claim 1 or a pharmaceutically acceptable salt, solvate or ester thereof.

62. The method of claim 61, wherein the cellular proliferative disease is cancer, hyperplasia, cardiac hypertrophy, autoimmune diseases, fungal disorders, arthritis, graft rejection, inflammatory bowel disease, immune disorders, inflammation, cellular proliferation induced after medical procedures.

63. The method of claim 62, wherein the cancer is selected from cancers of the brain, genitourinary tract, cardiac, gastrointestine, liver, bone, nervous system, and lung.

64. The method of claim 62, wherein the cancer is selected from lung adenocarcinama, small cell lung cancer, pancreatic cancer, and breast carcinoma.

65. The method of claim 61, further comprising radiation therapy.

66. The method of claim 61, further comprising administering to the subject at least one compound selected from the group consisting of an anti-cancer agent, a PPAR-γ agonist, a PPAR-δ agonist, an inhibitor of inherent multidrug resistance, an anti-emetic agent, and an immunologic-enhancing drug.

67. The method of claim 66, wherein the disease is cancer.

68. The method of claim 67, further comprising radiation therapy.

69. The method of claim 66, wherein the anti-cancer agent is selected from the group consisting of an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, an inhibitor of cell proliferation and survival signaling, an agent that interferes with a cell cycle checkpoint, and an apoptosis inducing agent.

70. The method of claim 66, further comprising one or more anti-cancer agent selected from the group consisting of cytostatic agent, cytotoxic agent, taxane, topoisomerase II inhibitor, topoisomerase I inhibitor, tubulin interacting agent, hormonal agent, thymidilate synthase inhibitor, anti-metabolite, alkylating agent, farnesyl protein transferase inhibitor, signal transduction inhibitor, EGFR kinase inhibitor, antibody to EGFR, C-abl kinase inhibitor, hormonal therapy combination, and aromatase combination.

71. The method of any one of claim 66, further comprising one or more agents selected from the group consisting of Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin, Pentostatine, Vinblastine, Vincristine, Vindesine, Bleomycin, Dactinomycin, Daunorubicin, Doxomubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin, cyclophosphamide, gemcitabine, interferons, pegylated interferons, Erbitux and mixtures thereof.
